(19) United States
(12) Patent Application Publication
(10) Pub. No.: US 2026/0098038 A1
(43) Pub. Date: Apr. 9, 2026

(54) NOVEL DERIVATIVES HAVING 2,3-DIHYDRO-1H-INDENE OR 2,3-DIHYDROBENZOFURAN MOIETY OR PHARMACEUTICALLY ACCEPTABLE SALT THEREOF AND PHARMACEUTICAL COMPOSITIONS COMPRISING THE SAME

(71) Applicants: YUHAN CORPORATION, Seoul (KR); GREEN CROSS CORPORATION, Yongin-si (KR)

(72) Inventors: Dong-Hoon KIM, Suwon-si (KR); Jae-Eun JOO, Yongin-si (KR); Eui-Chul LEE, Yongin-si (KR); Tae-Dong HAN, Yongin-si (KR); Seung-Yub SHIN, Osan-si (KR); Sool-Ki KWON, Yongin-si (KR); Ji-Hye KIM, Yongin-si (KR)

(73) Assignees: YUHAN CORPORATION, Seoul (KR); GREEN CROSS CORPORATION, Yongin-si (KR)

(21) Appl. No.: 19/418,159

(22) Filed: Dec. 12, 2025

Related U.S. Application Data
(62) Division of application No. 17/755,843, now Pat. No. 12,522,602.

(30) Foreign Application Priority Data
Nov. 15, 2019 (KR) 10-2019-0146679

(51) Int. Cl. C07D 471/08

(52) U.S. Cl. CPC C07D 471/08

(57) ABSTRACT

Provided are a compound having a 2,3-dihydro-1H-indene or 2,3-dihydrobenzofuran moiety or pharmaceutically acceptable salt thereof, a process for the preparation thereof, a pharmaceutical composition comprising the same and a use thereof, where the. The compound having a 2,3-dihydro-1H-indene or 2,3-dihydrobenzofuran moiety or pharmaceutically acceptable salt thereof has an inhibitory activity against glucosylceramide synthase (GCS), and therefore can be usefully applied for preventing or treating various diseases associated with GCS, such as Gaucher disease, Fabry disease, Tay-Sachs disease, Parkinson's disease, etc.

### TECHNICAL FIELD

The present invention relates to a novel compound having an inhibitory activity against glucosylceramide synthase (GCS), i.e., derivatives having a 2,3-dihydro-1H-indene or 2,3-dihydrobenzofuran moiety or pharmaceutically acceptable salt thereof, a process for the preparation thereof, a pharmaceutical composition comprising the same and a use thereof.

### BACKGROUND ART

Lysosomal storage disorders (LSDs) are the metabolic disorders that result from genetic lack or deficiency of certain enzymes in lysosomes. LSDs exhibit various pathological symptoms throughout the body, as the non-metabolized or non-degraded substrates are accumulated. Currently, about 50 types of LSDs are known and are largely classified into the diseases such as mucopolysaccaridose, oligosaccharidosis, and sphingolipidoses, depending on the substances accumulated.

Among the diseases, sphingolipidoses, which are a class of glycolipid storage disorders relating to sphingolipid metabolism, show pathologies due to the accumulation of various membrane glycosphingolipids (GSLs), such as glucosylceramide, trihexocylceramide, etc. For example, when the enzymes related to sphingolipid metabolism, such as beta-glucosidase, alpha-galactosidase, etc., do not have normal activity, the substrates thereof (e.g., glucosylceramide, trihexocylceramide, etc.) are accumulated, thereby exhibiting various pathologies, such as Gaucher disease, Fabry disease, etc.

There are known two types of therapies for the LSDs. The first method is to replace or supplement the insufficient or deficient metabolic enzymes. Although such an enzyme replacement therapy (ERT) is safe and effective, periodic intravenous administrations of the related enzyme(s) are required; the dose thereof should be adjusted according to the enzyme reaction(s); and the costs thereof are relatively high. Especially, since it is difficult to distribute the enzyme(s) toward the nervous system, the ERT does not show satisfactory efficacy in the treatment of symptoms related to the nervous system. In addition, there is also the problem that autoantibodies against the administered enzyme(s) are frequently generated.

The second method is a substrate reduction therapy (SRT) for inhibiting the syntheses of accumulated substrates. Glucosylceramide synthase (GCS) (also referred to as “UDP-glucose: ceramide glycosyltransferase”, “UDP-glucose: N-acylsphingosine D-glucosyltransferase”, or “EC 2.4.1.80”), which is an enzyme involved in sphingolipid metabolism, is involved in the reaction of ceramide with glucose to produce glucosylceramide. The resulting glucosylceramide is converted into various GSLs. Glucosylceramide synthase (GCS) inhibitors inhibit the activity of GCS, thereby reducing the production of glucosylceramide in the body and preventing abnormal accumulation of glycolipids, such as trihexocylceramide, GM1, and GM2, in cells or organs.

As such, GCS inhibitors inhibit the activity of GCS to prevent the accumulation of glycolipids and thus may be usefully applied to the treatment of lysosomal storage disorders, especially glycolipid storage disorders, such as GM1 gangliosidosis, Tay-Sachs disease, Sandhoff disease, Gaucher disease, Fabry disease, Niemann-Pick disease (types A and B), metachromatic leukodystrophy, Krabbe disease, etc. GCS inhibitors may be also used in the treatment of the secondary diseases associated with glycolipid storage, such as Niemann-Pick disease (type C), mucopolysaccharidosis, and mucolipidosis type IV (see, Chen C S, et al., Abnormal transport along the lysosomal pathway in mucolipidosis, type IV disease Proc Natl Acad Sci USA. 1998 May 26; 95(11):6373-8; and Goodman L A, et al., Ectopic dendrites occur only on cortical pyramidal cells containing elevated GM2 ganglioside in alpha-mannosidosis, Proc Natl Acad Sci USA. 1991 Dec. 15; 88(24):11330-4). In addition, it has been reported to be useful in the treatment of diseases associated with the accumulation of glycolipids, such as renal hypertrophy (e.g., diabetic kidney disease); hyperglycemia or hyperinsulinemia; cancers with abnormal glycolipid synthesis; infectious diseases caused by the organisms using cell-surface glycolipids as a receptor; infectious diseases where the synthesis of glucosylceramide is essential or important; diseases in which excessive glycolipid synthesis occurs (e.g., atherosclerosis, polycystic kidney disease, and renal hypertrophy); neurological disorders and/or damages associated with the replenishment and activity of macrophages (e.g., Alzheimer's disease, epilepsy, stroke, spinal cord diseases, Parkinson's disease, etc.); inflammatory diseases or disorders (e.g., rheumatoid arthritis, Crohn's disease, asthma, sepsis); and diabetes and obesity (see, WO 2006/053043). And, overexpression of GCS interferes with ceramide-induced apoptosis (see, Liu Y Y, et al., Uncoupling ceramide glycosylation by transfection of glucosylceramide synthase antisense reverses adriamycin resistance, J Biol Chem. 2000 Mar. 10; 275(10):7138-43). Therefore, GCS inhibitors may be useful fortreating proliferative diseases, such as cancer, by inducing apoptosis in diseased cells.

Various studies have been conducted to develop GCS inhibitors. For example, various compounds having an inhibitory activity against GCS have been disclosed in WO 2005/068426, WO 2006/053043, WO 2008/150486, WO 2009/117150, WO 2010/014554, WO 2014/043068, etc.

### DISCLOSURE

### Technical Problem

The present inventors have found that a derivative having a 5,6-fused bicyclic moiety, i.e., a 2,3-dihydro-1H-indene or 2,3-dihydrobenzofuran moiety, or pharmaceutically acceptable salt thereof has an excellent inhibitory activity against glucosylceramide synthase (GCS). Therefore, the derivative having a 2,3-dihydro-1H-indene or 2,3-dihydrobenzofuran moiety or pharmaceutically acceptable salt thereof can be usefully applied for preventing or treating various diseases associated with GCS, such as Gaucher disease, Fabry disease, Tay-Sachs disease, Parkinson's disease, etc.

Therefore, the present invention provides the above derivative having a 2,3-dihydro-1H-indene or 2,3-dihydrobenzofuran moiety or pharmaceutically acceptable salt thereof, a process for the preparation thereof, a pharmaceutical composition comprising the same, and a use thereof.

### Technical Solution

According to an aspect of the present invention, there is provided a derivative having a 2,3-dihydro-1H-indene or 2,3-dihydrobenzofuran moiety or pharmaceutically acceptable salt thereof.

According to another aspect of the present invention, there is provided a process for preparing said derivative having a 2,3-dihydro-1H-indene or 2,3-dihydrobenzofuran moiety or pharmaceutically acceptable salt thereof.

According to still another aspect of the present invention, there is provided a pharmaceutical composition comprising said derivative having a 2,3-dihydro-1H-indene or 2,3-dihydrobenzofuran moiety or pharmaceutically acceptable salt thereof as an active ingredient.

According to still another aspect of the present invention, there is provided a therapeutic method comprising administering said derivative having a 2,3-dihydro-1H-indene or 2,3-dihydrobenzofuran moiety or pharmaceutically acceptable salt thereof.

According to still another aspect of the present invention, there is provided a use of said derivative having a 2,3-dihydro-1H-indene or 2,3-dihydrobenzofuran moiety or pharmaceutically acceptable salt thereof for the manufacture of a medicament for inhibiting glucosylceramide synthase.

### Advantageous Effects

It has been found by the present invention that the derivative having a 5,6-fused bicyclic moiety, i.e., a 2,3-dihydro-1H-indene or 2,3-dihydrobenzofuran moiety, or pharmaceutically acceptable salt thereof has an excellent inhibitory activity against glucosylceramide synthase (GCS). Therefore, the compound or pharmaceutically acceptable salt thereof according to the present invention can be usefully applied for preventing or treating various diseases associated with GCS, such as Gaucher disease, Fabry disease, Tay-Sachs disease, Parkinson's disease, etc.

### BEST MODE FOR CARRYING OUT THE INVENTION

As used herein, the term “alkyl” refers to a straight or branched aliphatic hydrocarbon radical. For example, the C₁-C₆ alkyl means a straight or branched aliphatic hydrocarbon having 1 to 6 carbon atoms, such as methyl, ethyl, propyl, n-butyl, n-pentyl, n-hexyl, isopropyl, isobutyl, sec-butyl, tert-butyl, neopentyl, and isopentyl.

The term “hydroxy” refers to the ‘—OH’ group. The term “alkoxy” refers to a radical formed by substituting the hydrogen atom in the hydroxyl group with an alkyl. For example, the C₁-C₆ alkoxy includes methoxy, ethoxy, propoxy, n-butoxy, n-pentyloxy, isopropoxy, sec-butoxy, tert-butoxy, neopentyloxy, and isopentyloxy.

The term “halogen” refers to the fluoro, bromo, chloro, or iodo group.

The term “cycloalkyl” refers to a saturated aliphatic 3- to 10-membered ring, preferably 3- to 7-membered ring, unless otherwise defined. Typical cycloalkyl groups include cyclopropyl, cyclobutyl, cyclopentyl, cyclohexyl, and the like, but are not limited thereto.

The term “aryl” refers to an organic radical derived from an aromatic hydrocarbon, through removing one hydrogen atom therefrom, including mono or poly-fused ring systems such as 5- to 14-membered substituted or unsubstituted rings and a form in which a plurality of aryls are connected by a single bond. The “aryl” includes, for example, phenyl, naphthyl, biphenyl, terphenyl, anthryl, indenyl, fluorenyl, phenanthryl, triphenylenyl, pyrenyl, perylenyl, chrysenyl, naphthacenyl, fluoranthenyl, and the like, but are not limited thereto.

The term “heteroaryl” refers to a 5- to 12-membered aromatic radical having one to three heteroatoms selected from the group consisting of nitrogen (N) atom, oxygen (O) atom, and sulfur (S) atom, including a 5- or 6-membered monocyclic heteroaryl radical and a bicyclic heteroaryl radical formed by fusing the 5- or 6-membered monocyclic heteroaryl radical with a benzene or pyridine ring. And, the term “heterocycle” refers to a 3- to 12-membered mono- or poly-cyclic ring having one or more, preferably one to four, same or different heteroatoms selected from oxygen (O) atom, nitrogen (N) atom, and sulfur (S) atom, but not containing an aromatic ring. Non-limiting examples of heteroaryl or heterocyclic rings include oxetane, pyrrolidine, pyrrole, tetrahydrofuran, furan, tetrahydrothiophene, thiophene, imidazolidine, imidazole, pyrazolidine, pyrazole, pyrrolizine, oxazolidine, oxazole, isoxazolidine, isoxazole, thiazolidine, thiazole, isothiazolidine, isothiazole, dioxolane, dithiolane, oxadiazole, thiadiazole, dithiazole, tetrazole, oxatetrazole, thiatetrazole, piperidine, pyridine, pyrimidine, tetrahydropyran, pyran, thiane, thiopyran, piperazine, diazine, morpholine, oxazine, dioxane, indole, indoline, benzodioxole, benzothiophene, benzofuran, benzimidazole, bezoxazole, benzisoxazole, benzothiazole, benzothiadiazole, benzotriazole, quinoline, isoquinoline, purine, furopyridine, mono- or di-azabicycles (such as quinuclidine, diazabicycloheptane, monoazabicyclooctane, diazaspiroundecane, etc.), hexahydropyrrolopyrrole, pyrrolopyrrole, pyrrolopyridine, imidazopyridazine, dihydrobenzodioxine, dihydrobenzofuran, and the like, but are not limited thereto.

The term “amino” refers to the ‘—NH₂’ group. The term “alkylamino” refers to an amino group substituted with mono- or di-alkyl. For example, the C₁-C₆ alkylamino group includes an amino group substituted with mono- or di-C₁-C₆ alkyl group.

The term “alkylthio” refers to the ‘—SR’ group, in which R is an alkyl. The term “cyano” refers to the ‘—CN’.

The present invention provides a derivative having a 5,6-fused bicyclic moiety, i.e., a 2,3-dihydro-1H-indene or 2,3-dihydrobenzofuran moiety or pharmaceutically acceptable salt thereof, that is a compound of Formula 1 or pharmaceutically acceptable salt thereof:

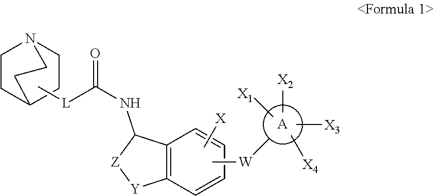

wherein,
L is —O—, —CO—, —CR₁R₂—, or —NR₃—,
Y and Z are, independently each other, —CR₁R₂—; —NR₃—; —O—; or —S—,
R₁ and R₂ are, independently each other, hydrogen; halogen; C₁-C₆ alkyl; C₁-C₆ alkyl having a nitrogen, oxygen, or sulfur atom; C₃-C₁₀ cycloalkyl; 3- to 12-membered heterocyclic; or C₁-C₆ alkoxy; or R₁ and R₂ form C₃-C₁₀ cycloalkyl together with the carbon atom to which they are attached,
R₃ is hydrogen; C₁-C₆ alkyl; C₁-C₆ alkyl having a nitrogen, oxygen, or sulfur atom; C₃-Clocycloalkyl; 3- to 12-membered heterocyclic; or C₁-C₆ alkoxy,
X is hydrogen; halogen; C₁-C₆ alkyl; C₁-C₆ alkyl substituted with 1 to 3 halogens; C₁-C₆ alkyl having a nitrogen, oxygen, or sulfur atom; C₁-C₆ alkoxy; or C₁-C₆ alkoxy substituted with 1 to 3 halogens,
W is a bond, —CH₂—, —O—, —NH—, —CH₂CH₂—, —CH═CH—, or —C≡C—,
A ring is 6- to 12-membered aryl; 5- to 12-membered heteroaryl; C₃-C₁₀ cycloalkyl; or 3- to 12-membered heterocyclic, and
X₁, X₂, X₃, and X₄ are, independently each other, hydrogen; cyano; halogen; C₁-C₆ alkyl; C₁-C₆ alkoxy-C₁-C₆ alkyl; C₁-C₆ alkyl substituted with 1 to 3 halogens; C₃-C₁₀ cycloalkyl; 3- to 12-membered heterocyclic; C₁-C₆ alkoxy; C₁-C₆ alkoxy substituted with 1 to 3 halogens; C₁-C₆ alkoxy- C₁-C₆ alkoxy; morpholinyl-C₁-C₆ alkoxy; mono- or di-C₁-C₆ alkylamino-C₁-C₆ alkoxy; C₃-C₁₀ cycloalkyl-C₁-C₆ alkoxy; C₁-C₆ alkylthio; amino; mono- or di-C₁-C₆ alkylamino; C₁-C₆ alkylcarbonyl; hydroxy; or nitro.

In the compound of Formula 1 or pharmaceutically acceptable salt thereof according to the present invention, L may be —O—.

In the compound of Formula 1 or pharmaceutically acceptable salt thereof according to the present invention, Y and Z may be, independently each other, —O— or —CR₁R₂—.

In the compound of Formula 1 or pharmaceutically acceptable salt thereof according to the present invention, R₁ and R₂ may be, independently each other, hydrogen or C₁-C₆ alkyl; or may form C₃-C₁₀ cycloalkyl together with the carbon atom to which they are attached. In an embodiment, Y may be —O—; and Z may be —CR₁R₂—.

In the compound of Formula 1 or pharmaceutically acceptable salt thereof according to the present invention, X may be hydrogen, halogen, or C₁-C₆ alkoxy.

In the compound of Formula 1 or pharmaceutically acceptable salt thereof according to the present invention, W may be a bond (i.e., the A ring is directly bound to the 2,3-dihydro-1H-indene or 2,3-dihydrobenzofuran moiety) or —CH═CH—.

In the compound of Formula 1 or pharmaceutically acceptable salt thereof according to the present invention, the A ring may be 6- to 12-membered aryl; 5- to 12-membered heteroaryl; or 3- to 12-membered heterocyclic. In an embodiment, the 6- to 12-membered aryl may be is phenyl, naphthalenyl, or biphenyl. In another embodiment, the 5- to 12-membered heteroaryl may be benzodioxolyl, pyridinyl, thiophenyl, quinolinyl, isoquinolinyl, benzofuranyl, benzo[b]thiophenyl, quinoxalinyl, or furanyl. In still another embodiment, the 3- to 12-membered heterocyclic may be 2,3-dihydrobenzodioxinyl, 2,3-dihydrobenzofuranyl, 3,4-dihydro-2H-benzo[b][1,4]oxazinyl, chromanyl, or 3,4-dihydro-2H-benzo[b][1,4]dioxepinyl.

In the compound of Formula 1 or pharmaceutically acceptable salt thereof according to the present invention, X₁, X₂, X₃, and X₄ may be, independently each other, hydrogen; cyano; halogen; C₁-C₆ alkyl; C₁-C₆ alkoxy-C₁-C₆ alkyl; C₁-C₆ alkyl substituted with 1 to 3 halogens; C₃-C₁₀ cycloalkyl; morpholinyl; C₁-C₆ alkoxy; C₁-C₆ alkoxy substituted with 1 to 3 halogens; C₁-C₆ alkoxy-C₁-C₆ alkoxy; morpholinyl-C₁-C₆ alkoxy; mono- or di-C₁-C₆ alkylamino-C₁-C₆ alkoxy; C₃-C₁₀ cycloalkyl-C₁-C₆ alkoxy; C₁-C₆ alkylthio; mono- or di-C₁-C₆ alkylamino; or C₁-C₆ alkylcarbonyl.

In a preferred embodiment of the present invention,
L is —O—,
Y and Z are, independently each other, —O— or —CR₁R₂—,
R₁ and R₂ are, independently each other, hydrogen or C₁-C₆ alkyl; or R₁ and R₂ form C₃-C₁₀ cycloalkyl together with the carbon atom to which they are attached,
X is hydrogen, halogen, or C₁-C₆ alkoxy,
W is a bond or —CH═CH—,
A ring is 6- to 12-membered aryl, 5- to 12-membered heteroaryl, or 3- to 12-membered heterocyclic, and
X₁, X₂, X₃, and X₄ are, independently each other, hydrogen; cyano; halogen; C₁-C₆ alkyl; C₁-C₆ alkoxy-C₁-C₆ alkyl; C₁-C₆ alkyl substituted with 1 to 3 halogens; C₃-C₁₀ cycloalkyl; morpholinyl; C₁-C₆ alkoxy; C₁-C₆ alkoxy substituted with 1 to 3 halogens; C₁-C₆ alkoxy-C₁-C₆ alkoxy; morpholinyl-C₁-C₆ alkoxy; mono- or di-C₁-C₆ alkylamino-C₁-C₆ alkoxy; C₃-C₁₀ cycloalkyl-C₁-C₆ alkoxy; C₁-C₆ alkylthio; mono- or di-C₁-C₆ alkylamino; or C₁-C₆ alkylcarbonyl.

In said embodiment, Y may be —O—; and Z is —CR₁R₂—.

In said embodiment, the A ring may be phenyl, naphthalenyl, or biphenyl.

In said embodiment, the A ring may be benzodioxolyl, pyridinyl, thiophenyl, quinolinyl, isoquinolinyl, benzofuranyl, benzo[b]thiophenyl, quinoxalinyl, or furanyl.

In said embodiment, the A ring may be 2,3-dihydrobenzodioxinyl, 2,3-dihydrobenzofuranyl, 3,4-dihydro-2H-benzo[b][1,4]oxazinyl, chromanyl, or 3,4-dihydro-2H-benzo[b][1,4]dioxepinyl.

In the compound of Formula 1 or pharmaceutically acceptable salt, preferable compounds include a compound, including a pharmaceutically acceptable salt thereof, selected from the group consisting of:
(S)-quinuclidin-3-yl (5-(4-fluorophenyl)-2,3-dihydro-1H-inden-1-yl)carbamate;
(S)-quinuclidin-3-yl (5-(4-chlorophenyl)-2,3-dihydro-1H-inden-1-yl)carbamate;
(S)-quinuclidin-3-yl (5-(4-(trifluoromethyl)phenyl)-2,3-dihydro-1H-inden-1-yl)carbamate;
(S)-quinuclidin-3-yl (5-(4-(trifluoromethoxy)phenyl)-2,3-dihydro-1H-inden-1-yl)carbamate;
(S)-quinuclidin-3-yl (5-(3-fluorophenyl)-2,3-dihydro-1H-inden-1-yl)carbamate;
(S)-quinuclidin-3-yl (5-(3-chlorophenyl)-2,3-dihydro-1H-inden-1-yl)carbamate;
(S)-quinuclidin-3-yl (5-(3-(trifluoromethyl)phenyl)-2,3-dihydro-1H-inden-1-yl)carbamate;
(S)-quinuclidin-3-yl (5-(3-(trifluoromethoxy)phenyl)-2,3-dihydro-1H-inden-1-yl)carbamate;
(S)-quinuclidin-3-yl (5-(2-fluorophenyl)-2,3-dihydro-1H-inden-1-yl)carbamate;
(S)-quinuclidin-3-yl (5-(2-chlorophenyl)-2,3-dihydro-1H-inden-1-yl)carbamate;
(S)-quinuclidin-3-yl (5-(2-(trifluoromethyl)phenyl)-2,3-dihydro-1H-inden-1-yl)carbamate;
(S)-quinuclidin-3-yl (5-(2-(trifluoromethoxy)phenyl)-2,3-dihydro-1H-inden-1-yl)carbamate;
(S)-quinuclidin-3-yl (5-(p-tolyl)-2,3-dihydro-1H-inden-1-yl)carbamate;
(S)-quinuclidin-3-yl (5-(4-(difluoromethoxy)phenyl)-2,3-dihydro-1H-inden-1-yl)carbamate;
(S)-quinuclidin-3-yl (5-(3,4-difluorophenyl)-2,3-dihydro-1H-inden-1-yl)carbamate;
(S)-quinuclidin-3-yl (5-(2,4-difluorophenyl)-2,3-dihydro-1H-inden-1-yl)carbamate;
(S)-quinuclidin-3-yl (5-(2,3-difluorophenyl)-2,3-dihydro-1H-inden-1-yl)carbamate;
(S)-quinuclidin-3-yl (5-(2,5-difluorophenyl)-2,3-dihydro-1H-inden-1-yl)carbamate;
(S)-quinuclidin-3-yl (5-(3,5-difluorophenyl)-2,3-dihydro-1H-inden-1-yl)carbamate;
(S)-quinuclidin-3-yl (5-(2,3,4-trifluorophenyl)-2,3-dihydro-1H-inden-1-yl)carbamate;
(S)-quinuclidin-3-yl (5-(2,4,5-trifluorophenyl)-2,3-dihydro-1H-inden-1-yl)carbamate;
(S)-quinuclidin-3-yl (5-(3,5-dichlorophenyl)-2,3-dihydro-1H-inden-1-yl)carbamate;
(S)-quinuclidin-3-yl (5-(3,4-dichlorophenyl)-2,3-dihydro-1H-inden-1-yl)carbamate;
(S)-quinuclidin-3-yl (5-(2,5-dichlorophenyl)-2,3-dihydro-1H-inden-1-yl)carbamate;
(S)-quinuclidin-3-yl (6-(4-fluorophenyl)-2,3-dihydro-1H-inden-1-yl)carbamate;
(S)-quinuclidin-3-yl (6-(4-chlorophenyl)-2,3-dihydro-1H-inden-1-yl)carbamate;
(S)-quinuclidin-3-yl (6-(4-(trifluoromethyl)phenyl)-2,3-dihydro-1H-inden-1-yl)carbamate;
(S)-quinuclidin-3-yl (6-(4-(trifluoromethoxy)phenyl)-2,3-dihydro-1H-inden-1-yl)carbamate;
(S)-quinuclidin-3-yl (6-(3-fluorophenyl)-2,3-dihydro-1H-inden-1-yl)carbamate;
(S)-quinuclidin-3-yl (6-(3-chlorophenyl)-2,3-dihydro-1H-inden-1-yl)carbamate;
(S)-quinuclidin-3-yl (6-(3-(trifluoromethyl)phenyl)-2,3-dihydro-1H-inden-1-yl)carbamate;
(S)-quinuclidin-3-yl (6-(3-(trifluoromethoxy)phenyl)-2,3-dihydro-1H-inden-1-yl)carbamate;
(S)-quinuclidin-3-yl (6-(2-fluorophenyl)-2,3-dihydro-1H-inden-1-yl)carbamate;
(S)-quinuclidin-3-yl (6-(2-chlorophenyl)-2,3-dihydro-1H-inden-1-yl)carbamate;
(S)-quinuclidin-3-yl (6-(2-(trifluoromethyl)phenyl)-2,3-dihydro-1H-inden-1-yl)carbamate;
(S)-quinuclidin-3-yl (6-(2-(trifluoromethoxy)phenyl)-2,3-dihydro-1H-inden-1-yl)carbamate;
(S)-quinuclidin-3-yl (6-(4-(difluoromethoxy)phenyl)-2,3-dihydro-1H-inden-1-yl)carbamate;
(S)-quinuclidin-3-yl (6-(p-tolyl)-2,3-dihydro-1H-inden-1-yl)carbamate;
(S)-quinuclidin-3-yl (6-(3,4-difluorophenyl)-2,3-dihydro-1H-inden-1-yl)carbamate;
(S)-quinuclidin-3-yl (6-(3,5-difluorophenyl)-2,3-dihydro-1H-inden-1-yl)carbamate;
(S)-quinuclidin-3-yl (6-(2,5-difluorophenyl)-2,3-dihydro-1H-inden-1-yl)carbamate;
(S)-quinuclidin-3-yl (6-(2,3-difluorophenyl)-2,3-dihydro-1H-inden-1-yl)carbamate;
(S)-quinuclidin-3-yl (6-(2,4-difluorophenyl)-2,3-dihydro-1H-inden-1-yl)carbamate;
(S)-quinuclidin-3-yl (6-(2,4,5-trifluorophenyl)-2,3-dihydro-1H-inden-1-yl)carbamate;
(S)-quinuclidin-3-yl (6-(2,3,4-trifluorophenyl)-2,3-dihydro-1H-inden-1-yl)carbamate;
(S)-quinuclidin-3-yl (6-(2,5-dichlorophenyl)-2,3-dihydro-1H-inden-1-yl)carbamate;
(S)-quinuclidin-3-yl (6-(3,4-dichlorophenyl)-2,3-dihydro-1H-inden-1-yl)carbamate;
(S)-quinuclidin-3-yl (6-(3,5-dichlorophenyl)-2,3-dihydro-1H-inden-1-yl)carbamate;
(S)-quinuclidin-3-yl (6-(4-fluorophenyl)-2,2-dimethyl-2,3-dihydro-1H-inden-1-yl)carbamate;
(S)-quinuclidin-3-yl (6-(4-chlorophenyl)-2,2-dimethyl-2,3-dihydro-1H-inden-1-yl)carbamate;
(S)-quinuclidin-3-yl (2,2-dimethyl-6-(4-(trifluoromethyl)phenyl)-2,3-dihydro-1H-inden-1-yl)carbamate;
(S)-quinuclidin-3-yl (2,2-dimethyl-6-(4-(trifluoromethoxy)phenyl)-2,3-dihydro-1H-inden-1-yl)carbamate;
(S)-quinuclidin-3-yl (6-(3-fluorophenyl)-2,2-dimethyl-2,3-dihydro-1H-inden-1-yl)carbamate;
(S)-quinuclidin-3-yl (6-(3-chlorophenyl)-2,2-dimethyl-2,3-dihydro-1H-inden-1-yl)carbamate;
(S)-quinuclidin-3-yl (2,2-dimethyl-6-(3-(trifluoromethyl)phenyl)-2,3-dihydro-1H-inden-1-yl)carbamate;
(S)-quinuclidin-3-yl (2,2-dimethyl-6-(3-(trifluoromethoxy)phenyl)-2,3-dihydro-1H-inden-1-yl)carbamate;
(S)-quinuclidin-3-yl (6-(2-fluorophenyl)-2,2-dimethyl-2,3-dihydro-1H-inden-1-yl)carbamate;
(S)-quinuclidin-3-yl (6-(2-chlorophenyl)-2,2-dimethyl-2,3-dihydro-1H-inden-1-yl)carbamate;
(S)-quinuclidin-3-yl (2,2-dimethyl-6-(2-(trifluoromethyl)phenyl)-2,3-dihydro-1H-inden-1-yl)carbamate;
(S)-quinuclidin-3-yl (2,2-dimethyl-6-(p-tolyl)-2,3-dihydro-1H-inden-1-yl)carbamate;
(S)-quinuclidin-3-yl (6-(4-(difluoromethoxy)phenyl)-2,2-dimethyl-2,3-dihydro-1H-inden-1-yl)carbamate;
(S)-quinuclidin-3-yl (6-(3,4-difluorophenyl)-2,2-dimethyl-2,3-dihydro-1H-inden-1-yl)carbamate;
(S)-quinuclidin-3-yl (6-(2,4-difluorophenyl)-2,2-dimethyl-2,3-dihydro-1H-inden-1-yl)carbamate;
(S)-quinuclidin-3-yl (6-(2,3-difluorophenyl)-2,2-dimethyl-2,3-dihydro-1H-inden-1-yl)carbamate;
(S)-quinuclidin-3-yl (6-(2,5-difluorophenyl)-2,2-dimethyl-2,3-dihydro-1H-inden-1-yl)carbamate;
(S)-quinuclidin-3-yl (6-(3,5-difluorophenyl)-2,2-dimethyl-2,3-dihydro-1H-inden-1-yl)carbamate;
(S)-quinuclidin-3-yl (2,2-dimethyl-6-(2,3,4-trifluorophenyl)-2,3-dihydro-1H-inden-1-yl)carbamate;
(S)-quinuclidin-3-yl (2,2-dimethyl-(2,4,5-trifluorophenyl)-2,3-dihydro-1H-inden-1-yl)carbamate;
(S)-quinuclidin-3-yl (6-(3,5-dichlorophenyl)-2,2-dimethyl-2,3-dihydro-1H-inden-1-yl)carbamate;
(S)-quinuclidin-3-yl (6-(3,4-dichlorophenyl)-2,2-dimethyl-2,3-dihydro-1H-inden-1-yl)carbamate;
(S)-quinuclidin-3-yl (6-(2,5-dichlorophenyl)-2,2-dimethyl-2,3-dihydro-1H-inden-1-yl)carbamate;
(S)-quinuclidin-3-yl (6-(4-(2-methoxyethoxy)phenyl)-2,2-dimethyl-2,3-dihydro-1H-inden-1-yl)carbamate;
(S)-quinuclidin-3-yl (5-(4-fluorophenyl)-2,2-dimethyl-2,3-dihydro-1H-inden-1-yl)carbamate;
(S)-quinuclidin-3-yl (5-(4-chlorophenyl)-2,2-dimethyl-2,3-dihydro-1H-inden-1-yl)carbamate;
(S)-quinuclidin-3-yl (2,2-dimethyl-5-(4-(trifluoromethyl)phenyl)-2,3-dihydro-1H-inden-1-yl)carbamate;
(S)-quinuclidin-3-yl (2,2-dimethyl-5-(4-(trifluoromethoxy)phenyl)-2,3-dihydro-1H-inden-1-yl)carbamate;
(S)-quinuclidin-3-yl (5-(3-fluorophenyl)-2,2-dimethyl-2,3-dihydro-1H-inden-1-yl)carbamate;
(S)-quinuclidin-3-yl (5-(3-chlorophenyl)-2,2-dimethyl-2,3-dihydro-1H-inden-1-yl)carbamate;
(S)-quinuclidin-3-yl (2,2-dimethyl-5-(3-(trifluoromethyl)phenyl)-2,3-dihydro-1H-inden-1-yl)carbamate;
(S)-quinuclidin-3-yl (2,2-dimethyl-6-(3-(trifluoromethoxy)phenyl)-2,3-dihydro-1H-inden-1-yl)carbamate;
(S)-quinuclidin-3-yl (5-(2-fluorophenyl)-2,2-dimethyl-2,3-dihydro-1H-inden-1-yl)carbamate;
(S)-quinuclidin-3-yl (5-(2-chlorophenyl)-2,2-dimethyl-2,3-dihydro-1H-inden-1-yl)carbamate;
(S)-quinuclidin-3-yl (2,2-dimethyl-5-(2-(trifluoromethyl)phenyl)-2,3-dihydro-1H-inden-1-yl)carbamate;
(S)-quinuclidin-3-yl (2,2-dimethyl-5-(2-(trifluoromethoxy)phenyl)-2,3-dihydro-1H-inden-1-yl)carbamate;
(S)-quinuclidin-3-yl (2,2-dimethyl-5-(p-tolyl)-2,3-dihydro-1H-inden-1-yl)carbamate;
(S)-quinuclidin-3-yl (5-(4-(difluoromethoxy)phenyl)-2,2-dimethyl-2,3-dihydro-1H-inden-1-yl)carbamate;
(S)-quinuclidin-3-yl (5-(3,4-difluorophenyl)-2,2-dimethyl-2,3-dihydro-1H-inden-1-yl)carbamate;
(S)-quinuclidin-3-yl (5-(2,5-difluorophenyl)-2,2-dimethyl-2,3-dihydro-1H-inden-1-yl)carbamate;
(S)-quinuclidin-3-yl (5-(2,4-difluorophenyl)-2,2-dimethyl-2,3-dihydro-1H-inden-1-yl)carbamate;
(S)-quinuclidin-3-yl (5-(3,5-difluorophenyl)-2,2-dimethyl-2,3-dihydro-1H-inden-1-yl)carbamate;
(S)-quinuclidin-3-yl (5-(2,3-difluorophenyl)-2,2-dimethyl-2,3-dihydro-1H-inden-1-yl)carbamate;
(S)-quinuclidin-3-yl (5-(3,4-dichlorophenyl)-2,2-dimethyl-2,3-dihydro-1H-inden-1-yl)carbamate;
(S)-quinuclidin-3-yl (5-(2,5-dichlorophenyl)-2,2-dimethyl-2,3-dihydro-1H-inden-1-yl)carbamate;
(S)-quinuclidin-3-yl (5-(3,5-dichlorophenyl)-2,2-dimethyl-2,3-dihydro-1H-inden-1-yl)carbamate;
(S)-quinuclidin-3-yl (2,2-dimethyl-5-(2,3,4-trifluorophenyl)-2,3-dihydro-1H-inden-1-yl)carbamate;
(S)-quinuclidin-3-yl (2,2-dimethyl-5-(2,4,5-trifluorophenyl)-2,3-dihydro-1H-inden-1-yl)carbamate;
(S)-quinuclidin-3-yl (5-(4-(2-methoxyethoxy)phenyl)-2,2-dimethyl-2,3-dihydro-1H-inden-1-yl)carbamate;
(S)-quinuclidin-3-yl (5-(3-(2-methoxyethoxy)phenyl)-2,2-dimethyl-2,3-dihydro-1H-inden-1-yl)carbamate;
(S)-quinuclidin-3-yl (5-(2-(2-methoxyethoxy)phenyl)-2,2-dimethyl-2,3-dihydro-1H-inden-1-yl)carbamate;
(S)-quinuclidin-3-yl (5-(4-(methoxymethoxy)phenyl)-2,2-dimethyl-2,3-dihydro-1H-inden-1-yl)carbamate;
(S)-quinuclidin-3-yl (5-(3-(methoxymethoxy)phenyl)-2,2-dimethyl-2,3-dihydro-1H-inden-1-yl)carbamate;
(S)-quinuclidin-3-yl (5-(2-(methoxymethoxy)phenyl)-2,2-dimethyl-2,3-dihydro-1H-inden-1-yl)carbamate;
(S)-quinuclidin-3-yl (5-(3-methoxyphenyl)-2,2-dimethyl-2,3-dihydro-1H-inden-1-yl)carbamate;
(S)-quinuclidin-3-yl (5-(3-ethoxyphenyl)-2,2-dimethyl-2,3-dihydro-1H-inden-1-yl)carbamate;
(S)-quinuclidin-3-yl (5-(3-isopropoxyphenyl)-2,2-dimethyl-2,3-dihydro-1H-inden-1-yl)carbamate;
(S)-quinuclidin-3-yl (5-(3-(dimethylamino)phenyl)-2,2-dimethyl-2,3-dihydro-1H-inden-1-yl)carbamate;
(S)-quinuclidin-3-yl (2,2-dimethyl-5-(3-(methylthio)phenyl)-2,3-dihydro-1H-inden-1-yl)carbamate;
(S)-quinuclidin-3-yl (5-(3-ethylphenyl)-2,2-dimethyl-2,3-dihydro-1H-inden-1-yl)carbamate;
(S)-quinuclidin-3-yl (5-(3-isopropylphenyl)-2,2-dimethyl-2,3-dihydro-1H-inden-1-yl)carbamate;
(S)-quinuclidin-3-yl (5-(3-(tert-butyl)phenyl)-2,2-dimethyl-2,3-dihydro-1H-inden-1-yl)carbamate;
(S)-quinuclidin-3-yl (5-(2,2-difluorobenzo dioxol-5-yl)-2,2-dimethyl-2,3-dihydro-1H-inden-1-yl)carbamate;
(S)-quinuclidin-3-yl (5-(2,3-dihydrobenzo dioxin-6-yl)-2,2-dimethyl-2,3-dihydro-1H-inden-1-yl)carbamate;
(S)-quinuclidin-3-yl (5-(2,3-dihydrobenzofuran-5-yl)-2,2-dimethyl-2,3-dihydro-1H-inden-1-yl)carbamate;
(S)-quinuclidin-3-yl (2,2-dimethyl-5-(3-(2-morpholinoethoxy)phenyl)-2,3-dihydro-1H-inden-1-yl)carbamate;
(S)-quinuclidin-3-yl (5-(4-(2-fluoroethoxy)phenyl)-2,2-dimethyl-2,3-dihydro-1H-inden-1-yl)carbamate;
(S)-quinuclidin-3-yl (2,2-dimethyl-5-(4-(2,2,2-trifluoroethoxy)phenyl)-2,3-dihydro-1H-inden-1-yl)carbamate;
(S)-quinuclidin-3-yl (2,2-dimethyl-5-(4-(2-morpholinoethoxy)phenyl)-2,3-dihydro-1H-inden-1-yl)carbamate;
(S)-quinuclidin-3-yl (2,2-dimethyl-5-(2-(trifluoromethyl)pyridin-4-yl)-2,3-dihydro-1H-inden-1-yl)carbamate;
(S)-quinuclidin-3-yl (5-(2,5-dichloropyridin-4-yl)-2,2-dimethyl-2,3-dihydro-1H-inden-1-yl)carbamate;
(S)-quinuclidin-3-yl (2,2-dimethyl-5-(4-methyl-3,4-dihydro-2H-benzo oxazin-7-yl)-2,3-dihydro-1H-inden-1-yl)carbamate;
(S)-quinuclidin-3-yl (5-(2-chloro-5-methoxyphenyl)-2,2-dimethyl-2,3-dihydro-1H-inden-1-yl)carbamate;
(S)-quinuclidin-3-yl (5-(3-chloro-4-methoxyphenyl)-2,2-dimethyl-2,3-dihydro-1H-inden-1-yl)carbamate;
(S)-quinuclidin-3-yl (5-(2-chloro-4-methoxyphenyl)-2,2-dimethyl-2,3-dihydro-1H-inden-1-yl)carbamate;
(S)-quinuclidin-3-yl (5-(2-chloro-3-methoxyphenyl)-2,2-dimethyl-2,3-dihydro-1H-inden-1-yl)carbamate;
(S)-quinuclidin-3-yl (5-(4-chloro-3-methoxyphenyl)-2,2-dimethyl-2,3-dihydro-1H-inden-1-yl)carbamate;
(S)-quinuclidin-3-yl (5-(2,3-dichloro-4-methoxyphenyl)-2,2-dimethyl-2,3-dihydro-1H-inden-1-yl)carbamate;
(S)-quinuclidin-3-yl (5-(3-chloro-4-isopropoxyphenyl)-2,2-dimethyl-2,3-dihydro-1H-inden-1-yl)carbamate;
(S)-quinuclidin-3-yl (5-(2-fluoro-4-methoxyphenyl)-2,2-dimethyl-2,3-dihydro-1H-inden-1-yl)carbamate;
(S)-quinuclidin-3-yl (5-(3-(dimethylamino)-4-fluorophenyl)-2,2-dimethyl-2,3-dihydro-1H-inden-1-yl)carbamate;
(S)-quinuclidin-3-yl (5-(chroman-6-yl)-2,2-dimethyl-2,3-dihydro-1H-inden-1-yl)carbamate;
(S)-quinuclidin-3-yl (5-(4-cyclopropylphenyl)-2,2-dimethyl-2,3-dihydro-1H-inden-1-yl)carbamate;
(S)-quinuclidin-3-yl (5-(4-(dimethylamino)phenyl)-2,2-dimethyl-2,3-dihydro-1H-inden-1-yl)carbamate;
(S)-quinuclidin-3-yl (5-(4-ethoxy-3-fluorophenyl)-2,2-dimethyl-2,3-dihydro-1H-inden-1-yl)carbamate;
(S)-quinuclidin-3-yl (5-(4-methoxy-3-(trifluoromethyl)phenyl)-2,2-dimethyl-2,3-dihydro-1H-inden-1-yl)carbamate;
(S)-quinuclidin-3-yl (2,2-dimethyl-5-(4-propoxyphenyl)-2,3-dihydro-1H-inden-1-yl)carbamate;
(S)-quinuclidin-3-yl (5-(4-isobutoxyphenyl)-2,2-dimethyl-2,3-dihydro-1H-inden-1-yl)carbamate;
(S)-quinuclidin-3-yl (2,2-dimethyl-5-(thiophen-3-yl)-2,3-dihydro-1H-inden-1-yl)carbamate;
(S)-quinuclidin-3-yl (2,2-dimethyl-5-(4-methylthiophen-3-yl)-2,3-dihydro-1H-inden-1-yl)carbamate;
(S)-quinuclidin-3-yl (5-(3-(2-(dimethylamino)ethoxy)phenyl)-2,2-dimethyl-2,3-dihydro-1H-inden-1-yl)carbamate;
(S)-quinuclidin-3-yl (5-(isoquinolin-8-yl)-2,2-dimethyl-2,3-dihydro-1H-inden-1-yl)carbamate;
(S)-quinuclidin-3-yl (5-(isoquinolin-5-yl)-2,2-dimethyl-2,3-dihydro-1H-inden-1-yl)carbamate;
(S)-quinuclidin-3-yl (5-(isoquinolin-4-yl)-2,2-dimethyl-2,3-dihydro-1H-inden-1-yl)carbamate;
(S)-quinuclidin-3-yl (2,2-dimethyl-5-(quinolin-3-yl)-2,3-dihydro-1H-inden-1-yl)carbamate;
(S)-quinuclidin-3-yl (5-(4-isopropoxyphenyl)-2,2-dimethyl-2,3-dihydro-1H-inden-1-yl)carbamate;
(S)-quinuclidin-3-yl (5-(2-isopropoxyphenyl)-2,2-dimethyl-2,3-dihydro-1H-inden-1-yl)carbamate;
(S)-quinuclidin-3-yl (5-(4-(tert-butyl)phenyl)-2,2-dimethyl-2,3-dihydro-1H-inden-1-yl)carbamate;
(S)-quinuclidin-3-yl (5-(4-ethylphenyl)-2,2-dimethyl-2,3-dihydro-1H-inden-1-yl)carbamate;
(S)-quinuclidin-3-yl (2,2-dimethyl-5-(4-morpholinophenyl)-2,3-dihydro-1H-inden-1-yl)carbamate;
(S)-quinuclidin-3-yl (5-(4-(methoxymethyl)phenyl)-2,2-dimethyl-2,3-dihydro-1H-inden-1-yl)carbamate;
(S)-quinuclidin-3-yl (5-(4-(2,2-difluoroethoxy)phenyl)-2,2-dimethyl-2,3-dihydro-1H-inden-1-yl)carbamate;
(S)-quinuclidin-3-yl (2,2-dimethyl-5-(3-methyl-4-morpholinophenyl)-2,3-dihydro-1H-inden-1-yl)carbamate;
(S)-quinuclidin-3-yl (5-(3-fluoro-4-morpholinophenyl)-2,2-dimethyl-2,3-dihydro-1H-inden-1-yl)carbamate;
(S)-quinuclidin-3-yl (5-(3-chloro-4-morpholinophenyl)-2,2-dimethyl-2,3-dihydro-1H-inden-1-yl)carbamate;
(S)-quinuclidin-3-yl (5-(3,4-dihydro-2H-benzo dioxepin-7-yl)-2,2-dimethyl-2,3-dihydro-1H-inden-1-yl)carbamate;
(S)-quinuclidin-3-yl (5-(3-(difluoromethyl)phenyl)-2,2-dimethyl-2,3-dihydro-1H-inden-1-yl)carbamate;
(S)-quinuclidin-3-yl (2,2-dimethyl-5-(quinolin-8-yl)-2,3-dihydro-1H-inden-1-yl)carbamate;
(S)-quinuclidin-3-yl (5-(benzofuran-3-yl)-2,2-dimethyl-2,3-dihydro-1H-inden-1-yl)carbamate;
(S)-quinuclidin-3-yl (5-(benzofuran-2-yl)-2,2-dimethyl-2,3-dihydro-1H-inden-1-yl)carbamate;
(S)-quinuclidin-3-yl (5-(4-methoxyphenyl)-2,2-dimethyl-2,3-dihydro-1H-inden-1-yl)carbamate;
(S)-quinuclidin-3-yl (5-(4-ethoxyphenyl)-2,2-dimethyl-2,3-dihydro-1H-inden-1-yl)carbamate;
(S)-quinuclidin-3-yl (5-(4-isobutylphenyl)-2,2-dimethyl-2,3-dihydro-1H-inden-1-yl)carbamate;
(S)-quinuclidin-3-yl (5-(2,4-dichloro-3-methoxyphenyl)-2,2-dimethyl-2,3-dihydro-1H-inden-1-yl)carbamate;
(S)-quinuclidin-3-yl (2,2-dimethyl-5-(4-propylphenyl)-2,3-dihydro-1H-inden-1-yl)carbamate;
(S)-quinuclidin-3-yl (5-(4-butylphenyl)-2,2-dimethyl-2,3-dihydro-1H-inden-1-yl)carbamate;
(S)-quinuclidin-3-yl (5-(2,3-dimethoxyphenyl)-2,2-dimethyl-2,3-dihydro-1H-inden-1-yl)carbamate;
(S)-quinuclidin-3-yl (5-(2,4-dimethoxyphenyl)-2,2-dimethyl-2,3-dihydro-1H-inden-1-yl)carbamate;
(S)-quinuclidin-3-yl (5-(2,5-dimethoxyphenyl)-2,2-dimethyl-2,3-dihydro-1H-inden-1-yl)carbamate;
(S)-quinuclidin-3-yl (5-(2,6-dimethoxyphenyl)-2,2-dimethyl-2,3-dihydro-1H-inden-1-yl)carbamate;
(S)-quinuclidin-3-yl (5-(3,5-dimethoxyphenyl)-2,2-dimethyl-2,3-dihydro-1H-inden-1-yl)carbamate;
(S)-quinuclidin-3-yl (5-(4-ethoxy-3-(trifluoromethyl)phenyl)-2,2-dimethyl-2,3-dihydro-1H-inden-1-yl)carbamate;
(S)-quinuclidin-3-yl (5-(2,5-difluoro-4-methoxyphenyl)-2,2-dimethyl-2,3-dihydro-1H-inden-1-yl)carbamate;
(S)-quinuclidin-3-yl (5-(2-fluoro-5-isopropoxyphenyl)-2,2-dimethyl-2,3-dihydro-1H-inden-1-yl)carbamate;
(S)-quinuclidin-3-yl (5-(3,5-dimethyl-4-propoxyphenyl)-2,2-dimethyl-2,3-dihydro-1H-inden-1-yl)carbamate;
(S)-quinuclidin-3-yl (5-(4-ethoxy-3,5-dimethylphenyl)-2,2-dimethyl-2,3-dihydro-1H-inden-1-yl)carbamate;
(S)-quinuclidin-3-yl (5-(2-ethylphenyl)-2,2-dimethyl-2,3-dihydro-1H-inden-1-yl)carbamate;
(S)-quinuclidin-3-yl (5-(2-methoxyphenyl)-2,2-dimethyl-2,3-dihydro-1H-inden-1-yl)carbamate;
(S)-quinuclidin-3-yl (5-(2-ethoxyphenyl)-2,2-dimethyl-2,3-dihydro-1H-inden-1-yl)carbamate;
(S)-quinuclidin-3-yl (5-(2-chloro-5-(trifluoromethoxy)phenyl)-2,2-dimethyl-2,3-dihydro-1H-inden-1-yl)carbamate;
(S)-quinuclidin-3-yl (5-(4-fluoro-2-methoxyphenyl)-2,2-dimethyl-2,3-dihydro-1H-inden-1-yl)carbamate;
(S)-quinuclidin-3-yl (5-(4-methoxy-2-methylphenyl)-2,2-dimethyl-2,3-dihydro-1H-inden-1-yl)carbamate;
(S)-quinuclidin-3-yl (5-(4-(tert-butoxymethyl)phenyl)-2,2-dimethyl-2,3-dihydro-1H-inden-1-yl)carbamate;
(S)-quinuclidin-3-yl (5-(2-chloro-3-(trifluoromethyl)phenyl)-2,2-dimethyl-2,3-dihydro-1H-inden-1-yl)carbamate;
(S)-quinuclidin-3-yl (5-(2-chloro-4-(trifluoromethyl)phenyl)-2,2-dimethyl-2,3-dihydro-1H-inden-1-yl)carbamate;
(S)-quinuclidin-3-yl (5-(2,6-dimethoxypyridin-3-yl)-2,2-dimethyl-2,3-dihydro-1H-inden-1-yl)carbamate;
(S)-quinuclidin-3-yl (5-(6-(cyclopropylmethoxy)pyridin-3-yl)-2,2-dimethyl-2,3-dihydro-1H-inden-1-yl)carbamate;
(S)-quinuclidin-3-yl (5-(benzo thiophen-2-yl)-2,2-dimethyl-2,3-dihydro-1H-inden-1-yl)carbamate;
(S)-quinuclidin-3-yl (5-(2-(tert-butoxy)pyridin-4-yl)-2,2-dimethyl-2,3-dihydro-1H-inden-1-yl)carbamate;
(S)-quinuclidin-3-yl (5-(6-methoxy-5-(trifluoromethyl)pyridin-3-yl)-2,2-dimethyl-2,3-dihydro-1H-inden-1-yl)carbamate;
(S)-quinuclidin-3-yl (5-(6-methoxynaphthalen-2-yl)-2,2-dimethyl-2,3-dihydro-1H-inden-1-yl)carbamate;
(S)-quinuclidin-3-yl (5-(-3-yl)-2,2-dimethyl-2,3-dihydro-1H-inden-1-yl)carbamate;
(S)-quinuclidin-3-yl (2,2-dimethyl-5-(quinoxalin-6-yl)-2,3-dihydro-1H-inden-1-yl)carbamate;
(S)-quinuclidin-3-yl (5-(3,5-dimethylphenyl)-2,2-dimethyl-2,3-dihydro-1H-inden-1-yl)carbamate;
(S)-quinuclidin-3-yl (5-(furan-2-yl)-2,2-dimethyl-2,3-dihydro-1H-inden-1-yl)carbamate;
(S)-quinuclidin-3-yl (5-(4-isopropoxy-3,5-dimethylphenyl)-2,2-dimethyl-2,3-dihydro-1H-inden-1-yl)carbamate;
(S)-quinuclidin-3-yl (5-(4-methoxy-3,5-dimethylphenyl)-2,2-dimethyl-2,3-dihydro-1H-inden-1-yl)carbamate;
(S)-quinuclidin-3-yl (5-(6-isopropoxypyridin-3-yl)-2,2-dimethyl-2,3-dihydro-1H-inden-1-yl)carbamate;
(S)-quinuclidin-3-yl (5-(3-(2-fluoroethoxy)phenyl)-2,2-dimethyl-2,3-dihydro-1H-inden-1-yl)carbamate;
(S)-quinuclidin-3-yl (5-(2-ethoxy-5-methylphenyl)-2,2-dimethyl-2,3-dihydro-1H-inden-1-yl)carbamate;
(S)-quinuclidin-3-yl (2,2-dimethyl-5-(3-propoxyphenyl)-2,3-dihydro-1H-inden-1-yl)carbamate;
(S)-quinuclidin-3-yl (5-(2-methoxy-5-methylphenyl)-2,2-dimethyl-2,3-dihydro-1H-inden-1-yl)carbamate;
(S)-quinuclidin-3-yl (5-(2-butoxy-5-fluorophenyl)-2,2-dimethyl-2,3-dihydro-1H-inden-1-yl)carbamate;
(S)-quinuclidin-3-yl (5-(4-butoxy-3-chlorophenyl)-2,2-dimethyl-2,3-dihydro-1H-inden-1-yl)carbamate;
(S)-quinuclidin-3-yl (5-(2,4-dipropoxyphenyl)-2,2-dimethyl-2,3-dihydro-1H-inden-1-yl)carbamate;
(S)-quinuclidin-3-yl (5-(3-chloro-4-ethoxyphenyl)-2,2-dimethyl-2,3-dihydro-1H-inden-1-yl)carbamate;
(S)-quinuclidin-3-yl (5-(3-isobutoxyphenyl)-2,2-dimethyl-2,3-dihydro-1H-inden-1-yl)carbamate;
(S)-quinuclidin-3-yl (5-(4-ethoxy-2-methylphenyl)-2,2-dimethyl-2,3-dihydro-1H-inden-1-yl)carbamate;
(S)-quinuclidin-3-yl (5-(3-chloro-4-propoxyphenyl)-2,2-dimethyl-2,3-dihydro-1H-inden-1-yl)carbamate;
(S)-quinuclidin-3-yl (5-(5-fluoro-2-isopropoxyphenyl)-2,2-dimethyl-2,3-dihydro-1H-inden-1-yl)carbamate;
(S)-quinuclidin-3-yl (5-(3-fluoro-4-isopropoxyphenyl)-2,2-dimethyl-2,3-dihydro-1H-inden-1-yl)carbamate;
(S)-quinuclidin-3-yl (5-(5-chloro-2-methoxyphenyl)-2,2-dimethyl-2,3-dihydro-1H-inden-1-yl)carbamate;
(S)-quinuclidin-3-yl (5-(2-fluoro-5-methylphenyl)-2,2-dimethyl-2,3-dihydro-1H-inden-1-yl)carbamate;
(S)-quinuclidin-3-yl (5-(5-chloro-2-ethoxyphenyl)-2,2-dimethyl-2,3-dihydro-1H-inden-1-yl)carbamate;
(S)-quinuclidin-3-yl (5-(5-fluoro-2-propoxyphenyl)-2,2-dimethyl-2,3-dihydro-1H-inden-1-yl)carbamate;
(S)-quinuclidin-3-yl (5-(2-fluoro-6-propoxyphenyl)-2,2-dimethyl-2,3-dihydro-1H-inden-1-yl)carbamate;
(S)-quinuclidin-3-yl (5-(3-fluoro-4-methylphenyl)-2,2-dimethyl-2,3-dihydro-1H-inden-1-yl)carbamate;
(S)-quinuclidin-3-yl (5-(3-fluoro-4-propoxyphenyl)-2,2-dimethyl-2,3-dihydro-1H-inden-1-yl)carbamate;
(S)-quinuclidin-3-yl (5-(5-chloro-2-isopropoxyphenyl)-2,2-dimethyl-2,3-dihydro-1H-inden-1-yl)carbamate;
(S)-quinuclidin-3-yl (5-(3-butoxyphenyl)-2,2-dimethyl-2,3-dihydro-1H-inden-1-yl)carbamate;
(S)-quinuclidin-3-yl (5-(3-fluoro-5-methylphenyl)-2,2-dimethyl-2,3-dihydro-1H-inden-1-yl)carbamate;
(S)-quinuclidin-3-yl (5-(5-fluoro-2-methylphenyl)-2,2-dimethyl-2,3-dihydro-1H-inden-1-yl)carbamate;
(S)-quinuclidin-3-yl (5-(2-chloro-5-(trifluoromethyl)phenyl)-2,2-dimethyl-2,3-dihydro-1H-inden-1-yl)carbamate;
(S)-quinuclidin-3-yl (5-(3-fluoro-5-methoxyphenyl)-2,2-dimethyl-2,3-dihydro-1H-inden-1-yl)carbamate;
(S)-quinuclidin-3-yl (5-(2-chloro-5-methylphenyl)-2,2-dimethyl-2,3-dihydro-1H-inden-1-yl)carbamate;
(S)-quinuclidin-3-yl (5-(3-methoxy-5-(trifluoromethyl)phenyl)-2,2-dimethyl-2,3-dihydro-1H-inden-1-yl)carbamate;
(S)-quinuclidin-3-yl (5-(2-chloro-4-fluorophenyl)-2,2-dimethyl-2,3-dihydro-1H-inden-1-yl)carbamate;
(S)-quinuclidin-3-yl (5-(4-fluoro-3-methoxyphenyl)-2,2-dimethyl-2,3-dihydro-1H-inden-1-yl)carbamate;
(S)-quinuclidin-3-yl (5-(3,4-dimethylphenyl)-2,2-dimethyl-2,3-dihydro-1H-inden-1-yl)carbamate;
(S)-quinuclidin-3-yl (5-(4-fluoro-2-methylphenyl)-2,2-dimethyl-2,3-dihydro-1H-inden-1-yl)carbamate;
(S)-quinuclidin-3-yl (5-(2-fluoro-4-(trifluoromethyl)phenyl)-2,2-dimethyl-2,3-dihydro-1H-inden-1-yl)carbamate;
(S)-quinuclidin-3-yl (5-(2-fluoro-5-methoxyphenyl)-2,2-dimethyl-2,3-dihydro-1H-inden-1-yl)carbamate;
(S)-quinuclidin-3-yl (5-(2-fluoro-3-methoxyphenyl)-2,2-dimethyl-2,3-dihydro-1H-inden-1-yl)carbamate;
(S)-quinuclidin-3-yl (5-(5-isopropyl-2-methoxyphenyl)-2,2-dimethyl-2,3-dihydro-1H-inden-1-yl)carbamate;
(S)-quinuclidin-3-yl (5-(2-fluoro-4-methylphenyl)-2,2-dimethyl-2,3-dihydro-1H-inden-1-yl)carbamate;
(S)-quinuclidin-3-yl (5-(5-fluoro-2-methoxyphenyl)-2,2-dimethyl-2,3-dihydro-1H-inden-1-yl)carbamate;
(S)-quinuclidin-3-yl (2,2-dimethyl-5-(5-methylfuran-3-yl)-2,3-dihydro-1H-inden-1-yl)carbamate;
(S)-quinuclidin-3-yl (5-(3-fluoro-2-methoxyphenyl)-2,2-dimethyl-2,3-dihydro-1H-inden-1-yl)carbamate;
(S)-quinuclidin-3-yl (5-(2-chloro-3-fluorophenyl)-2,2-dimethyl-2,3-dihydro-1H-inden-1-yl)carbamate;
(S)-quinuclidin-3-yl (5-(benzo thiophen-3-yl)-2,2-dimethyl-2,3-dihydro-1H-inden-1-yl)carbamate;
(S)-quinuclidin-3-yl (5-(4-fluoro-2-propoxyphenyl)-2,2-dimethyl-2,3-dihydro-1H-inden-1-yl)carbamate;
(S)-quinuclidin-3-yl (5-(3-(tert-butyl)-5-methylphenyl)-2,2-dimethyl-2,3-dihydro-1H-inden-1-yl)carbamate;
(S)-quinuclidin-3-yl (5-(2-chloro-5-ethoxyphenyl)-2,2-dimethyl-2,3-dihydro-1H-inden-1-yl)carbamate;
(S)-quinuclidin-3-yl (5-(4-chloro-3-fluorophenyl)-2,2-dimethyl-2,3-dihydro-1H-inden-1-yl)carbamate;
(S)-quinuclidin-3-yl (5-(2-fluoro-3-methylphenyl)-2,2-dimethyl-2,3-dihydro-1H-inden-1-yl)carbamate;
(S)-quinuclidin-3-yl (5-(5-cyano-2-methoxyphenyl)-2,2-dimethyl-2,3-dihydro-1H-inden-1-yl)carbamate;
(S)-quinuclidin-3-yl (5-(3-ethoxy-5-(trifluoromethyl)phenyl)-2,2-dimethyl-2,3-dihydro-1H-inden-1-yl)carbamate;
(S)-quinuclidin-3-yl (5-(3-fluoro-5-(trifluoromethyl)phenyl)-2,2-dimethyl-2,3-dihydro-1H-inden-1-yl)carbamate;
(S)-quinuclidin-3-yl (2,2-dimethyl-5-(3-methyl-4-(trifluoromethoxy)phenyl)-2,3-dihydro-1H-inden-1-yl)carbamate;
(S)-quinuclidin-3-yl (2,2-dimethyl-5-(3-(methylthio)-5-(trifluoromethyl)phenyl)-2,3-dihydro-1H-inden-1-yl)carbamate;
(S)-quinuclidin-3-yl (5-(3-fluoro-4-(trifluoromethoxy)phenyl)-2,2-dimethyl-2,3-dihydro-1H-inden-1-yl)carbamate;
(S)-quinuclidin-3-yl (5-(2-ethoxy-4-(trifluoromethyl)phenyl)-2,2-dimethyl-2,3-dihydro-1H-inden-1-yl)carbamate;
(S)-quinuclidin-3-yl (5-(2-methoxy-5-(trifluoromethyl)phenyl)-2,2-dimethyl-2,3-dihydro-1H-inden-1-yl)carbamate;
(S)-quinuclidin-3-yl (5-(2,4-bis(trifluoromethyl)phenyl)-2,2-dimethyl-2,3-dihydro-1H-inden-1-yl)carbamate;
(S)-quinuclidin-3-yl (5-(3-chloro-2-methylphenyl)-2,2-dimethyl-2,3-dihydro-1H-inden-1-yl)carbamate;
(S)-quinuclidin-3-yl (5-(3-methoxy-4-methylphenyl)-2,2-dimethyl-2,3-dihydro-1H-inden-1-yl)carbamate;
(S)-quinuclidin-3-yl (5-(4-fluoro-3-methylphenyl)-2,2-dimethyl-2,3-dihydro-1H-inden-1-yl)carbamate;
(S)-quinuclidin-3-yl (5-(2-ethoxy-5-fluorophenyl)-2,2-dimethyl-2,3-dihydro-1H-inden-1-yl)carbamate;
(S)-quinuclidin-3-yl (5-(3-fluoro-5-propoxyphenyl)-2,2-dimethyl-2,3-dihydro-1H-inden-1-yl)carbamate;
(S)-quinuclidin-3-yl (5-(3,5-bis(trifluoromethyl)phenyl)-2,2-dimethyl-2,3-dihydro-1H-inden-1-yl)carbamate;
(S)-quinuclidin-3-yl (5-(3-chloro-4-methylphenyl)-2,2-dimethyl-2,3-dihydro-1H-inden-1-yl)carbamate;
(S)-quinuclidin-3-yl (5-(2,5-dimethylphenyl)-2,2-dimethyl-2,3-dihydro-1H-inden-1-yl)carbamate;
(S)-quinuclidin-3-yl (5-(3-chloro-4-fluorophenyl)-2,2-dimethyl-2,3-dihydro-1H-inden-1-yl)carbamate;
(S)-quinuclidin-3-yl (5-(6-ethoxypyridin-3-yl)-2,2-dimethyl-2,3-dihydro-1H-inden-1-yl)carbamate;
(S)-quinuclidin-3-yl (5-(2-ethoxypyridin-4-yl)-2,2-dimethyl-2,3-dihydro-1H-inden-1-yl)carbamate;
(S)-quinuclidin-3-yl (5-(2-isopropoxypyridin-4-yl)-2,2-dimethyl-2,3-dihydro-1H-inden-1-yl)carbamate;
(S)-quinuclidin-3-yl (2,2-dimethyl-5-(6-propoxypyridin-3-yl)-2,3-dihydro-1H-inden-1-yl)carbamate;
(S)-quinuclidin-3-yl (5-(6-butoxypyridin-3-yl)-2,2-dimethyl-2,3-dihydro-1H-inden-1-yl)carbamate;
(S)-quinuclidin-3-yl (5-(6-(2-methoxyethoxy)pyridin-3-yl)-2,2-dimethyl-2,3-dihydro-1H-inden-1-yl)carbamate;
(S)-quinuclidin-3-yl (5-(6-isobutoxypyridin-3-yl)-2,2-dimethyl-2,3-dihydro-1H-inden-1-yl)carbamate;
(S)-quinuclidin-3-yl (5-(3-isobutylphenyl)-2,2-dimethyl-2,3-dihydro-1H-inden-1-yl)carbamate;
(S)-quinuclidin-3-yl (5-(4-butoxyphenyl)-2,2-dimethyl-2,3-dihydro-1H-inden-1-yl)carbamate;
(S)-quinuclidin-3-yl (5-(2,4-dimethoxypyridin-3-yl)-2,2-dimethyl-2,3-dihydro-1H-inden-1-yl)carbamate;
(S)-quinuclidin-3-yl (5-(2-acetylthiophen-3-yl)-2,2-dimethyl-2,3-dihydro-1H-inden-1-yl)carbamate;
(S)-quinuclidin-3-yl (5-((E)-3-fluorostyryl)-2,2-dimethyl-2,3-dihydro-1H-inden-1-yl)carbamate;
(S)-quinuclidin-3-yl (5-((E)-4-ethylstyryl)-2,2-dimethyl-2,3-dihydro-1H-inden-1-yl)carbamate;
(S)-quinuclidin-3-yl (5-((E)-2-cyclohexylvinyl)-2,2-dimethyl-2,3-dihydro-1H-inden-1-yl)carbamate;
(S)-quinuclidin-3-yl (2,2-diethyl-5-(4-fluorophenyl)-2,3-dihydro-1H-inden-1-yl)carbamate;
(S)-quinuclidin-3-yl (2,2-diethyl-5-(4-ethylphenyl)-2,3-dihydro-1H-inden-1-yl)carbamate;
(S)-quinuclidin-3-yl (2,2-diethyl-5-(4-propylphenyl)-2,3-dihydro-1H-inden-1-yl)carbamate;
(S)-quinuclidin-3-yl (5-(4-(tert-butyl)phenyl)-2,2-diethyl-2,3-dihydro-1H-inden-1-yl)carbamate;
(S)-quinuclidin-3-yl (5-(4-butylphenyl)-2,2-diethyl-2,3-dihydro-1H-inden-1-yl)carbamate;
(S)-quinuclidin-3-yl (5-(4-cyclopropylphenyl)-2,2-diethyl-2,3-dihydro-1H-inden-1-yl)carbamate;
(S)-quinuclidin-3-yl (2,2-diethyl-5-(4-isopropylphenyl)-2,3-dihydro-1H-inden-1-yl)carbamate;
(S)-quinuclidin-3-yl (2,2-diethyl-5-(4-methoxyphenyl)-2,3-dihydro-1H-inden-1-yl)carbamate;
(S)-quinuclidin-3-yl (5-(4-ethoxyphenyl)-2,2-diethyl-2,3-dihydro-1H-inden-1-yl)carbamate;
(S)-quinuclidin-3-yl (2,2-diethyl-5-(4-propoxyphenyl)-2,3-dihydro-1H-inden-1-yl)carbamate;
(S)-quinuclidin-3-yl (2,2-diethyl-5-(4-isopropoxyphenyl)-2,3-dihydro-1H-inden-1-yl)carbamate;
(S)-quinuclidin-3-yl (2,2-diethyl-5-(4-isobutylphenyl)-2,3-dihydro-1H-inden-1-yl)carbamate;
(S)-quinuclidin-3-yl (2,2-diethyl-5-(3-fluorophenyl)-2,3-dihydro-1H-inden-1-yl)carbamate;
(S)-quinuclidin-3-yl (2,2-diethyl-5-(3-methoxyphenyl)-2,3-dihydro-1H-inden-1-yl)carbamate;
(S)-quinuclidin-3-yl (5-(3-ethoxyphenyl)-2,2-diethyl-2,3-dihydro-1H-inden-1-yl)carbamate;
(S)-quinuclidin-3-yl (2,2-diethyl-5-(3-isopropoxyphenyl)-2,3-dihydro-1H-inden-1-yl)carbamate;
(S)-quinuclidin-3-yl (2,2-diethyl-5-(3-ethylphenyl)-2,3-dihydro-1H-inden-1-yl)carbamate;
(S)-quinuclidin-3-yl (2,2-diethyl-5-(3-isopropylphenyl)-2,3-dihydro-1H-inden-1-yl)carbamate;
(S)-quinuclidin-3-yl (5-(3-(tert-butyl)phenyl)-2,2-diethyl-2,3-dihydro-1H-inden-1-yl)carbamate;
(S)-quinuclidin-3-yl (5-(2,3-dimethoxyphenyl)-2,2-diethyl-2,3-dihydro-1H-inden-1-yl)carbamate;
(S)-quinuclidin-3-yl (5-(2,4-dimethoxyphenyl)-2,2-diethyl-2,3-dihydro-1H-inden-1-yl)carbamate;
(S)-quinuclidin-3-yl (5-(2,5-dimethoxyphenyl)-2,2-diethyl-2,3-dihydro-1H-inden-1-yl)carbamate;
(S)-quinuclidin-3-yl (5-(3,4-dimethoxyphenyl)-2,2-diethyl-2,3-dihydro-1H-inden-1-yl)carbamate;
(S)-quinuclidin-3-yl (5-(3,5-dimethoxyphenyl)-2,2-diethyl-2,3-dihydro-1H-inden-1-yl)carbamate;
(S)-quinuclidin-3-yl (5-(2,6-dimethoxyphenyl)-2,2-diethyl-2,3-dihydro-1H-inden-1-yl)carbamate;
(S)-quinuclidin-3-yl (5-(3-(difluoromethyl)phenyl)-2,2-diethyl-2,3-dihydro-1H-inden-1-yl)carbamate;
(S)-quinuclidin-3-yl (5-(3-(dimethylamino)phenyl)-2,2-diethyl-2,3-dihydro-1H-inden-1-yl)carbamate;
(S)-quinuclidin-3-yl (5-(4-ethoxy-3,5-dimethylphenyl)-2,2-diethyl-2,3-dihydro-1H-inden-1-yl)carbamate;
(S)-quinuclidin-3-yl (5-(3,5-dimethyl-4-propoxyphenyl)-2,2-diethyl-2,3-dihydro-1H-inden-1-yl)carbamate;
(S)-quinuclidin-3-yl (5-(4-butoxy-3,5-dimethylphenyl)-2,2-diethyl-2,3-dihydro-1H-inden-1-yl)carbamate;
(S)-quinuclidin-3-yl (2,2-diethyl-5-(thiophen-3-yl)-2,3-dihydro-1H-inden-1-yl)carbamate;
(S)-quinuclidin-3-yl (2,2-diethyl-5-(furan-3-yl)-2,3-dihydro-1H-inden-1-yl)carbamate;
(S)-quinuclidin-3-yl (5-(6-(cyclopropylmethoxy)pyridin-3-yl)-2,2-diethyl-2,3-dihydro-1H-inden-1-yl)carbamate;
(S)-quinuclidin-3-yl (5-(2,2-difluorobenzo[d][1,3]dioxol-5-yl)-2,2-diethyl-2,3-dihydro-1H-inden-1-yl)carbamate;
(S)-quinuclidin-3-yl (2,2-diethyl-5-(2-fluoro-4-methoxyphenyl)-2,3-dihydro-1H-inden-1-yl)carbamate;
(S)-quinuclidin-3-yl (5-(3-chloro-4-isopropoxyphenyl)-2,2-diethyl-2,3-dihydro-1H-inden-1-yl)carbamate;
(S)-quinuclidin-3-yl (5-(4-ethylphenyl)-6-methoxy-2,2-dimethyl-2,3-dihydro-1H-inden-1-yl)carbamate;
(S)-quinuclidin-3-yl (6-methoxy-2,2-dimethyl-5-(4-propylphenyl)-2,3-dihydro-1H-inden-1-yl)carbamate;
(S)-quinuclidin-3-yl (5-(4-isopropylphenyl)-6-methoxy-2,2-dimethyl-2,3-dihydro-1H-inden-1-yl)carbamate;
(S)-quinuclidin-3-yl (5-(4-isobutylphenyl)-6-methoxy-2,2-dimethyl-2,3-dihydro-1H-inden-1-yl)carbamate;
(S)-quinuclidin-3-yl (5-(4-(tert-butyl)phenyl)-6-methoxy-2,2-dimethyl-2,3-dihydro-1H-inden-1-yl)carbamate;
(S)-quinuclidin-3-yl (5-(4-cyclopropylphenyl)-6-methoxy-2,2-dimethyl-2,3-dihydro-1H-inden-1-yl)carbamate;
(S)-quinuclidin-3-yl (5-(4-ethoxyphenyl)-6-methoxy-2,2-dimethyl-2,3-dihydro-1H-inden-1-yl)carbamate;
(S)-quinuclidin-3-yl (6-methoxy-2,2-dimethyl-5-(4-propoxyphenyl)-2,3-dihydro-1H-inden-1-yl)carbamate;
(S)-quinuclidin-3-yl (5-(4-isopropoxyphenyl)-6-methoxy-2,2-dimethyl-2,3-dihydro-1H-inden-1-yl)carbamate;
(S)-quinuclidin-3-yl (5-(3,5-dimethyl-4-propoxyphenyl)-6-methoxy-2,2-dimethyl-2,3-dihydro-1H-inden-1-yl)carbamate;
(S)-quinuclidin-3-yl (5-(3-chloro-4-isopropoxyphenyl)-6-methoxy-2,2-dimethyl-2,3-dihydro-1H-inden-1-yl)carbamate;
(S)-quinuclidin-3-yl (5-(2-fluoro-4-methoxyphenyl)-6-methoxy-2,2-dimethyl-2,3-dihydro-1H-inden-1-yl)carbamate;
(S)-quinuclidin-3-yl (5-(4-butoxyphenyl)-6-methoxy-2,2-dimethyl-2,3-dihydro-1H-inden-1-yl)carbamate;
(S)-quinuclidin-3-yl (5-(4-isobutylphenyl)-6-methoxy-2,2-dimethyl-2,3-dihydro-1H-inden-1-yl)carbamate;
(S)-quinuclidin-3-yl (5-(6-(cyclopropylmethoxy)pyridin-3-yl)-6-methoxy-2,2-dimethyl-2,3-dihydro-1H-inden-1-yl)carbamate;
(S)-quinuclidin-3-yl (5-(5-chloro-2-methoxyphenyl)-6-methoxy-2,2-dimethyl-2,3-dihydro-1H-inden-1-yl)carbamate;
(S)-quinuclidin-3-yl (5-(4-butylphenyl)-6-methoxy-2,2-dimethyl-2,3-dihydro-1H-inden-1-yl)carbamate;
(S)-quinuclidin-3-yl (6-methoxy-5-(4-methoxy-3,5-dimethylphenyl)-2,2-dimethyl-2,3-dihydro-1H-inden-1-yl)carbamate;
(S)-quinuclidin-3-yl (5-(4-ethoxy-3,5-dimethylphenyl)-6-methoxy-2,2-dimethyl-2,3-dihydro-1H-inden-1-yl)carbamate;
(S)-quinuclidin-3-yl (5-(4-butoxy-3,5-dimethylphenyl)-6-methoxy-2,2-dimethyl-2,3-dihydro-1H-inden-1-yl)carbamate;
(S)-quinuclidin-3-yl (6-methoxy-5-(2-methoxypyridin-4-yl)-2,2-dimethyl-2,3-dihydro-1H-inden-1-yl)carbamate;
(S)-quinuclidin-3-yl (5-(3-fluorophenyl)-6-methoxy-2,2-dimethyl-2,3-dihydro-1H-inden-1-yl)carbamate;
(S)-quinuclidin-3-yl (5-(3-ethylphenyl)-6-methoxy-2,2-dimethyl-2,3-dihydro-1H-inden-1-yl)carbamate;
(S)-quinuclidin-3-yl (5-(3-isobutylphenyl)-6-methoxy-2,2-dimethyl-2,3-dihydro-1H-inden-1-yl)carbamate;
(S)-quinuclidin-3-yl (5-(3-isopropylphenyl)-6-methoxy-2,2-dimethyl-2,3-dihydro-1H-inden-1-yl)carbamate;
(S)-quinuclidin-3-yl (5-(3-(tert-butyl)phenyl)-6-methoxy-2,2-dimethyl-2,3-dihydro-1H-inden-1-yl)carbamate;
(S)-quinuclidin-3-yl (6-methoxy-5-(3-methoxyphenyl)-2,2-dimethyl-2,3-dihydro-1H-inden-1-yl)carbamate;
(S)-quinuclidin-3-yl (5-(3-ethoxyphenyl)-6-methoxy-2,2-dimethyl-2,3-dihydro-1H-inden-1-yl)carbamate;
(S)-quinuclidin-3-yl (6-methoxy-2,2-dimethyl-5-(3-propoxyphenyl)-2,3-dihydro-1H-inden-1-yl)carbamate;
(S)-quinuclidin-3-yl (5-(3-isopropoxyphenyl)-6-methoxy-2,2-dimethyl-2,3-dihydro-1H-inden-1-yl)carbamate;
(S)-quinuclidin-3-yl (5-(3-butoxyphenyl)-6-methoxy-2,2-dimethyl-2,3-dihydro-1H-inden-1-yl)carbamate;
(S)-quinuclidin-3-yl (5-(3-isobutoxyphenyl)-6-methoxy-2,2-dimethyl-2,3-dihydro-1H-inden-1-yl)carbamate;
(S)-quinuclidin-3-yl (5-(2,5-dimethoxyphenyl)-6-methoxy-2,2-dimethyl-2,3-dihydro-1H-inden-1-yl)carbamate;
(S)-quinuclidin-3-yl (5-(3,5-dimethoxyphenyl)-6-methoxy-2,2-dimethyl-2,3-dihydro-1H-inden-1-yl)carbamate;
(S)-quinuclidin-3-yl (5-(2-chloro-4-methoxyphenyl)-6-methoxy-2,2-dimethyl-2,3-dihydro-1H-inden-1-yl)carbamate;
(S)-quinuclidin-3-yl (5-(4-ethoxy-3-fluorophenyl)-6-methoxy-2,2-dimethyl-2,3-dihydro-1H-inden-1-yl)carbamate;
(S)-quinuclidin-3-yl (5-(4-isopropoxy-3,5-dimethylphenyl)-6-methoxy-2,2-dimethyl-2,3-dihydro-1H-inden-1-yl)carbamate;
(S)-quinuclidin-3-yl (5-(4-ethylphenyl)-6-fluoro-2,2-dimethyl-2,3-dihydro-1H-inden-1-yl)carbamate;
(S)-quinuclidin-3-yl (6-fluoro-2,2-dimethyl-5-(4-propylphenyl)-2,3-dihydro-1H-inden-1-yl)carbamate;
(S)-quinuclidin-3-yl (6-fluoro-5-(4-isopropylphenyl)-2,2-dimethyl-2,3-dihydro-1H-inden-1-yl)carbamate;
(S)-quinuclidin-3-yl (6-fluoro-5-(4-isobutylphenyl)-2,2-dimethyl-2,3-dihydro-1H-inden-1-yl)carbamate;
(S)-quinuclidin-3-yl (5-(4-(tert-butyl)phenyl)-6-fluoro-2,2-dimethyl-2,3-dihydro-1H-inden-1-yl)carbamate;
(S)-quinuclidin-3-yl (5-(4-cyclopropylphenyl)-6-fluoro-2,2-dimethyl-2,3-dihydro-1H-inden-1-yl)carbamate;
(S)-quinuclidin-3-yl (6-fluoro-5-(4-methoxyphenyl)-2,2-dimethyl-2,3-dihydro-1H-inden-1-yl)carbamate;
(S)-quinuclidin-3-yl (5-(4-ethoxyphenyl)-6-fluoro-2,2-dimethyl-2,3-dihydro-1H-inden-1-yl)carbamate;
(S)-quinuclidin-3-yl (6-fluoro-2,2-dimethyl-5-(4-propoxyphenyl)-2,3-dihydro-1H-inden-1-yl)carbamate;
(S)-quinuclidin-3-yl (6-fluoro-5-(4-isopropoxyphenyl)-2,2-dimethyl-2,3-dihydro-1H-inden-1-yl)carbamate;
(S)-quinuclidin-3-yl (5-(3-chloro-4-isopropoxyphenyl)-6-fluoro-2,2-dimethyl-2,3-dihydro-1H-inden-1-yl)carbamate;
(S)-quinuclidin-3-yl (6-fluoro-5-(2-fluoro-4-methoxyphenyl)-2,2-dimethyl-2,3-dihydro-1H-inden-1-yl)carbamate;
(S)-quinuclidin-3-yl (5-(4-butoxyphenyl)-6-fluoro-2,2-dimethyl-2,3-dihydro-1H-inden-1-yl)carbamate;
(S)-quinuclidin-3-yl (6-fluoro-5-(4-isobutoxyphenyl)-2,2-dimethyl-2,3-dihydro-1H-inden-1-yl)carbamate;
(S)-quinuclidin-3-yl (5-(6-(cyclopropylmethoxy)pyridin-3-yl)-6-fluoro-2,2-dimethyl-2,3-dihydro-1H-inden-1-yl)carbamate;
(S)-quinuclidin-3-yl (5-(5-chloro-2-methoxyphenyl)-6-fluoro-2,2-dimethyl-2,3-dihydro-1H-inden-1-yl)carbamate;
(S)-quinuclidin-3-yl (5-(4-butylphenyl)-6-fluoro-2,2-dimethyl-2,3-dihydro-1H-inden-1-yl)carbamate;
(S)-quinuclidin-3-yl (6-fluoro-5-(4-methoxy-3,5-dimethylphenyl)-2,2-dimethyl-2,3-dihydro-1H-inden-1-yl)carbamate;
(S)-quinuclidin-3-yl (5-(4-ethoxy-3,5-dimethylphenyl)-6-fluoro-2,2-dimethyl-2,3-dihydro-1H-inden-1-yl)carbamate;
(S)-quinuclidin-3-yl (5-(4-butoxy-3,5-dimethylphenyl)-6-fluoro-2,2-dimethyl-2,3-dihydro-1H-inden-1-yl)carbamate;
(S)-quinuclidin-3-yl (6-fluoro-5-(2-methoxypyridin-4-yl)-2,2-dimethyl-2,3-dihydro-1H-inden-1-yl)carbamate;
(S)-quinuclidin-3-yl (6-fluoro-5-(3-fluorophenyl)-2,2-dimethyl-2,3-dihydro-1H-inden-1-yl)carbamate;
(S)-quinuclidin-3-yl (5-(3-ethylphenyl)-6-fluoro-2,2-dimethyl-2,3-dihydro-1H-inden-1-yl)carbamate;
(S)-quinuclidin-3-yl (6-fluoro-5-(3-isobutylphenyl)-2,2-dimethyl-2,3-dihydro-1H-inden-1-yl)carbamate;
(S)-quinuclidin-3-yl (6-fluoro-5-(3-isopropylphenyl)-2,2-dimethyl-2,3-dihydro-1H-inden-1-yl)carbamate;
(S)-quinuclidin-3-yl (5-(3-(tert-butyl)phenyl)-6-fluoro-2,2-dimethyl-2,3-dihydro-1H-inden-1-yl)carbamate;
(S)-quinuclidin-3-yl (6-fluoro-5-(3-methoxyphenyl)-2,2-dimethyl-2,3-dihydro-1H-inden-1-yl)carbamate;
(S)-quinuclidin-3-yl (5-(3-ethoxyphenyl)-6-fluoro-2,2-dimethyl-2,3-dihydro-1H-inden-1-yl)carbamate;
(S)-quinuclidin-3-yl (6-fluoro-2,2-dimethyl-5-(3-propoxyphenyl)-2,3-dihydro-1H-inden-1-yl)carbamate;
(S)-quinuclidin-3-yl (6-fluoro-5-(3-isopropoxyphenyl)-2,2-dimethyl-2,3-dihydro-1H-inden-1-yl)carbamate;
(S)-quinuclidin-3-yl (5-(3-butoxyphenyl)-6-fluoro-2,2-dimethyl-2,3-dihydro-1H-inden-1-yl)carbamate;
(S)-quinuclidin-3-yl (6-fluoro-5-(3-isobutoxyphenyl)-2,2-dimethyl-2,3-dihydro-1H-inden-1-yl)carbamate;
(S)-quinuclidin-3-yl (5-(2,4-dimethoxyphenyl)-6-fluoro-2,2-dimethyl-2,3-dihydro-1H-inden-1-yl)carbamate;
(S)-quinuclidin-3-yl (5-(2,5-dimethoxyphenyl)-6-fluoro-2,2-dimethyl-2,3-dihydro-1H-inden-1-yl)carbamate;
(S)-quinuclidin-3-yl (5-(2,6-dimethoxyphenyl)-6-fluoro-2,2-dimethyl-2,3-dihydro-1H-inden-1-yl)carbamate;
(S)-quinuclidin-3-yl (5-(3,5-dimethoxyphenyl)-6-fluoro-2,2-dimethyl-2,3-dihydro-1H-inden-1-yl)carbamate;
(S)-quinuclidin-3-yl (5-(3,4-dimethoxyphenyl)-6-fluoro-2,2-dimethyl-2,3-dihydro-1H-inden-1-yl)carbamate;
(S)-quinuclidin-3-yl (5-(2-chloro-4-methoxyphenyl)-6-fluoro-2,2-dimethyl-2,3-dihydro-1H-inden-1-yl)carbamate;
(S)-quinuclidin-3-yl (5-(4-ethoxy-3-fluorophenyl)-6-fluoro-2,2-dimethyl-2,3-dihydro-1H-inden-1-yl)carbamate;
(S)-quinuclidin-3-yl (6-fluoro-5-(4-isopropoxy-3,5-dimethylphenyl)-2,2-dimethyl-2,3-dihydro-1H-inden-1-yl)carbamate;
(S)-quinuclidin-3-yl (5′-(4-fluorophenyl)-1′,3′-dihydrospiro[cyclopropane-1,2′-inden]-1′-yl)carbamate;
(S)-quinuclidin-3-yl (5′-(4-chlorophenyl)-1′,3′-dihydrospiro[cyclopropane-1,2′-inden]-1′-yl)carbamate;
(S)-quinuclidin-3-yl (5′-(4-(trifluoromethyl)phenyl)-1′,3′-dihydrospiro[cyclopropane-1,2′-inden]-1′-yl)carbamate;
(S)-quinuclidin-3-yl (5′-(4-(trifluoromethoxy)phenyl)-1′,3′-dihydrospiro[cyclopropane-1,2′-inden]-1′-yl)carbamate;
(S)-quinuclidin-3-yl (5′-(3-chlorophenyl)-1′,3′-dihydrospiro[cyclopropane-1,2′-inden]-1′-yl)carbamate;
(S)-quinuclidin-3-yl (5′-(3-(trifluoromethyl)phenyl)-1′,3′-dihydrospiro[cyclopropane-1,2′-inden]-1′-yl)carbamate;
(S)-quinuclidin-3-yl (5′-(4-(2-methoxyethoxy)phenyl)-1′,3′-dihydrospiro[cyclopropane-1,2′-inden]-1′-yl)carbamate;
(S)-quinuclidin-3-yl (5′-(3-(2-methoxyethoxy)phenyl)-1′,3′-dihydrospiro[cyclopropane-1,2′-inden]-1′-yl)carbamate;
(S)-quinuclidin-3-yl (5′-(4-(methoxymethoxy)phenyl)-1′,3′-dihydrospiro[cyclopropane-1,2′-inden]-1′-yl)carbamate;
(S)-quinuclidin-3-yl (5′-(3-(methoxymethoxy)phenyl)-1′,3′-dihydrospiro[cyclopropane-1,2′-inden]-1′-yl)carbamate;
(S)-quinuclidin-3-yl (5-(3-chlorophenyl)-3,3-dimethyl-2,3-dihydro-1H-inden-1-yl)carbamate;
(S)-quinuclidin-3-yl (5-(2-chlorophenyl)-3,3-dimethyl-2,3-dihydro-1H-inden-1-yl)carbamate;
(S)-quinuclidin-3-yl (3,3-dimethyl-5-(3-(trifluoromethyl)phenyl)-2,3-dihydro-1H-inden-1-yl)carbamate;
(S)-quinuclidin-3-yl (3,3-dimethyl-5-(3-(trifluoromethoxy)phenyl)-2,3-dihydro-1H-inden-1-yl)carbamate;
(S)-quinuclidin-3-yl (3,3-dimethyl-5-(2-(trifluoromethoxy)phenyl)-2,3-dihydro-1H-inden-1-yl)carbamate;
(S)-quinuclidin-3-yl (6-(3-(trifluoromethyl)phenyl)-2,3-dihydrobenzofuran-3-yl)carbamate;
(S)-quinuclidin-3-yl (6-(3-(trifluoromethoxy)phenyl)-2,3-dihydrobenzofuran-3-yl)carbamate;
(S)-quinuclidin-3-yl (5-(3-fluorophenyl)-2,2-dimethyl-2,3-dihydrobenzofuran-3-yl)carbamate;
(S)-quinuclidin-3-yl (6-(3-fluorophenyl)-2,2-dimethyl-2,3-dihydrobenzofuran-3-yl)carbamate;
(S)-quinuclidin-3-yl (6-(3-chlorophenyl)-2,2-dimethyl-2,3-dihydrobenzofuran-3-yl)carbamate;
(S)-quinuclidin-3-yl (2,2-dimethyl-6-(3-(trifluoromethyl)phenyl)-2,3-dihydrobenzofuran-3-yl)carbamate;
(S)-quinuclidin-3-yl (2,2-dimethyl-6-(3-(trifluoromethoxy)phenyl)-2,3-dihydrobenzofuran-3-yl)carbamate; and
(S)-quinuclidin-3-yl (6-(3-(2-methoxyethoxy)phenyl)-2,2-dimethyl-2,3-dihydrobenzofuran-3-yl)carbamate.

The compound of Formula 1 or pharmaceutically acceptable salt thereof may be in the form of cis- or trans-geometrical isomer. The compound of Formula 1 or its pharmaceutically acceptable salt comprises both cis- and trans-geometrical isomers, unless otherwise indicated. The compound of Formula 1 or pharmaceutically acceptable salt thereof may also have substituents containing asymmetric atom and therefore be in the form of racemic mixture (RS) or in the form of an optical isomer, such as (R) or (S) isomer. The compound of Formula 1 or pharmaceutically acceptable salt thereof comprises both the racemic mixture (RS) and each optical isomer such as (R) or (S) isomer, unless otherwise indicated. In addition, the compound of Formula 1 or pharmaceutically acceptable salt thereof may have two or more chiral centers and therefore be in the form of one or more diastereomers or in the form of the mixture thereof. The compound of Formula 1 or pharmaceutically acceptable salt thereof comprises both each diastereomer and the mixture thereof, unless otherwise indicated.

The compound of Formula 1 of the present invention may be in a pharmaceutically acceptable salt form. The salt may be a conventional acid addition salt form, which includes e.g., salts derived from an inorganic acid such as hydrochloric acid, sulfuric acid, nitric acid, phosphoric acid, perchloric acid, or hydrobromic acid; and salts derived from an organic acid such as formic acid, acetic acid, propionic acid, oxalic acid, succinic acid, benzoic acid, citric acid, maleic acid, malonic acid, malic acid, tartaric acid, gluconic acid, lactic acid, gentisic acid, fumaric acid, lactobionic acid, salicylic acid, phthalic acid, embonic acid, aspartic acid, glutamic acid, or acetylsalicylic acid. And, the salt also includes, e.g., salts derived from an amino acid such as glycine, alanine, valine, isoleucine, serine, cysteine, cystine, aspartic acid, glutamine, lysine, arginine, tyrosine, or proline. In addition, the salt includes, e.g., salts derived from a sulfonic acid such as methanesulfonic acid, ethanesulfonic acid, benzenesulfonic acid, or toluenesulfonic acid.

The compound of Formula 1 or pharmaceutically acceptable salt thereof according the present invention may be prepared according to various methods.

For example, the compound of Formula 1a or pharmaceutically acceptable salt thereof according the present invention may be prepared by a process which comprises reacting a compound of Formula 2 or salt thereof with ethyl chloroformate to obtain a compound of Formula 3; coupling the compound of Formula 3 with a compound of Formula 4 to obtain a compound of Formula 5; reacting the compound of Formula 5 with a A-W-boronic acid substituted with X₁, X₂, X₃, or X₄ to obtain a compound of Formula 1a; and optionally converting the compound of Formula 1a to a pharmaceutically acceptable salt thereof, as shown in the following Reaction Scheme 1.

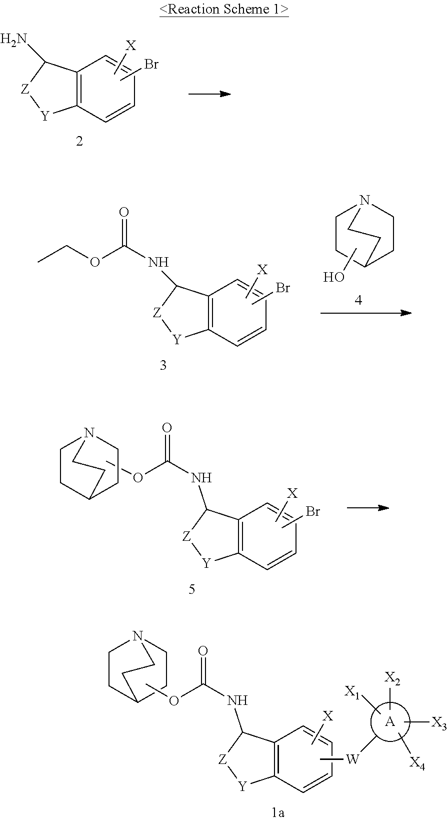

In the Reaction Scheme 1, X, Y, Z, W, A ring, X₁, X₂, X₃, and X₄ are the same as defined in the above.

The compound of Formula 2, which is commercially available or a known compound, may be synthesized according to literatures. The reaction between the compound of Formula 2 or salt thereof and ethyl chloroformate may be carried out in the presence of an organic base such as triethylamine, N,N-diisopropylethylamine, etc. or an inorganic base such as potassium carbonate, etc. The base may be used in an amount ranging from 1 to 1.5 equivalents based on 1 equivalent of the compound of Formula 2 or in an amount ranging from 3 to 5 equivalents based on 1 equivalent of the salt of the compound of Formula 2 (e.g., hydrochloride). The reaction may be carried out in a solvent such as dichloromethane or tetrahydrofuran preferably at 0° C. to room temperature.

The coupling between the compound of Formula 3 and the compound of Formula 4 (i.e., quinuclidinol) may be carried out through a condensation reaction including the removal of ethanol. The reaction may be carried out preferably in the presence of a base such as sodium hydride. In addition, the reaction may be carried out in a non-polar organic solvent such as toluene at 120° C. to 140° C.

The reaction between the compound of Formula 5 and the A-W-boronic acid substituted with X₁, X₂, X₃, or X₄ may be carried out according to the Suzuki reaction. Said Suzuki reaction may be carried out using a palladium catalyst, such as tetrakis(triphenylphosphine)palladium(O) (Pd(PPh₃)₄), palladium(II) acetate (Pd(OAc)₂), [1,1′-bis(diphenylphosphino)ferrocene]dichloropalladium(II) (Pd(dppf)Cl₂), etc. In addition, said Suzuki reaction may be carried out in the presence of an inorganic base such as cesium carbonate (Cs₂CO₃), sodium carbonate (Na₂CO₃), potassium carbonate (K₂CO₃), potassium phosphate (K₃PO₄), etc. Said Suzuki reaction may be carried out in a non-polar organic solvent such as toluene or a polar organic solvent such as 1,4-dioxane, tetrahydrofuran, acetonitrile, 1,2-dimethoxyethane, or N,N-dimethylformamide, at 50° C. to 150° C., preferably 80° C. to 120° C. Other reaction conditions including a reaction time may be determined according to known methods for the Suzuki reaction.

The conversion of the compound of Formula 1a to a pharmaceutically acceptable salt thereof may be carried out according to conventional methods. For example, a pharmaceutically acceptable salt of the compound of Formula 1a may be prepared by dissolving the compound of Formula 1a in a water-miscible solvent such as methanol, ethanol, acetone or 1,4-dioxane and then added a free acid or a free base thereto for the crystallization thereof.

In addition, the compound of Formula 1a or pharmaceutically acceptable salt thereof according the present invention may be prepared by a process which comprises reacting a compound of Formula 4 with bis(4-nitrophenyl) carbonate to obtain a compound of Formula 7; reacting the compound of Formula 7 with a compound of Formula 2 to obtain a compound of Formula 5; reacting the compound of Formula 5 with a A-W-boronic acid substituted with X₁, X₂, X₃, or X₄ to obtain a compound of Formula 1a; and optionally converting the compound of Formula 1a to a pharmaceutically acceptable salt thereof, as shown in the following Reaction Scheme 2.

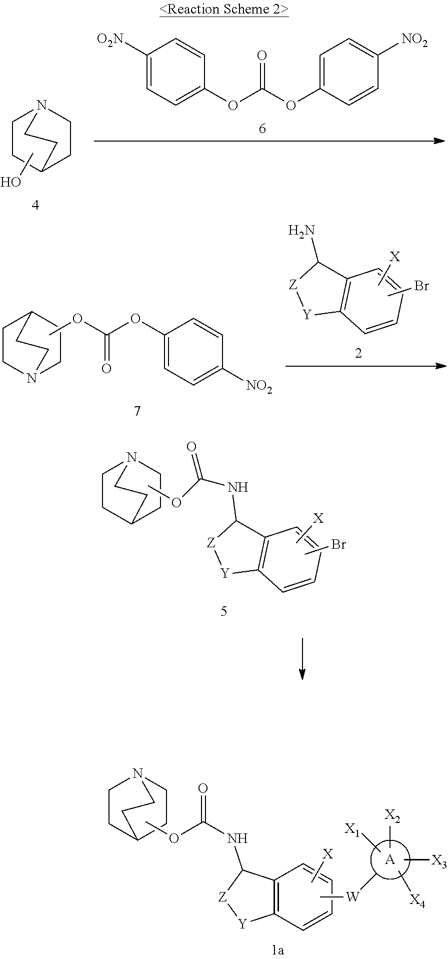

In the Reaction Scheme 2, X, Y, Z, W, A ring, X₁, X₂, X₃, and X₄ are the same as defined in the above.

The reaction between the compound of Formula 4 (i.e., quinuclidinol) and bis(4-nitrophenyl) carbonate may be carried out in a polar solvent such as N,N-dimethylformamide preferably at 0° C. to 25° C. The compound of Formula 7 may be also prepared by reacting the compound of Formula 4 with 4-nitrophenyl chloroformate, in the presence of a base such as triethylamine, in a solvent such as dichloromethane, acetonitrile, etc.

The reaction between the compound of Formula 7 and the compound of Formula 2 may be carried out in a solvent such as N,N-dimethylformamide, tetrahydrofuran or acetonitrile, in the presence of a base such as N,N-diisopropylethylamine or triethylamine, at 0° C. to 25° C.

The reaction between the compound of Formula 5 and the A-W-boronic acid substituted with X₁, X₂, X₃, or X₄ may be carried out according to the Suzuki reaction, as described in the above. In addition, the conversion of the compound of Formula 1a to a pharmaceutically acceptable salt thereof may be carried out according to conventional methods, as described in the above.

The compound having a 2,3-dihydro-1H-indene or 2,3-dihydrobenzofuran moiety according to the present invention, i.e., the compound of Formula 1 or pharmaceutically acceptable salt thereof has an excellent inhibitory activity against glucosylceramide synthase (GCS), and therefore can be usefully applied for preventing or treating treating various diseases associated with GCS.

Therefore, the present invention includes, within its scope, a pharmaceutical composition for inhibiting glucosylceramide synthase (GCS), comprising a therapeutically effective amount of the compound of Formula 1 or pharmaceutically acceptable salt thereof as an active ingredient. In an embodiment, the present invention provides a pharmaceutical composition for preventing or treating the diseases associated with GCS, such as Gaucher disease, Fabry disease, Tay-Sachs disease, Parkinson's disease, etc., comprising a therapeutically effective amount of the compound of Formula 1 or pharmaceutically acceptable salt thereof as an active ingredient.

The pharmaceutical composition of the present invention may comprise a pharmaceutically acceptable carrier, such as diluents, disintegrants, sweeteners, lubricants, or flavoring agents. The pharmaceutical composition may be formulated to an oral dosage form such as tablets, capsules, powders, granules, suspensions, emulsions, or syrups; or a parenteral dosage form such as solutions for external use, suspensions for external use, emulsions for external use, gels (e.g., ointment), inhalations, nebulizations, injections. The dosage form may be various forms, e.g., dosage forms for single administration or for multiple administrations.

The pharmaceutical composition of the present invention may comprise, for example, a diluent (e.g., lactose, corn starch, etc); a lubricant (e.g., magnesium stearate); an emulsifying agent; a suspending agent; a stabilizer; and/or an isotonic agent. If necessary, the composition further comprises sweeteners and/or flavoring agents.

The composition of the present invention may be administered orally or parenterally, including inhalant, intravenous, intraperitoneal, subcutaneous, rectal and topical routes of administration. Therefore, the composition of the present invention may be formulated into various forms such as tablets, capsules, aqueous solutions or suspensions. In the case of tablets for oral administration, carriers such as lactose, corn starch, and lubricating agents, e.g. magnesium stearate, are conventionally used. In the case of capsules for oral administration, lactose and/or dried corn starch can be used as a diluent. When an aqueous suspension is required for oral administration, the active ingredient may be combined with emulsifying and/or suspending agents. If desired, certain sweetening and/or flavoring agents may be used. For intramuscular, intraperitoneal, subcutaneous and intravenous administration, sterile solutions of the active ingredient are usually prepared, and the pH of the solutions should be suitably adjusted and buffered. For intravenous administration, the total concentration of solutes should be controlled in order to render the preparation isotonic. The composition of the present invention may be in the form of an aqueous solution containing pharmaceutically acceptable carriers, e.g., saline having a pH level of 7.4. The solutions may be introduced into a patient's intramuscular blood-stream by local bolus injection.

The compound of Formula 1 or pharmaceutically acceptable salt thereof may be administered in a therapeutically effective amount ranging from about 0.0001 mg/kg to about 100 mg/kg per day to a subject patient. Of course, the dosage may be changed according to the patient's age, weight, susceptibility, symptom, or activity of the compound.

The present invention includes, within its scope, a method for inhibiting glucosylceramide synthase (GCS) in a mammal, comprising administering a therapeutically effective amount of the compound of Formula 1 or pharmaceutically acceptable salt thereof to the mammal in need thereof. In an embodiment, the present invention provides a method for treating the diseases associated with GCS, such as Gaucher disease, Fabry disease, Tay-Sachs disease, Parkinson's disease, etc., comprising administering a therapeutically effective amount of the compound of Formula 1 or pharmaceutically acceptable salt thereof to the mammal in need thereof.

The present invention also provides a use of the compound of Formula 1 or pharmaceutically acceptable salt thereof for the manufacture of a medicament for inhibiting glucosylceramide synthase (GCS) in a mammal. In an embodiment, the present invention provides a use of the compound of Formula 1 or pharmaceutically acceptable salt thereof for the manufacture of a medicament for preventing or treating the diseases associated with GCS, such as Gaucher disease, Fabry disease, Tay-Sachs disease, Parkinson's disease, etc.

The following examples and experimental examples are provided for illustration purposes only, and are not intended to limit the scope of the invention.

In the following examples, brine refers to a saturated aqueous sodium chloride solution. Unless otherwise indicated, all temperatures are in degrees Celsius (° C.). All reactions were carried out at room temperature unless otherwise indicated.

The analyses of the compounds prepared in the following Preparations and Examples were carried out as follows: Nuclear magnetic resonance (NMR) spectrum analysis was carried out using Bruker 400 MHz spectrometer and chemical shifts thereof were analyzed in ppm. Column chromatography was carried out on silica gel (Merck, 70-230 mesh). Each starting material is a known compound which was synthesized according to literatures or purchased commercially. All reactions and chromatographic fractions were analyzed by thin layer chromatography (TLC) on a 250 nm silica gel plate and visualized with ultraviolet or iodine (I2) staining.

Preparation 1. (S)-quinuclidin-3-yl (5-bromo-2,3-dihydro-1H-inden-1-yl)carbamate
Step 1. ethyl (5-bromo-2,3-dihydro-1H-inden-1-yl)carbamate

5-Bromo-2,3-dihydro-1H-inden-1-amine (2 g, 9.4 mmol) was dissolved in dichloromethane (50 ml) and then triethylamine (1.45 ml, 10.4 mmol) and ethyl chloroformate (1 ml, 10.4 mmol) were slowly added thereto. The reaction mixture was stirred at room temperature for 4 hours. Water was added to the reaction mixture, which was then extracted with dichloromethane. The organic layer was dried over anhydrous magnesium sulfate and then filtered and concentrated under reduced pressure. The resulting residue in the pale yellow oil form (2.6 g) was used in subsequent reaction without further purification.

Step 2. (S)-quinuclidin-3-yl (5-bromo-2,3-dihydro-1H-inden-1-yl)carbamate

(S)-(+)-3-Quinuclidinol (3.5 g, 27.5 mmol) was dissolved in toluene (80 ml) and then sodium hydride (219.6 mg, 9.2 mmol) was added thereto at (° C.). The reaction mixture was stirred for 15 minutes and then ethyl (5-bromo-2,3-dihydro-1H-inden-1-yl)carbamate (2.6 g, 9.2 mmol) prepared in Step 1 was added thereto. The reaction mixture was refluxed under stirring at 140° C. for 21 hours and then cooled to room temperature. Brine was added to the reaction mixture, which was then extracted with ethyl acetate. The organic layer was washed with brine two times, dried over anhydrous magnesium sulfate, and then filtered and concentrated under reduced pressure. The resulting residue was purified by column chromatography (ethyl acetate/(methanol/ammonia water=1/1)=9/1, v/v) to give the titled compound as a white solid. (1.3 g, Yield: 46%)

¹H-NMR (400 MHz, CDCl₃) δ 7.34 (s, 1H), 7.30 (m, 1H), 7.15 (m, 1H), 5.50 (br, 1H), 5.13 (m, 1H), 4.75 (m, 2H), 3.18 (m, 2H), 2.82-2.68 (m, 7H), 1.96 (m, 1H), 1.80 (m, 1H), 1.66 (m, 1H), 1.55 (m, 1H), 1.37 (m, 1H)

Preparation 2. (S)-quinuclidin-3-yl (6-bromo-2,3-dihydro-1H-inden-1-yl)carbamate

The titled compound was prepared in accordance with the same procedures as in Preparation 1, using 6-bromo-2,3-dihydro-1H-inden-1-amine as a starting material.

¹H-NMR (400 MHz, CDCl₃) δ 7.40 (s, 1H), 7.30 (m, 1H), 7.08 (d, 1H), 5.51 (br, 1H), 5.19 (m, 1H), 4.75 (m, 2H), 3.20 (m, 2H), 2.82-2.69 (m, 7H), 1.97 (m, 1H), 1.80 (m, 1H), 1.67 (m, 1H), 1.55 (m, 1H), 1.38 (m, 1H)

Preparation 3. (S)-quinuclidin-3-yl (6-bromo-2,2-dimethyl-2,3-dihydro-1H-inden-1-yl)carbamate

The titled compound was prepared in accordance with the same procedures as in Preparation 1, using 6-bromo-2,2-dimethyl-2,3-dihydro-1H-inden-1-amine as a starting material.

¹H-NMR (400 MHz, CDCl₃) δ 7.28 (m, 2H), 7.00 (d, 1H), 4.89 (m, 1H), 4.73 (m, 2H), 3.15 (m, 1H), 2.81-2.70 (m, 7H), 1.95 (m, 1H), 1.80 (br, 1H), 1.64 (br, 1H), 1.53 (br, 1H), 1.39 (br, 1H), 0.85 (d, 6H)

Preparation 4. (S)-quinuclidin-3-yl (5-bromo-2,2-dimethyl-2,3-dihydro-1H-inden-1-yl)carbamate
Step 1. 5-bromo-2,2-dimethyl-2,3-dihydro-1H-inden-1-amine hydrochloride

5-Bromo-2,2-dimethyl-2,3-dihydro-1H-inden-1-one (100 g, 418.2 mmol) was dissolved in methanol (500 ml) and isopropanol (500 ml) and then ammonium acetate (515.8 g, 6691.5 mmol) and sodium cyanoborohydride (157.7 g, 2509.3 mmol) were slowly added thereto at room temperature. The reaction mixture was stirred at room temperature for 4 hours and then refluxed under stirring at 80° C. for 22 hours. The reaction mixture was cooled to room temperature, basified to pH>12 with a 1N sodium hydroxide solution, and then extracted with dichloromethane. The organic layer was dried over anhydrous magnesium sulfate and then filtered and concentrated under reduced pressure. Ethyl acetate was added to the resulting residue and then a hydrochloric acid solution (4M) in 1,4-dioxane was added thereto. The mixture was stirred at room temperature for 1 hour. The resulting solid was filtered under reduced pressure, washed with ethyl acetate, and then dried to give the titled compound as a white solid. (100 g, Yield: 86.5%)

¹H-NMR (400 MHz, CD₃OD) δ 7.50 (m, 2H), 7.39 (d, 1H), 4.28 (s, 1H), 3.02-2.82 (m, 2H), 1.24 (s, 3H), 1.20 (s, 3H)

Step 2. (S)-quinuclidin-3-yl (5-bromo-2,2-dimethyl-2,3-dihydro-1H-inden-1-yl)carbamate

A solution of (S)-(+)-3-quinuclidinol (38.6 g, 303.7 mmol) and bis(4-nitrophenyl) carbonate (92.4 g, 303.7 mmol) in N,N-dimethylformamide (700 ml) was stirred at room temperature for 8 hours. 5-Bromo-2,2-dimethyl-2,3-dihydro-1H-inden-1-amine hydrochloride (70 g, 253.1 mmol) prepared in Step 1 and diisopropylethylamine (132.3 ml, 759.2 mmol) were added to the solution, which was then stirred at room temperature overnight. The reaction mixture was diluted with water (500 ml), acidified to pH 1-2 with a 1N aqueous hydrochloric acid solution, and then extracted with diisopropyl ether (300 ml×2). The aqueous layer was basified to pH 12 with ammonia and then extracted with ethyl acetate. The organic layer was washed with water, dried over anhydrous magnesium sulfate, and then filtered and concentrated under reduced pressure. The resulting residue was dissolved in isopropyl acetate and then heptane was added thereto. The mixture was stirred at room temperature overnight. The resulting solid was filtered under reduced pressure, washed with heptane, and dried to give the titled compound as a white solid. (56 g, Yield: 56.3%)

¹H-NMR (400 MHz, CDCl₃) δ 7.32 (m, 2H), 7.09 (m, 1H), 4.89 (m, 1H), 4.86 (m, 1H), 4.77 (m, 1H), 3.25 (m, 1H), 2.87-2.75 (m, 2H), 2.74-2.69 (m, 5H), 2.07 (m, 1H), 1.83 (br, 1H), 1.69 (br, 1H), 1.57 (br, 1H), 1.41 (br, 1H), 1.27 (s, 3H), 0.92 (s, 3H)

Preparation 5. (S)-quinuclidin-3-yl (5-bromo-2,2-diethyl-2,3-dihydro-1H-inden-1-yl)carbamate
Step 1. 5-bromo-2,2-diethyl-2,3-dihydro-1H-inden-1-one

5-Bromo-1-indanone (20 g, 94.8 mmol) was dissolved in tetrahydrofuran (100 ml) and then 60% sodium hydride (19 g, 473.8 mmol) was added thereto at 0° C. The reaction mixture was stirred for 15 minutes and then iodoethane (19.1 ml, 236.9 mmol) was slowly added thereto at the same temperature. The reaction mixture was stirred at room temperature overnight. Water was added to the reaction mixture, which was then extracted with ethyl acetate. The organic layer was dried over anhydrous magnesium sulfate and then filtered and concentrated under reduced pressure. The resulting residue was purified by column chromatography (n-hexane/ethyl acetate=10/1, v/v) to give the titled compound as a white solid. (17 g, Yield: 67%)

¹H-NMR (400 MHz, CDCl₃) δ 7.61 (s, 1H), 7.58 (m, 1H), 7.51 (m, 1H), 2.97 (s, 2H), 1.65 (m, 2H), 1.62 (m, 2H), 0.77 (t, 6H)

Step 2. 5-bromo-2,2-diethyl-2,3-dihydro-1H-inden-1-amine hydrochloride

The titled compound was prepared in accordance with the same procedures as in Step 1 of Preparation 4, using 5-bromo-2,2-diethyl-2,3-dihydro-1H-inden-1-one prepared in Step 1.

¹H-NMR (400 MHz, MeOD) δ 7.49 (s, 1H), 7.45 (m, 2H), 4.38 (s, 1H), 3.07 (m, 1H), 2.83 (m, 1H), 1.70 (m, 2H), 1.40 (m, 2H), 1.09 (t, 3H), 0.88 (t, 3H)

Step 3. (S)-quinuclidin-3-yl (5-bromo-2,2-diethyl-2,3-dihydro-1H-inden-1-yl)carbamate

The titled compound was prepared in accordance with the same procedures as in Step 2 of Preparation 4, using 5-bromo-2,2-diethyl-2,3-dihydro-1H-inden-1-amine hydrochloride prepared in Step 2.

¹H-NMR (400 MHz, CDCl₃) δ 7.32 (m, 2H), 7.09 (m, 1H), 5.01 (m, 1H), 4.85 (m, 1H), 4.76 (m, 1H), 3.25 (m, 1H), 2.89-2.70 (m, 7H), 2.10 (br, 1H), 1.80 (br, 1H), 1.69 (br, 1H), 1.55 (m, 3H), 1.49 (m, 2H), 1.40 (br, 1H), 0.95 (m, 3H), 0.81 (m, 3H)

Preparation 6. (S)-quinuclidin-3-yl (5-bromo-6-methoxy-2,2-dimethyl-2,3-dihydro-1H-inden-1-yl)carbamate
Step 1. 5-bromo-6-methoxy-2,2-dimethyl-2,3-dihydro-1H-inden-1-one

5-Bromo-6-methoxy-2,3-dihydro-1H-inden-1-one (10 g, 43.7 mmol) was dissolved in tetrahydrofuran (50 ml) and then 60% sodium hydride (8.7 g, 218.3 mmol) was added thereto at 0° C. The reaction mixture was stirred for 15 minutes and then iodomethane (10.9 ml, 174.6 mmol) was slowly added thereto at the same temperature. The reaction mixture was stirred at room temperature overnight. Water was added to the reaction mixture, which was then extracted with ethyl acetate. The organic layer dried over anhydrous magnesium sulfate and then filtered and concentrated under reduced pressure. The resulting residue was purified by column chromatography (n-hexane/ethyl acetate=10/1, v/v) to give the titled compound as a white solid. (10 g, Yield: 89%)

¹H-NMR (400 MHz, CDCl₃) δ 7.66 (s, 1H), 7.21 (s, 1H), 3.94 (s, 3H), 2.93 (s, 2H), 1.24 (s, 6H)

Step 2. 5-bromo-6-methoxy-2,2-dimethyl-2,3-dihydro-1H-inden-1-amine hydrochloride

The titled compound was prepared in accordance with the same procedures as in Step 1 of Preparation 4, using 5-bromo-6-methoxy-2,2-dimethyl-2,3-dihydro-1H-inden-1-one prepared in Step 1.

¹H-NMR (400 MHz, MeOD) δ 7.47 (s, 1H), 7.23 (s, 1H), 4.29 (s, 1H), 4.11 (s, 3H), 2.95-2.92 (m, 1H), 2.78-2.74 (m, 1H), 1.29 (m, 3H), 0.98 (m, 3H)

Step 3. (S)-quinuclidin-3-yl (5-bromo-6-methoxy-2,2-dimethyl-2,3-dihydro-1H-inden-1-yl)carbamate

The titled compound was prepared in accordance with the same procedures as in Step 2 of Preparation 4, using 5-bromo-6-methoxy-2,2-dimethyl-2,3-dihydro-1H-inden-1-amine hydrochloride prepared in Step 2.

¹H-NMR (400 MHz, CDCl₃) δ 7.34 (s, 1H), 6.77 (m, 1H), 4.87 (s, 2H), 4.78 (m, 1H), 3.85 (m, 3H), 3.25 (m, 1H), 2.78-2.61 (m, 7H), 2.10 (br, 1H), 1.85 (br, 1H), 1.69 (br, 1H), 1.57 (br, 1H), 1.44 (br, 1H), 1.21 (m, 3H), 0.95 (m, 3H)

Preparation 7. (S)-quinuclidin-3-yl (5-bromo-6-fluoro-2,2-dimethyl-2,3-dihydro-1H-inden-1-yl)carbamate

The titled compound was prepared in accordance with the same procedures as in Preparation 6, using 5-bromo-6-fluoro-2,3-dihydro-1H-inden-1-one as a starting material.

¹H-NMR (400 MHz, CDCl₃) δ 7.34 (d, 1H), 7.01 (d, 1H), 4.91 (m, 1H), 4.84 (m, 1H), 4.69 (m, 1H), 3.25 (m, 1H), 2.93-2.72 (m, 7H), 2.10 (br, 1H), 1.85 (br, 1H), 1.70 (br, 1H), 1.59 (br, 1H), 1.40 (br, 1H), 1.27 (m, 3H), 0.92 (m, 3H)

Preparation 8. (S)-quinuclidin-3-yl (5′-bromo-1′,3′-dihydrospiro[cyclopropane-1,2′-inden]-1′-yl)carbamate
Step 1. 5′-bromospiro[cyclopropane-1,2′-inden]-1′(3′H)-one

5-Bromo-1-indanone (5 g, 23.7 mmol) was dissolved in NN-dimethylformamide (100 ml) and then 60% sodium hydride (2.7 g, 35.5 mmol) was added thereto at 0° C. The reaction mixture was stirred for 15 minutes and then 1,2-dibromomethane (5.1 ml, 35.5 mmol) was slowly added thereto at the same temperature. The reaction mixture was stirred at room temperature overnight. Water was added to the reaction mixture, which was then extracted with ethyl acetate. The organic layer was dried over anhydrous magnesium sulfate and then filtered and concentrated under reduced pressure. The resulting residue was purified by column chromatography (n-hexane/ethyl acetate=10/1, v/v) to give the titled compound as a white solid. (3.5 g, Yield: 62%)

¹H-NMR (400 MHz, CDCl₃) δ 7.69 (s, 1H), 7.65 (m, 1H), 7.55 (d, 1H), 3.15 (s, 2H), 1.47 (d, 2H), 1.19 (d, 2H)

Step 2. 5′-bromo-1′,3′-dihydrospiro[cyclopropane-1,2′-inden]-1′-amine hydrochloride

The titled compound was prepared in accordance with the same procedures as in Step 1 of Preparation 4, using 5′-bromospiro[cyclopropane-1,2′-inden]-1′(3′H)-one prepared in Step 1.

¹H-NMR (400 MHz, MeOD) δ 7.56 (s, 1H), 7.52 (m, 1H), 7.45 (m, 1H), 4.25 (s, 1H), 3.45 (m, 1H), 2.71 (m, 1H), 1.15 (m, 1H), 0.92 (m, 2H), 0.82 (m, 1H)

Step 3. (S)-quinuclidin-3-yl (5′-bromo-1′,3′-dihydrospiro[cyclopropane-1,2′-inden]-1′-yl)carbamate

The titled compound was prepared in accordance with the same procedures as in Preparation 1, using 5′-bromo-1′,3′-dihydrospiro[cyclopropane-1,2′-inden]-1′-amine hydrochloride prepared in Step 2.

¹H-NMR (400 MHz, CDCl₃) δ 7.36 (m, 2H), 7.24 (m, 1H), 4.92 (m, 1H), 4.91 (m, 1H), 4.74 (m, 1H), 3.25 (m, 1H), 3.00 (m, 2H), 2.87-2.68 (m, 5H), 1.97 (br, 1H), 1.79 (m, 1H), 1.67 (br, 1H), 1.56 (m, 1H), 1.41 (br, 1H), 0.91 (m, 2H), 0.81 (m, 1H), 0.59 (m, 1H)

Preparation 9. (S)-quinuclidin-3-yl (5-bromo-3,3-dimethyl-2,3-dihydro-1H-inden-1-yl)carbamate
Step 1. 6-bromo-1,1-dimethyl-2,3-dihydro-1H-indene

A titanium(IV) chloride solution (1.0M) in dichloromethane (28.4 ml) was slowly added to dichloromethane (20 ml) at −30° C. under nitrogen atmosphere. A dimethylzinc solution (2.0M) in toluene (16.6 ml) was slowly added at the same temperature to the mixture, which was then stirred at 30 minutes. A solution of 6-bromo-2,3-dihydro-1H-inden-1-one (2 g, 9.5 mmol) in dichloromethane (10 ml) was slowly added to the reaction mixture, which was then stirred at −10° C. for 4 hours. The reaction was quenched by adding ice water to the reaction mixture, which was then extracted with ethyl acetate. The organic layer was washed with brine, dried over anhydrous magnesium sulfate, and then filtered and concentrated under reduced pressure. The resulting residue was purified by silica gel column chromatography (n-hexane/ethyl acetate=5/1, v/v) to give the titled compound as a white solid. (2.1 g, Yield: 99%)

¹H NMR (400 MHz, CDCl₃) δ 7.28 (s, 1H), 7.20 (d, 1H), 7.07 (d, 1H), 2.85 (t, 2H), 1.94 (t, 2H), 1.26 (s, 6H)

Step 2. 5-bromo-3,3-dimethyl-2,3-dihydro-1H-inden-1-one

6-Bromo-1,1-dimethyl-2,3-dihydro-1H-indene (1.9 g, 8.4 mmol) prepared in Step 1 was dissolved in acetone (20 ml) and then a solution of anhydrous magnesium sulfate (2.5 g, 21.1 mmol) in water (10 ml) was slowly added thereto. Potassium permanganate (4 g, 25.3 mmol) was slowly added to the reaction mixture, which was then refluxed under stirring for 5 hours. The reaction mixture was cooled to room temperature and then filtered and concentrated under reduced pressure. The resulting residue was diluted with ethyl acetate, washed with brine, dried over anhydrous magnesium sulfate, and then filtered and concentrated under reduced pressure. The resulting residue was purified by silica gel column chromatography (n-hexane/ethyl acetate=5/1, v/v) to give the titled compound as a white solid. (1.85 g, Yield: 92%)

¹H NMR (400 MHz, CDCl₃) δ 7.65 (s, 1H), 7.58-7.47 (m, 2H), 2.58 (s, 2H), 1.42 (s, 6H)

Step 3. 5-bromo-3,3-dimethyl-2,3-dihydro-1H-inden-1-amine hydrochloride

The titled compound was prepared in accordance with the same procedures as in Step 1 of Preparation 4, using 5-bromo-3,3-dimethyl-2,3-dihydro-1H-inden-1-one prepared in Step 2. The product was used in subsequent reaction without further purification.

Step 4. ethyl (5-bromo-3,3-dimethyl-2,3-dihydro-1H-inden-1-yl)carbamate

The titled compound was prepared in accordance with the same procedures as in Step 1 of Preparation 1, using 5-bromo-3,3-dimethyl-2,3-dihydro-1H-inden-1-aminehydrochloride prepared in Step 3.

¹H NMR (400 MHz, CDCl₃) δ 7.35 (d, 1H), 7.30 (s, 1H), 7.16 (d, 1H), 5.24(q, 1H), 4.81(brd, 1H), 4.17(q, 2H), 2.46 (m, 1H), 1.73-1.68 (m, 1H), 1.37 (s, 3H), 1.28 (t, 3H), 1.22 (s, 3H)

Step 5. (S)-quinuclidin-3-yl (5-bromo-3,3-dimethyl-2,3-dihydro-1H-inden-1-yl)carbamate

The titled compound was prepared in accordance with the same procedures as in Step 2 of Preparation 1, using ethyl (5-bromo-3,3-dimethyl-2,3-dihydro-1H-inden-1-yl)carbamate prepared in Step 4.

¹H NMR (400 MHz, MeOD) δ 7.38-7.33 (m, 2H), 7.14 (dd, 1H), 5.14 (t, 1H), 4.80-4.75 (m, 1H), 3.30-3.21 (m, 1H), 2.90-2.69 (m, 5H), 2.39-2.32 (m, 1H), 2.11-2.04 (m, 1H), 1.99-1.90 (m, 1H), 1.84-1.74 (m, 2H), 1.70-1.61 (m, 1H), 1.55-1.45 (m, 1H), 1.39 (s, 3H), 1.22 (s, 3H)

Preparation 10. (S)-quinuclidin-3-yl (6-bromo-2,3-dihydrobenzofuran-3-yl)carbamate

The titled compound was prepared in accordance with the same procedures as in Preparation 1, using 6-bromo-2,3-dihydrobenzofuran-3-amine a starting material.

¹H-NMR (400 MHz, CDCl₃) δ 7.21 (t, 1H), 7.08-7.06 (m, 1H), 7.02 (s, 1H), 5.35-5.31 (m, 2H), 4.73-4.68 (m, 2H), 4.41-4.37 (m, 1H), 3.24-3.19 (m, 1H), 2.82-2.66 (m, 5H), 2.05-1.98 (m, 1H), 1.75-1.66 (m, 2H), 1.56-1.55 (m, 1H), 1.40-1.38 (m, 1H)

Preparation 11. (S)-quinuclidin-3-yl (5-bromo-2,2-dimethyl-2,3-dihydrobenzofuran-3-yl)carbamate
Step 1. 5-bromo-2,2-dimethylbenzofuran-3(2H)-one

5-Bromobenzofuran-3-one (2.75 g, 12.9 mmol) was dissolved in tetrahydrofuran (43 ml) and then 60% sodium hydride (2.07 g, 51.6 mmol) was added thereto at 0° C. The reaction mixture was stirred for 15 minutes and then iodomethane (9.15 g, 64.5 mmol) was slowly added thereto at the same temperature. The reaction mixture was stirred at room temperature overnight. Water was added to the reaction mixture, which was then extracted with ethyl acetate. The organic layer was dried over anhydrous magnesium sulfate, and then filtered and concentrated under reduced pressure. The resulting residue was purified by column chromatography (n-hexane/ethyl acetate=10/1, v/v) to give the titled compound as a white solid. (2.5 g, Yield: 80%)

¹H-NMR (400 MHz, CDCl₃) δ 7.81 (s, 1H), 7.79 (d, 1H), 6.98 (d, 1H), 1.47 (s, 6H)

Step 2. 5-bromo-2,2-dimethyl-2,3-dihydrobenzofuran-3-amine

The titled compound was prepared in accordance with the same procedures as in Step 1 of Preparation 4, using 5-bromo-2,2-dimethylbenzofuran-3(2H)-one prepared in Step 1.

¹H-NMR (400 MHz, CDCl₃) δ 7.40 (s, 1H), 7.27 (d, 1H), 6.63 (d, 1H), 4.12 (s, 1H), 1.41 (d, 6H)

Step 3. (S)-quinuclidin-3-yl (5-bromo-2,2-dimethyl-2,3-dihydrobenzofuran-3-yl)carbamate

The titled compound was prepared in accordance with the same procedures as in Preparation 1, using 5-bromo-2,2-dimethyl-2,3-dihydrobenzofuran-3-amine prepared in Step 2.

¹H-NMR (400 MHz, CDCl₃) δ 7.40 (d, 1H), 7.31 (d, 1H), 6.65 (d, 1H), 5.36 (t, 1H), 5.00 (d, 1H), 4.78-4.69 (m, 1H), 3.25-3.15 (m, 1H), 2.89-2.59 (m, 5H), 2.06-2.00 (m, 1H), 1.83-1.74 (m, 1H), 1.72-1.63 (m, 1H), 1.60-1.51 (m, 1H), 1.48 (s, 3H), 1.41-1.36 (m, 4H)

Preparation 12. (S)-quinuclidin-3-yl (6-bromo-2,2-dimethyl-2,3-dihydrobenzofuran-3-yl)carbamate
Step 1. 6-bromo-2,2-dimethylbenzofuran-3(2H)-one

The titled compound was prepared in accordance with the same procedures as in Step 1 of Preparation 11, using 6-bromobenzofuran-3(2H)-one as a starting material.

¹H-NMR (400 MHz, CDCl₃) δ 7.53 (d, 1H), 7.30-7.27 (m, 1H), 7.21 (dd, 1H), 1.47 (s, 6H)

Step 2. (S)-quinuclidin-3-yl (6-bromo-2,2-dimethyl-2,3-dihydrobenzofuran-3-yl)carbamate

The titled compound was prepared in accordance with the same procedures as in Preparation 4, using 6-bromo-2,2-dimethylbenzofuran-3(2H)-one prepared in Step 1.

¹H-NMR (400 MHz, CDCl₃) δ 7.14 (dd, 1H), 7.05-6.99 (m, 1H), 6.91 (s, 1H), 5.59-5.46 (m, 1H), 4.94 (d, 1H), 4.77-4.66 (m, 1H), 3.27-3.14 (m, 1H), 2.91-2.61 (m, 5H), 2.12-2.01 (m, 1H), 1.86-1.64 (m, 2H), 1.61-1.52 (m, 1H), 1.45 (s, 3H), 1.43-1.32 (m, 4H)

### Example 1. (S)-quinuclidin-3-yl (5-(4-fluorophenyl)-2,3-dihydro-1H-inden-1-yl)carbamate

To a mixture of (S)-quinuclidin-3-yl (5-bromo-2,3-dihydro-1H-inden-1-yl)carbamate (30 mg, 0.082 mmol) prepared in Preparation 1,4-fluorophenylboronic acid (17.2 mg, 0.12 mmol), potassium phosphate (52.3 mg, 0.25 mmol), and tetrakis(triphenylphosphine)palladium(0) (9.4 mg, 10 mol %), were slowly added 1,4-dioxane (1 ml) and water (0.5 ml). The reaction mixture was refluxed under stirring at 120° C. overnight and then cooled to room temperature. Water was added to the reaction mixture, which was then extracted with ethyl acetate. The organic layer was dried over anhydrous magnesium sulfate and then filtered and concentrated under reduced pressure. The resulting residue was purified by silica gel column chromatography (ethyl acetate/(methanol/ammonia water=1/1)=9/1, v/v) to give the titled compound as a white solid. (19 mg, Yield: 64%)

¹H-NMR (400 MHz, CDCl₃) δ 7.55 (m, 2H), 7.42 (m, 3H), 7.13 (m, 2H), 5.29 (m, 1H), 5.00 (m, 1H), 4.81 (br, 1H), 3.28 (m, 1H), 3.05 (m, 1H), 2.94 (m, 3H), 2.82 (m, 3H), 2.65 (m, 1H), 2.06 (m, 1H), 1.93-1.88 (m, 2H), 1.71 (m, 1H), 1.60 (br, 1H), 1.42 (br, 1H)

### Examples 2 to 24

The titled compounds of Examples 2 to 24 were prepared in accordance with the same procedures as in Example 1, using (S)-quinuclidin-3-yl (5-bromo-2,3-dihydro-1H-inden-1-yl)carbamate prepared in Preparation 1; and the corresponding substituted-phenylboronic acids, respectively.

### Example 2. (S)-quinuclidin-3-yl (5-(4-chlorophenyl)-2,3-dihydro-1H-inden-1-yl)carbamate

¹H-NMR (400 MHz, CDCl₃) δ 7.52 (m, 2H), 7.42 (m, 5H), 5.29 (m, 1H), 5.01 (m, 1H), 4.80 (br, 1H), 3.28 (m, 1H), 3.03 (m, 1H), 2.85 (m, 3H), 2.78-2.67 (m, 4H), 2.09 (m, 1H), 1.98 (m, 1H), 1.87 (br, 1H), 1.71 (br, 1H), 1.60 (br, 1H), 1.41 (br, 1H)

### Example 3. (S)-quinuclidin-3-yl (5-(4-(trifluoromethyl)phenyl)-2,3-dihydro-1H-inden-1-yl)carbamate

¹H-NMR (400 MHz, CDCl₃) δ 7.69 (s, 4H), 7.47 (m, 3H), 5.31 (m, 1H), 5.01 (m, 1H), 4.81 (br, 1H), 3.31 (m, 1H), 3.04 (m, 1H), 2.94 (m, 3H), 2.78 (m, 4H), 2.03 (m, 2H), 1,91(m, 1H), 1.71 (br, 1H), 1.60 (br, 1H), 1.41 (br, 1H)

### Example 4. (S)-quinuclidin-3-yl (5-(4-(trifluoromethoxy)phenyl)-2,3-dihydro-1H-inden-1-yl)carbamate

¹H-NMR (400 MHz, CDCl₃) δ 7.58 (d, 2H), 7.43 (m, 3H), 7.29 (d, 2H), 5.29 (m, 1H), 4.99 (m, 1H), 4.80 (br, 1H), 3.31 (m, 1H), 3.05 (m, 1H), 2.95-2.75 (m, 7H), 2.07 (m, 1H), 1.84 (m, 2H), 1.71 (br, 1H), 1.60 (br, 1H), 1.41 (br, 1H)

### Example 5. (S)-quinuclidin-3-yl (5-(3-fluorophenyl)-2,3-dihydro-1H-inden-1-yl)carbamate

¹H-NMR (400 MHz, CDCl₃) δ 7.70 (m, 1H), 7.45 (m, 5H), 7.05 (m, 1H), 5.28 (m, 1H), 4.98 (m, 1H), 4.80 (br, 1H), 3.31 (m, 1H), 2.94 (m, 1H), 2.82-2.75 (m, 7H), 2.09 (m, 1H), 1.82 (m, 2H), 1.71 (br, 1H), 1.60 (br, 1H), 1.42 (br, 1H)

### Example 6. (S)-quinuclidin-3-yl (5-(3-chlorophenyl)-2,3-dihydro-1H-inden-1-yl)carbamate

¹H-NMR (400 MHz, CDCl₃) δ 7.57 (s, 1H), 7.45 (m, 4H), 7.34 (m, 2H), 5.29 (m, 1H), 4.98 (m, 1H), 4.81 (m, 1H), 3.30 (m, 1H), 3.04 (m, 1H), 2.95-2.66 (m, 7H), 2.09 (m, 1H), 1.82 (m, 2H), 1.71 (br, 1H), 1.60 (br, 1H), 1.42 (br, 1H)

### Example 7. (S)-quinuclidin-3-yl (5-(3-(trifluoromethyl)phenyl)-2,3-dihydro-1H-inden-1-yl)carbamate

¹H-NMR (400 MHz, CDCl₃) δ 7.82 (s, 1H), 7.76 (d, 1H), 7.58 (m, 2H), 7.47 (m, 3H), 5.30 (m, 1H), 4.98 (m, 1H), 4.81 (m, 1H), 3.30 (m, 1H), 3.05 (m, 1H), 3.05-2.68 (m, 7H), 2.09 (m, 1H), 1.91 (br, 1H), 1.79 (m, 1H), 1.72 (br, 1H), 1.60 (br, 1H), 1.42 (br, 1H)

### Example 8. (S)-quinuclidin-3-yl (5-(3-(trifluoromethoxy)phenyl)-2,3-dihydro-1H-inden-1-yl)carbamate

¹H-NMR (400 MHz, CDCl₃) δ 7.52-7.42 (m, 6H), 7.21 (m, 1H), 5.29 (m, 1H), 5.01 (m, 1H), 4.81 (m, 1H), 3.31 (m, 1H), 3.04 (m, 1H), 2.95-2.75 (m, 7H), 2.09 (m, 1H), 1.92 (m, 1H), 1.84 (br, 1H), 1.71 (br, 1H), 1.60 (br, 1H), 1.41 (br, 1H)

### Example 9. (S)-quinuclidin-3-yl (5-(2-fluorophenyl)-2,3-dihydro-1H-inden-1-yl)carbamate

¹H-NMR (400 MHz, CDCl₃) δ 7.42 (m, 3H), 7.28-7.16 (m, 4H), 5.29 (m, 1H), 4.98 (m, 1H), 4.81 (m, 1H), 3.29 (m, 1H), 2.95 (m, 1H), 2.89-2.66 (m, 7H), 2.06 (m, 1H), 1.83 (m, 1H), 1.74 (m, 2H), 1.60 (br, 1H), 1.41 (br, 1H)

### Example 10. (S)-quinuclidin-3-yl (5-(2-chlorophenyl)-2,3-dihydro-1H-inden-1-yl)carbamate

¹H-NMR (400 MHz, CDCl₃) δ 7.49 (d, 1H), 7.41 (d, 1H), 7.32 (m, 5H), 5.32 (m, 1H), 4.99 (m, 1H), 4.81 (br, 1H), 3.30 (m, 1H), 3.03 (m, 1H), 2.95-2.67 (m, 7H), 2.06 (m, 1H), 1.91 (br, 1H), 1.83 (br, 1H), 1.73 (m, 1H), 1.61 (br, 1H), 1.41 (br, 1H)

### Example 11. (S)-quinuclidin-3-yl (5-(2-(trifluoromethyl)phenyl)-2,3-dihydro-1H-inden-1-yl)carbamate

¹H-NMR (400 MHz, CDCl₃) δ 7.75 (d, 1H), 7.54 (m, 1H), 7.49 (m, 1H), 7.36 (m, 4H), 5.31 (m, 1H), 5.12 (m, 1H), 4.81 (br, 1H), 3.29 (m, 1H), 3.01 (m, 1H), 2.93-2.66 (m, 7H), 2.09 (br, 1H), 1.95 (m, 1H), 1.88 (br, 1H), 1.71 (br, 1H), 1.60 (br, 1H), 1.41 (br, 1H)

### Example 12. (S)-quinuclidin-3-yl (5-(2-(trifluoromethoxy)phenyl)-2,3-dihydro-1H-inden-1-yl)carbamate

¹H-NMR (400 MHz, CDCl₃) δ 7.41 (m, 6H), 7.26 (m, 1H), 5.29 (m, 1H), 5.04 (m, 1H), 4.81 (m, 1H), 3.31 (m, 1H), 3.02 (m, 1H), 2.94-2.76 (m, 7H), 2.06 (m, 1H), 1.84 (m, 2H), 1.71 (br, 1H), 1.61 (br, 1H), 1.42 (br, 1H)

### Example 13. (S)-quinuclidin-3-yl (5-(p-tolyl)-2,3-dihydro-1H-inden-1-yl)carbamate

¹H-NMR (400 MHz, CDCl₃) δ 7.46 (m, 5H), 7.25 (m, 2H), 5.29 (m, 1H), 4.98 (m, 1H), 4.80 (m, 1H), 3.30 (m, 1H), 3.04 (m, 1H), 2.96-2.67 (m, 7H), 2.40 (s, 3H), 2.09 (m, 1H), 1.85 (m, 2H), 1.71 (br, 1H), 1.60 (br, 1H), 1.42 (br, 1H)

### Example 14. (S)-quinuclidin-3-yl (5-(4-(difluoromethoxy)phenyl)-2,3-dihydro-1H-inden-1-yl)carbamate

¹H-NMR (400 MHz, CDCl₃) δ 7.58 (m, 2H), 7.41 (m, 3H), 7.20 (d, 2H), 6.75-6.38 (t, 1H), 5.29 (m, 1H), 4.98 (m, 1H), 4.80 (m, 1H), 3.30 (m, 1H), 2.95-2.66 (m, 7H), 2.08 (m, 1H), 1.85 (m, 2H), 1.71 (br, 1H), 1.60 (br, 1H), 1.42 (br, 1H)

### Example 15. (S)-quinuclidin-3-yl (5-(3,4-difluorophenyl)-2,3-dihydro-1H-inden-1-yl)carbamate

¹H-NMR (400 MHz, CDCl₃) δ 7.40 (m, 4H), 7.23 (m, 2H), 5.28 (m, 1H), 5.00 (m, 1H), 4.80 (m, 1H), 3.30 (m, 1H), 3.03 (m, 1H), 2.94-2.66 (m, 7H), 2.06 (m, 1H), 1.87 (m, 2H), 1.71 (br, 1H), 1.60 (br, 1H), 1.41 (br, 1H)

### Example 16. (S)-quinuclidin-3-yl (5-(2,4-difluorophenyl)-2,3-dihydro-1H-inden-1-yl)carbamate

¹H-NMR (400 MHz, CDCl₃) δ 7.40 (m, 4H), 6.96 (m, 2H), 5.30 (m, 1H), 5.00 (m, 1H), 4.80 (m, 1H), 3.28 (m, 1H), 3.03 (m, 1H), 2.95-2.66 (m, 7H), 2.06 (m, 1H), 1.82 (m, 2H), 1.71 (br, 1H), 1.60 (br, 1H), 1.41 (br, 1H)

### Example 17. (S)-quinuclidin-3-yl (5-(2,3-difluorophenyl)-2,3-dihydro-1H-inden-1-yl)carbamate

¹H-NMR (400 MHz, CDCl₃) δ 7.42 (m, 3H), 7.16 (m, 3H), 5.31 (m, 1H), 5.00 (m, 1H), 4.81 (m, 1H), 3.27 (m, 1H), 3.04 (m, 1H), 2.95-2.67 (m, 7H), 2.09 (m, 1H), 1.86 (m, 2H), 1.71 (br, 1H), 1.60 (br, 1H), 1.41 (br, 1H)

### Example 18. (S)-quinuclidin-3-yl (5-(2,5-difluorophenyl)-2,3-dihydro-1H-inden-1-yl)carbamate

¹H-NMR (400 MHz, CDCl₃) δ 7.41 (m, 3H), 7.12 (m, 2H), 7.01 (m, 1H), 5.30 (m, 1H), 5.02 (m, 1H), 4.81 (m, 1H), 3.30 (m, 1H), 3.03 (m, 1H), 2.95-2.66 (m, 7H), 2.09 (m, 1H), 1.89 (m, 2H), 1.71 (br, 1H), 1.60 (br, 1H), 1.41 (br, 1H)

### Example 19. (S)-quinuclidin-3-yl (5-(3,5-difluorophenyl)-2,3-dihydro-1H-inden-1-yl)carbamate

¹H-NMR (400 MHz, CDCl₃) δ 7.42 (m, 3H), 7.09 (d, 2H), 6.79 (t, 1H), 5.29 (m, 1H), 4.98 (m, 1H), 4.81 (m, 1H), 3.30 (m, 1H), 3.04 (m, 1H), 2.97-2.67 (m, 7H), 2.09 (m, 1H), 1.90 (m, 1H), 1.82 (m, 1H), 1.71 (br, 1H), 1.61 (br, 1H), 1.42 (br, 1H)

### Example 20. (S)-quinuclidin-3-yl (5-(2,3,4-trifluorophenyl)-2,3-dihydro-1H-inden-1-yl)carbamate

¹H-NMR (400 MHz, CDCl₃) δ 7.43 (m, 1H), 7.37 (m, 2H), 7.13 (m, 1H), 7.04 (m, 1H), 5.29 (m, 1H), 5.02 (m, 1H), 4.80 (m, 1H), 3.28 (m, 1H), 3.03 (m, 1H), 2.97-2.67 (m, 7H), 2.06 (m, 1H), 1.91 (m, 1H), 1.84 (m, 1H), 1.71 (br, 1H), 1.60 (br, 1H), 1.42 (br, 1H)

### Example 21. (S)-quinuclidin-3-yl (5-(2,4,5-trifluorophenyl)-2,3-dihydro-1H-inden-1-yl)carbamate

¹H-NMR (400 MHz, CDCl₃) δ 7.45 (d, 1H), 7.36 (s, 2H), 7.21 (m, 1H), 7.02 (m, 1H), 5.30 (m, 1H), 5.01 (m, 1H), 4.81 (m, 1H), 3.28 (m, 1H), 3.04 (m, 1H), 2.95-2.67 (m, 7H), 2.09 (m, 1H), 1.90 (m, 2H), 1.71 (br, 1H), 1.60 (br, 1H), 1.41 (br, 1H)

### Example 22. (S)-quinuclidin-3-yl (5-(3,5-dichlorophenyl)-2,3-dihydro-1H-inden-1-yl)carbamate

¹H-NMR (400 MHz, CDCl₃) δ 7.57 (m, 1H), 7.49-7.42 (m, 5H), 5.29 (m, 1H), 5.00 (m, 1H), 4.81 (m, 1H), 3.28 (m, 1H), 3.05 (m, 1H), 2.89-2.67 (m, 7H), 2.06 (m, 1H), 1.83 (m, 2H), 1.71 (br, 1H), 1.61 (br, 1H), 1.42 (br, 1H)

### Example 23. (S)-quinuclidin-3-yl (5-(3,4-dichlorophenyl)-2,3-dihydro-1H-inden-1-yl)carbamate

¹H-NMR (400 MHz, CDCl₃) δ 7.66 (s, 1H), 7.52 (m, 2H), 7.42 (m, 4H), 5.30 (m, 1H), 4.98 (m, 1H), 4.81 (m, 1H), 3.29 (m, 1H), 3.05 (m, 1H), 2.97-2.67 (m, 7H), 2.06 (m, 1H), 1.93 (m, 1H), 1.80 (m, 1H), 1.72 (br, 1H), 1.61 (br, 1H), 1.41 (br, 1H)

### Example 24. (S)-quinuclidin-3-yl (5-(2,5-dichlorophenyl)-2,3-dihydro-1H-inden-1-yl)carbamate

¹H-NMR (400 MHz, CDCl₃) δ 7.42 (m, 1H), 7.32 (m, 2H), 7.28 (m, 3H), 5.31 (m, 1H), 5.07 (m, 1H), 4.80 (m, 1H), 3.28 (m, 1H), 3.03 (m, 1H), 2.94-2.66 (m, 7H), 2.06 (m, 1H), 1.94 (m, 1H), 1.87 (br, 1H), 1.71 (br, 1H), 1.60 (br, 1H), 1.41 (br, 1H)

### Examples 25 to 48

The titled compounds of Examples 25 to 48 were prepared in accordance with the same procedures as in Example 1, using (S)-quinuclidin-3-yl (6-bromo-2,3-dihydro-1H-inden-1-yl)carbamate prepared in Preparation 2; and the corresponding substituted-phenylboronic acids, respectively.

### Example 25. (S)-quinuclidin-3-yl (6-(4-fluorophenyl)-2,3-dihydro-1H-inden-1-yl)carbamate

¹H-NMR (400 MHz, CDCl₃) δ 7.54 (m, 3H), 7.42 (m, 1H), 7.33 (m, 1H), 7.13 (t, 2H), 5.31 (m, 1H), 5.00 (m, 1H), 4.80 (m, 1H), 3.28 (m, 1H), 3.02 (m, 1H), 3.00-2.72 (m, 7H), 2.06 (m, 2H), 1.93 (m, 1H), 1.70 (br, 1H), 1.60 (br, 1H), 1.41 (br, 1H)

### Example 26. (S)-quinuclidin-3-yl (6-(4-chlorophenyl)-2,3-dihydro-1H-inden-1-yl)carbamate

¹H-NMR (400 MHz, CDCl₃) δ 7.52 (m, 3H), 7.40 (m, 3H), 7.33 (m, 1H), 5.31 (m, 1H), 5.03 (m, 1H), 4.80 (br, 1H), 3.28 (m, 1H), 3.00 (m, 1H), 2.93-2.66 (m, 7H), 1.91 (m, 2H), 1.85 (m, 1H), 1.72 (br, 1H), 1.60 (br, 1H), 1.40 (br, 1H)

### Example 27. (S)-quinuclidin-3-yl (6-(4-(trifluoromethyl)phenyl)-2,3-dihydro-1H-inden-1-yl)carbamate

¹H-NMR (400 MHz, CDCl₃) δ 7.69 (s, 4H), 7.55 (s, 1H), 7.49 (d, 1H), 7.36 (d, 1H), 5.33 (m, 1H), 5.04 (m, 1H), 4.81 (br, 1H), 3.28 (m, 1H), 3.02 (m, 1H), 2.94-2.67 (m, 7H), 1.93 (m, 2H), 1.83 (m, 1H), 1.71 (br, 1H), 1.60 (br, 1H), 1.41 (br, 1H)

### Example 28. (S)-quinuclidin-3-yl (6-(4-(trifluoromethoxy)phenyl)-2,3-dihydro-1H-inden-1-yl)carbamate

¹H-NMR (400 MHz, CDCl₃) δ 7.60 (d, 2H), 7.58 (s, 1H), 7.46 (d, 1H), 7.34 (d, 1H), 7.28 (d, 2H), 5.32 (m, 1H), 5.04 (m, 1H), 4.80 (br, 1H), 3.28 (m, 1H), 3.09 (m, 1H), 2.93-2.86 (m, 3H), 2.76 (m, 4H), 1.97 (m, 1H), 1.90 (m, 1H), 1.83 (m, 1H), 1.71 (br, 1H), 1.60 (br, 1H), 1.39 (br, 1H)

### Example 29. (S)-quinuclidin-3-yl (6-(3-fluorophenyl)-2,3-dihydro-1H-inden-1-yl)carbamate

¹H-NMR (400 MHz, CDCl₃) δ 7.52 (s, 1H), 7.46 (d, 1H), 7.37 (m, 3H), 7.28 (m, 1H), 7.04 (m, 1H), 5.31 (m, 1H), 5.02 (m, 1H), 4.81 (br, 1H), 3.31 (m, 1H), 3.04 (m, 1H), 2.88 (m, 3H), 2.77 (m, 4H), 2.06 (m, 1H), 1.93 (m, 1H), 1.84 (m, 1H), 1.68 (m, 1H), 1.60 (br, 1H), 1.41 (br, 1H)

### Example 30. (S)-quinuclidin-3-yl (6-(3-chlorophenyl)-2,3-dihydro-1H-inden-1-yl)carbamate

¹H-NMR (400 MHz, CDCl₃) δ 7.56-7.45 (m, 4H), 7.37-7.28 (m, 3H), 5.32 (m, 1H), 5.02 (m, 1H), 4.81 (br, 1H), 3.27 (m, 1H), 3.03 (m, 1H), 2.93 (m, 3H), 2.77 (m, 4H), 1.92 (m, 1H), 1.86 (m, 1H), 1.85 (m, 1H), 1.71 (br, 1H), 1.60 (br, 1H), 1.41 (br, 1H)

### Example 31. (S)-quinuclidin-3-yl (6-(3-(trifluoromethyl)phenyl)-2,3-dihydro-1H-inden-1-yl)carbamate

¹H-NMR (400 MHz, CDCl₃) δ 7.81 (s, 1H), 7.77 (m, 1H), 7.60-7.54 (m, 3H), 7.50 (m, 1H), 7.36 (m, 1H), 5.33 (m, 1H), 5.04 (m, 1H), 4.80 (br, 1H), 3.27 (m, 1H), 3.03 (m, 1H), 2.94 (m, 3H), 2.76 (m, 4H), 2.09 (m, 1H), 1.96 (m, 1H), 1.83 (m, 1H), 1.70 (br, 1H), 1.60 (br, 1H), 1.41 (br, 1H)

### Example 32. (S)-quinuclidin-3-yl (6-(3-(trifluoromethoxy)phenyl)-2,3-dihydro-1H-inden-1-yl)carbamate

¹H-NMR (400 MHz, CDCl₃) δ 7.51 (d, 2H), 7.46 (m, 2H), 7.42 (s, 1H), 7.35 (d, 1H), 7.21 (d, 1H), 5.30 (m, 1H), 5.05 (m, 1H), 4.80 (br, 1H), 3.30 (m, 1H), 3.02 (m, 1H), 2.87 (m, 3H), 2.70 (m, 4H), 2.08 (m, 1H), 2.00 (m, 1H), 1.90 (m, 1H), 1.70 (br, 1H), 1.60 (br, 1H), 1.40 (br, 1H)

### Example 33. (S)-quinuclidin-3-yl (6-(2-fluorophenyl)-2,3-dihydro-1H-inden-1-yl)carbamate

¹H-NMR (400 MHz, CDCl₃) δ 7.49 (s, 1H), 7.43 (s, 2H), 7.33 (m, 2H), 7.22 (m, 1H), 7.18 (m, 1H), 5.31 (m, 1H), 5.05 (m, 1H), 4,78(br, 1H), 3.27 (m, 1H), 3.03 (m, 1H), 2,91(m, 3H), 2.75 (m, 4H), 1.95 (m, 2H), 1.88 (br, 1H), 1.69 (br, 1H), 1.58 (br, 1H), 1.39 (br, 1H)

### Example 34. (S)-quinuclidin-3-yl (6-(2-chlorophenyl)-2,3-dihydro-1H-inden-1-yl)carbamate

¹H-NMR (400 MHz, CDCl₃) δ 7.48 (d, 1H), 7.39 (s, 1H), 7.32 (m, 5H), 5.31 (m, 1H), 5.07 (m, 1H), 4.77 (br, 1H), 3.24 (m, 1H), 3.04 (m, 1H), 2.94-2.71 (m, 7H), 1.99 (m, 1H), 1.90 (m, 1H), 1.81 (br, 1H), 1.68 (br, 1H), 1.57 (br, 1H), 1.38 (br, 1H)

### Example 35. (S)-quinuclidin-3-yl (6-(2-(trifluoromethyl)phenyl)-2,3-dihydro-1H-inden-1-yl)carbamate

¹H-NMR (400 MHz, CDCl₃) δ 7.76 (d, 1H), 7.56 (m, 1H), 7.49 (m, 1H), 7.34 (m, 1H), 7.27 (m, 2H), 7.22 (m, 1H), 5.31 (m, 1H), 5.06 (m, 1H), 4.76 (br, 1H), 3.25 (m, 1H), 2.94 (m, 1H), 2.84 (m, 2H), 2.75-2.71 (m, 5H), 1.93 (m, 1H), 1.91 (m, 1H), 1.81 (m, 1H), 1.67 (m, 1H), 1.56 (br, 1H), 1.37 (br, 1H)

### Example 36. (S)-quinuclidin-3-yl (6-(2-(trifluoromethoxy)phenyl)-2,3-dihydro-1H-inden-1-yl)carbamate

¹H-NMR (400 MHz, CDCl₃) δ 7.42 (s, 2H), 7.36 (m, 4H), 7.28 (s, 1H), 5.32 (m, 1H), 5.00 (m, 1H), 4.78 (br, 1H), 3.27 (m, 1H), 3.04 (m, 1H), 2.94 (m, 3H), 2.72 (m, 4H), 1.92 (m, 2H), 1.82 (m, 1H), 1.69 (br, 1H), 1.59 (br, 1H), 1.39 (br, 1H)

### Example 37. (S)-quinuclidin-3-yl (6-(4-(difluoromethoxy)phenyl)-2,3-dihydro-1H-inden-1-yl)carbamate

¹H-NMR (400 MHz, CDCl₃) δ 7.58 (d, 2H), 7.50 (s, 1H), 7.45 (d, 1H), 7.33 (d, 1H), 7.21 (d, 2H), 6.75-6.38 (t, 1H), 5.31 (m, 1H), 5.02 (m, 1H), 4.79 (br, 1H), 3.28 (m, 1H), 3.01 (m, 1H), 2.90 (m, 3H), 2.72 (m, 4H), 2.06 (m, 1H), 1.93 (br, 1H), 1.83 (m, 1H), 1.70 (m, 1H), 1.58 (m, 1H), 1.40 (br, 1H)

### Example 38. (S)-quinuclidin-3-yl (6-(p-tolyl)-2,3-dihydro-1H-inden-1-yl)carbamate

¹H-NMR (400 MHz, CDCl₃) δ 7.53 (s, 1H), 7.48 (m, 3H), 7.32 (d, 1H), 7.25 (m, 2H), 5.31 (m, 1H), 5.01 (m, 1H), 4.80 (br, 1H), 3.28 (m, 1H), 3.00 (m, 1H), 2.76 (m, 3H), 2.68 (m, 4H), 2.41 (s, 3H), 2.06 (m, 1H), 1.92-1.82 (m, 2H), 1.69 (m, 1H), 1.59 (br, 1H), 1.40 (br, 1H)

### Example 39. (S)-quinuclidin-3-yl (6-(3,4-difluorophenyl)-2,3-dihydro-1H-inden-1-yl)carbamate

¹H-NMR (400 MHz, CDCl₃) δ 7.47 (s, 1H), 7.42 (m, 2H), 7.37 (m, 2H), 7.21 (m, 1H), 5.31(m, 1H), 5.01 (m, 1H), 4.80 (br, 1H), 3.27 (m, 1H), 3.02 (m, 1H), 2.93 (m, 3H), 2.76 (m, 4H), 2.08 (m, 1H), 1.93 (m, 1H), 1.89 (m, 1H), 1.70 (br, 1H), 1.60 (br, 1H), 1.41 (br, 1H)

### Example 40. (S)-quinuclidin-3-yl (6-(3,5-difluorophenyl)-2,3-dihydro-1H-inden-1-yl)carbamate

¹H-NMR (400 MHz, CDCl₃) δ 7.50 (s, 1H), 7.44 (d, 1H), 7.32 (d, 1H), 7.10 (d, 2H), 6.79 (m, 1H), 5.31 (m, 1H), 5.03 (m, 1H), 4.80 (br, 1H), 3.28 (m, 1H), 3.03 (m, 1H), 2.93-2.67 (m, 7H), 2.09 (m, 1H), 1.92 (m, 1H), 1.84 (m, 1H), 1.70 (m, 1H), 1.61 (br, 1H), 1.40 (br, 1H)

### Example 41. (S)-quinuclidin-3-yl (6-(2,5-difluorophenyl)-2,3-dihydro-1H-inden-1-yl)carbamate

¹H-NMR (400 MHz, CDCl₃) δ 7.47 (s, 1H), 7.42 (d, 1H), 7.34 (m, 1H), 7.10 (m, 2H), 7.01 (m, 1H), 5.30 (m, 1H), 5.04 (m, 1H), 4.79 (m, 1H), 3.26 (m, 1H), 3.04 (m, 1H), 2.87-2.75 (m, 7H), 2.06 (s, 1H), 1.93 (m, 1H), 1.88 (br, 1H), 1.69 (br, 1H), 1.59 (br, 1H), 1.40 (br, 1H)

### Example 42. (S)-quinuclidin-3-yl (6-(2,3-difluorophenyl)-2,3-dihydro-1H-inden-1-yl)carbamate

¹H-NMR (400 MHz, CDCl₃) δ 7.48 (s, 1H), 7.43 (m, 1H), 7.34 (d, 1H), 7.15 (m, 3H), 5.31 (m, 1H), 5.04 (m, 1H), 4.79 (m, 1H), 3.26 (m, 1H), 3.04 (m, 1H), 2.94-2.67 (m, 7H), 2.09 (m, 1H), 1.93 (m, 1H), 1.83 (br, 1H), 1.69 (br, 1H), 1.58 (br, 1H), 1.39 (br, 1H)

### Example 43. (S)-quinuclidin-3-yl (6-(2,4-difluorophenyl)-2,3-dihydro-1H-inden-1-yl)carbamate

¹H-NMR (400 MHz, CDCl₃) δ 7.44 (s, 1H), 7.40 (m, 2H), 7.32 (m, 1H), 6.94 (m, 2H), 5.31 (m, 1H), 5.02 (m, 1H), 4.78 (m, 1H), 3.27 (m, 1H), 3.03 (m, 1H), 2.93-2.66 (m, 7H), 2.06 (m, 1H), 1.89 (m, 1H), 1.83 (br, 1H), 1.69 (br, 1H), 1.59 (br, 1H), 1.40 (br, 1H)

### Example 44. (S)-quinuclidin-3-yl (6-(2,4,5-trifluorophenyl)-2,3-dihydro-1H-inden-1-yl)carbamate

¹H-NMR (400 MHz, CDCl₃) δ 7.43 (s, 1H), 7.36 (m, 2H), 7.23 (m, 1H), 7.04 (m, 1H), 5.31 (m, 1H), 5.02 (m, 1H), 4.79 (m, 1H), 3.26 (m, 1H), 3.02 (m, 1H), 2.93-2.72 (m, 7H), 2.06 (m, 1H), 1.88 (m, 1H), 1.83 (br, 1H), 1.70 (br, 1H), 1.59 (br, 1H), 1.41 (br, 1H)

### Example 45. (S)-quinuclidin-3-yl (6-(2,3,4-trifluorophenyl)-2,3-dihydro-1H-inden-1-yl)carbamate

¹H-NMR (400 MHz, CDCl₃) δ 7.44 (s, 1H), 7.33 (m, 2H), 7.13 (m, 1H), 7.04 (m, 1H), 5.31 (m, 1H), 5.01(, 1H), 4.79 (m, 1H), 3.29 (m, 1H), 3.05 (m, 1H), 2.96-2.67 (m, 7H), 2.06 (m, 1H), 1.88 (m, 1H), 1.84 (br, 1H), 1.70 (br, 1H), 1.59 (br, 1H), 1.40 (br, 1H)

### Example 46. (S)-quinuclidin-3-yl (6-(2,5-dichlorophenyl)-2,3-dihydro-1H-inden-1-yl)carbamate

¹H-NMR (400 MHz, CDCl₃) δ 7.49 (m, 2H), 7.32 (m, 3H), 7.25 (m, 1H), 5.32 (m, 1H), 5.01 (m, 1H), 4.79 (m, 1H), 3.27 (m, 1H), 3.04 (m, 1H), 2.97-2.68 (m, 7H), 2.06 (m, 1H), 1.93 (br, 1H), 1.88 (m, 1H), 1.69 (br, 1H), 1.58 (br, 1H), 1.39 (br, 1H)

### Example 47. (S)-quinuclidin-3-yl (6-(3,4-dichlorophenyl)-2,3-dihydro-1H-inden-1-yl)carbamate

¹H-NMR (400 MHz, CDCl₃) δ 7.66 (s, 1H), 7.52 (m, 2H), 7.44 (m, 2H), 7.34 (m, 1H), 5.31 (m, 1H), 4.99 (m, 1H), 4.81 (br, 1H), 3.28 (m, 1H), 3.01 (m, 1H), 2.88 (m, 3H), 2.77 (m, 4H), 2.08 (m, 1H), 1.89 (m, 1H), 1.81 (m, 1H), 1.71 (m, 1H), 1.61 (m, 1H), 1.42 (br, 1H)

### Example 48. (S)-quinuclidin-3-yl (6-(3,5-dichlorophenyl)-2,3-dihydro-1H-inden-1-yl)carbamate

¹H-NMR (400 MHz, CDCl₃) δ 7.48 (m, 2H), 7.45 (m, 2H), 7.34 (m, 2H), 5.31 (m, 1H), 5.00 (m, 1H), 4.81 (m, 1H), 3.30 (m, 1H), 3.03 (m, 1H), 2.95-2.68 (m, 7H), 2.10 (m, 1H), 1.91 (m, 1H), 1.82 (m, 1H), 1.70 (m, 1H), 1.59 (br, 1H), 1.41 (br, 1H)

### Examples 49 to 72

The titled compounds of Examples 49 to 72 were prepared in accordance with the same procedures as in Example 1, using (S)-quinuclidin-3-yl (6-bromo-2,2-dimethyl-2,3-dihydro-1H-inden-1-yl)carbamate prepared in Preparation 3; and the corresponding substituted-phenylboronic acids, respectively.

### Example 49. (S)-quinuclidin-3-yl (6-(4-fluorophenyl)-2,2-dimethyl-2,3-dihydro-1H-inden-1-yl)carbamate

¹H-NMR (400 MHz, CDCl₃) δ 7.54 (m, 2H), 7.40 (m, 2H), 7.24 (t, 1H), 7.14 (t, 2H), 5.01 (m, 1H), 4.88 (m, 1H), 4.81 (m, 1H), 3.31 (m, 1H), 2.81 (m, 2H), 2.77 (m, 5H), 2.10 (m, 1H), 1.80 (br, 1H), 1.75 (br, 1H), 1.61 (br, 1H), 1.43 (br, 1H), 1.32 (d, 3H), 0.99 (d, 3H)

### Example 50. (S)-quinuclidin-3-yl (6-(4-chlorophenyl)-2,2-dimethyl-2,3-dihydro-1H-inden-1-yl)carbamate

¹H-NMR (400 MHz, CDCl₃) δ 7.50 (m, 2H), 7.42 (m, 3H), 7.26 (m, 2H), 5.02 (m, 1H), 4.89 (m, 1H), 4.81 (m, 1H), 3.30 (m, 1H), 2.89 (m, 2H), 2.77 (m, 5H), 2.14 (m, 1H), 1.86 (br, 1H), 1.73 (br, 1H), 1.62 (br, 1H), 1.43 (br, 1H), 1.32 (d, 3H), 0.99 (d, 3H)

### Example 51. (S)-quinuclidin-3-yl (2,2-dimethyl-6-(4-(trifluoromethyl)phenyl)-2,3-dihydro-1H-inden-1-yl)carbamate

¹H-NMR (400 MHz, CDCl₃) δ 7.69 (s, 4H), 7.45 (m, 2H), 7.29 (d, 1H), 5.04 (m, 1H), 4.94 (m, 1H), 4.80 (m, 1H), 3.30 (m, 1H), 2.91-2.66 (m, 6H), 2.09 (s, 1H), 2.00 (br, 1H), 1.84 (br, 1H), 1.71 (br, 1H), 1.60 (br, 1H), 1.42 (br, 1H), 1.31 (d, 3H), 0.99 (d, 3H)

### Example 52. (S)-quinuclidin-3-yl (2,2-dimethyl-6-(4-(trifluoromethoxy)phenyl)-2,3-dihydro-1H-inden-1-yl)carbamate

¹H-NMR (400 MHz, CDCl₃) δ 7.60 (m, 2H), 7.41 (m, 2H), 7.29 (m, 3H), 5.03 (m, 1H), 4.95 (m, 1H), 4.80 (m, 1H), 3.29 (m, 1H), 2.88-2.66 (m, 6H), 2.09 (s, 1H), 2.00 (br, 1H), 1.85 (br, 1H), 1.71 (br, 1H), 1.59 (br, 1H), 1.42 (br, 1H), 1.34 (d, 3H), 0.99 (d, 3H)

### Example 53. (S)-quinuclidin-3-yl (6-(3-fluorophenyl)-2,2-dimethyl-2,3-dihydro-1H-inden-1-yl)carbamate

¹H-NMR (400 MHz, CDCl₃) δ 7.43 (s, 2H), 7.39 (m, 2H), 7.25 (m, 2H), 7.04 (t, 1H), 5.03 (m, 1H), 4.89 (m, 1H), 4.82 (m, 1H), 3.30 (m, 1H), 2.90 (m, 2H), 2.78 (m, 5H), 2.14 (m, 1H), 1.85 (br, 1H), 1.76 (br, 1H), 1.61 (br, 1H), 1.41 (br, 1H), 1.32 (d, 3H), 0.99 (d, 3H)

### Example 54. (S)-quinuclidin-3-yl (6-(3-chlorophenyl)-2,2-dimethyl-2,3-dihydro-1H-inden-1-yl)carbamate

¹H-NMR (400 MHz, CDCl₃) δ 7.57 (s, 1H), 7.43 (m, 2H), 7.38 (m, 1H), 7.33 (t, 1H), 7.25 (m, 2H), 5.03 (m, 1H), 4.90 (m, 1H), 4.83 (m, 1H), 3.31 (m, 1H), 2.83 (m, 2H), 2.78 (m, 5H), 2.14 (m, 1H), 1.88 (br, 1H), 1.75 (br, 1H), 1.60 (br, 1H), 1.44 (br, 1H), 1.32 (d, 3H), 0.99 (d, 3H)

### Example 55. (S)-quinuclidin-3-yl (2,2-dimethyl-6-(3-(trifluoromethyl)phenyl)-2,3-dihydro-1H-inden-1-yl)carbamate

¹H-NMR (400 MHz, CDCl₃) δ 7.82 (s, 1H), 7.76 (m, 1H), 7.58 (m, 2H), 7.45 (m, 2H), 7.30 (m, 1H), 5.04 (m, 1H), 4.91 (m, 1H), 4.81 (m, 1H), 3.29 (m, 1H), 2.87 (m, 2H), 2.79 (m, 5H), 2.14 (m, 1H), 1.85 (br, 1H), 1.76 (br, 1H), 1.61 (br, 1H), 1.42 (br, 1H), 1.32 (d, 3H), 1.00 (d, 3H)

### Example 56. (S)-quinuclidin-3-yl (2,2-dimethyl-6-(3-(trifluoromethoxy)phenyl)-2,3-dihydro-1H-inden-1-yl)carbamate

¹H-NMR (400 MHz, CDCl₃) δ 7.48 (m, 5H), 7.21 (m, 2H), 5.04 (m, 1H), 4.90 (m, 1H), 4.82 (m, 1H), 3.30 (m, 1H), 2.89 (m, 2H), 2.78 (m, 5H), 2.11 (m, 1H), 1.86 (br, 1H), 1.75 (br, 1H), 1.61 (br, 1H), 1.44 (br, 1H), 1.32 (d, 3H), 1.00 (d, 3H)

### Example 57. (S)-quinuclidin-3-yl (6-(2-fluorophenyl)-2,2-dimethyl-2,3-dihydro-1H-inden-1-yl)carbamate

¹H-NMR (400 MHz, CDCl₃) δ 7.43 (m, 2H), 7.31-7.16 (m, 5H), 5.03 (m, 1H), 4.91 (m, 1H), 4.80 (m, 1H), 3.30 (m, 1H), 2.89 (m, 2H), 2.78 (m, 5H), 2.09 (br, 1H), 1.85 (br, 1H), 1.74 (br, 1H), 1.59 (br, 1H), 1.42 (br, 1H), 1.31 (d, 3H), 1.00 (d, 3H)

### Example 58. (S)-quinuclidin-3-yl (6-(2-chlorophenyl)-2,2-dimethyl-2,3-dihydro-1H-inden-1-yl)carbamate

¹H-NMR (400 MHz, CDCl₃) δ 7.48 (m, 1H), 7.30 (m, 6H), 5.04 (m, 1H), 4.91 (m, 1H), 4.79 (br, 1H), 3.30 (m, 1H), 2.87-2.75 (m, 7H), 2.08 (br, 1H), 1.79 (br, 1H), 1.69 (br, 1H), 1.58 (br, 1H), 1.42 (br, 1H), 1.32 (d, 3H), 0.99 (d, 3H)

### Example 59. (S)-quinuclidin-3-yl (2,2-dimethyl-6-(2-(trifluoromethyl)phenyl)-2,3-dihydro-1H-inden-1-yl)carbamate

¹H-NMR (400 MHz, CDCl₃) δ 7.76 (d, 1H), 7.56 (m, 1H), 7.49 (m, 1H), 7.34 (m, 1H), 7.20 (m, 3H), 5.03 (m, 1H), 4.89 (m, 1H), 4.79 (m, 1H), 3.26 (m, 1H), 2.88-2.71 (m, 7H), 2.09 (m, 1H), 1.81 (br, 1H), 1.71 (br, 1H), 1.58 (br, 1H), 1.41 (br, 1H), 1.33 (d, 3H), 0.99 (d, 3H)

### Example 60. (S)-quinuclidin-3-yl (2,2-dimethyl-6-(p-tolyl)-2,3-dihydro-1H-inden-1-yl)carbamate

¹H-NMR (400 MHz, CDCl₃) δ 7.44 (m, 4H), 7.26 (m, 3H), 5.02 (m, 1H), 4.88 (m, 1H), 4.82 (m, 1H), 3.29 (m, 1H), 2.89-2.77 (m, 7H), 2.41 (s, 3H), 2.08 (br, 1H), 1.85 (br, 1H), 1.75 (br, 1H), 1.61 (br, 1H), 1.43 (br, 1H), 1.31 (d, 3H), 0.99 (d, 3H)

### Example 61. (S)-quinuclidin-3-yl (6-(4-(difluoromethoxy)phenyl)-2,2-dimethyl-2,3-dihydro-1H-inden-1-yl)carbamate

¹H-NMR (400 MHz, CDCl₃) δ 7.58 (m, 2H), 7.50 (m, 2H), 7.25 (m, 1H), 7.20 (m, 2H), 6.75-6.38 (t, 1H), 5.03 (m, 1H), 4.89 (m, 1H), 4.81 (m, 1H), 3.31 (m, 1H), 2.90 (m, 2H), 2.78 (m, 5H), 2.11 (m, 1H), 1.87 (m, 1H), 1.71 (br, 1H), 1.61 (br, 1H), 1.43 (br, 1H), 1.31 (d, 3H), 1.00 (d, 3H)

### Example 62. (S)-quinuclidin-3-yl (6-(3,4-difluorophenyl)-2,2-dimethyl-2,3-dihydro-1H-inden-1-yl)carbamate

¹H-NMR (400 MHz, CDCl₃) δ 7.69 (m, 1H), 7.50 (m, 1H), 7.38 (m, 3H), 7.21 (m, 1H), 5.02 (m, 1H), 4.89 (m, 1H), 4.83 (m, 1H), 3.30 (m, 1H), 2.86 (m, 2H), 2.78 (m, 5H), 2.14 (m, 1H), 1.87 (br, 1H), 1.73 (br, 1H), 1.61 (br, 1H), 1.43 (br, 1H), 1.32 (d, 3H), 0.99 (d, 3H)

### Example 63. (S)-quinuclidin-3-yl (6-(2,4-difluorophenyl)-2,2-dimethyl-2,3-dihydro-1H-inden-1-yl)carbamate

¹H-NMR (400 MHz, CDCl₃) δ 7.48 (m, 3H), 7.28 (m, 1H), 6.94 (m, 2H), 5.03 (m, 1H), 4.90 (m, 1H), 4.81 (m, 1H), 3.28 (m, 1H), 2.88 (m, 3H), 2.78 (m, 4H), 2.14 (m, 1H), 1.87 (br, 1H), 1.75 (br, 1H), 1.60 (br, 1H), 1.42 (br, 1H), 1.32 (d, 3H), 0.99 (d, 3H)

### Example 64. (S)-quinuclidin-3-yl (6-(2,3-difluorophenyl)-2,2-dimethyl-2,3-dihydro-1H-inden-1-yl)carbamate

¹H-NMR (400 MHz, CDCl₃) δ 7.49 (m, 1H), 7.39 (m, 2H), 7.16 (m, 3H), 5.04 (m, 1H), 4.91 (m, 1H), 4.88 (m, 1H), 3.29 (m, 1H), 2.86 (m, 2H), 2.79 (m, 5H), 2.10 (m, 1H), 1.85 (br, 1H), 1.73 (br, 1H), 1.59 (br, 1H), 1.43 (br, 1H), 1.35 (d, 3H), 0.99 (d, 3H)

### Example 65. (S)-quinuclidin-3-yl (6-(2,5-difluorophenyl)-2,2-dimethyl-2,3-dihydro-1H-inden-1-yl)carbamate

¹H-NMR (400 MHz, CDCl₃) δ 7.60 (m, 1H), 7.39 (m, 2H), 7.13 (m, 2H), 7.02 (m, 1H), 5.03 (m, 1H), 4.91 (m, 1H), 4.81 (m, 1H), 3.31 (m, 1H), 2.86 (m, 2H), 2.78 (m, 5H), 2.14 (m, 1H), 1.87 (br, 1H), 1.75 (br, 1H), 1.60 (br, 1H), 1.43 (br, 1H), 1.32 (d, 3H), 0.99 (d, 3H)

### Example 66. (S)-quinuclidin-3-yl (6-(3,5-difluorophenyl)-2,2-dimethyl-2,3-dihydro-1H-inden-1-yl)carbamate

¹H-NMR (400 MHz, CDCl₃) δ 7.69-7.49 (m, 1H), 7.42 (m, 2H), 7.09 (m, 2H), 6.79 (m, 1H), 5.03 (m, 1H), 4.90 (m, 1H), 4.82 (m, 1H), 3.31 (m, 1H), 2.95 (m, 2H), 2.78 (m, 5H), 2.14 (m, 1H), 1.86 (br, 1H), 1.73 (br, 1H), 1.61 (br, 1H), 1.44 (br, 1H), 1.32 (d, 3H), 0.99 (d, 3H)

### Example 67. (S)-quinuclidin-3-yl (2,2-dimethyl-6-(2,3,4-trifluorophenyl)-2,3-dihydro-1H-inden-1-yl)carbamate

¹H-NMR (400 MHz, CDCl₃) δ 7.34 (m, 1H), 7.28 (m, 2H), 7.13 (m, 1H), 7.07 (m, 1H), 5.03 (m, 1H), 4.91 (m, 1H), 4.80 (m, 1H), 3.29 (m, 1H), 2.88 (m, 2H), 2.79 (m, 5H), 2.10 (m, 1H), 1.85 (br, 1H), 1.70 (m, 1H), 1.61 (br, 1H), 1.42 (br, 1H), 1.31 (d, 3H), 1.00 (d, 3H)

### Example 68. (S)-quinuclidin-3-yl (2,2-dimethyl-(2,4,5-trifluorophenyl)-2,3-dihydro-1H-inden-1-yl)carbamate

¹H-NMR (400 MHz, CDCl₃) δ 7.34 (m, 2H), 7.28 (m, 2H), 7.04 (m, 1H), 5.03 (m, 1H), 4.90 (m, 1H), 4.80 (m, 1H), 3.28 (m, 1H), 2.98 (m, 2H), 2.78 (m, 5H), 2.10 (m, 1H), 1.85 (br, 1H), 1.73 (br, 1H), 1.60 (br, 1H), 1.43 (br, 1H), 1.32 (d, 3H), 0.98 (d, 3H)

### Example 69. (S)-quinuclidin-3-yl (6-(3,5-dichlorophenyl)-2,2-dimethyl-2,3-dihydro-1H-inden-1-yl)carbamate

¹H-NMR (400 MHz, CDCl₃) δ 7.71 (m, 1H), 7.66 (m, 1H), 7.50 (m, 1H), 7.45 (m, 1H), 7.40 (m, 1H), 7.33 (m, 1H), 5.03 (m, 1H), 4.89 (m, 1H), 4.82 (m, 1H), 3.30 (m, 1H), 2.91-2.74 (m, 7H), 2.14 (m, 1H), 1.88 (br, 1H), 1.72 (br, 1H), 1.67 (br, 1H), 1.44 (br, 1H), 1.32 (d, 3H), 0.99 (d, 3H)

### Example 70. (S)-quinuclidin-3-yl (6-(3,4-dichlorophenyl)-2,2-dimethyl-2,3-dihydro-1H-inden-1-yl)carbamate

¹H-NMR (400 MHz, CDCl₃) δ 7.69 (m, 2H), 7.50 (m, 2H), 7.39 (m, 2H), 5.02 (m, 1H), 4.89 (m, 1H), 4.82 (m, 1H), 3.31 (m, 1H), 2.90 (m, 2H), 2.78 (m, 5H), 2.14 (m, 1H), 1.87 (br, 1H), 1.74 (br, 1H), 1.62 (br, 1H), 1.44 (br, 1H), 1.32 (d, 3H), 0.99 (d, 3H)

### Example 71. (S)-quinuclidin-3-yl (6-(2,5-dichlorophenyl)-2,2-dimethyl-2,3-dihydro-1H-inden-1-yl)carbamate

¹H-NMR (400 MHz, CDCl₃) δ 7.41 (m, 1H), 7.39 (m, 1H), 7.28 (m, 4H), 5.01 (m, 1H), 4.92 (m, 1H), 4.80 (m, 1H), 3.27 (m, 1H), 2.78 (m, 7H), 2.09 (m, 1H), 1.85 (br, 1H), 1.69 (br, 1H), 1.58 (br, 1H), 1.41 (br, 1H), 1.32 (d, 3H), 0.99 (d, 3H)

### Example 72. (S)-quinuclidin-3-yl (6-(4-(2-methoxyethoxy)phenyl)-2,2-dimethyl-2,3-dihydro-1H-inden-1-yl)carbamate

¹H-NMR (400 MHz, CDCl₃) δ 7.51 (m, 2H), 7.40 (m, 2H), 7.21 (d, 1H), 6.99 (m, 2H), 4.98 (m, 1H), 4.84 (m, 1H), 4.80 (br, 1H), 4.17 (m, 2H), 3.78 (m, 2H), 3.47 (s, 3H), 3.25 (m, 1H), 2.88-2.71 (m, 7H), 2.10 (br, 1H), 1.86 (br, 1H), 1.71 (br, 1H), 1.60 (br, 1H), 1.42 (br, 1H), 1.30 (m, 3H), 0.98 (m, 3H)

### Examples 73 to 275

The titled compounds of Examples 73 to 275 were prepared in accordance with the same procedures as in Example 1, using (S)-quinuclidin-3-yl (5-bromo-2,2-dimethyl-2,3-dihydro-1H-inden-1-yl)carbamate prepared in Preparation 4; and the corresponding substituted-phenylboronic acids, respectively.

### Example 73. (S)-quinuclidin-3-yl (5-(4-fluorophenyl)-2,2-dimethyl-2,3-dihydro-1H-inden-1-yl)carbamate

¹H-NMR (400 MHz, CDCl₃) δ 7.53 (m, 2H), 7.40 (m, 2H), 7.30 (m, 1H), 7.13 (t, 2H), 5.00 (m, 1H), 4.88 (m, 1H), 4.83 (m, 1H), 3.29 (m, 1H), 2.91-2.86 (m, 2H), 2.82-2.75 (m, 5H), 2.12 (br, 1H), 1.82 (br, 1H), 1.72 (br, 1H), 1.61 (br, 1H), 1.44 (br, 1H), 1.31 (d, 3H), 0.99 (d, 3H)

### Example 74. (S)-quinuclidin-3-yl (5-(4-chlorophenyl)-2,2-dimethyl-2,3-dihydro-1H-inden-1-yl)carbamate

¹H-NMR (400 MHz, CDCl₃) δ 7.51 (d, 2H), 7.41 (d, 2H), 7.36 (m, 2H), 7.31 (m, 1H), 5.00 (m, 1H), 4.90 (m, 1H), 4.81 (br, 1H), 3.31 (m, 1H), 2.91-2.87 (m, 2H), 2.82-2.75 (m, 5H), 2.12 (br, 1H), 1.86 (br, 1H), 1.72 (br, 1H), 1.61 (br, 1H), 1.44 (br, 1H), 1.31 (d, 3H), 0.99 (d, 3H)

### Example 75. (S)-quinuclidin-3-yl (2,2-dimethyl-5-(4-(trifluoromethyl)phenyl)-2,3-dihydro-1H-inden-1-yl)carbamate

¹H-NMR (400 MHz, CDCl₃) δ 7.67 (s, 4H), 7.46-7.44 (m, 1H), 7.42 (s, 1H), 7.35-7.27 (m, 1H), 5.02 (m, 1H), 4.89 (m, 1H), 4.82 (m, 1H), 3.29 (m, 1H), 2.90-2.85 (m, 2H), 2.81-2.77 (m, 5H), 2.13 (br, 1H), 1.83 (br, 1H), 1.72 (br, 1H), 1.62 (br, 1H), 1.46 (br, 1H), 1.33 (d, 3H), 1.01 (d, 3H)

### Example 76. (S)-quinuclidin-3-yl (2,2-dimethyl-5-(4-(trifluoromethoxy)phenyl)-2,3-dihydro-1H-inden-1-yl)carbamate

¹H-NMR (400 MHz, CDCl₃) δ 7.58 (d, 2H), 7.42-7.37 (m, 2H), 7.29 (m, 3H), 5.01 (m, 1H), 4.89 (m, 1H), 4.82 (m, 1H), 3.29 (m, 1H), 2.90-2.87 (m, 2H), 2.83-2.77 (m, 5H), 2.12 (br, 1H), 1.83 (br, 1H), 1.73 (br, 1H), 1.61 (br, 1H), 1.44 (br, 1H), 1.32 (d, 3H), 1.00 (d, 3H)

### Example 77. (S)-quinuclidin-3-yl (5-(3-fluorophenyl)-2,2-dimethyl-2,3-dihydro-1H-inden-1-yl)carbamate

¹H-NMR (400 MHz, CDCl₃) δ 7.44-7.37 (m, 3H), 7.34 (m, 1H), 7.31 (m, 2H), 7.04 (t, 1H), 5.01 (m, 1H), 4.90 (m, 1H), 4.81 (m, 1H), 3.29 (m, 1H), 2.89-2.87 (m, 2H), 2.83-2.75 (m, 5H), 2.11 (br, 1H), 1.86 (br, 1H), 1.71 (br, 1H), 1.61 (br, 1H), 1.43 (br, 1H), 1.32 (d, 3H), 1.00 (d, 3H)

### Example 78. (S)-quinuclidin-3-yl (5-(3-chlorophenyl)-2,2-dimethyl-2,3-dihydro-1H-inden-1-yl)carbamate

¹H-NMR (400 MHz, CDCl₃) δ 7.56 (s, 1H), 7.45 (m, 2H), 7.40 (m, 2H), 7.34 (m, 1H), 7.30 (m, 1H), 5.01 (m, 1H), 4.96 (m, 1H), 4.81 (m, 1H), 3.28 (m, 1H), 2.89-2.86 (m, 2H), 2.82-2.75 (m, 5H), 2.11 (br, 1H), 1.86 (br, 1H), 1,72(br, 1H), 1.61 (br, 1H), 1.43 (br, 1H), 1.32 (d, 3H), 0.99 (d, 3H)

### Example 79. (S)-quinuclidin-3-yl (2,2-dimethyl-5-(3-(trifluoromethyl)phenyl)-2,3-dihydro-1H-inden-1-yl)carbamate

¹H-NMR (400 MHz, CDCl₃) δ 7.82 (s, 1H), 7.75 (d, 1H), 7.60-7.56 (m, 2H), 7.43 (m, 2H), 7.35 (m, 1H), 5.03 (m, 1H), 4.92 (m, 1H), 4.81 (br, 1H), 3.29 (m, 1H), 2.89-2.84 (m, 2H), 2.81-2.77 (m, 5H), 2.12 (br, 1H), 1.84 (br, 1H), 1.72 (br, 1H), 1.61 (br, 1H), 1.44 (br, 1H), 1.33 (d, 3H), 1.00 (d, 3H)

### Example 80. (S)-quinuclidin-3-yl (2,2-dimethyl-6-(3-(trifluoromethoxy)phenyl)-2,3-dihydro-1H-inden-1-yl)carbamate

¹H-NMR (400 MHz, CDCl₃) δ 7.52-7.45 (m, 1H), 7.41 (m, 4H), 7.32 (m, 1H), 7.20 (d, 1H), 5.02 (m, 1H), 4.89 (m, 1H), 4.82 (m, 1H), 3.28 (m, 1H), 2.90-2.84 (m, 2H), 2.79-2.76 (m, 5H), 2.12 (br, 1H), 1.82 (br, 1H), 1.70 (br, 1H), 1.61 (br, 1H), 1.44 (br, 1H), 1.32 (d, 3H), 1.00 (d, 3H)

### Example 81. (S)-quinuclidin-3-yl (5-(2-fluorophenyl)-2,2-dimethyl-2,3-dihydro-1H-inden-1-yl)carbamate

¹H-NMR (400 MHz, CDCl₃) δ 7.43-7.37 (m, 3H), 7.33-7.28 (m, 2H), 7.23 (m, 1H), 7.18 (m, 1H), 5.01 (m, 1H), 4.91 (m, 1H), 4.81 (br, 1H), 3.30 (m, 1H), 2.89-2.87 (m, 2H), 2.83-2.76 (m, 5H), 2.12 (br, 1H), 1.85 (br, 1H), 1.72 (br, 1H), 1.61 (br, 1H), 1.43 (br, 1H), 1.32 (d, 3H), 1.00 (d, 3H)

### Example 82. (S)-quinuclidin-3-yl (5-(2-chlorophenyl)-2,2-dimethyl-2,3-dihydro-1H-inden-1-yl)carbamate

¹H-NMR (400 MHz, CDCl₃) δ 7.47 (d, 1H), 7.33-7.25 (m, 6H), 5.02 (m, 1H), 4.94 (m, 1H), 4.82 (br, 1H), 3.28 (m, 1H), 2.89-2.87 (m, 2H), 2.83-2.75 (m, 5H), 2.13 (br, 1H), 1.85 (br, 1H), 1.72 (br, 1H), 1.61 (br, 1H), 1.43 (br, 1H), 1.34 (d, 3H), 1.00 (d, 3H)

### Example 83. (S)-quinuclidin-3-yl (2,2-dimethyl-5-(2-(trifluoromethyl)phenyl)-2,3-dihydro-1H-inden-1-yl)carbamate

¹H-NMR (400 MHz, CDCl₃) δ 7.74 (d, 1H), 7.55 (m, 1H), 7.48 (m, 1H), 7.33 (d, 1H), 7.27 (m, 1H), 7.17 (m, 1H), 7.14 (s, 1H), 5.03 (m, 2H), 4.82 (br, 1H), 3.31 (m, 1H), 2.89-2.85 (m, 2H), 2.82-2.73 (m, 5H), 2.11 (br, 1H), 1.85 (br, 1H), 1.71 (br, 1H), 1.61 (br, 1H), 1.44 (br, 1H), 1.32 (d, 3H), 1.00 (d, 3H)

### Example 84. (S)-quinuclidin-3-yl (2,2-dimethyl-5-(2-(trifluoromethoxy)phenyl)-2,3-dihydro-1H-inden-1-yl)carbamate

¹H-NMR (400 MHz, CDCl₃) δ 7.42 (m, 1H), 7.36 (m, 3H), 7.27 (m, 3H), 5.03 (m, 1H), 4.94 (m, 1H), 4.82 (m, 1H), 3.28 (m, 1H), 2.90-2.87(m 2H), 2.83-2.75 (m, 5H), 2.12 (br, 1H), 1.85 (br, 1H), 1.72 (br, 1H), 1.62 (br, 1H), 1.44 (br, 1H), 1.32 (d, 3H), 1.00 (d, 3H)

### Example 85. (S)-quinuclidin-3-yl (2,2-dimethyl-5-(p-tolyl)-2,3-dihydro-1H-inden-1-yl)carbamate

¹H-NMR (400 MHz, CDCl₃) δ 7.49 (d, 2H), 7.42 (m, 2H), 7.26 (m, 3H), 5.00 (m, 1H), 4.89 (m, 1H), 4.80 (m, 1H), 3.29 (m, 1H), 2.89-2.85 (m, 2H), 2.82-2.75 (m, 5H), 2.41 (s, 3H), 2.11 (br, 1H), 1.82 (br, 1H), 1.72 (br, 1H), 1.60 (br, 1H), 1.44 (br, 1H), 1.31 (d, 3H), 1.00 (d, 3H)

### Example 86. (S)-quinuclidin-3-yl (5-(4-(difluoromethoxy)phenyl)-2,2-dimethyl-2,3-dihydro-1H-inden-1-yl)carbamate

¹H-NMR (400 MHz, CDCl₃) δ 7.56 (d, 2H), 7.37 (m, 2H), 7.32 (m, 1H), 7.19 (d, 2H), 6.75-6.38 (t, 1H), 4.99 (m, 1H), 2.90 (m, 1H), 4.82 (br, 1H), 3.29 (m, 1H), 2.90-2.87 (m, 2H), 2.83-2.79 (m, 5H), 2.11 (br, 1H), 1.82 (br, 1H), 1.72 (br, 1H), 1.60 (br, 1H), 1.44 (br, 1H), 1.32 (s, 3H), 0.99 (d, 3H)

### Example 87. (S)-quinuclidin-3-yl (5-(3,4-difluorophenyl)-2,2-dimethyl-2,3-dihydro-1H-inden-1-yl)carbamate

¹H-NMR (400 MHz, CDCl₃) δ 7.37 (m, 3H), 7.29 (m, 2H), 7.23 (m, 1H), 5.01 (m, 1H), 4.90 (m, 1H), 4.82 (m, 1H), 3.32 (m, 1H), 2.89-2.86 (m, 2H), 2.82-2.75 (m, 5H), 2.11 (m, 1H), 1.90 (br, 1H), 1.72 (br, 1H), 1.60 (br, 1H), 1.43 (br, 1H), 1.34 (d, 3H), 0.99 (d, 3H)

### Example 88. (S)-quinuclidin-3-yl (5-(2,5-difluorophenyl)-2,2-dimethyl-2,3-dihydro-1H-inden-1-yl)carbamate

¹H-NMR (400 MHz, CDCl₃) δ 7.36 (m, 3H), 7.12 (m, 2H), 6.99 (m, 1H), 5.02 (m, 1H), 4.90 (m, 1H), 4.80 (m, 1H), 3.29 (m, 1H), 2.89-2.87 (m, 2H), 2.83-2.75 (m, 5H), 2.11 (m, 1H), 1.86 (br, 1H), 1.72 (br, 1H), 1.61 (br, 1H), 1.43 (br, 1H), 1.32 (d, 3H), 1.00 (d, 3H)

### Example 89. (S)-quinuclidin-3-yl (5-(2,4-difluorophenyl)-2,2-dimethyl-2,3-dihydro-1H-inden-1-yl)carbamate

¹H-NMR (400 MHz, CDCl₃) δ 7.40 (m, 1H), 7.35 (m, 3H), 6.95 (m, 2H), 5.01 (m, 1H), 4.91 (m, 1H), 4.80 (m, 1H), 3.29 (m, 1H), 2.89-2.86 (m, 2H), 2.83-2.75 (m, 5H), 2.11 (m, 1H), 1.85 (br, 1H), 1.75 (br, 1H), 1.60 (br, 1H), 1.43 (br, 1H), 1.32 (d, 3H), 0.99 (d, 3H)

### Example 90. (S)-quinuclidin-3-yl (5-(3,5-difluorophenyl)-2,2-dimethyl-2,3-dihydro-1H-inden-1-yl)carbamate

¹H-NMR (400 MHz, CDCl₃) δ 7.50 (m, 3H), 7.10 (m, 2H), 6.78((m, 1H), 5.02 (m, 1H), 4.89 (m, 1H), 4.81 (m, 1H), 3.30 (m, 1H), 2.90-2.87 (m, 2H), 2.83-2.75 (m, 5H), 2.12 (m, 1H), 1.86 (br, 1H), 1.75 (br, 1H), 1.61 (br, 1H), 1.45 (br, 1H), 1.32 (d, 3H), 0.99 (d, 3H)

### Example 91. (S)-quinuclidin-3-yl (5-(2,3-difluorophenyl)-2,2-dimethyl-2,3-dihydro-1H-inden-1-yl)carbamate

¹H-NMR (400 MHz, CDCl₃) δ 7.38-7.28 (m, 3H), 7.16 (m, 3H), 5.02 (m, 1H), 4.91 (m, 1H), 4.82 (m, 1H), 3.30 (m, 1H), 2.88 (m, 2H), 2.83-2.76 (m, 5H), 2.12 (m, 1H), 1.85 (br, 1H), 1.72 (br, 1H), 1.60 (br, 1H), 1.44 (br, 1H), 1.32 (d, 3H), 0.99 (d, 3H)

### Example 92. (S)-quinuclidin-3-yl (5-(3,4-dichlorophenyl)-2,2-dimethyl-2,3-dihydro-1H-inden-1-yl)carbamate

¹H-NMR (400 MHz, CDCl₃) δ 7.71 (m, 2H), 7.56 (m, 1H), 7.48 (m, 2H), 7.39 (m, 1H), 5.03 (m, 1H), 4.86 (m, 2H), 3.31 (m, 1H), 2.91 (m, 2H), 2.86-2.78 (m, 5H), 2.13 (m, 1H), 1.86 (br, 1H), 1.75 (br, 1H), 1.68 (br, 1H), 1.46 (br, 1H), 1.32(d 3H), 0.99 (d, 3H)

### Example 93. (S)-quinuclidin-3-yl (5-(2,5-dichlorophenyl)-2,2-dimethyl-2,3-dihydro-1H-inden-1-yl)carbamate

¹H-NMR (400 MHz, CDCl₃) δ 7.41 (d, 1H), 7.33-7.23 (m, 5H), 5.03 (m, 1H), 4.94 (m, 1H), 4.82 (m, 1H), 3.28 (m, 1H), 2.90-2.87 (m, 2H), 2.83-2.75 (m, 5H), 2.11 (m, 1H), 1.85 (br, 1H), 1.71 (br, 1H), 1.61 (br, 1H), 1.44 (br, 1H), 1.32 (d, 3H), 0.99 (d, 3H)

### Example 94. (S)-quinuclidin-3-yl (5-(3,5-dichlorophenyl)-2,2-dimethyl-2,3-dihydro-1H-inden-1-yl)carbamate

¹H-NMR (400 MHz, CDCl₃) δ 7.50 (m, 1H), 7.45 (m, 1H), 7.39 (s, 2H), 7.35 (d, 2H), 5.02 (m, 1H), 4.87 (m, 2H), 3.30 (m, 1H), 2.90-2.82 (m, 2H), 2.83-2.79 (m, 5H), 2.13 (br, 1H), 2.06 (br, 1H), 1.88 (br, 1H), 1.69 (br, 1H), 1.48 (br, 1H), 1.32 (d, 3H), 0.99 (d, 3H)

### Example 95. (S)-quinuclidin-3-yl (2,2-dimethyl-5-(2,3,4-trifluorophenyl)-2,3-dihydro-1H-inden-1-yl)carbamate

¹H-NMR (400 MHz, CDCl₃) δ 7.31 (m, 3H), 7.13 (m, 1H), 7.06 (m, 1H), 5.02 (m, 1H), 4.90 (m, 1H), 4.83 (m, 1H), 3.30 (m, 1H), 2.94-2.88 (m, 2H), 2.83-2.76 (m, 5H), 2.12 (m, 1H), 1.87 (br, 1H), 1.73 (m, 1H), 1.61 (br, 1H), 1.47 (br, 1H), 1.31 (d, 3H), 1.00 (d, 3H)

### Example 96. (S)-quinuclidin-3-yl (2,2-dimethyl-5-(2,4,5-trifluorophenyl)-2,3-dihydro-1H-inden-1-yl)carbamate

¹H-NMR (400 MHz, CDCl₃) δ 7.32 (m, 3H), 7.23 (m, 1H), 7.03 (m, 1H), 5.02 (m, 1H), 4.89 (m, 1H), 4.81 (m, 1H), 3.32 (m, 1H), 2.91-2.87 (m, 2H), 2.83-2.75 (m, 5H), 2.11 (m, 1H), 1.85 (br, 1H), 1.72 (br, 1H), 1.61 (br, 1H), 1.44 (br, 1H), 1.32 (d, 3H), 0.99 (d, 3H)

### Example 97. (S)-quinuclidin-3-yl (5-(4-(2-methoxyethoxy)phenyl)-2,2-dimethyl-2,3-dihydro-1H-inden-1-yl)carbamate

¹H-NMR (400 MHz, CDCl₃) δ 7.49 (d, 2H), 7.39 (m, 1H), 7.36 (s, 1H), 7.27 (m, 1H), 7.00 (d, 2H), 4.98 (m, 1H), 4.84 (m, 1H), 4.80 (br, 1H), 4.18 (m, 2H), 3.78 (m, 2H), 3.48 (s, 3H), 3.25 (m, 1H), 2.90-2.75 (m, 7H), 2.10 (br, 1H), 1.86 (br, 1H), 1.71 (br, 1H), 1.60 (br, 1H), 1.42 (br, 1H), 1.30 (m, 3H), 0.98 (m, 3H)

### Example 98. (S)-quinuclidin-3-yl (5-(3-(2-methoxyethoxy)phenyl)-2,2-dimethyl-2,3-dihydro-1H-inden-1-yl)carbamate

¹H-NMR (400 MHz, CDCl₃) δ 7.42 (m, 1H), 7.36 (s, 1H), 7.31-7.27 (m, 2H), 7.18 (m, 2H), 6.91 (d, 1H), 4.99 (m, 1H), 4.84-4.80 (m, 2H), 4.18 (m, 2H), 3.79 (m, 2H), 3.48 (s, 3H), 3.25 (m, 1H), 2.90-2.75 (m, 7H), 2.10 (br, 1H), 1.86 (br, 1H), 1.71 (br, 1H), 1.60 (br, 1H), 1.42 (br, 1H), 1.30 (m, 3H), 0.98 (m, 3H)

### Example 99. (S)-quinuclidin-3-yl (5-(2-(2-methoxyethoxy)phenyl)-2,2-dimethyl-2,3-dihydro-1H-inden-1-yl)carbamate

¹H-NMR (400 MHz, CDCl₃) δ 7.40 (m, 2H), 7.32-7.27 (m, 3H), 7.04-7.98 (m, 2H), 4.99 (m, 1H), 4.84 (m, 1H), 4.80 (m, 1H), 4.10 (m, 2H), 3.68 (m, 2H), 3.38 (s, 3H), 3.25 (m, 1H), 2.90-2.75 (m, 7H), 2.10 (br, 1H), 1.86 (br, 1H), 1.71 (br, 1H), 1.60 (br, 1H), 1.42 (br, 1H), 1.30 (m, 3H), 0.98 (m, 3H)

### Example 100. (S)-quinuclidin-3-yl (5-(4-(methoxymethoxy)phenyl)-2,2-dimethyl-2,3-dihydro-1H-inden-1-yl)carbamate

¹H-NMR (400 MHz, CDCl₃) δ 7.51 (d, 2H), 7.38 (m, 1H), 7.35 (s, 1H), 7.27 (m, 1H), 7.10 (d, 2H), 5.22 (s, 2H), 4.99 (m, 1H), 4.84 (m, 1H), 4.80 (m, 1H), 3.31 (s, 3H), 3.25 (m, 1H), 2.90-2.75 (m, 7H), 2.10 (br, 1H), 1.86 (br, 1H), 1.71 (br, 1H), 1.60 (br, 1H), 1.42 (br, 1H), 1.30 (m, 3H), 0.98 (m, 3H)

### Example 101. (S)-quinuclidin-3-yl (5-(3-(methoxymethoxy)phenyl)-2,2-dimethyl-2,3-dihydro-1H-inden-1-yl)carbamate

¹H-NMR (400 MHz, MeOD) δ 7.41 (m, 2H), 7.28 (m, 1H), 7.26 (m, 1H), 7.05 (d, 1H), 7.00 (s, 1H), 6.75 (d, 1H), 5.07 (br, 1H), 4.87 (s, 2H), 3.72 (m, 1H), 3.67 (s, 3H), 3.39 (m, 2H), 3.39-3.29 (m, 5H), 2.81 (s, 2H), 2.45 (m, 1H), 2.30 (br, 1H), 2.10 (br, 1H), 1.93 (br, 1H), 1.24 (d, 3H), 0.98 (d, 3H)

### Example 102. (S)-quinuclidin-3-yl (5-(2-(methoxymethoxy)phenyl)-2,2-dimethyl-2,3-dihydro-1H-inden-1-yl)carbamate

¹H-NMR (400 MHz, CDCl₃) δ 7.39 (m, 1H), 7.35 (m, 4H), 7.21 (m, 1H), 7.09 (m, 1H), 5.12 (s, 2H), 4.99 (m, 1H), 4.84 (m, 1H), 4.80 (m, 1H), 3.41 (s, 3H), 3.25 (m, 1H), 2.90-2.75 (m, 7H), 2.10 (br, 1H), 1.86 (br, 1H), 1.71 (br, 1H), 1.60 (br, 1H), 1.42 (br, 1H), 1.30 (m, 3H), 0.98 (m, 3H)

### Example 103. (S)-quinuclidin-3-yl (5-(3-methoxyphenyl)-2,2-dimethyl-2,3-dihydro-1H-inden-1-yl)carbamate

¹H-NMR (400 MHz, CDCl₃) δ 7.44 (m, 1H), 7.39 (s, 1H), 7.31-7.27 (m, 2H), 7.15 (d, 1H), 7.10 (s, 1H), 6.89 (d, 1H), 4.98 (m, 1H), 4.88 (m, 1H), 4.79 (br, 1H), 3.87 (s, 3H), 3.25 (m, 1H), 2.90-2.74 (m, 7H), 2.10 (br, 1H), 1.86 (br, 1H), 1.71 (br, 1H), 1.60 (br, 1H), 1.42 (br, 1H), 1.30 (m, 3H), 0.98 (m, 3H)

### Example 104. (S)-quinuclidin-3-yl (5-(3-ethoxyphenyl)-2,2-dimethyl-2,3-dihydro-1H-inden-1-yl)carbamate

¹H-NMR (400 MHz, CDCl₃) δ 7.44 (m, 1H), 7.39 (s, 1H), 7.31-7.27 (m, 2H), 7.15 (d, 1H), 7.10 (s, 1H), 6.89 (d, 1H), 4.98 (m, 1H), 4.88 (m, 1H), 4.79 (br, 1H), 4.11 (m, 2H), 3.25 (m, 1H), 2.90-2.74 (m, 7H), 2.10 (br, 1H), 1.86 (br, 1H), 1.71 (br, 1H), 1.60 (br, 1H), 1.45 (t, 4H), 1.29 (m, 3H), 0.98 (m, 3H)

### Example 105. (S)-quinuclidin-3-yl (5-(3-isopropoxyphenyl)-2,2-dimethyl-2,3-dihydro-1H-inden-1-yl)carbamate

¹H-NMR (400 MHz, CDCl₃) δ 7.43 (m, 1H), 7.39 (s, 1H), 7.35-7.27 (m, 2H), 7.14 (d, 1H), 7.10 (s, 1H), 6.88 (d, 1H), 5.00 (m, 1H), 4.85 (m, 1H), 4.80 (br, 1H), 4.62 (m, 1H), 3.25 (m, 1H), 2.90-2.74 (m, 7H), 2.10 (br, 1H), 1.86 (br, 1H), 1.71 (br, 1H), 1.60 (br, 1H), 1.42 (br, 1H), 1.38 (d, 6H), 1.29 (m, 3H), 0.98 (m, 3H)

### Example 106. (S)-quinuclidin-3-yl (5-(3-(dimethylamino)phenyl)-2,2-dimethyl-2,3-dihydro-1H-inden-1-yl)carbamate

¹H-NMR (400 MHz, CDCl₃) δ 7.44 (m, 1H), 7.40 (s, 1H), 7.32-7.27 (m, 2H), 6.91 (m, 2H), 6.74 (d, 1H), 4.99 (m, 1H), 4.86 (m, 1H), 4.80 (br, 1H), 3.25 (m, 1H), 3.01 (s, 6H), 2.92-2.75 (m, 7H), 2.10 (br, 1H), 1.86 (br, 1H), 1.71 (br, 1H), 1.60 (br, 1H), 1.42 (br, 1H), 1.30 (m, 3H), 0.97 (m, 3H)

### Example 107. (S)-quinuclidin-3-yl (2,2-dimethyl-5-(3-(methylthio)phenyl)-2,3-dihydro-1H-inden-1-yl)carbamate

¹H-NMR (400 MHz, CDCl₃) δ 7.45 (s, 1H), 7.41-7.23 (m, 6H), 4.99 (m, 1H), 4.87 (m, 1H), 4.80 (br, 1H), 3.25 (m, 1H), 2.92-2.75 (m, 7H), 2.54 (s, 3H), 2.10 (br, 1H), 1.86 (br, 1H), 1.71 (br, 1H), 1.60 (br, 1H), 1.42 (br, 1H), 1.31 (m, 3H), 0.98 (m, 3H)

### Example 108. (S)-quinuclidin-3-yl (5-(3-ethylphenyl)-2,2-dimethyl-2,3-dihydro-1H-inden-1-yl)carbamate

¹H-NMR (400 MHz, CDCl₃) δ 7.45 (d, 1H), 7.42 (s, 2H), 7.40-7.28 (m, 3H), 7.19 (d, 1H), 5.00 (m, 1H), 4.88 (m, 1H), 4.80 (br, 1H), 3.25 (m, 1H), 2.92-2.75 (m, 7H), 2.70 (m, 2H), 2.10 (br, 1H), 1.86 (br, 1H), 1.71 (br, 1H), 1.60 (br, 1H), 1.42 (br, 1H), 1.31 (t, 3H), 1.29 (m, 3H), 0.98 (m, 3H)

### Example 109. (S)-quinuclidin-3-yl (5-(3-isopropylphenyl)-2,2-dimethyl-2,3-dihydro-1H-inden-1-yl)carbamate

¹H-NMR (400 MHz, CDCl₃) δ 7.43-7.34 (m, 5H), 7.28 (m, 1H), 7.22 (d, 1H), 5.00 (m, 1H), 4.88 (m, 1H), 4.80 (br, 1H), 3.25 (m, 1H), 2.98 (m, 1H), 2.92-2.75 (m, 7H), 2.10 (br, 1H), 1.86 (br, 1H), 1.71 (br, 1H), 1.60 (br, 1H), 1.42 (br, 1H), 1.30 (m, 9H), 0.98 (m, 3H)

### Example 110. (S)-quinuclidin-3-yl (5-(3-(tert-butyl)phenyl)-2,2-dimethyl-2,3-dihydro-1H-inden-1-yl)carbamate

¹H-NMR (400 MHz, CDCl₃) δ 7.58 (s, 1H), 7.44 (d, 1H), 7.38 (s, 4H), 7.28 (m, 1H), 5.00 (m, 1H), 4.88 (m, 1H), 4.79 (br, 1H), 3.25 (m, 1H), 2.92-2.75 (m, 7H), 2.10 (br, 1H), 1.86 (br, 1H), 1.71 (br, 1H), 1.60 (br, 1H), 1.42 (br, 1H), 1.38 (s, 9H), 1.29 (m, 3H), 0.98 (m, 3H)

### Example 111. (S)-quinuclidin-3-yl (5-(2,2-difluorobenzo[d][1,3]dioxol-5-yl)-2,2-dimethyl-2,3-dihydro-1H-inden-1-yl)carbamate

¹H-NMR (400 MHz, CDCl₃) δ 7.34-7.27 (m, 3H), 7.25 (m, 2H), 7.10 (d, 1H), 5.00 (m, 1H), 4.88 (m, 1H), 4.79 (br, 1H), 3.25 (m, 1H), 2.92-2.75 (m, 7H), 2.10 (br, 1H), 1.86 (br, 1H), 1.71 (br, 1H), 1.60 (br, 1H), 1.42 (br, 1H), 1.30 (m, 3H), 0.98 (m, 3H)

### Example 112. (S)-quinuclidin-3-yl (5-(2,3-dihydrobenzo[b][1,4]dioxin-6-yl)-2,2-dimethyl-2,3-dihydro-1H-inden-1-yl)carbamate

¹H-NMR (400 MHz, CDCl₃) δ 7.38 (m, 1H), 7.36 (s, 1H), 7.25 (m, 1H), 7.09 (s, 1H), 7.05 (d, 1H), 6.91 (d, 1H), 4.98 (m, 1H), 4.88 (m, 1H), 4.79 (br, 1H), 4.29 (s, 4H), 3.25 (m, 1H), 2.92-2.73 (m, 7H), 2.10 (br, 1H), 1.86 (br, 1H), 1.71 (br, 1H), 1.60 (br, 1H), 1.42 (br, 1H), 1.29 (m, 3H), 0.97 (m, 3H)

### Example 113. (S)-quinuclidin-3-yl (5-(2,3-dihydrobenzofuran-5-yl)-2,2-dimethyl-2,3-dihydro-1H-inden-1-yl)carbamate

¹H-NMR (400 MHz, CDCl₃) δ 7.41 (s, 1H), 7.36-7.30 (m, 3H), 7.25 (m, 1H), 6.84 (d, 1H), 4.98 (m, 1H), 4.88 (m, 1H), 4.79 (br, 1H), 4.62 (t, 2H), 3.25 (m, 3H), 2.92-2.73 (m, 7H), 2.10 (br, 1H), 1.86 (br, 1H), 1.71 (br, 1H), 1.60 (br, 1H), 1.42 (br, 1H), 1.29 (m, 3H), 0.97 (m, 3H)

### Example 114. (S)-quinuclidin-3-yl (2,2-dimethyl-5-(3-(2-morpholinoethoxy)phenyl)-2,3-dihydro-1H-inden-1-yl)carbamate

¹H-NMR (400 MHz, CDCl₃) δ 7.43 (d, 1H), 7.39 (s, 1H), 7.38-7.30 (m, 2H), 7.17 (d, 1H), 7.11 (s, 1H), 6.89 (d, 1H), 4.99 (m, 1H), 4.88 (m, 1H), 4.79 (br, 1H), 4.19 (t, 2H), 3.75 (m, 4H), 3.25 (m, 3H), 2.92-2.73 (m, 9H), 2.60 (m, 4H), 2.10 (br, 1H), 1.86 (br, 1H), 1.71 (br, 1H), 1.60 (br, 1H), 1.42 (br, 1H), 1.30 (m, 3H), 0.98 (m, 3H)

### Example 115. (S)-quinuclidin-3-yl (5-(4-(2-fluoroethoxy)phenyl)-2,2-dimethyl-2,3-dihydro-1H-inden-1-yl)carbamate

¹H-NMR (400 MHz, CDCl₃) δ 7.52 (d, 2H), 7.35 (m, 2H), 7.26 (m, 1H), 7.00 (d, 2H), 4.99 (m, 1H), 4.87 (m, 2H), 4.81 (m, 1H), 4.71 (br, 1H), 4.31-4.22 (m, 2H), 3.25 (m, 3H), 2.90-2.74 (m, 7H), 2.10 (br, 1H), 1.86 (br, 1H), 1.71 (br, 1H), 1.59 (br, 1H), 1.42 (br, 1H), 1.30 (m, 3H), 0.98 (m, 3H)

### Example 116. (S)-quinuclidin-3-yl (2,2-dimethyl-5-(4-(2,2,2-trifluoroethoxy)phenyl)-2,3-dihydro-1H-inden-1-yl)carbamate

¹H-NMR (400 MHz, CDCl₃) δ 7.43-7.36 (m, 3H), 7.31-7.25 (m, 2H), 7.15 (s, 1H), 6.91 (d, 1H), 4.99 (m, 1H), 4.88 (m, 1H), 4.79 (br, 1H), 4.42(q, 2H), 3.25 (m, 3H), 2.92-2.73 (m, 7H), 2.10 (br, 1H), 1.86 (br, 1H), 1.71 (br, 1H), 1.60 (br, 1H), 1.42 (br, 1H), 1.30 (m, 3H), 0.98 (m, 3H)

### Example 117. (S)-quinuclidin-3-yl (2,2-dimethyl-5-(4-(2-morpholinoethoxy)phenyl)-2,3-dihydro-1H-inden-1-yl)carbamate

¹H-NMR (400 MHz, CDCl₃) δ 7.50 (d, 2H), 7.39 (m, 2H), 7.24 (m, 1H), 6.97 (d, 2H), 4.98 (m, 1H), 4.88 (m, 1H), 4.79 (br, 1H), 4.16 (t, 2H), 3.76 (m, 4H), 3.25 (m, 3H), 2.92-2.73 (m, 9H), 2.60 (m, 4H), 2.10 (br, 1H), 1.86 (br, 1H), 1.71 (br, 1H), 1.60 (br, 1H), 1.42 (br, 1H), 1.30 (m, 3H), 0.98 (m, 3H)

### Example 118. (S)-quinuclidin-3-yl (2,2-dimethyl-5-(2-(trifluoromethyl)pyridin-4-yl)-2,3-dihydro-1H-inden-1-yl)carbamate

¹H-NMR (400 MHz, CDCl₃) δ 8.76 (d, 1H), 7.87 (s, 1H), 7.67 (d, 1H), 7.49 (m, 2H), 7.37 (m, 1H), 5.00 (m, 1H), 4.93 (m, 1H), 4.80 (br, 1H), 3.25 (m, 1H), 2.92-2.73 (m, 7H), 2.10 (br, 1H), 1.86 (br, 1H), 1.71 (br, 1H), 1.60 (br, 1H), 1.42 (br, 1H), 1.31 (m, 3H), 0.98 (m, 3H)

### Example 119. (S)-quinuclidin-3-yl (5-(2,5-dichloropyridin-4-yl)-2,2-dimethyl-2,3-dihydro-1H-inden-1-yl)carbamate

¹H-NMR (400 MHz, CDCl₃) δ 8.44 (s, 1H), 7.30 (m, 1H), 7.19 (m, 3H), 4.96 (m, 1H), 4.86 (m, 1H), 4.80 (br, 1H), 3.25 (m, 1H), 2.92-2.73 (m, 7H), 2.10 (br, 1H), 1.86 (br, 1H), 1.71 (br, 1H), 1.60 (br, 1H), 1.42 (br, 1H), 1.31 (m, 3H), 0.98 (m, 3H)

### Example 120. (S)-quinuclidin-3-yl (2,2-dimethyl-5-(4-methyl-3,4-dihydro-2H-benzo[b][1,4]oxazin-7-yl)-2,3-dihydro-1H-inden-1-yl)carbamate

¹H-NMR (400 MHz, CDCl₃) δ 7.39 (m, 1H), 7.33 (s, 1H), 7.25 (m, 1H), 7.07 (d, 1H), 7.03 (s, 1H), 6.72 (d, 1H), 4.99 (m, 1H), 4.88 (m, 1H), 4.79 (br, 1H), 4.33 (m, 2H), 3.29 (m, 2H), 3.25 (m, 1H), 2.92 (s, 3H), 2.91-2.73 (m, 7H), 2.10 (br, 1H), 1.86 (br, 1H), 1.71 (br, 1H), 1.60 (br, 1H), 1.42 (br, 1H), 1.30 (m, 3H), 0.98 (m, 3H)

### Example 121. (S)-quinuclidin-3-yl (5-(2-chloro-5-methoxyphenyl)-2,2-dimethyl-2,3-dihydro-1H-inden-1-yl)carbamate

¹H-NMR (400 MHz, CDCl₃) δ 7.36 (d, 1H), 7.28 (m, 3H), 6.86 (m, 1H), 6.81 (m, 1H), 5.00 (m, 2H), 4.80 (br, 1H), 3.81 (s, 3H), 3.25 (m, 1H), 2.91-2.74 (m, 7H), 2.10 (br, 1H), 1.86 (br, 1H), 1.71 (br, 1H), 1.60 (br, 1H), 1.42 (br, 1H), 1.31 (m, 3H), 0.99 (m, 3H)

### Example 122. (S)-quinuclidin-3-yl (5-(3-chloro-4-methoxyphenyl)-2,2-dimethyl-2,3-dihydro-1H-inden-1-yl)carbamate

¹H-NMR (400 MHz, CDCl₃) δ 7.59 (d, 1H), 7.44 (d, 1H), 7.37 (m, 1H), 7.25 (m, 1H), 6.98 (d, 1H), 4.98 (m, 2H), 4.80 (br, 1H), 3.94 (s, 3H), 3.25 (m, 1H), 2.88-2.73 (m, 7H), 2.10 (br, 1H), 1.86 (br, 1H), 1.71 (br, 1H), 1.60 (br, 1H), 1.42 (br, 1H), 1.31 (m, 3H), 0.98 (m, 3H)

### Example 123. (S)-quinuclidin-3-yl (5-(2-chloro-4-methoxyphenyl)-2,2-dimethyl-2,3-dihydro-1H-inden-1-yl)carbamate

¹H-NMR (400 MHz, CDCl₃) δ 7.24 (m, 1H), 7.22 (m, 3H), 7.01 (s, 1H), 6.85 (d, 1H), 4.99 (s, 2H), 4.79 (br, 1H), 3.84 (s, 3H), 3.25 (m, 1H), 2.91-2.73 (m, 7H), 2.10 (br, 1H), 1.86 (br, 1H), 1.71 (br, 1H), 1.60 (br, 1H), 1.42 (br, 1H), 1.31 (m, 3H), 0.97 (m, 3H)

### Example 124. (S)-quinuclidin-3-yl (5-(2-chloro-3-methoxyphenyl)-2,2-dimethyl-2,3-dihydro-1H-inden-1-yl)carbamate

¹H-NMR (400 MHz, CDCl₃) δ 7.31-7.22 (m, 4H), 6.94 (d, 2H), 4.99 (s, 2H), 4.79 (br, 1H), 3.95 (s, 3H), 3.25 (m, 1H), 2.91-2.74 (m, 7H), 2.10 (br, 1H), 1.86 (br, 1H), 1.71 (br, 1H), 1.60 (br, 1H), 1.42 (br, 1H), 1.31 (m, 3H), 0.98 (m, 3H)

### Example 125. (S)-quinuclidin-3-yl (5-(4-chloro-3-methoxyphenyl)-2,2-dimethyl-2,3-dihydro-1H-inden-1-yl)carbamate

¹H-NMR (400 MHz, CDCl₃) δ 7.41-7.37 (m, 3H), 7.27 (m, 1H), 7.08 (m, 2H), 5.00 (m, 1H), 4.91 (m, 1H), 4.80 (br, 1H), 3.97 (s, 3H), 3.25 (m, 1H), 2.91-2.75 (m, 7H), 2.10 (br, 1H), 1.86 (br, 1H), 1.71 (br, 1H), 1.60 (br, 1H), 1.42 (br, 1H), 1.30 (m, 3H), 0.98 (m, 3H)

### Example 126. (S)-quinuclidin-3-yl (5-(2,3-dichloro-4-methoxyphenyl)-2,2-dimethyl-2,3-dihydro-1H-inden-1-yl)carbamate

¹H-NMR (400 MHz, CDCl₃) δ 7.27 (m, 1H), 7.21 (m, 1H), 7.19 (d, 2H), 6.90 (d, 1H), 5.00 (s, 2H), 4.80 (br, 1H), 3.95 (s, 3H), 3.25 (m, 1H), 2.91-2.73 (m, 7H), 2.10 (br, 1H), 1.86 (br, 1H), 1.71 (br, 1H), 1.60 (br, 1H), 1.42 (br, 1H), 1.30 (m, 3H), 0.98 (m, 3H)

### Example 127. (S)-quinuclidin-3-yl (5-(3-chloro-4-isopropoxyphenyl)-2,2-dimethyl-2,3-dihydro-1H-inden-1-yl)carbamate

¹H-NMR (400 MHz, CDCl₃) δ 7.59 (s, 1H), 7.39 (m, 2H), 7.33 (s, 1H), 7.26 (m, 1H), 7.00 (d, 1H), 4.96 (m, 2H), 4.79 (br, 1H), 4.60 (m, 1H), 3.25 (m, 1H), 2.88-2.73 (m, 7H), 2.10 (br, 1H), 1.86 (br, 1H), 1.71 (br, 1H), 1.60 (br, 1H), 1.40 (br, 1H), 1.38 (d, 6H), 1.30 (m, 3H), 0.97 (m, 3H)

### Example 128. (S)-quinuclidin-3-yl (5-(2-fluoro-4-methoxyphenyl)-2,2-dimethyl-2,3-dihydro-1H-inden-1-yl)carbamate

¹H-NMR (400 MHz, CDCl₃) δ 7.35-7.27 (m, 4H), 6.77 (d, 1H), 6.69 (d, 1H), 4.97 (m, 2H), 4.80 (br, 1H), 3.84 (s, 3H), 3.25 (m, 1H), 2.89-2.73 (m, 7H), 2.10 (br, 1H), 1.86 (br, 1H), 1.71 (br, 1H), 1.60 (br, 1H), 1.40 (br, 1H), 1.38 (d, 6H), 1.30 (m, 3H), 0.98 (m, 3H)

### Example 129. (S)-quinuclidin-3-yl (5-(3-(dimethylamino)-4-fluorophenyl)-2,2-dimethyl-2,3-dihydro-1H-inden-1-yl)carbamate

¹H-NMR (400 MHz, CDCl₃) δ 7.39 (m, 1H), 7.30 (s, 1H), 7.25 (m, 1H), 7.09 (s, 1H), 7.04 (m, 2H), 4.99 (m, 1H), 4.88 (m, 1H), 4.80 (br, 1H), 3.25 (m, 1H), 2.90 (s, 6H), 2.89-2.74 (m, 7H), 2.10 (br, 1H), 1.86 (br, 1H), 1.71 (br, 1H), 1.60 (br, 1H), 1.40 (br, 1H), 1.38 (d, 6H), 1.30 (m, 3H), 0.98 (m, 3H)

### Example 130. (S)-quinuclidin-3-yl (5-(chroman-6-yl)-2,2-dimethyl-2,3-dihydro-1H-inden-1-yl)carbamate

¹H-NMR (400 MHz, CDCl₃) δ 7.37 (m, 1H), 7.33 (s, 1H), 7.25 (m, 3H), 6.85 (d, 1H), 4.99 (m, 1H), 4.88 (m, 1H), 4.80 (br, 1H), 4.23 (m, 2H), 3.25 (m, 1H), 2.89-2.74 (m, 9H), 2.10 (br, 1H), 1.86 (br, 1H), 1.71 (br, 1H), 1.60 (br, 1H), 1.40 (br, 1H), 1.30 (m, 3H), 0.98 (m, 3H)

### Example 131. (S)-quinuclidin-3-yl (5-(4-cyclopropylphenyl)-2,2-dimethyl-2,3-dihydro-1H-inden-1-yl)carbamate

¹H-NMR (400 MHz, CDCl₃) δ 7.47 (d, 2H), 7.42 (m, 1H), 7.38 (s, 1H), 7.25 (m, 1H), 7.13 (d, 2H), 4.99 (m, 1H), 4.88 (m, 1H), 4.80 (br, 1H), 3.25 (m, 1H), 2.89-2.74 (m, 7H), 2.10 (br, 1H), 1.91 (m, 1H), 1.86 (br, 1H), 1.71 (br, 1H), 1.60 (br, 1H), 1.40 (br, 1H), 1.30 (m, 3H), 0.98 (m, 6H), 0.74 (m, 2H)

### Example 132. (S)-quinuclidin-3-yl (5-(4-(dimethylamino)phenyl)-2,2-dimethyl-2,3-dihydro-1H-inden-1-yl)carbamate

¹H-NMR (400 MHz, CDCl₃) δ 7.48 (d, 2H), 7.39 (m, 1H), 7.36 (s, 1H), 7.21 (m, 2H), 6.80 (d, 2H), 4.99 (m, 1H), 4.88 (m, 1H), 4.80 (br, 1H), 3.25 (m, 1H), 3.00 (s, 6H), 2.89-2.73 (m, 7H), 2.10 (br, 1H), 1.86 (br, 1H), 1.71 (br, 1H), 1.60 (br, 1H), 1.40 (br, 1H), 1.30 (m, 3H), 0.99 (m, 3H)

### Example 133. (S)-quinuclidin-3-yl (5-(4-ethoxy-3-fluorophenyl)-2,2-dimethyl-2,3-dihydro-1H-inden-1-yl)carbamate

¹H-NMR (400 MHz, CDCl₃) δ 7.38-7.32 (m, 2H), 7.27 (m, 3H), 6.99 (t, 1H), 4.99 (m, 1H), 4.88 (m, 1H), 4.80 (br, 1H), 4.15 (m, 2H), 3.25 (m, 1H), 2.89-2.73 (m, 7H), 2.10 (br, 1H), 1.86 (br, 1H), 1.71 (br, 1H), 1.60 (br, 1H), 1.48 (t, 3H), 1.40 (br, 1H), 1.30 (m, 3H), 0.98 (m, 3H)

### Example 134. (S)-quinuclidin-3-yl (5-(4-methoxy-3-(trifluoromethyl)phenyl)-2,2-dimethyl-2,3-dihydro-1H-inden-1-yl)carbamate

¹H-NMR (400 MHz, CDCl₃) δ 7.76 (s, 1H), 7.69 (d, 1H), 7.31 (m, 2H), 7.27 (m, 1H), 7.07 (d, 1H), 4.99 (m, 1H), 4.88 (m, 1H), 4.80 (br, 1H), 4.95 (s, 3H), 3.25 (m, 1H), 2.89-2.73 (m, 7H), 2.10 (br, 1H), 1.86 (br, 1H), 1.71 (br, 1H), 1.60 (br, 1H), 1.40 (br, 1H), 1.31 (m, 3H), 0.98 (m, 3H)

### Example 135. (S)-quinuclidin-3-yl (2,2-dimethyl-5-(4-propoxyphenyl)-2,3-dihydro-1H-inden-1-yl)carbamate

¹H-NMR (400 MHz, CDCl₃) δ 7.50 (d, 2H), 7.38 (m, 1H), 7.35 (s, 1H), 7.26 (m, 1H), 6.96 (d, 2H), 4.99 (m, 1H), 4.88 (m, 1H), 4.80 (br, 1H), 3.96(q, 2H), 3.25 (m, 1H), 2.89-2.73 (m, 7H), 2.10 (br, 1H), 1.86 (br, 1H), 1.84 (m, 2H), 1.71 (br, 1H), 1.60 (br, 1H), 1.40 (br, 1H), 1.31 (m, 3H), 1.06 (t, 3H), 0.98 (m, 3H)

### Example 136. (S)-quinuclidin-3-yl (5-(4-isobutoxyphenyl)-2,2-dimethyl-2,3-dihydro-1H-inden-1-yl)carbamate

¹H-NMR (400 MHz, CDCl₃) δ 7.50 (d, 2H), 7.39 (m, 1H), 7.36 (s, 1H), 7.26 (m, 1H), 6.96 (d, 2H), 4.99 (m, 1H), 4.88 (m, 1H), 4.80 (br, 1H), 3.77 (d, 2H), 3.25 (m, 1H), 2.89-2.73 (m, 7H), 2.10 (m, 2H), 1.86 (br, 1H), 1.71 (br, 1H), 1.60 (br, 1H), 1.40 (br, 1H), 1.31 (m, 3H), 1.05 (d, 6H), 0.98 (m, 3H)

### Example 137. (S)-quinuclidin-3-yl (2,2-dimethyl-5-(thiophen-3-yl)-2,3-dihydro-1H-inden-1-yl)carbamate

¹H-NMR (400 MHz, CDCl₃) δ 7.46-7.41 (m, 3H), 7.38 (m, 2H), 7.21 (m, 1H), 4.98 (m, 1H), 4.87 (m, 1H), 4.80 (br, 1H), 3.25 (m, 1H), 2.89-2.73 (m, 7H), 2.10 (br, 1H), 1.86 (br, 1H), 1.71 (br, 1H), 1.60 (br, 1H), 1.40 (br, 1H), 1.31 (m, 3H), 0.98 (m, 3H)

### Example 138. (S)-quinuclidin-3-yl (2,2-dimethyl-5-(4-methylthiophen-3-yl)-2,3-dihydro-1H-inden-1-yl)carbamate

¹H-NMR (400 MHz, CDCl₃) δ 7.28-7.16 (m, 4H), 7.02 (s, 1H), 4.99 (m, 1H), 4.87 (m, 1H), 4.80 (br, 1H), 3.25 (m, 1H), 2.26 (s, 3H), 2.89-2.73 (m, 7H), 2.10 (br, 1H), 1.86 (br, 1H), 1.71 (br, 1H), 1.60 (br, 1H), 1.40 (br, 1H), 1.31 (m, 3H), 0.98 (m, 3H)

### Example 139. (S)-quinuclidin-3-yl (5-(3-(2-(dimethylamino)ethoxy)phenyl)-2,2-dimethyl-2,3-dihydro-1H-inden-1-yl)carbamate

¹H-NMR (400 MHz, CDCl₃) δ 7.28 (m, 1H), 7.24 (m, 4H), 6.94 (m, 2H), 4.99 (m, 1H), 4.87 (m, 1H), 4.80 (br, 1H), 4.07 (m, 2H), 3.25 (m, 1H), 2.89-2.73 (m, 9H), 2.35 (s, 6H), 2.10 (br, 1H), 1.86 (br, 1H), 1.71 (br, 1H), 1.60 (br, 1H), 1.40 (br, 1H), 1.31 (m, 3H), 0.98 (m, 3H)

### Example 140. (S)-quinuclidin-3-yl (5-(isoquinolin-8-yl)-2,2-dimethyl-2,3-dihydro-1H-inden-1-yl)carbamate

¹H-NMR (400 MHz, CDCl₃) δ 9.31 (s, 1H), 8.51 (d, 1H), 7.82 (d, 1H), 7.72 (m, 2H), 7.52 (d, 1H), 7.36 (m, 3H), 5.24 (m, 1H), 5.07 (m, 1H), 4.81 (m, 1H), 3.25 (m, 1H), 2.89-2.73 (m, 7H), 2.10 (br, 1H), 1.86 (br, 1H), 1.71 (br, 1H), 1.60 (br, 1H), 1.40 (br, 1H), 1.35 (m, 3H), 0.98 (m, 3H)

### Example 141. (S)-quinuclidin-3-yl (5-(isoquinolin-5-yl)-2,2-dimethyl-2,3-dihydro-1H-inden-1-yl)carbamate

¹H-NMR (400 MHz, CDCl₃) δ 9.31 (s, 1H), 8.47 (d, 1H), 7.99 (d, 1H), 7.71 (d, 1H), 7.65 (m, 2H), 7.39-7.27 (m, 3H), 5.05 (m, 2H), 4.82 (m, 1H), 3.25 (m, 1H), 2.89-2.73 (m, 7H), 2.10 (br, 1H), 1.86 (br, 1H), 1.71 (br, 1H), 1.60 (br, 1H), 1.40 (br, 1H), 1.34 (m, 3H), 0.98 (m, 3H)

### Example 142. (S)-quinuclidin-3-yl (5-(isoquinolin-4-yl)-2,2-dimethyl-2,3-dihydro-1H-inden-1-yl)carbamate

¹H-NMR (400 MHz, CDCl₃) δ 9.25 (s, 1H), 8.44 (s, 1H), 8.04 (d, 1H), 7.91 (d, 1H), 7.65 (m, 2H), 7.38 (m, 3H), 5.09 (m, 2H), 4.82 (m, 1H), 3.25 (m, 1H), 2.89-2.73 (m, 7H), 2.10 (br, 1H), 1.86 (br, 1H), 1.71 (br, 1H), 1.60 (br, 1H), 1.40 (br, 1H), 1.35 (m, 3H), 0.99 (m, 3H)

### Example 143. (S)-quinuclidin-3-yl (2,2-dimethyl-5-(quinolin-3-yl)-2,3-dihydro-1H-inden-1-yl)carbamate

¹H-NMR (400 MHz, CDCl₃) δ 9.16 (s, 1H), 8.30 (s, 1H), 8.14 (d, 1H), 7.88 (d, 1H), 7.72 (m, 1H), 7.61-7.53 (m, 3H), 7.39 (m, 1H), 5.05-4.94 (m, 2H), 4.82 (m, 1H), 3.25 (m, 1H), 2.91-2.77 (m, 7H), 2.10 (br, 1H), 1.86 (br, 1H), 1.71 (br, 1H), 1.60 (br, 1H), 1.40 (br, 1H), 1.34 (m, 3H), 1.00 (m, 3H)

### Example 144. (S)-quinuclidin-3-yl (5-(4-isopropoxyphenyl)-2,2-dimethyl-2,3-dihydro-1H-inden-1-yl)carbamate

¹H-NMR (400 MHz, CDCl₃) δ 7.48 (d, 1H), 7.37 (m, 1H), 7.35 (s, 1H), 7.25 (m, 1H), 6.95 (d, 2H), 4.98 (m, 1H), 4.88 (m, 1H), 4.80 (br, 1H), 4.61 (m, 1H), 3.25 (m, 1H), 2.89-2.74 (m, 7H), 2.10 (br, 1H), 1.86 (br, 1H), 1.71 (br, 1H), 1.60 (br, 1H), 1.40 (br, 1H), 1.36 (d, 6H), 1.30 (m, 3H), 0.98 (m, 3H)

### Example 145. (S)-quinuclidin-3-yl (5-(2-isopropoxyphenyl)-2,2-dimethyl-2,3-dihydro-1H-inden-1-yl)carbamate

¹H-NMR (400 MHz, CDCl₃) δ 7.42 (m, 1H), 7.36 (s, 1H), 7.30-7.21 (m, 3H), 7.01 (m, 2H), 4.98 (m, 1H), 4.88 (m, 1H), 4.80 (br, 1H), 4.42 (m, 1H), 3.25 (m, 1H), 2.91-2.72 (m, 7H), 2.10 (br, 1H), 1.86 (br, 1H), 1.71 (br, 1H), 1.60 (br, 1H), 1.40 (br, 1H), 1.29 (m, 3H), 1.26 (d, 6H), 0.98 (m, 3H)

### Example 146. (S)-quinuclidin-3-yl (5-(4-(tert-butyl)phenyl)-2,2-dimethyl-2,3-dihydro-1H-inden-1-yl)carbamate

¹H-NMR (400 MHz, CDCl₃) δ 7.53 (m, 2H), 7.47 (m, 2H), 7.26 (m, 2H), 7.24 (m, 1H), 4.98 (m, 1H), 4.88 (m, 1H), 4.80 (br, 1H), 3.25 (m, 1H), 2.91-2.72 (m, 7H), 2.10 (br, 1H), 1.86 (br, 1H), 1.71 (br, 1H), 1.60 (br, 1H), 1.40 (br, 1H), 1.36 (s, 9H), 1.29 (m, 3H), 0.98 (m, 3H)

### Example 147. (S)-quinuclidin-3-yl (5-(4-ethylphenyl)-2,2-dimethyl-2,3-dihydro-1H-inden-1-yl)carbamate

¹H-NMR (400 MHz, CDCl₃) δ 7.50 (d, 2H), 7.43 (m, 1H), 7.38 (s, 1H), 7.27 (m, 3H), 4.98 (m, 1H), 4.88 (m, 1H), 4.80 (br, 1H), 3.25 (m, 1H), 2.91-2.72 (m, 7H), 2.69 (m, 2H), 2.10 (br, 1H), 1.86 (br, 1H), 1.71 (br, 1H), 1.60 (br, 1H), 1.40 (br, 1H), 1.30 (m, 6H), 0.98 (m, 3H)

### Example 148. (S)-quinuclidin-3-yl (2,2-dimethyl-5-(4-morpholinophenyl)-2,3-dihydro-1H-inden-1-yl)carbamate

¹H-NMR (400 MHz, CDCl₃) δ 7.51 (d, 2H), 7.39 (m, 1H), 7.36 (s, 1H), 7.27 (m, 1H), 6.97 (d, 2H), 4.98 (m, 1H), 4.88 (m, 1H), 4.80 (br, 1H), 3.89 (m, 4H), 3.25 (m, 1H), 3.21 (m, 4H), 2.91-2.72 (m, 7H), 2.10 (br, 1H), 1.86 (br, 1H), 1.71 (br, 1H), 1.60 (br, 1H), 1.40 (br, 1H), 1.30 (m, 3H), 0.98 (m, 3H)

### Example 149. (S)-quinuclidin-3-yl (5-(4-(methoxymethyl)phenyl)-2,2-dimethyl-2,3-dihydro-1H-inden-1-yl)carbamate

¹H-NMR (400 MHz, CDCl₃) δ 7.56 (d, 2H), 7.42 (m, 4H), 7.27 (m, 1H), 4.98 (m, 1H), 4.88 (m, 1H), 4.80 (br, 1H), 4.50 (s, 2H), 3.42 (s, 3H), 3.25 (m, 1H), 2.89-2.74 (m, 7H), 2.10 (br, 1H), 1.86 (br, 1H), 1.71 (br, 1H), 1.60 (br, 1H), 1.40 (br, 1H), 1.29 (m, 3H), 0.98 (m, 3H)

### Example 150. (S)-quinuclidin-3-yl (5-(4-(2,2-difluoroethoxy)phenyl)-2,2-dimethyl-2,3-dihydro-1H-inden-1-yl)carbamate

¹H-NMR (400 MHz, CDCl₃) δ 7.53 (d, 2H), 7.39 (m, 2H), 7.27 (m, 1H), 6.99 (d, 2H), 6.25-5.98 (t, 1H), 4.98 (m, 2H), 4.79 (br, 1H), 4.21 (m, 2H), 3.25 (m, 1H), 2.90-2.73 (m, 7H), 2.10 (br, 1H), 1.86 (br, 1H), 1.71 (br, 1H), 1.60 (br, 1H), 1.40 (br, 1H), 1.29 (m, 3H), 0.97 (m, 3H)

### Example 151. (S)-quinuclidin-3-yl (2,2-dimethyl-5-(3-methyl-4-morpholinophenyl)-2,3-dihydro-1H-inden-1-yl)carbamate

¹H-NMR (400 MHz, CDCl₃) δ 7.40-7.36 (m, 4H), 7.24 (m, 1H), 7.07 (d, 1H), 4.95 (m, 2H), 4.78 (br, 1H), 3.89 (m, 4H), 3.25 (m, 1H), 2.95 (m, 4H), 2.91-2.73 (m, 7H), 2.38 (s, 3H), 2.10 (br, 1H), 1.86 (br, 1H), 1.71 (br, 1H), 1.60 (br, 1H), 1.40 (br, 1H), 1.30 (m, 3H), 0.98 (m, 3H)

### Example 152. (S)-quinuclidin-3-yl (5-(3-fluoro-4-morpholinophenyl)-2,2-dimethyl-2,3-dihydro-1H-inden-1-yl)carbamate

¹H-NMR (400 MHz, CDCl₃) δ 7.34 (m, 2H), 7.26 (m, 2H), 7.23 (m, 1H), 6.98 (t, 1H), 4.96 (m, 1H), 4.91 (br, 1H), 4.78 (br, 1H), 3.90 (m, 4H), 3.25 (m, 1H), 3.14 (m, 4H), 2.91-2.73 (m, 7H), 2.10 (br, 1H), 1.86 (br, 1H), 1.71 (br, 1H), 1.60 (br, 1H), 1.40 (br, 1H), 1.30 (m, 3H), 0.98 (m, 3H)

### Example 153. (S)-quinuclidin-3-yl (5-(3-chloro-4-morpholinophenyl)-2,2-dimethyl-2,3-dihydro-1H-inden-1-yl)carbamate

¹H-NMR (400 MHz, CDCl₃) δ 7.60 (s, 1H), 7.45 (d, 1H), 7.36 (m, 2H), 7.26 (m, 1H), 7.10 (d, 1H), 4.99 (m, 1H), 4.91 (br, 1H), 4.78 (br, 1H), 3.90 (m, 4H), 3.25 (m, 1H), 3.09 (m, 4H), 2.91-2.73 (m, 7H), 2.10 (br, 1H), 1.86 (br, 1H), 1.71 (br, 1H), 1.60 (br, 1H), 1.40 (br, 1H), 1.30 (m, 3H), 0.98 (m, 3H)

### Example 154. (S)-quinuclidin-3-yl (5-(3,4-dihydro-2H-benzo[b][1,4]dioxepin-7-yl)-2,2-dimethyl-2,3-dihydro-1H-inden-1-yl)carbamate

¹H-NMR (400 MHz, CDCl₃) δ 7.37 (m, 1H), 7.34 (s, 1H), 7.24 (m, 1H), 7.20 (s, 1H), 7.15 (d, 1H), 7.02 (d, 1H), 4.99 (m, 1H), 4.91 (br, 1H), 4.78 (br, 1H), 4.23 (m, 4H), 3.25 (m, 1H), 2.91-2.73 (m, 7H), 2.22 (m, 2H), 2.10 (br, 1H), 1.86 (br, 1H), 1.71 (br, 1H), 1.60 (br, 1H), 1.40 (br, 1H), 1.29 (m, 3H), 0.97 (m, 3H)

### Example 155. (S)-quinuclidin-3-yl (5-(3-(difluoromethyl)phenyl)-2,2-dimethyl-2,3-dihydro-1H-inden-1-yl)carbamate

¹H-NMR (400 MHz, CDCl₃) δ 7.71 (m, 2H), 7.54 (m, 2H), 7.41 (m, 2H), 7.30 (m, 1H), 6.85-6.57 (t, 1H), 5.01 (m, 1H), 4.92 (br, 1H), 4.80 (br, 1H), 3.25 (m, 1H), 2.91-2.73 (m, 7H), 2.10 (br, 1H), 1.86 (br, 1H), 1.71 (br, 1H), 1.60 (br, 1H), 1.40 (br, 1H), 1.29 (m, 3H), 0.97 (m, 3H)

### Example 156. (S)-quinuclidin-3-yl (2,2-dimethyl-5-(quinolin-8-yl)-2,3-dihydro-1H-inden-1-yl)carbamate

¹H-NMR (400 MHz, CDCl₃) δ 8.96 (m, 1H), 8.22 (d, 1H), 7.84 (d, 1H), 7.72 (m, 1H), 7.62 (d, 1H), 7.58 (m, 1H), 7.50 (s, 1H), 7.44 (d, 1H), 7.35 (d, 1H), 5.00 (m, 1H), 4.92 (br, 1H), 4.80 (br, 1H), 3.25 (m, 1H), 2.91-2.73 (m, 7H), 2.10 (br, 1H), 1.86 (br, 1H), 1.71 (br, 1H), 1.60 (br, 1H), 1.40 (br, 1H), 1.28 (m, 3H), 0.95 (m, 3H)

### Example 157. (S)-quinuclidin-3-yl (5-(benzofuran-3-yl)-2,2-dimethyl-2,3-dihydro-1H-inden-1-yl)carbamate

¹H-NMR (400 MHz, CDCl₃) δ 7.84 (d, 1H), 7.78 (s, 1H), 7.55 (d, 1H), 7.49 (m, 1H), 7.45 (s, 1H), 7.34 (m, 3H), 5.02 (m, 1H), 4.92 (br, 1H), 4.80 (br, 1H), 3.25 (m, 1H), 2.91-2.73 (m, 7H), 2.10 (br, 1H), 1.86 (br, 1H), 1.71 (br, 1H), 1.60 (br, 1H), 1.40 (br, 1H), 1.32 (m, 3H), 0.97 (m, 3H)

### Example 158. (S)-quinuclidin-3-yl (5-(benzofuran-2-yl)-2,2-dimethyl-2,3-dihydro-1H-inden-1-yl)carbamate

¹H-NMR (400 MHz, CDCl₃) δ 7.73 (d, 1H), 7.71 (s, 1H), 7.59 (d, 1H), 7.53 (d, 1H), 7.26 (m, 3H), 5.02 (m, 1H), 4.92 (br, 1H), 4.80 (br, 1H), 3.25 (m, 1H), 2.91-2.74 (m, 7H), 2.10 (br, 1H), 1.86 (br, 1H), 1.71 (br, 1H), 1.60 (br, 1H), 1.40 (br, 1H), 1.31 (m, 3H), 0.97 (m, 3H)

### Example 159. (S)-quinuclidin-3-yl (5-(4-methoxyphenyl)-2,2-dimethyl-2,3-dihydro-1H-inden-1-yl)carbamate

¹H-NMR (400 MHz, CDCl₃) δ 7.50 (d, 2H), 7.38 (m, 1H), 7.35 (s, 1H), 7.26 (m, 1H), 6.97 (d, 2H), 4.99 (m, 1H), 4.92 (br, 1H), 4.79 (br, 1H), 3.85 (s, 3H), 3.25 (m, 1H), 2.91-2.74 (m, 7H), 2.10 (br, 1H), 1.86 (br, 1H), 1.71 (br, 1H), 1.60 (br, 1H), 1.40 (br, 1H), 1.30 (m, 3H), 0.98 (m, 3H)

### Example 160. (S)-quinuclidin-3-yl (5-(4-ethoxyphenyl)-2,2-dimethyl-2,3-dihydro-1H-inden-1-yl)carbamate

¹H-NMR (400 MHz, CDCl₃) δ 7.50 (d, 2H), 7.38 (m, 1H), 7.35 (s, 1H), 7.26 (m, 1H), 6.95 (d, 2H), 4.99 (m, 1H), 4.92 (br, 1H), 4.79 (br, 1H), 4.08 (m, 2H), 3.25 (m, 1H), 2.91-2.74 (m, 7H), 2.10 (br, 1H), 1.86 (br, 1H), 1.71 (br, 1H), 1.60 (br, 1H), 1.42 (t, 3H), 1.40 (br, 1H), 1.30 (m, 3H), 0.98 (m, 3H)

### Example 161. (S)-quinuclidin-3-yl (5-(4-isobutylphenyl)-2,2-dimethyl-2,3-dihydro-1H-inden-1-yl)carbamate

¹H-NMR (400 MHz, CDCl₃) δ 7.50 (d, 2H), 7.44 (m, 1H), 7.39 (s, 1H), 7.26 (m, 1H), 7.20 (d, 2H), 4.99 (m, 1H), 4.92 (br, 1H), 4.79 (br, 1H), 3.25 (m, 1H), 2.91-2.74 (m, 7H), 2.51 (d, 2H), 2.10 (br, 1H), 1.90 (m, 1H), 1.87 (br, 1H), 1.71 (br, 1H), 1.60 (br, 1H), 1.40 (br, 1H), 1.30 (m, 3H), 0.98 (m, 3H), 0.93 (d, 6H)

### Example 162. (S)-quinuclidin-3-yl (5-(2,4-dichloro-3-methoxyphenyl)-2,2-dimethyl-2,3-dihydro-1H-inden-1-yl)carbamate

¹H-NMR (400 MHz, CDCl₃) δ 7.34 (d, 2H), 7.26 (m, 2H), 7.20 (s, 1H), 7.02 (d, 1H), 4.99 (m, 2H), 4.79 (br, 1H), 3.94 (s, 3H), 3.25 (m, 1H), 2.91-2.74 (m, 7H), 2.10 (br, 1H), 1.87 (br, 1H), 1.71 (br, 1H), 1.60 (br, 1H), 1.40 (br, 1H), 1.30 (m, 3H), 0.98 (m, 3H)

### Example 163. (S)-quinuclidin-3-yl (2,2-dimethyl-5-(4-propylphenyl)-2,3-dihydro-1H-inden-1-yl)carbamate

¹H-NMR (400 MHz, CDCl₃) δ 7.50 (d, 2H), 7.42 (m, 1H), 7.39 (s, 1H), 7.25 (m, 3H), 4.99 (m, 1H), 4.92 (br, 1H), 4.79 (br, 1H), 3.25 (m, 1H), 2.91-2.74 (m, 7H), 2.61 (m, 2H), 2.10 (br, 1H), 1.87 (br, 1H), 1.67 (m, 3H), 1.59 (br, 1H), 1.40 (br, 1H), 1.30 (m, 3H), 0.98 (m, 6H)

### Example 164. (S)-quinuclidin-3-yl (5-(4-butylphenyl)-2,2-dimethyl-2,3-dihydro-1H-inden-1-yl)carbamate

¹H-NMR (400 MHz, CDCl₃) δ 7.50 (d, 2H), 7.42 (m, 1H), 7.39 (s, 1H), 7.25 (m, 3H), 4.99 (m, 1H), 4.92 (br, 1H), 4.79 (br, 1H), 3.25 (m, 1H), 2.91-2.74 (m, 7H), 2.65 (m, 2H), 2.10 (br, 1H), 1.87 (br, 1H), 1.70 (br, 1H), 1.64 (m, 3H), 1.40 (m, 3H), 1.30 (m, 3H), 0.98 (m, 6H)

### Example 165. (S)-quinuclidin-3-yl (5-(2,3-dimethoxyphenyl)-2,2-dimethyl-2,3-dihydro-1H-inden-1-yl)carbamate

¹H-NMR (400 MHz, CDCl₃) δ 7.38 (m, 1H), 7.36 (s, 1H), 7.23 (m, 1H), 7.10 (m, 1H), 6.92 (d, 2H), 4.99 (m, 1H), 4.92 (br, 1H), 4.79 (br, 1H), 3.91 (s, 3H), 3.58 (s, 3H), 3.25 (m, 1H), 2.91-2.74 (m, 7H), 2.10 (br, 1H), 1.87 (br, 1H), 1.70 (br, 1H), 1.64 (br, 1H), 1.40 (br, 1H), 1.30 (m, 3H), 0.98 (m, 3H)

### Example 166. (S)-quinuclidin-3-yl (5-(2,4-dimethoxyphenyl)-2,2-dimethyl-2,3-dihydro-1H-inden-1-yl)carbamate

¹H-NMR (400 MHz, CDCl₃) δ 7.35 (m, 1H), 7.33 (s, 1H), 7.23 (m, 1H), 7.20 (d, 2H), 4.99 (m, 1H), 4.92 (br, 1H), 4.79 (br, 1H), 3.85 (s, 3H), 3.80 (s, 3H), 3.25 (m, 1H), 2.91-2.74 (m, 7H), 2.10 (br, 1H), 1.87 (br, 1H), 1.70 (br, 1H), 1.64 (br, 1H), 1.40 (br, 1H), 1.30 (m, 3H), 0.98 (m, 3H)

### Example 167. (S)-quinuclidin-3-yl (5-(2,5-dimethoxyphenyl)-2,2-dimethyl-2,3-dihydro-1H-inden-1-yl)carbamate

¹H-NMR (400 MHz, CDCl₃) δ 7.39 (m, 1H), 7.33 (s, 1H), 7.26 (m, 1H), 6.87 (m, 3H), 4.99 (m, 1H), 4.92 (br, 1H), 4.79 (br, 1H), 3.81 (s, 3H), 3.76 (s, 3H), 3.25 (m, 1H), 2.91-2.74 (m, 7H), 2.10 (br, 1H), 1.87 (br, 1H), 1.70 (br, 1H), 1.64 (br, 1H), 1.40 (br, 1H), 1.30 (m, 3H), 0.98 (m, 3H)

### Example 168. (S)-quinuclidin-3-yl (5-(2,6-dimethoxyphenyl)-2,2-dimethyl-2,3-dihydro-1H-inden-1-yl)carbamate

¹H-NMR (400 MHz, CDCl₃) δ 7.26 (m, 2H), 7.19 (m, 1H), 7.15 (s, 1H), 6.65 (d, 2H), 4.99 (m, 1H), 4.92 (br, 1H), 4.79 (br, 1H), 3.71 (s, 6H), 3.25 (m, 1H), 2.91-2.74 (m, 7H), 2.10 (br, 1H), 1.87 (br, 1H), 1.70 (br, 1H), 1.64 (br, 1H), 1.40 (br, 1H), 1.30 (m, 3H), 0.98 (m, 3H)

### Example 169. (S)-quinuclidin-3-yl (5-(3,5-dimethoxyphenyl)-2,2-dimethyl-2,3-dihydro-1H-inden-1-yl)carbamate

¹H-NMR (400 MHz, CDCl₃) δ 7.43 (m, 1H), 7.38 (s, 1H), 7.26 (m, 1H), 6.71 (s, 2H), 6.46 (s, 1H), 4.99 (m, 1H), 4.92 (br, 1H), 4.79 (br, 1H), 3.85 (s, 6H), 3.25 (m, 1H), 2.91-2.74 (m, 7H), 2.10 (br, 1H), 1.87 (br, 1H), 1.70 (br, 1H), 1.64 (br, 1H), 1.40 (br, 1H), 1.30 (m, 3H), 0.98 (m, 3H)

### Example 170. (S)-quinuclidin-3-yl (5-(4-ethoxy-3-(trifluoromethyl)phenyl)-2,2-dimethyl-2,3-dihydro-1H-inden-1-yl)carbamate

¹H-NMR (400 MHz, CDCl₃) δ 7.75 (s, 1H), 7.66 (d, 1H), 7.34 (m, 2H), 7.26 (m, 1H), 7.04 (d, 1H), 5.00 (m, 1H), 4.92 (br, 1H), 4.79 (br, 1H), 4.16 (m, 2H), 3.25 (m, 1H), 2.91-2.74 (m, 7H), 2.10 (br, 1H), 1.87 (br, 1H), 1.70 (br, 1H), 1.64 (br, 1H), 1.45 (t, 3H), 1.40 (br, 1H), 1.30 (m, 3H), 0.98 (m, 3H)

### Example 171. (S)-quinuclidin-3-yl (5-(2,5-difluoro-4-methoxyphenyl)-2,2-dimethyl-2,3-dihydro-1H-inden-1-yl)carbamate

¹H-NMR (400 MHz, CDCl₃) δ 7.32 (m, 3H), 7.16 (m, 1H), 6.78 (m, 1H), 4.99 (m, 1H), 4.92 (br, 1H), 4.79 (br, 1H), 3.91 (s, 3H), 3.25 (m, 1H), 2.91-2.74 (m, 7H), 2.10 (br, 1H), 1.87 (br, 1H), 1.70 (br, 1H), 1.64 (br, 1H), 1.40 (br, 1H), 1.30 (m, 3H), 0.98 (m, 3H)

### Example 172. (S)-quinuclidin-3-yl (5-(2-fluoro-5-isopropoxyphenyl)-2,2-dimethyl-2,3-dihydro-1H-inden-1-yl)carbamate

¹H-NMR (400 MHz, CDCl₃) δ 7.37 (m, 2H), 7.26 (m, 1H), 7.04 (t, 1H), 6.91 (m, 1H), 6.80 (m, 1H), 4.99 (m, 1H), 4.92 (br, 1H), 4.79 (br, 1H), 4.51 (m, 1H), 3.25 (m, 1H), 2.91-2.74 (m, 7H), 2.10 (br, 1H), 1.87 (br, 1H), 1.70 (br, 1H), 1.64 (br, 1H), 1.40 (br, 1H), 1.33 (d, 6H), 1.30 (m, 3H), 0.98 (m, 3H)

### Example 173. (S)-quinuclidin-3-yl (5-(3,5-dimethyl-4-propoxyphenyl)-2,2-dimethyl-2,3-dihydro-1H-inden-1-yl)carbamate

¹H-NMR (400 MHz, CDCl₃) δ 7.39 (m, 1H), 7.35 (s, 1H), 7.25 (m, 1H), 7.21 (s, 2H), 4.99 (m, 1H), 4.92 (br, 1H), 4.79 (br, 1H), 3.78 (t, 2H), 3.25 (m, 1H), 2.91-2.74 (m, 7H), 2.34 (s, 6H), 2.10 (br, 1H), 1.85 (m, 3H), 1.70 (br, 1H), 1.64 (br, 1H), 1.40 (br, 1H), 1.30 (m, 3H), 1.10 (t, 3H), 0.98 (m, 3H)

### Example 174. (S)-quinuclidin-3-yl (5-(4-ethoxy-3,5-dimethylphenyl)-2,2-dimethyl-2,3-dihydro-1H-inden-1-yl)carbamate

¹H-NMR (400 MHz, CDCl₃) δ 7.39 (m, 1H), 7.35 (s, 1H), 7.25 (m, 1H), 7.22 (s, 2H), 4.99 (m, 1H), 4.92 (br, 1H), 4.79 (br, 1H), 3.91(q, 2H), 3.25 (m, 1H), 2.91-2.74 (m, 7H), 2.34 (s, 6H), 2.10 (br, 1H), 1.85 (br, 1H), 1.70 (br, 1H), 1.64 (br, 1H), 1.44 (t, 3H), 1.40 (br, 1H), 1.30 (m, 3H), 0.98 (m, 3H)

### Example 175. (S)-quinuclidin-3-yl (5-(2-ethylphenyl)-2,2-dimethyl-2,3-dihydro-1H-inden-1-yl)carbamate

¹H-NMR (400 MHz, CDCl₃) δ 7.31 (d, 2H), 7.25 (m, 2H), 7.18 (m, 2H), 7.11 (s, 1H), 4.99 (m, 1H), 4.92 (br, 1H), 4.79 (br, 1H), 4.12(q, 2H), 3.25 (m, 1H), 2.91-2.74 (m, 7H), 2.10 (br, 1H), 1.85 (br, 1H), 1.70 (br, 1H), 1.64 (br, 1H), 1.40 (br, 1H), 1.30 (m, 3H), 1.10 (t, 3H), 0.98 (m, 3H)

### Example 176. (S)-quinuclidin-3-yl (5-(2-methoxyphenyl)-2,2-dimethyl-2,3-dihydro-1H-inden-1-yl)carbamate

¹H-NMR (400 MHz, CDCl₃) δ 7.39 (m, 1H), 7.37-7.25 (m, 4H), 7.04-6.78 (m, 2H), 4.99 (m, 1H), 4.92 (br, 1H), 4.79 (br, 1H), 3.82 (s, 3H), 3.25 (m, 1H), 2.91-2.74 (m, 7H), 2.10 (br, 1H), 1.85 (br, 1H), 1.70 (br, 1H), 1.64 (br, 1H), 1.40 (br, 1H), 1.30 (m, 3H), 0.98 (m, 3H)

### Example 177. (S)-quinuclidin-3-yl (5-(2-ethoxyphenyl)-2,2-dimethyl-2,3-dihydro-1H-inden-1-yl)carbamate

¹H-NMR (400 MHz, CDCl₃) δ 7.43 (m, 1H), 7.35 (s, 1H), 7.32-7.23 (m, 3H), 7.03-6.96 (m, 2H), 4.99 (m, 1H), 4.92 (br, 1H), 4.79 (br, 1H), 4.05(q, 2H), 3.25 (m, 1H), 2.91-2.74 (m, 7H), 2.10 (br, 1H), 1.85 (br, 1H), 1.70 (br, 1H), 1.64 (br, 1H), 1.40 (br, 1H), 1.37 (t, 3H), 1.30 (m, 3H), 0.98 (m, 3H)

### Example 178. (S)-quinuclidin-3-yl (5-(2-chloro-5-(trifluoromethoxy)phenyl)-2,2-dimethyl-2,3-dihydro-1H-inden-1-yl)carbamate

¹H-NMR (400 MHz, CDCl₃) δ 7.48 (d, 1H), 7.26 (m, 2H), 7.21 (d, 2H), 7.14 (d, 1H), 4.99 (m, 1H), 4.92 (br, 1H), 4.79 (br, 1H), 3.25 (m, 1H), 2.91-2.74 (m, 7H), 2.10 (br, 1H), 1.85 (br, 1H), 1.70 (br, 1H), 1.64 (br, 1H), 1.40 (br, 1H), 1.30 (m, 3H), 0.98 (m, 3H)

### Example 179. (S)-quinuclidin-3-yl (5-(4-fluoro-2-methoxyphenyl)-2,2-dimethyl-2,3-dihydro-1H-inden-1-yl)carbamate

¹H-NMR (400 MHz, CDCl₃) δ 7.27 (m, 1H), 7.25 (m, 3H), 6.70 (m, 2H), 4.99 (m, 1H), 4.92 (br, 1H), 4.79 (br, 1H), 3.80 (s, 3H), 3.25 (m, 1H), 2.91-2.74 (m, 7H), 2.10 (br, 1H), 1.85 (br, 1H), 1.70 (br, 1H), 1.64 (br, 1H), 1.40 (br, 1H), 1.30 (m, 3H), 0.98 (m, 3H)

### Example 180. (S)-quinuclidin-3-yl (5-(4-methoxy-2-methylphenyl)-2,2-dimethyl-2,3-dihydro-1H-inden-1-yl)carbamate

¹H-NMR (400 MHz, CDCl₃) δ 7.26 (m, 1H), 7.15 (m, 3H), 6.77 (m, 2H), 4.99 (m, 1H), 4.92 (br, 1H), 4.79 (br, 1H), 3.84 (s, 3H), 3.25 (m, 1H), 2.91-2.74 (m, 7H), 2.26 (s, 3H), 2.10 (br, 1H), 1.85 (br, 1H), 1.70 (br, 1H), 1.64 (br, 1H), 1.40 (br, 1H), 1.30 (m, 3H), 0.98 (m, 3H)

### Example 181. (S)-quinuclidin-3-yl (5-(4-(tert-butoxymethyl)phenyl)-2,2-dimethyl-2,3-dihydro-1H-inden-1-yl)carbamate

¹H-NMR (400 MHz, CDCl₃) δ 7.54 (d, 2H), 7.40 (m, 4H), 7.26 (m, 1H), 4.99 (m, 1H), 4.92 (br, 1H), 4.79 (br, 1H), 4.48 (s, 2H), 3.25 (m, 1H), 2.91-2.74 (m, 7H), 2.10 (br, 1H), 1.85 (br, 1H), 1.70 (br, 1H), 1.64 (br, 1H), 1.40 (br, 1H), 1.32 (s, 9H), 1.30 (m, 3H), 0.99 (m, 3H)

### Example 182. (S)-quinuclidin-3-yl (5-(2-chloro-3-(trifluoromethyl)phenyl)-2,2-dimethyl-2,3-dihydro-1H-inden-1-yl)carbamate

¹H-NMR (400 MHz, CDCl₃) δ 7.72 (d, 1H), 7.49 (d, 1H), 7.40 (m, 1H), 7.32 (m, 1H), 7.22 (m, 2H), 5.00 (m, 2H), 4.79 (br, 1H), 3.25 (m, 1H), 2.91-2.74 (m, 7H), 2.10 (br, 1H), 1.85 (br, 1H), 1.70 (br, 1H), 1.64 (br, 1H), 1.40 (br, 1H), 1.30 (m, 3H), 0.98 (m, 3H)

### Example 183. (S)-quinuclidin-3-yl (5-(2-chloro-4-(trifluoromethyl)phenyl)-2,2-dimethyl-2,3-dihydro-1H-inden-1-yl)carbamate

¹H-NMR (400 MHz, CDCl₃) δ 7.74 (s, 1H), 7.57 (d, 1H), 7.43 (d, 1H), 7.33-7.23 (m, 3H), 5.00 (m, 2H), 4.79 (br, 1H), 3.25 (m, 1H), 2.91-2.74 (m, 7H), 2.10 (br, 1H), 1.85 (br, 1H), 1.70 (br, 1H), 1.64 (br, 1H), 1.40 (br, 1H), 1.30 (m, 3H), 0.98 (m, 3H)

### Example 184. (S)-quinuclidin-3-yl (5-(2,6-dimethoxypyridin-3-yl)-2,2-dimethyl-2,3-dihydro-1H-inden-1-yl)carbamate

¹H-NMR (400 MHz, CDCl₃) δ 7.56 (d, 1H), 7.36 (m, 1H), 7.49 (m, 1H), 7.32 (s, 1H), 6.38 (d, 1H), 4.99 (m, 1H), 4.86 (br, 1H), 4.79 (br, 1H), 3.97 (s, 6H), 3.25 (m, 1H), 2.91-2.74 (m, 7H), 2.10 (br, 1H), 1.85 (br, 1H), 1.70 (br, 1H), 1.64 (br, 1H), 1.40 (br, 1H), 1.30 (m, 3H), 0.98 (m, 3H)

### Example 185. (S)-quinuclidin-3-yl (5-(6-(cyclopropylmethoxy)pyridin-3-yl)-2,2-dimethyl-2,3-dihydro-1H-inden-1-yl)carbamate

¹H-NMR (400 MHz, CDCl₃) δ 8.32 (s, 1H), 7.78 (d, 1H), 7.33 (m, 3H), 6.83 (d, 1H), 4.99 (m, 1H), 4.86 (br, 1H), 4.79 (br, 1H), 4.15 (d, 2H), 3.25 (m, 1H), 2.91-2.74 (m, 7H), 2.10 (br, 1H), 1.85 (br, 1H), 1.70 (br, 1H), 1.64 (br, 1H), 1.40 (br, 1H), 1.30 (m, 3H), 0.98 (m, 3H), 0.63 (m, 2H), 0.37 (m, 2H)

### Example 186. (S)-quinuclidin-3-yl (5-(benzo[b]thiophen-2-yl)-2,2-dimethyl-2,3-dihydro-1H-inden-1-yl)carbamate ¹H-NMR (400 MHz, CDCl₃) δ 7.67 (m, 2H), 7.57 (m, 3H), 7.48 (m, 2H), 7.30 (m, 1H), 4.99 (m, 1H), 4.86 (br, 1H), 4.79 (br, 1H), 3.25 (m, 1H), 2.91-2.74 (m, 7H), 2.10 (br, 1H), 1.85 (br, 1H), 1.70 (br, 1H), 1.64 (br, 1H), 1.40 (br, 1H), 1.30 (m, 3H), 0.98 (m, 3H)

### Example 187. (S)-quinuclidin-3-yl (5-(2-(tert-butoxy)pyridin-4-yl)-2,2-dimethyl-2,3-dihydro-1H-inden-1-yl)carbamate ¹H-NMR (400 MHz, CDCl₃) δ 8.16 (d, 1H), 7.45 (m, 2H), 7.28 (m, 1H), 7.03 (d, 1H), 6.85 (s, 1H), 5.00 (m, 1H), 4.86 (br, 1H), 4.79 (br, 1H), 3.25 (m, 1H), 2.91-2.74 (m, 7H), 2.10 (br, 1H), 1.85 (br, 1H), 1.70 (br, 1H), 1.64 (br, 1H), 1.61 (s, 9H), 1.40 (br, 1H), 1.30 (m, 3H), 0.98 (m, 3H)

### Example 188. (S)-quinuclidin-3-yl (5-(6-methoxy-5-(trifluoromethyl)pyridin-3-yl)-2,2-dimethyl-2,3-dihydro-1H-inden-1-yl)carbamate

¹H-NMR (400 MHz, CDCl₃) δ 8.50 (s, 1H), 8.03 (s, 1H), 7.33 (m, 3H), 5.00 (m, 1H), 4.86 (br, 1H), 4.79 (br, 1H), 4.09 (s, 3H), 3.25 (m, 1H), 2.91-2.74 (m, 7H), 2.10 (br, 1H), 1.85 (br, 1H), 1.70 (br, 1H), 1.64 (br, 1H), 1.61 (s, 9H), 1.40 (br, 1H), 1.30 (m, 3H), 0.98 (m, 3H)

### Example 189. (S)-quinuclidin-3-yl (5-(6-methoxynaphthalen-2-yl)-2,2-dimethyl-2,3-dihydro-1H-inden-1-yl)carbamate ¹H-NMR (400 MHz, CDCl₃) δ 7.96 (s, 1H), 7.81 (m, 2H), 7.70 (m, 1H), 7.54 (m, 2H), 7.32 (m, 1H), 7.18 (m, 2H), 5.02 (m, 1H), 4.86 (br, 1H), 4.79 (br, 1H), 3.96 (s, 3H), 3.25 (m, 1H), 2.91-2.74 (m, 7H), 2.10 (br, 1H), 1.85 (br, 1H), 1.70 (br, 1H), 1.64 (br, 1H), 1.40 (br, 1H), 1.32 (m, 3H), 1.00 (m, 3H)

### Example 190. (S)-quinuclidin-3-yl (5-([1,1′-biphenyl]-3-yl)-2,2-dimethyl-2,3-dihydro-1H-inden-1-yl)carbamate

¹H-NMR (400 MHz, CDCl₃) δ 7.78 (s, 1H), 7.66 (m, 2H), 7.56 (m, 1H), 7.50 (m, 5H), 7.39 (m, 2H), 7.33 (m, 1H), 5.05 (m, 1H), 4.92 (br, 1H), 4.82 (br, 1H), 3.25 (m, 1H), 2.91˜-2.74 (m, 7H), 2.10 (br, 1H), 1.85 (br, 1H), 1.70 (br, 1H), 1.64 (br, 1H), 1.40 (br, 1H), 1.32 (m, 3H), 1.00 (m, 3H)

### Example 191. (S)-quinuclidin-3-yl (2,2-dimethyl-5-(quinoxalin-6-yl)-2,3-dihydro-1H-inden-1-yl)carbamate

¹H-NMR (400 MHz, CDCl₃) δ 8.85 (d, 2H), 8.28 (s, 1H), 8.16 (m, 1H), 8.04 (m, 1H), 7.59 (m, 2H), 7.33 (m, 1H), 5.02 (m, 2H), 4.79 (br, 1H), 3.25 (m, 1H), 2.91-2.74 (m, 7H), 2.10 (br, 1H), 1.85 (br, 1H), 1.70 (br, 1H), 1.64 (br, 1H), 1.40 (br, 1H), 1.32 (m, 3H), 1.00 (m, 3H)

### Example 192. (S)-quinuclidin-3-yl (5-(3,5-dimethylphenyl)-2,2-dimethyl-2,3-dihydro-1H-inden-1-yl)carbamate

¹H-NMR (400 MHz, CDCl₃) δ 7.41 (m, 2H), 7.26 (m, 1H), 7.19 (s, 2H), 6.99 (s, 1H), 4.99 (m, 1H), 4.81 (br, 1H), 4.79 (br, 1H), 3.25 (m, 1H), 2.91-2.74 (m, 7H), 2.38 (s, 6H), 2.10 (br, 1H), 1.85 (br, 1H), 1.70 (br, 1H), 1.64 (br, 1H), 1.40 (br, 1H), 1.31 (m, 3H), 0.99 (m, 3H)

### Example 193. (S)-quinuclidin-3-yl (5-(furan-2-yl)-2,2-dimethyl-2,3-dihydro-1H-inden-1-yl)carbamate

¹H-NMR (400 MHz, CDCl₃) δ 7.54 (m, 1H), 7.49 (s, 1H), 7.45 (s, 1H), 7.23 (m, 1H), 6.63 (m, 1H), 6.47 (m, 1H), 4.99 (m, 1H), 4.81 (br, 1H), 4.79 (br, 1H), 3.25 (m, 1H), 2.91-2.74 (m, 7H), 2.38 (s, 6H), 2.10 (br, 1H), 1.85 (br, 1H), 1.70 (br, 1H), 1.64 (br, 1H), 1.40 (br, 1H), 1.31 (m, 3H), 0.99 (m, 3H)

### Example 194. (S)-quinuclidin-3-yl (5-(4-isopropoxy-3,5-dimethylphenyl)-2,2-dimethyl-2,3-dihydro-1H-inden-1-yl)carbamate

¹H-NMR (400 MHz, CDCl₃) δ 7.40 (m, 1H), 7.36 (s, 1H), 7.25 (m, 3H), 4.98 (m, 1H), 4.81 (br, 1H), 4.79 (br, 1H), 4.13 (m, 1H), 3.25 (m, 1H), 2.91-2.74 (m, 7H), 2.33 (s, 6H), 2.10 (br, 1H), 1.85 (br, 1H), 1.70 (br, 1H), 1.64 (br, 1H), 1.40 (br, 1H), 1.33 (d, 6H), 1.31 (m, 3H), 0.99 (m, 3H)

### Example 195. (S)-quinuclidin-3-yl (5-(4-methoxy-3,5-dimethylphenyl)-2,2-dimethyl-2,3-dihydro-1H-inden-1-yl)carbamate

¹H-NMR (400 MHz, CDCl₃) δ 7.39 (m, 1H), 7.35 (s, 1H), 7.27 (m, 3H), 4.99 (m, 1H), 4.81 (br, 1H), 4.79 (br, 1H), 3.76 (s, 3H), 3.25 (m, 1H), 2.91-2.74 (m, 7H), 2.35 (s, 6H), 2.10 (br, 1H), 1.85 (br, 1H), 1.70 (br, 1H), 1.64 (br, 1H), 1.40 (br, 1H), 1.33 (d, 6H), 1.31 (m, 3H), 0.99 (m, 3H)

### Example 196. (S)-quinuclidin-3-yl (5-(6-isopropoxypyridin-3-yl)-2,2-dimethyl-2,3-dihydro-1H-inden-1-yl)carbamate

¹H-NMR (400 MHz, CDCl₃) δ 8.34 (s, 1H), 7.76 (d, 1H), 7.30 (m, 3H), 6.74 (d, 1H), 5.35 (m, 1H), 4.99 (m, 1H), 4.81 (br, 1H), 4.79 (br, 1H), 3.25 (m, 1H), 2.91-2.74 (m, 7H), 2.10 (br, 1H), 1.85 (br, 1H), 1.70 (br, 1H), 1.64 (br, 1H), 1.40 (br, 1H), 1.38 (d, 6H), 1.31 (m, 3H), 0.99 (m, 3H)

### Example 197. (S)-quinuclidin-3-yl (5-(3-(2-fluoroethoxy)phenyl)-2,2-dimethyl-2,3-dihydro-1H-inden-1-yl)carbamate

¹H-NMR (400 MHz, CDCl₃) δ 7.42 (m, 1H), 7.34 (m, 1H), 7.27 (m, 1H), 7.19 (d, 1H), 7.14 (s, 1H), 6.91 (d, 1H), 5.00 (m, 1H), 4.96 (m, 3H), 4.79 (m, 1H), 4.33 (m, 1H), 4.25 (m, 1H), 3.25 (m, 1H), 2.91-2.74 (m, 7H), 2.10 (br, 1H), 1.85 (br, 1H), 1.70 (br, 1H), 1.60 (br, 1H), 1.40 (br, 1H), 1.31 (m, 3H), 1.00 (m, 3H)

### Example 198. (S)-quinuclidin-3-yl (5-(2-ethoxy-5-methylphenyl)-2,2-dimethyl-2,3-dihydro-1H-inden-1-yl)carbamate

¹H-NMR (400 MHz, CDCl₃) δ 7.41 (m, 1H), 7.34 (s, 1H), 7.22 (m, 1H), 7.09 (s, 1H), 7.07 (d, 1H), 6.88 (d, 1H), 4.99 (m, 1H), 4.96 (m, 1H), 4.79 (m, 1H), 4.00 (m, 2H), 3.25 (m, 1H), 2.91-2.73 (m, 7H), 2.33 (s, 3H), 2.10 (br, 1H), 1.85 (br, 1H), 1.70 (br, 1H), 1.60 (br, 1H), 1.40 (br, 1H), 1.31 (m, 3H), 1.29 (m, 3H), 1.00 (m, 3H)

### Example 199. (S)-quinuclidin-3-yl (2,2-dimethyl-5-(3-propoxyphenyl)-2,3-dihydro-1H-inden-1-yl)carbamate

¹H-NMR (400 MHz, CDCl₃) δ 7.44 (m, 1H), 7.35 (s, 1H), 7.31 (m, 2H), 7.15 (m, 1H), 7.11 (s, 1H), 6.88 (m, 1H), 5.00 (m, 1H), 4.96 (m, 1H), 4.79 (m, 1H), 4.01 (t, 2H), 3.25 (m, 1H), 2.91-2.73 (m, 7H), 2.10 (br, 1H), 1.85 (m, 3H), 1.70 (br, 1H), 1.60 (br, 1H), 1.40 (br, 1H), 1.30 (m, 3H), 1.06 (t, 3H), 0.99 (m, 3H)

### Example 200. (S)-quinuclidin-3-yl (5-(2-methoxy-5-methylphenyl)-2,2-dimethyl-2,3-dihydro-1H-inden-1-yl)carbamate

¹H-NMR (400 MHz, CDCl₃) δ 7.36 (m, 1H), 7.31 (s, 1H), 7.27 (m, 1H), 7.10 (m, 2H), 6.98 (d, 1H), 4.98 (m, 1H), 4.96 (m, 1H), 4.79 (m, 1H), 3.79 (s, 3H), 3.25 (m, 1H), 2.91-2.73 (m, 7H), 2.33 (s, 3H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 201. (S)-quinuclidin-3-yl (5-(2-butoxy-5-fluorophenyl)-2,2-dimethyl-2,3-dihydro-1H-inden-1-yl)carbamate

¹H-NMR (400 MHz, CDCl₃) δ 7.36 (m, 1H), 7.34 (s, 1H), 7.25 (m, 1H), 7.05 (m, 1H), 6.95 (m, 1H), 6.89 (m, 1H), 4.98 (m, 1H), 4.96 (m, 1H), 4.79 (m, 1H), 3.90 (t, 2H), 3.25 (m, 1H), 2.91-2.73 (m, 7H), 2.10 (br, 1H), 1.85 (br, 1H), 1.70 (m, 3H), 1.60 (br, 1H), 1.40 (m, 3H), 1.30 (m, 3H), 0.99 (m, 3H), 0.91 (t, 3H)

### Example 202. (S)-quinuclidin-3-yl (5-(4-butoxy-3-chlorophenyl)-2,2-dimethyl-2,3-dihydro-1H-inden-1-yl)carbamate

¹H-NMR (400 MHz, CDCl₃) δ 7.59 (s, 1H), 7.39 (m, 2H), 7.33 (s, 1H), 7.26 (m, 1H), 6.97 (d, 1H), 4.98 (m, 1H), 4.96 (m, 1H), 4.79 (m, 1H), 4.09 (t, 2H), 3.25 (m, 1H), 2.91-2.73 (m, 7H), 2.10 (br, 1H), 1.85 (m, 3H), 1.70 (br, 1H), 1.60 (m, 3H), 1.40 (br, 3H), 1.30 (m, 3H), 0.99 (m, 3H), 0.97 (t, 3H)

### Example 203. (S)-quinuclidin-3-yl (5-(2,4-dipropoxyphenyl)-2,2-dimethyl-2,3-dihydro-1H-inden-1-yl)carbamate

¹H-NMR (400 MHz, CDCl₃) δ 7.38 (m, 1H), 7.33 (s, 1H), 7.22 (m, 2H), 6.55 (s, 1H), 6.54 (m, 1H), 4.98 (m, 1H), 4.96 (m, 1H), 4.79 (m, 1H), 3.95 (m, 2H), 3.91 (m, 2H), 3.25 (m, 1H), 2.91-2.73 (m, 7H), 2.10 (br, 1H), 1.85 (m, 3H), 1.70 (m, 3H), 1.60 (br, 1H), 1.40 (br, 3H), 1.30 (m, 3H), 1.05 (t, 3H), 0.99 (m, 6H)

### Example 204. (S)-quinuclidin-3-yl (5-(3-chloro-4-ethoxyphenyl)-2,2-dimethyl-2,3-dihydro-1H-inden-1-yl)carbamate

¹H-NMR (400 MHz, CDCl₃) δ 7.60 (s, 1H), 7.41 (m, 2H), 7.33 (s, 1H), 7.26 (m, 1H), 6.97 (d, 1H), 4.99 (m, 1H), 4.96 (m, 1H), 4.79 (m, 1H), 4.15 (m, 2H), 3.25 (m, 1H), 2.91-2.73 (m, 7H), 2.10 (br, 1H), 1.85 (br, 1H), 1.70 (br, 1H), 1.60 (br, 1H), 1.50 (t, 3H), 1.40 (br, 3H), 1.30 (m, 3H), 0.99 (m, 3H)

### Example 205. (S)-quinuclidin-3-yl (5-(3-isobutoxyphenyl)-2,2-dimethyl-2,3-dihydro-1H-inden-1-yl)carbamate

¹H-NMR (400 MHz, CDCl₃) δ 7.44 (m, 1H), 7.39 (s, 1H), 7.31-7.26 (m, 2H), 7.14 (d, 1H), 7.11 (s, 1H), 6.87 (d, 1H), 4.99 (m, 1H), 4.96 (m, 1H), 4.79 (m, 1H), 3.78 (d, 2H), 3.25 (m, 1H), 2.91-2.73 (m, 7H), 2.10 (m, 2H), 1.85 (br, 1H), 1.70 (br, 1H), 1.60 (br, 1H), 1.40 (br, 3H), 1.30 (m, 3H), 1.06 (d, 6H), 0.99 (m, 3H)

### Example 206. (S)-quinuclidin-3-yl (5-(4-ethoxy-2-methylphenyl)-2,2-dimethyl-2,3-dihydro-1H-inden-1-yl)carbamate

¹H-NMR (400 MHz, CDCl₃) δ 7.25 (m, 1H), 7.14 (m, 3H), 6.81 (s, 1H), 6.76 (d, 1H), 4.99 (m, 1H), 4.96 (m, 1H), 4.79 (m, 1H), 4.06 (m, 2H), 3.25 (m, 1H), 2.91-2.73 (m, 7H), 2.24 (s, 3H), 2.10 (br, 1H), 1.85 (br, 1H), 1.70 (br, 1H), 1.60 (br, 1H), 1.43 (m, 4H), 1.30 (m, 3H), 1.00 (m, 3H)

### Example 207. (S)-quinuclidin-3-yl (5-(3-chloro-4-propoxyphenyl)-2,2-dimethyl-2,3-dihydro-1H-inden-1-yl)carbamate

¹H-NMR (400 MHz, CDCl₃) δ 7.59 (s, 1H), 7.38 (m, 3H), 7.25 (m, 1H), 6.97 (d, 1H), 4.99 (m, 1H), 4.96 (m, 1H), 4.79 (m, 1H), 4.05 (m, 2H), 3.25 (m, 1H), 2.91-2.73 (m, 7H), 2.10 (br, 1H), 1.85 (m, 3H), 1.70 (br, 1H), 1.60 (br, 1H), 1.40 (br, 1H), 1.30 (m, 3H), 1.11 (t, 3H), 0.99 (m, 3H)

### Example 208. (S)-quinuclidin-3-yl (5-(5-fluoro-2-isopropoxyphenyl)-2,2-dimethyl-2,3-dihydro-1H-inden-1-yl)carbamate

¹H-NMR (400 MHz, CDCl₃) δ 7.38 (m, 1H), 7.34 (s, 1H), 7.25 (m, 1H), 7.02 (d, 1H), 6.92 (m, 2H), 4.99 (m, 1H), 4.96 (m, 1H), 4.79 (m, 1H), 4.24 (m, 1H), 3.25 (m, 1H), 2.91-2.73 (m, 7H), 2.10 (br, 1H), 1.85 (br, 1H), 1.70 (br, 1H), 1.60 (br, 1H), 1.40 (br, 1H), 1.30 (m, 3H), 1.20 (d, 6H), 0.99 (m, 3H)

### Example 209. (S)-quinuclidin-3-yl (5-(3-fluoro-4-isopropoxyphenyl)-2,2-dimethyl-2,3-dihydro-1H-inden-1-yl)carbamate

¹H-NMR (400 MHz, CDCl₃) δ 7.38 (m, 1H), 7.36 (s, 1H), 7.32-7.24 (m, 3H), 7.01 (t, 1H), 6.92 (m, 2H), 4.99 (m, 1H), 4.96 (m, 1H), 4.79 (m, 1H), 4.57 (m, 1H), 3.25 (m, 1H), 2.91-2.73 (m, 7H), 2.10 (br, 1H), 1.85 (br, 1H), 1.70 (br, 1H), 1.60 (br, 1H), 1.40 (br, 1H), 1.38 (d, 6H), 1.30 (m, 3H), 0.99 (m, 3H)

### Example 210. (S)-quinuclidin-3-yl (5-(5-chloro-2-methoxyphenyl)-2,2-dimethyl-2,3-dihydro-1H-inden-1-yl)carbamate

¹H-NMR (400 MHz, CDCl₃) δ 7.36 (m, 1H), 7.27 (m, 4H), 6.90 (d, 1H), 4.99 (m, 1H), 4.96 (m, 1H), 4.79 (m, 1H), 3.80 (s, 3H), 3.25 (m, 1H), 2.91-2.73 (m, 7H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 211. (S)-quinuclidin-3-yl (5-(2-fluoro-5-methylphenyl)-2,2-dimethyl-2,3-dihydro-1H-inden-1-yl)carbamate

¹H-NMR (400 MHz, CDCl₃) δ 7.37 (m, 2H), 7.29 (m, 1H), 7.20 (d, 1H), 7.07 (m, 1H), 7.00 (m, 1H), 4.99 (m, 1H), 4.96 (m, 1H), 4.79 (m, 1H), 3.25 (m, 1H), 2.91-2.73 (m, 7H), 2.36 (s, 3H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 212. (S)-quinuclidin-3-yl (5-(5-chloro-2-ethoxyphenyl)-2,2-dimethyl-2,3-dihydro-1H-inden-1-yl)carbamate

¹H-NMR (400 MHz, CDCl₃) δ 7.38 (m, 1H), 7.31-7.25 (m, 3H), 7.21 (m, 1H), 6.88 (d, 1H), 4.99 (m, 1H), 4.96 (m, 1H), 4.79 (m, 1H), 4.00 (m, 1H), 3.25 (m, 1H), 2.91-2.73 (m, 7H), 2.10 (br, 1H), 1.85 (br, 1H), 1.70 (br, 1H), 1.60 (br, 1H), 1.40 (br, 1H), 1.30 (m, 6H), 0.99 (m, 3H)

### Example 213. (S)-quinuclidin-3-yl (5-(5-fluoro-2-propoxyphenyl)-2,2-dimethyl-2,3-dihydro-1H-inden-1-yl)carbamate

¹H-NMR (400 MHz, CDCl₃) δ 7.37 (m, 1H), 7.34 (s, 1H), 7.25 (m, 1H), 7.03 (d, 1H), 6.93 (m, 1H), 6.88 (m, 1H), 5.00 (m, 1H), 4.96 (m, 1H), 4.79 (m, 1H), 3.88 (m, 1H), 3.25 (m, 1H), 2.91-2.73 (m, 7H), 2.10 (br, 1H), 1.85 (br, 1H), 1.70 (m, 3H), 1.60 (br, 1H), 1.40 (br, 1H), 1.30 (m, 3H), 0.99 (m, 6H)

### Example 214. (S)-quinuclidin-3-yl (5-(2-fluoro-6-propoxyphenyl)-2,2-dimethyl-2,3-dihydro-1H-inden-1-yl)carbamate

¹H-NMR (400 MHz, CDCl₃) δ 7.25 (m, 4H), 6.76 (m, 2H), 5.00 (m, 1H), 4.96 (m, 1H), 4.79 (m, 1H), 3.91 (m, 1H), 3.25 (m, 1H), 2.91-2.73 (m, 7H), 2.10 (br, 1H), 1.85 (br, 1H), 1.70 (m, 3H), 1.60 (br, 1H), 1.40 (br, 1H), 1.30 (m, 3H), 0.99 (m, 3H), 0.91 (t, 3H)

### Example 215. (S)-quinuclidin-3-yl (5-(3-fluoro-4-methylphenyl)-2,2-dimethyl-2,3-dihydro-1H-inden-1-yl)carbamate

¹H-NMR (400 MHz, CDCl₃) δ 7.40 (m, 1H), 7.36 (s, 1H), 7.25 (m, 4H), 5.00 (m, 1H), 4.96 (m, 1H), 4.79 (m, 1H), 3.25 (m, 1H), 2.91-2.73 (m, 7H), 2.31 (s, 3H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 216. (S)-quinuclidin-3-yl (5-(3-fluoro-4-propoxyphenyl)-2,2-dimethyl-2,3-dihydro-1H-inden-1-yl)carbamate

¹H-NMR (400 MHz, CDCl₃) δ 7.38 (m, 1H), 7.33 (s, 1H), 7.25 (m, 3H), 7.01 (t, 1H), 5.00 (m, 1H), 4.96 (m, 1H), 4.79 (m, 1H), 4.05 (t, 2H), 3.25 (m, 1H), 2.91-2.73 (m, 7H), 2.10 (br, 1H), 1.85 (m, 3H), 1.70 (br, 1H), 1.60 (br, 1H), 1.40 (br, 1H), 1.30 (m, 3H), 1.07 (t, 3H), 0.99 (m, 3H)

### Example 217. (S)-quinuclidin-3-yl (5-(5-chloro-2-isopropoxyphenyl)-2,2-dimethyl-2,3-dihydro-1H-inden-1-yl)carbamate

¹H-NMR (400 MHz, CDCl₃) δ 7.38 (m, 1H), 7.31 (s, 1H), 7.28 (m, 1H), 7.20 (d, 2H), 6.89 (d, 1H), 5.00 (m, 1H), 4.96 (m, 1H), 4.79 (m, 1H), 4.36 (m, 1H), 3.25 (m, 1H), 2.91-2.73 (m, 7H), 2.10 (br, 1H), 1.85 (br, 1H), 1.70 (br, 1H), 1.60 (br, 1H), 1.40 (br, 1H), 1.30 (m, 3H), 1.28 (d, 6H), 0.99 (m, 3H)

### Example 218. (S)-quinuclidin-3-yl (5-(3-butoxyphenyl)-2,2-dimethyl-2,3-dihydro-1H-inden-1-yl)carbamate

¹H-NMR (400 MHz, CDCl₃) δ 7.44 (m, 1H), 7.34 (s, 1H), 7.32 (m, 2H), 7.15 (d, 1H), 7.10 (s, 1H), 6.87 (m, 1H), 5.00 (m, 1H), 4.96 (m, 1H), 4.79 (m, 1H), 4.03 (t, 2H), 3.25 (m, 1H), 2.91-2.73 (m, 7H), 2.10 (br, 1H), 1.85 (m, 3H), 1.70 (br, 1H), 1.60 (m, 3H), 1.40 (br, 1H), 1.30 (m, 3H), 0.99 (m, 3H)

### Example 219. (S)-quinuclidin-3-yl (5-(3-fluoro-5-methylphenyl)-2,2-dimethyl-2,3-dihydro-1H-inden-1-yl)carbamate

¹H-NMR (400 MHz, CDCl₃) δ 7.41 (m, 1H), 7.36 (s, 1H), 7.27 (m, 1H), 7.16 (s, 1H), 7.05 (d, 1H), 6.85 (d, 1H), 4.99 (m, 1H), 4.88 (m, 1H), 4.80 (m, 1H), 3.25 (m, 1H), 2.91-2.73 (m, 7H), 2.41 (s, 3H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 220. (S)-quinuclidin-3-yl (5-(5-fluoro-2-methylphenyl)-2,2-dimethyl-2,3-dihydro-1H-inden-1-yl)carbamate

¹H-NMR (400 MHz, CDCl₃) δ 7.27 (m, 1H), 7.25 (m, 1H), 7.18 (m, 1H), 7.13 (s, 1H), 6.96 (m, 1H), 6.92 (d, 1H), 4.99 (m, 1H), 4.88 (m, 1H), 4.80 (m, 1H), 3.25 (m, 1H), 2.91-2.73 (m, 7H), 2.21 (s, 3H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 221. (S)-quinuclidin-3-yl (5-(2-chloro-5-(trifluoromethyl)phenyl)-2,2-dimethyl-2,3-dihydro-1H-inden-1-yl)carbamate

¹H-NMR (400 MHz, CDCl₃) δ 7.65 (m, 2H), 7.51 (m, 1H), 7.26 (m, 3H), 4.99 (m, 1H), 4.88 (m, 1H), 4.80 (m, 1H), 3.25 (m, 1H), 2.91-2.73 (m, 7H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 222. (S)-quinuclidin-3-yl (5-(3-fluoro-5-methoxyphenyl)-2,2-dimethyl-2,3-dihydro-1H-inden-1-yl)carbamate

¹H-NMR (400 MHz, CDCl₃) δ 7.41 (m, 1H), 7.36 (s, 1H), 7.26 (m, 1H), 6.89 (m, 2H), 6.58 (d, 1H), 4.99 (m, 1H), 4.88 (m, 1H), 4.80 (m, 1H), 3.86 (s, 3H), 3.25 (m, 1H), 2.91-2.73 (m, 7H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 223. (S)-quinuclidin-3-yl (5-(2-chloro-5-methylphenyl)-2,2-dimethyl-2,3-dihydro-1H-inden-1-yl)carbamate

¹H-NMR (400 MHz, CDCl₃) δ 7.33 (d, 1H), 7.26 (m, 2H), 7.23 (s, 1H), 7.14 (s, 1H), 7.07 (d, 1H), 4.99 (m, 1H), 4.88 (m, 1H), 4.80 (m, 1H), 3.25 (m, 1H), 2.91-2.73 (m, 7H), 2.34 (s, 3H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 224. (S)-quinuclidin-3-yl (5-(3-methoxy-5-(trifluoromethyl)phenyl)-2,2-dimethyl-2,3-dihydro-1H-inden-1-yl)carbamate

¹H-NMR (400 MHz, CDCl₃) δ 7.43 (m, 3H), 7.28 (m, 1H), 7.25 (s, 1H), 7.10 (s, 1H), 4.99 (m, 1H), 4.88 (m, 1H), 4.80 (m, 1H), 3.91 (s, 3H), 3.25 (m, 1H), 2.91-2.73 (m, 7H), 2.10 (br, 1H), 1.85 (br, 1H), 1.70 (br, 1H), 1.60 (br, 1H), 1.45 (t, 3H), 1.40 (br, 1H), 1.30 (m, 3H), 0.99 (m, 3H)

### Example 225. (S)-quinuclidin-3-yl (5-(2-chloro-4-fluorophenyl)-2,2-dimethyl-2,3-dihydro-1H-inden-1-yl)carbamate

¹H-NMR (400 MHz, CDCl₃) δ 7.29 (m, 2H), 2.22 (m, 3H), 7.02 (m, 1H), 4.99 (m, 1H), 4.88 (m, 1H), 4.80 (m, 1H), 3.25 (m, 1H), 2.91-2.73 (m, 7H), 2.10 (br, 1H), 1.85 (br, 1H), 1.70 (br, 1H), 1.60 (br, 1H), 1.40 (br, 1H), 1.31 (m, 3H), 0.99 (m, 3H)

### Example 226. (S)-quinuclidin-3-yl (5-(4-fluoro-3-methoxyphenyl)-2,2-dimethyl-2,3-dihydro-1H-inden-1-yl)carbamate

¹H-NMR (400 MHz, CDCl₃) δ 7.37 (m, 2H), 7.28 (m, 1H), 7.13 (m, 2H), 7.08 (m, 1H), 4.99 (m, 1H), 4.88 (m, 1H), 4.80 (m, 1H), 3.96 (s, 3H), 3.25 (m, 1H), 2.91-2.73 (m, 7H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 227. (S)-quinuclidin-3-yl (5-(3,4-dimethylphenyl)-2,2-dimethyl-2,3-dihydro-1H-inden-1-yl)carbamate

¹H-NMR (400 MHz, CDCl₃) δ 7.41 (m, 1H), 7.38 (m, 2H), 7.27 (m, 2H), 7.19 (d, 1H), 4.99 (m, 1H), 4.88 (m, 1H), 4.80 (m, 1H), 3.25 (m, 1H), 2.91-2.73 (m, 7H), 2.33 (s, 3H), 2.31 (s, 3H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 228. (S)-quinuclidin-3-yl (5-(4-fluoro-2-methylphenyl)-2,2-dimethyl-2,3-dihydro-1H-inden-1-yl)carbamate

¹H-NMR (400 MHz, CDCl₃) δ 7.25 (m, 1H), 7.17-7.08 (m, 3H), 6.98-6.90 (m, 2H), 4.99 (m, 1H), 4.88 (m, 1H), 4.80 (m, 1H), 3.25 (m, 1H), 2.91-2.73 (m, 7H), 2.25 (s, 3H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 229. (S)-quinuclidin-3-yl (5-(2-fluoro-4-(trifluoromethyl)phenyl)-2,2-dimethyl-2,3-dihydro-1H-inden-1-yl)carbamate

¹H-NMR (400 MHz, CDCl₃) δ 7.54 (m, 1H), 7.46 (m, 1H), 7.41-7.36 (m, 4H), 4.99 (m, 1H), 4.88 (m, 1H), 4.80 (m, 1H), 3.25 (m, 1H), 2.91-2.73 (m, 7H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 230. (S)-quinuclidin-3-yl (5-(2-fluoro-5-methoxyphenyl)-2,2-dimethyl-2,3-dihydro-1H-inden-1-yl)carbamate

¹H-NMR (400 MHz, CDCl₃) δ 7.39-7.27 (m, 3H), 7.04 (t, 1H), 6.91 (m, 1H), 6.83 (m, 1H), 4.99 (m, 1H), 4.88 (m, 1H), 4.80 (m, 1H), 3.83 (s, 3H), 3.25 (m, 1H), 2.91-2.73 (m, 7H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 231. (S)-quinuclidin-3-yl (5-(2-fluoro-3-methoxyphenyl)-2,2-dimethyl-2,3-dihydro-1H-inden-1-yl)carbamate

¹H-NMR (400 MHz, CDCl₃) δ 7.40-7.27 (m, 3H), 7.12 (t, 1H), 6.97 (m, 2H), 4.99 (m, 1H), 4.88 (m, 1H), 4.80 (m, 1H), 3.93 (s, 3H), 3.25 (m, 1H), 2.91-2.73 (m, 7H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 232. (S)-quinuclidin-3-yl (5-(5-isopropyl-2-methoxyphenyl)-2,2-dimethyl-2,3-dihydro-1H-inden-1-yl)carbamate

¹H-NMR (400 MHz, CDCl₃) δ 7.40 (m, 1H), 7.36 (s, 1H), 7.27 (m, 1H), 7.18 (m, 2H), 6.91 (d, 1H), 4.99 (m, 1H), 4.88 (m, 1H), 4.80 (m, 1H), 3.80 (s, 3H), 3.25 (m, 1H), 2.91 (m, 1H), 2.90-2.75 (m, 7H), 2.10 (br, 1H), 1.85 (br, 1H), 1.70 (br, 1H), 1.60 (br, 1H), 1.40 (br, 1H), 1.30 (m, 3H), 1.26 (d, 6H), 0.99 (m, 3H)

### Example 233. (S)-quinuclidin-3-yl (5-(2-fluoro-4-methylphenyl)-2,2-dimethyl-2,3-dihydro-1H-inden-1-yl)carbamate

¹H-NMR (400 MHz, CDCl₃) δ 7.36-7.27 (m, 4H), 6.98 (m, 2H), 4.99 (m, 1H), 4.88 (m, 1H), 4.80 (m, 1H), 3.25 (m, 1H), 2.91-2.74 (m, 7H), 2.39 (s, 3H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 234. (S)-quinuclidin-3-yl (5-(5-fluoro-2-methoxyphenyl)-2,2-dimethyl-2,3-dihydro-1H-inden-1-yl)carbamate

¹H-NMR (400 MHz, CDCl₃) δ 7.37 (m, 1H), 7.31 (s, 1H), 7.27 (m, 1H), 7.02 (m, 1H), 6.97 (m, 1H), 6.91 (d, 1H), 4.99 (m, 1H), 4.88 (m, 1H), 4.80 (m, 1H), 3.79 (s, 3H), 3.25 (m, 1H), 2.91-2.74 (m, 7H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 235. (S)-quinuclidin-3-yl (2,2-dimethyl-5-(5-methylfuran-3-yl)-2,3-dihydro-1H-inden-1-yl)carbamate

¹H-NMR (400 MHz, CDCl₃) δ 7.47 (m, 1H), 7.45 (s, 1H), 7.19 (m, 1H), 6.51 (s, 1H), 6.04 (s, 1H), 4.99 (m, 1H), 4.88 (m, 1H), 4.80 (m, 1H), 3.25 (m, 1H), 2.91-2.73 (m, 7H), 2.31 (s, 3H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 236. (S)-quinuclidin-3-yl (5-(3-fluoro-2-methoxyphenyl)-2,2-dimethyl-2,3-dihydro-1H-inden-1-yl)carbamate

¹H-NMR (400 MHz, CDCl₃) δ 7.36-7.26 (m, 3H), 7.07 (m, 3H), 4.99 (m, 1H), 4.88 (m, 1H), 4.80 (m, 1H), 3.73 (s, 3H), 3.25 (m, 1H), 2.91-2.74 (m, 7H), 2.10 (br, 1H), 1.85 (br, 1H), 1.70 (br, 1H), 1.60 (br, 1H), 1.40 (br, 1H), 1.30 (m, 3H), 0.99 (m, 6H)

### Example 237. (S)-quinuclidin-3-yl (5-(2-chloro-3-fluorophenyl)-2,2-dimethyl-2,3-dihydro-1H-inden-1-yl)carbamate

¹H-NMR (400 MHz, CDCl₃) δ 7.30-7.23 (m, 4H), 7.13 (m, 2H), 4.99 (m, 1H), 4.88 (m, 1H), 4.80 (m, 1H), 3.25 (m, 1H), 2.91-2.74 (m, 7H), 2.10 (br, 1H), 1.85 (br, 1H), 1.70 (br, 1H), 1.60 (br, 1H), 1.40 (br, 1H), 1.30 (m, 3H), 0.99 (m, 6H)

### Example 238. (S)-quinuclidin-3-yl (5-(benzo[b]thiophen-3-yl)-2,2-dimethyl-2,3-dihydro-1H-inden-1-yl)carbamate

¹H-NMR (400 MHz, CDCl₃) δ 7.92 (m, 2H), 7.46-7.33 (m, 6H), 4.99 (m, 1H), 4.88 (m, 1H), 4.80 (m, 1H), 3.25 (m, 1H), 2.91-2.73 (m, 7H), 2.10 (br, 1H), 1.85 (br, 1H), 1.70 (br, 1H), 1.60 (br, 1H), 1.40 (br, 1H), 1.30 (m, 3H), 0.99 (m, 3H)

### Example 239. (S)-quinuclidin-3-yl (5-(4-fluoro-2-propoxyphenyl)-2,2-dimethyl-2,3-dihydro-1H-inden-1-yl)carbamate

¹H-NMR (400 MHz, CDCl₃) δ 7.35 (m, 1H), 7.30 (s, 1H), 7.25 (m, 2H), 6.70 (m, 2H), 4.99 (m, 1H), 4.88 (m, 1H), 4.80 (m, 1H), 3.91 (t, 2H), 3.25 (m, 1H), 2.91-2.74 (m, 7H), 2.10 (br, 1H), 1.85 (br, 1H), 1.70 (m, 3H), 1.60 (br, 1H), 1.40 (br, 1H), 1.30 (m, 3H), 0.99 (m, 6H)

### Example 240. (S)-quinuclidin-3-yl (5-(3-(tert-butyl)-5-methylphenyl)-2,2-dimethyl-2,3-dihydro-1H-inden-1-yl)carbamate

¹H-NMR (400 MHz, CDCl₃) δ 7.44 (m, 1H), 7.40 (s, 2H), 7.26 (m, 1H), 7.20 (s, 2H), 4.99 (m, 1H), 4.88 (m, 1H), 4.80 (m, 1H), 3.25 (m, 1H), 2.91-2.74 (m, 7H), 2.42 (s, 3H), 2.10 (br, 1H), 1.85 (br, 1H), 1.70 (m, 3H), 1.60 (br, 1H), 1.40 (br, 1H), 1.37 (s, 9H), 1.30 (m, 3H), 0.99 (m, 6H)

### Example 241. (S)-quinuclidin-3-yl (5-(2-chloro-5-ethoxyphenyl)-2,2-dimethyl-2,3-dihydro-1H-inden-1-yl)carbamate

¹H-NMR (400 MHz, CDCl₃) δ 7.34 (d, 1H), 7.28 (d, 2H), 7.23 (s, 1H), 6.85 (s, 1H), 6.81 (d, 1H), 4.99 (m, 1H), 4.88 (m, 1H), 4.80 (m, 1H), 4.00(q, 2H), 3.25 (m, 1H), 2.91-2.74 (m, 7H), 2.10 (br, 1H), 1.85 (br, 1H), 1.70 (m, 3H), 1.60 (br, 1H), 1.41 (m, 4H), 1.30 (m, 3H), 0.99 (m, 6H)

### Example 242. (S)-quinuclidin-3-yl (5-(4-chloro-3-fluorophenyl)-2,2-dimethyl-2,3-dihydro-1H-inden-1-yl)carbamate

¹H-NMR (400 MHz, CDCl₃) δ 7.44 (m, 1H), 7.37 (m, 3H), 7.28 (m, 2H), 4.99 (m, 1H), 4.88 (m, 1H), 4.80 (m, 1H), 3.25 (m, 1H), 2.91-2.74 (m, 7H), 2.10 (br, 1H), 1.85 (br, 1H), 1.70 (br, 1H), 1.60 (br, 1H), 1.41 (br, 1H), 1.30 (m, 3H), 0.99 (m, 3H)

### Example 243. (S)-quinuclidin-3-yl (5-(2-fluoro-3-methylphenyl)-2,2-dimethyl-2,3-dihydro-1H-inden-1-yl)carbamate

¹H-NMR (400 MHz, CDCl₃) δ 7.38 (m, 2H), 7.35-7.23 (m, 2H), 7.14 (m, 1H), 7.08 (t, 1H), 4.99 (m, 1H), 4.88 (m, 1H), 4.80 (m, 1H), 3.25 (m, 1H), 2.91-2.74 (m, 7H), 2.34 (s, 3H), 2.10 (br, 1H), 1.85 (br, 1H), 1.70 (br, 1H), 1.60 (br, 1H), 1.41 (br, 1H), 1.30 (m, 3H), 0.99 (m, 3H)

### Example 244. (S)-quinuclidin-3-yl (5-(5-cyano-2-methoxyphenyl)-2,2-dimethyl-2,3-dihydro-1H-inden-1-yl)carbamate

¹H-NMR (400 MHz, CDCl₃) δ 7.64 (d, 1H), 7.57 (s, 1H), 7.31 (m, 3H), 7.02 (d, 1H), 4.99 (m, 1H), 4.88 (m, 1H), 4.80 (m, 1H), 3.88 (s, 3H), 3.25 (m, 1H), 2.91-2.74 (m, 7H), 2.10 (br, 1H), 1.85 (br, 1H), 1.70 (br, 1H), 1.60 (br, 1H), 1.41 (br, 1H), 1.30 (m, 3H), 0.99 (m, 3H)

### Example 245. (S)-quinuclidin-3-yl (5-(3-ethoxy-5-(trifluoromethyl)phenyl)-2,2-dimethyl-2,3-dihydro-1H-inden-1-yl)carbamate

¹H-NMR (400 MHz, CDCl₃) δ 7.43 (d, 1H), 7.38 (s, 2H), 7.30 (m, 2H), 7.09 (s, 1H), 4.99 (m, 1H), 4.88 (m, 1H), 4.80 (m, 1H), 4.13 (m, 2H), 3.25 (m, 1H), 2.91-2.74 (m, 7H), 2.10 (br, 1H), 1.85 (br, 1H), 1.70 (br, 1H), 1.60 (br, 1H), 1.41 (m, 3H), 1.30 (m, 3H), 0.99 (m, 3H)

### Example 246. (S)-quinuclidin-3-yl (5-(3-fluoro-5-(trifluoromethyl)phenyl)-2,2-dimethyl-2,3-dihydro-1H-inden-1-yl)carbamate

¹H-NMR (400 MHz, CDCl₃) δ 7.61 (s, 1H), 7.45 (m, 2H), 7.32 (s, 1H), 7.30-7.27 (m, 2H), 4.99 (m, 1H), 4.88 (m, 1H), 4.80 (m, 1H), 3.25 (m, 1H), 2.91-2.74 (m, 7H), 2.10 (br, 1H), 1.85 (br, 1H), 1.70 (br, 1H), 1.60 (br, 1H), 1.41 (br, 1H), 1.30 (m, 3H), 0.99 (m, 3H)

### Example 247. (S)-quinuclidin-3-yl (2,2-dimethyl-5-(3-methyl-4-(trifluoromethoxy)phenyl)-2,3-dihydro-1H-inden-1-yl)carbamate

¹H-NMR (400 MHz, CDCl₃) δ 7.43 (s, 1H), 7.38 (m, 3H), 7.26 (m, 3H), 4.99 (m, 1H), 4.88 (m, 1H), 4.80 (m, 1H), 3.25 (m, 1H), 2.91-2.74 (m, 7H), 2.38 (s, 3H), 2.10 (br, 1H), 1.85 (br, 1H), 1.70 (br, 1H), 1.60 (br, 1H), 1.41 (br, 1H), 1.30 (m, 3H), 0.99 (m, 3H)

### Example 248. (S)-quinuclidin-3-yl (2,2-dimethyl-5-(3-(methylthio)-5-(trifluoromethyl)phenyl)-2,3-dihydro-1H-inden-1-yl)carbamate

¹H-NMR (400 MHz, CDCl₃) δ 7.57 (d, 2H), 7.38 (m, 3H), 7.29 (m, 1H), 4.99 (m, 1H), 4.88 (m, 1H), 4.80 (m, 1H), 3.25 (m, 1H), 2.91-2.74 (m, 7H), 2.57 (s, 3H), 2.10 (br, 1H), 1.85 (br, 1H), 1.70 (br, 1H), 1.60 (br, 1H), 1.41 (br, 1H), 1.30 (m, 3H), 0.99 (m, 3H)

### Example 249. (S)-quinuclidin-3-yl (5-(3-fluoro-4-(trifluoromethoxy)phenyl)-2,2-dimethyl-2,3-dihydro-1H-inden-1-yl)carbamate

¹H-NMR (400 MHz, CDCl₃) δ 7.41-7.27 (m, 6H), 4.99 (m, 1H), 4.88 (m, 1H), 4.80 (m, 1H), 3.25 (m, 1H), 2.91-2.74 (m, 7H), 2.10 (br, 1H), 1.85 (br, 1H), 1.70 (br, 1H), 1.60 (br, 1H), 1.41 (br, 1H), 1.30 (m, 3H), 0.99 (m, 3H)

### Example 250. (S)-quinuclidin-3-yl (5-(2-ethoxy-4-(trifluoromethyl)phenyl)-2,2-dimethyl-2,3-dihydro-1H-inden-1-yl)carbamate

¹H-NMR (400 MHz, CDCl₃) δ 7.40 (m, 2H), 7.33 (s, 1H), 7.26 (m, 2H), 7.17 (s, 1H), 4.99 (m, 1H), 4.88 (m, 1H), 4.80 (m, 1H), 4.10 (m, 2H), 3.25 (m, 1H), 2.91-2.74 (m, 7H), 2.10 (br, 1H), 1.85 (br, 1H), 1.70 (br, 1H), 1.60 (br, 1H), 1.41 (m, 4H), 1.30 (m, 3H), 0.99 (m, 3H)

### Example 251. (S)-quinuclidin-3-yl (5-(2-methoxy-5-(trifluoromethyl)phenyl)-2,2-dimethyl-2,3-dihydro-1H-inden-1-yl)carbamate

¹H-NMR (400 MHz, CDCl₃) δ 7.57 (d, 1H), 7.54 (s, 1H), 7.37 (m, 1H), 7.29 (s, 2H), 7.02 (d, 1H), 4.99 (m, 1H), 4.88 (m, 1H), 4.80 (m, 1H), 3.86 (s, 3H), 3.25 (m, 1H), 2.91-2.74 (m, 7H), 2.10 (br, 1H), 1.85 (br, 1H), 1.70 (br, 1H), 1.59 (br, 1H), 1.41 (br, 1H), 1.30 (m, 3H), 0.99 (m, 3H)

### Example 252. (S)-quinuclidin-3-yl (5-(2,4-bis(trifluoromethyl)phenyl)-2,2-dimethyl-2,3-dihydro-1H-inden-1-yl)carbamate

¹H-NMR (400 MHz, CDCl₃) δ 8.00 (s, 1H), 7.81 (d, 1H), 7.48 (d, 1H), 7.26 (m, 1H), 7.14 (m, 1H), 7.12 (s, 1H), 4.99 (m, 1H), 4.88 (m, 1H), 4.80 (m, 1H), 3.25 (m, 1H), 2.91-2.74 (m, 7H), 2.10 (br, 1H), 1.85 (br, 1H), 1.70 (br, 1H), 1.59 (br, 1H), 1.41 (br, 1H), 1.30 (m, 3H), 0.99 (m, 3H)

### Example 253. (S)-quinuclidin-3-yl (5-(3-chloro-2-methylphenyl)-2,2-dimethyl-2,3-dihydro-1H-inden-1-yl)carbamate

¹H-NMR (400 MHz, CDCl₃) δ 7.36 (d, 1H), 7,26(m, 1H), 7.17-7.08 (m, 4H), 4.99 (m, 1H), 4.88 (m, 1H), 4.80 (m, 1H), 3.25 (m, 1H), 2.91-2.74 (m, 7H), 2.25 (s, 3H), 2.10 (br, 1H), 1.85 (br, 1H), 1.70 (br, 1H), 1.59 (br, 1H), 1.41 (br, 1H), 1.30 (m, 3H), 0.99 (m, 3H)

### Example 254. (S)-quinuclidin-3-yl (5-(3-methoxy-4-methylphenyl)-2,2-dimethyl-2,3-dihydro-1H-inden-1-yl)carbamate

¹H-NMR (400 MHz, CDCl₃) δ 7.44 (m, 1H), 7.40 (s, 1H), 7,26(m, 1H), 7.18 (d, 1H), 7.06 (d, 1H), 7.02 (s, 1H), 4.99 (m, 1H), 4.88 (m, 1H), 4.80 (m, 1H), 3.90 (s, 3H), 3.25 (m, 1H), 2.91-2.74 (m, 7H), 2.10 (br, 1H), 1.85 (br, 1H), 1.70 (br, 1H), 1.59 (br, 1H), 1.41 (br, 1H), 1.30 (m, 3H), 0.99 (m, 3H)

### Example 255. (S)-quinuclidin-3-yl (5-(4-fluoro-3-methylphenyl)-2,2-dimethyl-2,3-dihydro-1H-inden-1-yl)carbamate

¹H-NMR (400 MHz, CDCl₃) δ 7.38-7.29 (m, 4H), 7,26(m, 1H), 7.03 (t, 1H), 7.02 (s, 1H), 4.99 (m, 1H), 4.88 (m, 1H), 4.80 (m, 1H), 3.25 (m, 1H), 2.91-2.74 (m, 7H), 2.34 (s, 3H), 2.10 (br, 1H), 1.85 (br, 1H), 1.70 (br, 1H), 1.59 (br, 1H), 1.41 (br, 1H), 1.30 (m, 3H), 0.99 (m, 3H)

### Example 256. (S)-quinuclidin-3-yl (5-(2-ethoxy-5-fluorophenyl)-2,2-dimethyl-2,3-dihydro-1H-inden-1-yl)carbamate

¹H-NMR (400 MHz, CDCl₃) δ 7.40 (m, 1H), 7.33 (s, 1H), 7.25 (m, 1H), 7.05 (m, 1H), 6.95 (m, 1H), 6.88 (m, 1H), 4.99 (m, 1H), 4.88 (m, 1H), 4.80 (m, 1H), 3.99 (m, 2H), 3.25 (m, 1H), 2.91-2.74 (m, 7H), 2.10 (br, 1H), 1.85 (br, 1H), 1.70 (br, 1H), 1.59 (br, 1H), 1.41 (br, 1H), 1.30 (m, 6H), 0.99 (m, 3H)

### Example 257. (S)-quinuclidin-3-yl (5-(3-fluoro-5-propoxyphenyl)-2,2-dimethyl-2,3-dihydro-1H-inden-1-yl)carbamate

¹H-NMR (400 MHz, CDCl₃) δ 7.37 (m, 1H), 7.35 (s, 1H), 7.27 (m, 1H), 7.02 (t, 1H), 6.92 (m, 1H), 6.81 (m, 1H), 4.99 (m, 1H), 4.88 (m, 1H), 4.80 (m, 1H), 3.93 (m, 2H), 3.25 (m, 1H), 2.91-2.74 (m, 7H), 2.10 (br, 1H), 1.85 (m, 3H), 1.70 (br, 1H), 1.59 (br, 1H), 1.41 (br, 1H), 1.30 (m, 3H), 1.05 (t, 3H), 0.99 (m, 3H)

### Example 258. (S)-quinuclidin-3-yl (5-(3,5-bis(trifluoromethyl)phenyl)-2,2-dimethyl-2,3-dihydro-1H-inden-1-yl)carbamate

¹H-NMR (400 MHz, CDCl₃) δ 7.99 (s, 2H), 7.85 (s, 1H), 7.44 (m, 2H), 7.32 (m, 1H), 4.99 (m, 1H), 4.88 (m, 1H), 4.80 (m, 1H), 3.25 (m, 1H), 2.91-2.74 (m, 7H), 2.10 (br, 1H), 1.85 (br, 1H), 1.70 (br, 1H), 1.59 (br, 1H), 1.41 (br, 1H), 1.30 (m, 3H), 0.99 (m, 3H)

### Example 259. (S)-quinuclidin-3-yl (5-(3-chloro-4-methylphenyl)-2,2-dimethyl-2,3-dihydro-1H-inden-1-yl)carbamate

¹H-NMR (400 MHz, CDCl₃) δ 7.56 (s, 1H), 7.36 (m, 3H), 7.28 (m, 2H), 4.96 (s, 2H), 4.80 (br, 1H), 3.25 (m, 1H), 2.89-2.73 (m, 7H), 2.39 (s, 3H), 2.05 (br, 1H), 1.85 (br, 1H), 1.68 (br, 1H), 1.58 (br, 1H), 1.40 (br, 1H), 1.29 (m, 3H), 0.98 (m, 3H)

### Example 260. (S)-quinuclidin-3-yl (5-(2,5-dimethylphenyl)-2,2-dimethyl-2,3-dihydro-1H-inden-1-yl)carbamate

¹H-NMR (400 MHz, CDCl₃) δ 7.25 (m, 1H), 7.16 (t, 3H), 7.06 (m, 2H), 4.96 (s, 2H), 4.80 (br, 1H), 3.25 (m, 1H), 2.89-2.73 (m, 7H), 2.34 (s, 3H), 2.20 (s, 3H), 2.05 (br, 1H), 1.85 (br, 1H), 1.68 (br, 1H), 1.58 (br, 1H), 1.40 (br, 1H), 1.29 (m, 3H), 0.98 (m, 3H)

### Example 261. (S)-quinuclidin-3-yl (5-(3-chloro-4-fluorophenyl)-2,2-dimethyl-2,3-dihydro-1H-inden-1-yl)carbamate

¹H-NMR (400 MHz, CDCl₃) δ 7.60 (d, 1H), 7.42 (m, 1H), 7.35 (m, 3H), 7.19 (t, 1H), 4.96 (s, 2H), 4.80 (br, 1H), 3.25 (m, 1H), 2.89-2.73 (m, 7H), 2.05 (br, 1H), 1.85 (br, 1H), 1.68 (br, 1H), 1.58 (br, 1H), 1.40 (br, 1H), 1.29 (m, 3H), 0.98 (m, 3H)

### Example 262. (S)-quinuclidin-3-yl (5-(6-ethoxypyridin-3-yl)-2,2-dimethyl-2,3-dihydro-1H-inden-1-yl)carbamate

¹H-NMR (400 MHz, CDCl₃) δ 8.33 (s, 1H), 7.76 (d, 1H), 7.69 (m, 1H), 7.51 (m, 1H), 7.46 (m, 1H), 7.36-7.27 (m, 1H), 6.77 (d, 1H), 4.96 (s, 2H), 4.80 (br, 1H), 4.37 (m, 2H), 3.25 (m, 1H), 2.89-2.73 (m, 7H), 2.05 (br, 1H), 1.85 (br, 1H), 1.68 (br, 1H), 1.58 (br, 1H), 1.39 (m, 4H), 1.29 (m, 3H), 0.98 (m, 3H)

### Example 263. (S)-quinuclidin-3-yl (5-(2-ethoxypyridin-4-yl)-2,2-dimethyl-2,3-dihydro-1H-inden-1-yl)carbamate

¹H-NMR (400 MHz, CDCl₃) δ 8.17 (d, 1H), 7.46 (m, 2H), 7.30 (m, 1H), 7.06 (d, 1H), 6.91 (s, 1H), 4.97 (m, 2H), 4.80 (br, 1H), 4.38 (m, 2H), 3.24 (m, 1H), 2.89-2.73 (m, 7H), 2.05 (br, 1H), 1.85 (br, 1H), 1.68 (br, 1H), 1.58 (br, 1H), 1.40 (m, 4H), 1.30 (m, 3H), 0.96 (m, 3H)

### Example 264. (S)-quinuclidin-3-yl (5-(2-isopropoxypyridin-4-yl)-2,2-dimethyl-2,3-dihydro-1H-inden-1-yl)carbamate

¹H-NMR (400 MHz, CDCl₃) δ 8.17 (d, 1H), 7.44 (m, 2H), 7.31 (m, 1H), 7.04 (d, 1H), 6.87 (s, 1H), 5.34 (m, 1H), 4.97 (s, 2H), 4.80 (br, 1H), 3.24 (m, 1H), 2.89-2.73 (m, 7H), 2.05 (br, 1H), 1.85 (br, 1H), 1.68 (br, 1H), 1.58 (br, 1H), 1.40 (br, 1H), 1.36 (d, 6H), 1.28 (m, 3H), 0.97 (m, 3H)

### Example 265. (S)-quinuclidin-3-yl (2,2-dimethyl-5-(6-propoxypyridin-3-yl)-2,3-dihydro-1H-inden-1-yl)carbamate

¹H-NMR (400 MHz, CDCl₃) δ 8.34 (s, 1H), 7.76 (d, 1H), 7.34-7.32 (m, 3H), 6.79 (d, 1H), 4.96 (m, 2H), 4.80 (br, 1H), 4.28 (m, 2H), 3.25 (m, 1H), 2.89-2.74 (m, 7H), 2.05 (br, 1H), 1.85 (br, 1H), 1.81 (m, 2H), 1.68 (br, 1H), 1.58 (br, 1H), 1.40 (br, 1H), 1.29 (m, 3H), 1.06 (t, 3H), 0.98 (m, 3H)

### Example 266. (S)-quinuclidin-3-yl (5-(6-butoxypyridin-3-yl)-2,2-dimethyl-2,3-dihydro-1H-inden-1-yl)carbamate

¹H-NMR (400 MHz, CDCl₃) δ 8.33 (s, 1H), 7.76 (d, 1H), 7.34-7.31 (m, 3H), 6.78 (d, 1H), 4.96 (s, 2H), 4.80 (br, 1H), 4.31 (m, 2H), 3.25 (m, 1H), 2.89-2.73 (m, 7H), 2.05 (br, 1H), 1.85 (br, 1H), 1.78 (m, 2H), 1.68 (br, 1H), 1.58 (br, 1H), 1.48 (m, 2H), 1.40 (br, 1H), 1.29 (m, 3H), 1.06 (t, 3H), 0.98 (m, 6H)

### Example 267. (S)-quinuclidin-3-yl (5-(6-(2-methoxyethoxy)pyridin-3-yl)-2,2-dimethyl-2,3-dihydro-1H-inden-1-yl)carbamate

¹H-NMR (400 MHz, CDCl₃) δ 8.32 (s, 1H), 7.76 (d, 1H), 7.36-7.28 (m, 3H), 6.86 (d, 1H), 4.96 (m, 2H), 4.80 (br, 1H), 4.51 (m, 2H), 3.78 (m, 2H), 3.45 (s, 3H), 3.25 (m, 1H), 2.89-2.73 (m, 7H), 2.05 (br, 1H), 1.85 (br, 1H), 1.68 (br, 1H), 1.58 (br, 1H), 1.40 (br, 1H), 1.29 (m, 3H), 0.98 (m, 3H)

### Example 268. (S)-quinuclidin-3-yl (5-(6-isobutoxypyridin-3-yl)-2,2-dimethyl-2,3-dihydro-1H-inden-1-yl)carbamate

¹H-NMR (400 MHz, CDCl₃) δ 8.34 (s, 1H), 7.76 (d, 1H), 7.35-7.27 (m, 3H), 6.80 (d, 1H), 4.96 (m, 2H), 4.78 (br, 1H), 4.10 (m, 2H), 3.27 (m, 1H), 2.89-2.74 (m, 7H), 2.08 (br, 1H), 1.86 (br, 1H), 1.69 (br, 1H), 1.58 (br, 1H), 1.42 (br, 1H), 1.29 (m, 3H), 1.00 (d, 6H), 0.98 (m, 3H)

### Example 269. (S)-quinuclidin-3-yl (5-(3-isobutylphenyl)-2,2-dimethyl-2,3-dihydro-1H-inden-1-yl)carbamate

¹H-NMR (400 MHz, CDCl₃) δ 7.45-7.27 (m, 6H), 7.12 (d, 1H), 4.97 (m, 2H), 4.80 (br, 1H), 3.27 (m, 1H), 2.89-2.74 (m, 7H), 2.53 (d, 2H), 2.08 (br, 1H), 1.86 (br, 1H), 1.69 (br, 1H), 1.58 (br, 1H), 1.42 (br, 1H), 1.29 (m, 3H), 0.98 (m, 3H), 0.93 (d, 6H)

### Example 270. (S)-quinuclidin-3-yl (5-(4-butoxyphenyl)-2,2-dimethyl-2,3-dihydro-1H-inden-1-yl)carbamate

¹H-NMR (400 MHz, CDCl₃) δ 7.50 (d, 2H), 7.39 (m, 2H), 7.28 (m, 1H), 6.96 (d, 2H), 4.97 (m, 2H), 4.80 (br, 1H), 4.02 (m, 1H), 3.27 (m, 1H), 2.89-2.74 (m, 7H), 2.53 (d, 2H), 2.08 (br, 1H), 1.86 (br, 1H), 1.77 (m, 2H), 1.69 (br, 1H), 1.58 (br, 1H), 1.52 (m, 2H), 1.42 (br, 1H), 1.29 (m, 3H), 0.98 (m, 6H)

### Example 271. (S)-quinuclidin-3-yl (5-(2,4-dimethoxypyridin-3-yl)-2,2-dimethyl-2,3-dihydro-1H-inden-1-yl)carbamate

¹H-NMR (400 MHz, CDCl₃) δ 7.56 (d, 1H), 7.38 (m, 1H), 7.30 (s, 1H), 7.28 (m, 1H), 6.38 (d, 1H), 4.97 (m, 2H), 4.80 (br, 1H), 3.96 (s, 6H), 3.27 (m, 1H), 2.89-2.74 (m, 7H), 2.08 (br, 1H), 1.86 (br, 1H), 1.69 (br, 1H), 1.58 (br, 1H), 1.42 (br, 1H), 1.28 (m, 3H), 0.97 (m, 3H)

### Example 272. (S)-quinuclidin-3-yl (5-(2-acetylthiophen-3-yl)-2,2-dimethyl-2,3-dihydro-1H-inden-1-yl)carbamate

¹H-NMR (400 MHz, CDCl₃) δ 7.53 (d, 1H), 7.30-7.19 (m, 3H), 7.03 (d, 1H), 5.02 (m, 1H), 4.94 (m, 1H), 4.79 (br, 1H), 3.28 (m, 1H), 2.89-2.72 (m, 7H), 2.16 (s, 3H), 2.08 (br, 1H), 1.86 (br, 1H), 1.69 (br, 1H), 1.58 (br, 1H), 1.42 (br, 1H), 1.30 (m, 3H), 0.97 (m, 3H)

### Example 273. (S)-quinuclidin-3-yl (5-((E)-3-fluorostyryl)-2,2-dimethyl-2,3-dihydro-1H-inden-1-yl)carbamate

¹H-NMR (400 MHz, CDCl₃) δ 7.35-7.19 (m, 6H), 7.06 (d, 2H), 6.97 (m, 1H), 4.94 (m, 2H), 4.79 (br, 1H), 3.28 (m, 1H), 2.89-2.71 (m, 7H), 2.08 (br, 1H), 1.86 (br, 1H), 1.69 (br, 1H), 1.58 (br, 1H), 1.42 (br, 1H), 1.29 (m, 3H), 0.96 (m, 3H)

### Example 274. (S)-quinuclidin-3-yl (5-((E)-4-ethylstyryl)-2,2-dimethyl-2,3-dihydro-1H-inden-1-yl)carbamate

¹H-NMR (400 MHz, CDCl₃) δ 7.44 (d, 2H), 7.34 (s, 1H), 7.21 (m, 4H), 7.07 (s, 2H), 4.94 (m, 2H), 4.79 (br, 1H), 3.28 (m, 1H), 2.89-2.71 (m, 7H), 2.63 (m, 2H), 2.08 (br, 1H), 1.86 (br, 1H), 1.69 (br, 1H), 1.58 (br, 1H), 1.42 (br, 1H), 1.27 (m, 6H), 0.94 (m, 3H)

### Example 275. (S)-quinuclidin-3-yl (5-((E)-2-cyclohexylvinyl)-2,2-dimethyl-2,3-dihydro-1H-inden-1-yl)carbamate

¹H-NMR (400 MHz, CDCl₃) δ 7.18-7.12 (m, 3H), 6.35 (m, 1H), 6.16 (m, 1H), 4.94 (m, 2H), 4.79 (br, 1H), 3.28 (m, 1H), 2.89-2.71 (m, 7H), 2.08 (br, 1H), 1.86 (br, 1H), 1.76 (m, 5H), 1.69 (br, 2H), 1.58 (br, 1H), 1.42 (br, 1H), 1.27 (m, 3H), 1.15 (m, 3H), 0.94 (m, 3H)

### Examples 276 to 311

The titled compounds of Examples 276 to 311 were prepared in accordance with the same procedures as in Example 1, using (S)-quinuclidin-3-yl (5-bromo-2,2-diethyl-2,3-dihydro-1H-inden-1-yl)carbamate prepared in Preparation 5; and the corresponding substituted-boronic acids, respectively.

### Example 276. (S)-quinuclidin-3-yl (2,2-diethyl-5-(4-fluorophenyl)-2,3-dihydro-1H-inden-1-yl)carbamate

¹H-NMR (400 MHz, CDCl₃) δ 7.52 (m, 2H), 7.50-7.27 (m, 3H), 7.11 (t, 2H), 5.13 (m, 1H), 4.90 (m, 1H), 4.78 (m, 1H), 3.26 (m, 1H), 2.87-2.73 (m, 7H), 2.11 (br, 1H), 1.88 (br, 1H), 1.61 (br, 1H), 1.58 (m, 3H), 1.37 (br, 1H), 0.95 (m, 3H), 0.85 (m, 3H)

### Example 277. (S)-quinuclidin-3-yl (2,2-diethyl-5-(4-ethylphenyl)-2,3-dihydro-1H-inden-1-yl)carbamate

¹H-NMR (400 MHz, CDCl₃) δ 7.49 (d, 2H), 7.40 (m, 1H), 7.25 (m, 3H), 5.12 (m, 1H), 4.93 (m, 1H), 4.78 (m, 1H), 3.26 (m, 1H), 2.87-2.69 (m, 7H), 2.67 (m, 2H), 2.11 (br, 1H), 1.88 (br, 1H), 1.61 (br, 1H), 1.58 (m, 3H), 1.37 (br, 1H), 1.26 (t, 3H), 0.95 (m, 3H), 0.86 (m, 3H)

### Example 278. (S)-quinuclidin-3-yl (2,2-diethyl-5-(4-propylphenyl)-2,3-dihydro-1H-inden-1-yl)carbamate

¹H-NMR (400 MHz, CDCl₃) δ 7.48 (d, 2H), 7.40 (m, 1H), 7.27 (m, 1H), 7.25 (d, 2H), 5.11 (m, 1H), 4.96 (m, 1H), 4.78 (m, 1H), 3.26 (m, 1H), 2.87-2.72 (m, 7H), 2.62 (m, 2H), 2.11 (br, 1H), 1.88 (br, 1H), 1.69 (m, 3H), 1.58 (m, 3H), 1.37 (br, 1H), 0.97 (m, 6H), 0.85 (m, 3H)

### Example 279. (S)-quinuclidin-3-yl (5-(4-(tert-butyl)phenyl)-2,2-diethyl-2,3-dihydro-1H-inden-1-yl)carbamate ¹H-NMR (400 MHz, CDCl₃) δ 7.51 (d, 2H), 7.45 (d, 3H), 7.37 (m, 1H), 7.27 (m, 1H), 5.12 (m, 1H), 4.94 (m, 1H), 4.78 (m, 1H), 3.26 (m, 1H), 2.87-2.71 (m, 7H), 2.11 (br, 1H), 1.88 (br, 1H), 1.69 (br, 1H), 1.58 (m, 3H), 1.38 (br, 1H), 1.36 (s, 9H), 0.97 (m, 6H), 0.86 (m, 3H)

### Example 280. (S)-quinuclidin-3-yl (5-(4-butylphenyl)-2,2-diethyl-2,3-dihydro-1H-inden-1-yl)carbamate

¹H-NMR (400 MHz, CDCl₃) δ 7.48 (d, 2H), 7.42 (m, 2H), 7.30 (m, 1H), 7.26 (d, 2H), 5.11 (m, 1H), 4.94 (m, 1H), 4.78 (m, 1H), 3.26 (m, 1H), 2.87-2.71 (m, 7H), 2.63 (m, 2H), 2.11 (br, 1H), 1.85 (br, 1H), 1.69-1.57 (m, 6H), 1.38 (m, 3H), 0.94 (m, 6H), 0.87 (m, 3H)

### Example 281. (S)-quinuclidin-3-yl (5-(4-cyclopropylphenyl)-2,2-diethyl-2,3-dihydro-1H-inden-1-yl)carbamate

¹H-NMR (400 MHz, CDCl₃) δ 7.46 (d, 2H), 7.40 (m, 1H), 7.30 (m, 1H), 7.28 (m, 1H), 7.14 (d, 2H), 5.12 (m, 1H), 4.94 (m, 1H), 4.78 (m, 1H), 3.26 (m, 1H), 2.87-2.71 (m, 7H), 2.11 (br, 1H), 1.94 (m, 1H), 1.85 (br, 1H), 1.60 (br, 1H), 1.58 (m, 3H), 1.37 (br, 1H), 0.95 (m, 5H), 0.87 (m, 3H), 0.74 (m, 2H)

### Example 282. (S)-quinuclidin-3-yl (2,2-diethyl-5-(4-isopropylphenyl)-2,3-dihydro-1H-inden-1-yl)carbamate

¹H-NMR (400 MHz, CDCl₃) δ 7.50 (d, 2H), 7.41 (m, 1H), 7.30 (m, 1H), 7.28 (m, 3H), 5.11 (m, 1H), 4.95 (m, 1H), 4.78 (m, 1H), 3.26 (m, 1H), 2.94 (m, 1H), 2.88-2.73 (m, 7H), 2.11 (br, 1H), 1.85 (br, 1H), 1.68 (br, 1H), 1.58 (m, 3H), 1.37 (br, 1H), 1.29 (d, 6H), 0.98 (m, 3H), 0.87 (m, 3H)

### Example 283. (S)-quinuclidin-3-yl (2,2-diethyl-5-(4-methoxyphenyl)-2,3-dihydro-1H-inden-1-yl)carbamate

¹H-NMR (400 MHz, CDCl₃) δ 7.50 (d, 2H), 7.38 (m, 1H), 7.30 (m, 2H), 6.96 (d, 2H), 5.12 (m, 1H), 4.91 (m, 1H), 4.88 (m, 1H), 3.85 (s, 3H), 3.26 (m, 1H), 2.88-2.71 (m, 7H), 2.11 (br, 1H), 1.85 (br, 1H), 1.68 (br, 1H), 1.58 (m, 3H), 1.37 (br, 1H), 0.97 (m, 3H), 0.87 (m, 3H)

### Example 284. (S)-quinuclidin-3-yl (5-(4-ethoxyphenyl)-2,2-diethyl-2,3-dihydro-1H-inden-1-yl)carbamate

¹H-NMR (400 MHz, CDCl₃) δ 7.49 (d, 2H), 7.35 (m, 1H), 7.28 (m, 2H), 6.95 (d, 2H), 5.12 (m, 1H), 4.91 (m, 1H), 4.88 (m, 1H), 4.10 (m, 2H), 3.26 (m, 1H), 2.88-2.71 (m, 7H), 2.11 (br, 1H), 1.85 (br, 1H), 1.68 (br, 1H), 1.58 (m, 3H), 1.41 (t, 3H), 1.37 (br, 1H), 0.97 (m, 3H), 0.87 (m, 3H)

### Example 285. (S)-quinuclidin-3-yl (2,2-diethyl-5-(4-propoxyphenyl)-2,3-dihydro-1H-inden-1-yl)carbamate

¹H-NMR (400 MHz, CDCl₃) δ 7.49 (d, 2H), 7.35 (m, 1H), 7.28 (m, 2H), 6.96 (d, 2H), 5.12 (m, 1H), 4.91 (m, 1H), 4.88 (m, 1H), 3.98 (t, 2H), 3.26 (m, 1H), 2.88-2.71 (m, 7H), 2.11 (br, 1H), 1.85 (m, 3H), 1.68 (br, 1H), 1.58 (m, 3H), 1.37 (br, 1H), 1.06 (t, 3H), 0.97 (m, 3H), 0.87 (m, 3H)

### Example 286. (S)-quinuclidin-3-yl (2,2-diethyl-5-(4-isopropoxyphenyl)-2,3-dihydro-1H-inden-1-yl)carbamate

¹H-NMR (400 MHz, CDCl₃) δ 7.49 (d, 2H), 7.35 (m, 1H), 7.28 (m, 2H), 6.96 (d, 2H), 5.12 (m, 1H), 4.91 (m, 1H), 4.78 (m, 1H), 4.77 (m, 1H), 3.26 (m, 1H), 2.88-2.71 (m, 7H), 2.11 (br, 1H), 1.85 (br, 1H), 1.68 (br, 1H), 1.58 (m, 3H), 1.37 (br, 1H), 1.36 (d, 6H), 0.96 (m, 3H), 0.87 (m, 3H)

### Example 287. (S)-quinuclidin-3-yl (2,2-diethyl-5-(4-isobutylphenyl)-2,3-dihydro-1H-inden-1-yl)carbamate

¹H-NMR (400 MHz, CDCl₃) δ 7.48 (d, 2H), 7.38 (m, 1H), 7.28 (m, 2H), 7.20 (d, 2H), 5.13 (m, 1H), 4.92 (m, 1H), 4.78 (m, 1H), 3.26 (m, 1H), 2.88-2.73 (m, 7H), 2.51 (d, 2H), 2.11 (br, 1H), 1.92 (m, 1H), 1.85 (br, 1H), 1.68 (br, 1H), 1.58 (m, 3H), 1.37 (br, 1H), 0.96 (m, 3H), 0.93 (d, 6H), 0.86 (m, 3H)

### Example 288. (S)-quinuclidin-3-yl (2,2-diethyl-5-(3-fluorophenyl)-2,3-dihydro-1H-inden-1-yl)carbamate

¹H-NMR (400 MHz, CDCl₃) δ 7.41-7.02 (m, 6H), 7.02 (t, 1H), 5.14 (m, 1H), 4.92 (m, 1H), 4.79 (m, 1H), 3.24 (m, 1H), 2.88-2.74 (m, 7H), 2.11 (br, 1H), 1.85 (br, 1H), 1.68 (br, 1H), 1.58 (m, 3H), 1.37 (br, 1H), 0.96 (m, 3H), 0.86 (m, 3H)

### Example 289. (S)-quinuclidin-3-yl (2,2-diethyl-5-(3-methoxyphenyl)-2,3-dihydro-1H-inden-1-yl)carbamate

¹H-NMR (400 MHz, CDCl₃) δ 7.43 (m, 1H), 7.37-7.27 (m, 3H), 7.17 (d, 1H), 7.15 (s, 1H), 6.88 (d, 1H), 5.13 (m, 1H), 4.91 (m, 1H), 4.79 (m, 1H), 3.87 (s, 3H), 3.25 (m, 1H), 2.88-2.74 (m, 7H), 2.05 (br, 1H), 1.85 (br, 1H), 1.68 (br, 1H), 1.58 (m, 3H), 1.37 (br, 1H), 0.96 (m, 3H), 0.86 (m, 3H)

### Example 290. (S)-quinuclidin-3-yl (5-(3-ethoxyphenyl)-2,2-diethyl-2,3-dihydro-1H-inden-1-yl)carbamate

¹H-NMR (400 MHz, CDCl₃) δ 7.41 (m, 1H), 7.38-7.27 (m, 3H), 7.15 (d, 1H), 7.13 (s, 1H), 6.89 (d, 1H), 5.13 (m, 1H), 4.91 (m, 1H), 4.79 (m, 1H), 4.12 (m, 2H), 3.25 (m, 1H), 2.88-2.74 (m, 7H), 2.05 (br, 1H), 1.85 (br, 1H), 1.68 (br, 1H), 1.58 (m, 3H), 1.43 (t, 3H), 1.37 (br, 1H), 0.96 (m, 3H), 0.87 (m, 3H)

### Example 291. (S)-quinuclidin-3-yl (2,2-diethyl-5-(3-isopropoxyphenyl)-2,3-dihydro-1H-inden-1-yl)carbamate

¹H-NMR (400 MHz, CDCl₃) δ 7.41 (m, 1H), 7.39-7.27 (m, 3H), 7.12 (d, 1H), 7.09 (s, 1H), 6.88 (d, 1H), 5.13 (m, 1H), 4.91 (m, 1H), 4.78 (m, 1H), 4.62 (m, 1H), 3.25 (m, 1H), 2.88-2.73 (m, 7H), 2.05 (br, 1H), 1.85 (br, 1H), 1.68 (br, 1H), 1.58 (m, 3H), 1.29 (br, 1H), 1.26 (d, 6H), 0.97 (m, 3H), 0.86 (m, 3H)

### Example 292. (S)-quinuclidin-3-yl (2,2-diethyl-5-(3-ethylphenyl)-2,3-dihydro-1H-inden-1-yl)carbamate

¹H-NMR (400 MHz, CDCl₃) δ 7.44-7.27 (m, 6H), 7.18 (d, 1H), 5.13 (m, 1H), 4.93 (m, 1H), 4.78 (m, 1H), 3.27 (m, 1H), 2.85-2.71 (m, 7H), 2.68 (m, 2H), 2.05 (br, 1H), 1.85 (br, 1H), 1.68 (br, 1H), 1.58 (m, 3H), 1.39 (br, 1H), 1.29 (t, 3H), 0.97 (m, 3H), 0.86 (m, 3H)

### Example 293. (S)-quinuclidin-3-yl (2,2-diethyl-5-(3-isopropylphenyl)-2,3-dihydro-1H-inden-1-yl)carbamate

¹H-NMR (400 MHz, CDCl₃) δ 7.44-7.27 (m, 6H), 7.21 (d, 1H), 5.14 (m, 1H), 4.92 (m, 1H), 4.79 (m, 1H), 3.27 (m, 1H), 2.99 (m, 1H), 2.85-2.71 (m, 7H), 2.05 (br, 1H), 1.85 (br, 1H), 1.68 (br, 1H), 1.58 (m, 3H), 1.39 (br, 1H), 1.27 (d, 6H), 0.98 (m, 3H), 0.86 (m, 3H)

### Example 294. (S)-quinuclidin-3-yl (5-(3-(tert-butyl)phenyl)-2,2-diethyl-2,3-dihydro-1H-inden-1-yl)carbamate

¹H-NMR (400 MHz, CDCl₃) δ 7.58 (s, 1H), 7.41-7.27 (m, 6H), 5.11 (m, 1H), 4.92 (m, 1H), 4.79 (m, 1H), 3.27 (m, 1H), 2.86-2.75 (m, 7H), 2.05 (br, 1H), 1.85 (br, 1H), 1.68 (br, 1H), 1.58 (m, 3H), 1.39 (br, 1H), 1.38 (s, 9H), 0.96 (m, 3H), 0.87 (m, 3H)

### Example 295. (S)-quinuclidin-3-yl (5-(2,3-dimethoxyphenyl)-2,2-diethyl-2,3-dihydro-1H-inden-1-yl)carbamate

¹H-NMR (400 MHz, CDCl₃) δ 7.37-7.26 (m, 3H), 7.10 (m, 1H), 6.92 (d, 2H), 5.14 (m, 1H), 4.92 (m, 1H), 4.77 (m, 1H), 3.91 (s, 3H), 3.59 (s, 3H), 3.25 (m, 1H), 2.86-2.73 (m, 7H), 2.05 (br, 1H), 1.85 (br, 1H), 1.68 (br, 1H), 1.58 (m, 3H), 1.37 (br, 1H), 0.96 (m, 3H), 0.85 (m, 3H)

### Example 296. (S)-quinuclidin-3-yl (5-(2,4-dimethoxyphenyl)-2,2-diethyl-2,3-dihydro-1H-inden-1-yl)carbamate

¹H-NMR (400 MHz, CDCl₃) δ 7.33 (m, 1H), 7.28-7.21 (m, 3H), 6.57 (m, 2H), 5.11 (m, 1H), 4.90 (m, 1H), 4.85 (m, 1H), 3.85 (s, 3H), 3.80 (s, 3H), 3.24 (m, 1H), 2.86-2.71 (m, 7H), 2.05 (br, 1H), 1.85 (br, 1H), 1.68 (br, 1H), 1.58 (m, 3H), 1.37 (br, 1H), 0.95 (m, 3H), 0.87 (m, 3H)

### Example 297. (S)-quinuclidin-3-yl (5-(2,5-dimethoxyphenyl)-2,2-diethyl-2,3-dihydro-1H-inden-1-yl)carbamate

¹H-NMR (400 MHz, CDCl₃) δ 7.35 (m, 1H), 7.30-7.27 (m, 2H), 6.92-6.83 (m, 3H), 5.12 (m, 1H), 4.90 (m, 1H), 4.85 (m, 1H), 3.80 (s, 3H), 3.75 (s, 3H), 3.24 (m, 1H), 2.86-2.72 (m, 7H), 2.05 (br, 1H), 1.85 (br, 1H), 1.68 (br, 1H), 1.58 (m, 3H), 1.37 (br, 1H), 0.96 (m, 3H), 0.87 (m, 3H)

### Example 298. (S)-quinuclidin-3-yl (5-(3,4-dimethoxyphenyl)-2,2-diethyl-2,3-dihydro-1H-inden-1-yl)carbamate

¹H-NMR (400 MHz, CDCl₃) δ 7.39 (m, 1H), 7.31 (s, 1H), 7.28 (m, 1H), 7.13 (d, 1H), 7.08 (s, 1H), 6.93 (d, 1H), 5.12 (m, 1H), 4.91 (m, 1H), 4.85 (m, 1H), 3.95 (s, 3H), 3.92 (s, 3H), 3.24 (m, 1H), 2.86-2.72 (m, 7H), 2.05 (br, 1H), 1.85 (br, 1H), 1.68 (br, 1H), 1.58 (m, 3H), 1.37 (br, 1H), 0.95 (m, 3H), 0.86 (m, 3H)

### Example 299. (S)-quinuclidin-3-yl (5-(3,5-dimethoxyphenyl)-2,2-diethyl-2,3-dihydro-1H-inden-1-yl)carbamate

¹H-NMR (400 MHz, CDCl₃) δ 7.40 (m, 1H), 7.39 (s, 1H), 7.29 (m, 1H), 6.70 (s, 2H), 6.45 (m, 1H), 5.13 (m, 1H), 4.92 (m, 1H), 4.85 (brs, 1H), 3.85 (s, 6H), 3.24 (m, 1H), 2.86-2.73 (m, 7H), 2.05 (br, 1H), 1.85 (br, 1H), 1.68 (br, 1H), 1.58 (m, 3H), 1.37 (br, 1H), 0.96 (m, 3H), 0.87 (m, 3H)

### Example 300. (S)-quinuclidin-3-yl (5-(2,6-dimethoxyphenyl)-2,2-diethyl-2,3-dihydro-1H-inden-1-yl)carbamate

¹H-NMR (400 MHz, CDCl₃) δ 7.30-7.26 (m, 2H), 7.18 (d, 1H), 7.14 (s, 1H), 6.65 (d, 2H), 5.12 (m, 1H), 4.92 (m, 1H), 4.77 (m, 1H), 3.73 (s, 6H), 3.24 (m, 1H), 2.85-2.71 (m, 7H), 2.05 (br, 1H), 1.85 (br, 1H), 1.68 (br, 1H), 1.58 (m, 3H), 1.37 (br, 1H), 0.96 (m, 3H), 0.88 (m, 3H)

### Example 301. (S)-quinuclidin-3-yl (5-(3-(difluoromethyl)phenyl)-2,2-diethyl-2,3-dihydro-1H-inden-1-yl)carbamate

¹H-NMR (400 MHz, CDCl₃) δ 7.70 (m, 2H), 7.53-7.39 (m, 4H), 7.31 (m, 1H), 6.85-6.56 (t, 1H), 5.15 (m, 1H), 4.92 (m, 1H), 4.79 (m, 1H), 3.24 (m, 1H), 2.85-2.74 (m, 7H), 2.05 (br, 1H), 1.85 (br, 1H), 1.68 (br, 1H), 1.58 (m, 3H), 1.37 (br, 1H), 0.96 (m, 3H), 0.86 (m, 3H)

### Example 302. (S)-quinuclidin-3-yl (5-(3-(dimethylamino)phenyl)-2,2-diethyl-2,3-dihydro-1H-inden-1-yl)carbamate

¹H-NMR (400 MHz, CDCl₃) δ 7.43 (m, 1H), 7.38 (s, 1H), 7.27 (m, 2H), 6.91 (m, 2H), 6.73 (d, 1H), 5.13 (m, 1H), 4.88 (m, 1H), 4.79 (m, 1H), 3.24 (m, 1H), 3.00 (s, 6H), 2.85-2.74 (m, 7H), 2.05 (br, 1H), 1.85 (br, 1H), 1.68 (br, 1H), 1.58 (m, 3H), 1.37 (br, 1H), 0.96 (m, 3H), 0.87 (m, 3H)

### Example 303. (S)-quinuclidin-3-yl (5-(4-ethoxy-3,5-dimethylphenyl)-2,2-diethyl-2,3-dihydro-1H-inden-1-yl)carbamate

¹H-NMR (400 MHz, CDCl₃) δ 7.38-7.33 (m, 2H), 7.28 (m, 1H), 7.21 (s, 2H), 5.12 (m, 1H), 4.88 (m, 1H), 4.79 (m, 1H), 3.89(q, 2H), 3.25 (m, 1H), 2.85-2.74 (m, 7H), 2.33 (s, 6H), 2.05 (br, 1H), 1.85 (br, 1H), 1.68 (br, 1H), 1.58 (m, 3H), 1.41 (t, 3H), 1.37 (br, 1H), 0.95 (m, 3H), 0.85 (m, 3H)

### Example 304. (S)-quinuclidin-3-yl (5-(3,5-dimethyl-4-propoxyphenyl)-2,2-diethyl-2,3-dihydro-1H-inden-1-yl)carbamate

¹H-NMR (400 MHz, CDCl3) δ 7.38-7.30 (m, 2H), 7.28 (m, 1H), 7.20 (s, 2H), 5.11 (m, 1H), 4.93 (m, 1H), 4.79 (m, 1H), 3.77 (t, 2H), 3.25 (m, 1H), 2.85-2.74 (m, 7H), 2.33 (s, 6H), 2.05 (br, 1H), 1.85 (m, 3H), 1.68 (br, 1H), 1.58 (m, 3H), 1.37 (br, 1H), 1.09 (t, 3H), 0.95 (m, 3H), 0.85 (m, 3H)

### Example 305. (S)-quinuclidin-3-yl (5-(4-butoxy-3,5-dimethylphenyl)-2,2-diethyl-2,3-dihydro-1H-inden-1-yl)carbamate

¹H-NMR (400 MHz, CDCl₃) δ 7.38-7.30 (m, 2H), 7.28 (m, 1H), 7.20 (s, 2H), 5.12 (m, 1H), 4.91 (m, 1H), 4.79 (m, 1H), 3.78 (t, 2H), 3.25 (m, 1H), 2.85-2.74 (m, 7H), 2.33 (s, 6H), 2.05 (br, 1H), 1.85 (m, 3H), 1.68 (br, 1H), 1.58 (m, 5H), 1.37 (br, 1H), 0.99 (t, 3H), 0.95 (m, 3H), 0.86 (m, 3H)

### Example 306. (S)-quinuclidin-3-yl (2,2-diethyl-5-(thiophen-3-yl)-2,3-dihydro-1H-inden-1-yl)carbamate

¹H-NMR (400 MHz, CDCl₃) δ 7.45-7.36 (m, 5H), 7.26 (m, 1H), 5.11 (m, 1H), 4.88 (m, 1H), 4.77 (m, 1H), 3.25 (m, 1H), 2.85-2.72 (m, 7H), 2.05 (br, 1H), 1.85 (br, 1H), 1.68 (br, 1H), 1.58 (m, 3H), 1.37 (br, 1H), 0.95 (m, 3H), 0.85 (m, 3H)

### Example 307. (S)-quinuclidin-3-yl (2,2-diethyl-5-(furan-3-yl)-2,3-dihydro-1H-inden-1-yl)carbamate

¹H-NMR (400 MHz, CDCl₃) δ 7.70 (s, 1H), 7.46 (s, 1H), 7.30-7.20 (m, 3H), 6.68 (s, 1H), 5.10 (m, 1H), 4.88 (m, 1H), 4.78 (m, 1H), 3.25 (m, 1H), 2.85-2.69 (m, 7H), 2.05 (br, 1H), 1.85 (br, 1H), 1.68 (br, 1H), 1.58 (m, 3H), 1.37 (br, 1H), 0.94 (m, 3H), 0.85 (m, 3H)

### Example 308. (S)-quinuclidin-3-yl (5-(6-(cyclopropylmethoxy)pyridin-3-yl)-2,2-diethyl-2,3-dihydro-1H-inden-1-yl)carbamate

¹H-NMR (400 MHz, CDCl₃) δ 8.31 (s, 1H), 7.77 (d, 1H), 7.31 (m, 3H), 6.82 (d, 1H), 5.12 (m, 1H), 4.93 (m, 1H), 4.78 (m, 1H), 4.16 (m, 2H), 3.25 (m, 1H), 2.85-2.72 (m, 7H), 2.05 (br, 1H), 1.85 (br, 1H), 1.68 (br, 1H), 1.58 (m, 3H), 1.37 (br, 1H), 0.94 (m, 3H), 0.85 (m, 3H), 0.63 (m, 2H), 0.37 (m, 2H)

### Example 309. (S)-quinuclidin-3-yl (5-(2,2-difluorobenzo[d][1,3]dioxol-5-yl)-2,2-diethyl-2,3-dihydro-1H-inden-1-yl)carbamate

¹H-NMR (400 MHz, CDCl₃) δ 7.33-7.24 (m, 5H), 7.10 (m, 1H), 5.14 (m, 1H), 4.91 (m, 1H), 4.87 (m, 1H), 3.25 (m, 1H), 2.85-2.73 (m, 7H), 2.05 (br, 1H), 1.85 (br, 1H), 1.68 (br, 1H), 1.58 (m, 3H), 1.37 (br, 1H), 0.96 (m, 3H), 0.86 (m, 3H)

### Example 310. (S)-quinuclidin-3-yl (2,2-diethyl-5-(2-fluoro-4-methoxyphenyl)-2,3-dihydro-1H-inden-1-yl)carbamate

¹H-NMR (400 MHz, CDCl₃) δ 7.34-7.27 (m, 4H), 6.74 (m, 2H), 5.12 (m, 1H), 4.94 (m, 1H), 4.77 (m, 1H), 3.83 (s, 3H), 3.25 (m, 1H), 2.85-2.72 (m, 7H), 2.05 (br, 1H), 1.85 (br, 1H), 1.68 (br, 1H), 1.58 (m, 3H), 1.37 (br, 1H), 0.96 (m, 3H), 0.86 (m, 3H)

### Example 311. (S)-quinuclidin-3-yl (5-(3-chloro-4-isopropoxyphenyl)-2,2-diethyl-2,3-dihydro-1H-inden-1-yl)carbamate

¹H-NMR (400 MHz, CDCl₃) δ 7.59 (s, 1H), 7.40-7.01 (m, 4H), 7.00 (d, 1H), 5.13 (m, 1H), 4.94 (m, 1H), 4.77 (m, 1H), 3.83 (s, 3H), 3.25 (m, 1H), 2.85-2.72 (m, 7H), 2.05 (br, 1H), 1.85 (br, 1H), 1.68 (br, 1H), 1.58 (m, 3H), 1.37 (br, 1H), 0.96 (m, 3H), 0.86 (m, 3H)

### Examples 312 to 348

The titled compounds of Examples 312 to 348 were prepared in accordance with the same procedures as in Example 1, using (S)-quinuclidin-3-yl (5-bromo-6-methoxy-2,2-dimethyl-2,3-dihydro-1H-inden-1-yl)carbamate prepared in Preparation 6; and the corresponding substituted-boronic acids, respectively.

### Example 312. (S)-quinuclidin-3-yl (5-(4-ethylphenyl)-6-methoxy-2,2-dimethyl-2,3-dihydro-1H-inden-1-yl)carbamate

¹H-NMR (400 MHz, CDCl₃) δ 7.42 (d, 2H), 7.24 (m, 3H), 6.85 (d, 1H), 4.94 (m, 1H), 4.82 (m, 1H), 4.74 (br, 1H), 3.79 (d, 3H), 3.29 (m, 1H), 2.91 (m, 2H), 2.89-2.70 (m, 5H), 2.69 (m, 2H), 2.11 (br, 1H), 1.88 (br, 1H), 1.72 (br, 1H), 1.61 (br, 1H), 1.44 (br, 1H), 1.30 (m, 6H), 1.00 (m, 3H)

### Example 313. (S)-quinuclidin-3-yl (6-methoxy-2,2-dimethyl-5-(4-propylphenyl)-2,3-dihydro-1H-inden-1-yl)carbamate

¹H-NMR (400 MHz, CDCl₃) δ 7.41 (d, 2H), 7.22 (d, 2H), 7.11 (s, 1H), 6.85 (d, 1H), 4.95 (m, 1H), 4.82 (m, 1H), 4.74 (br, 1H), 3.79 (d, 3H), 3.29 (m, 1H), 2.90 (m, 2H), 2.89-2.71 (m, 5H), 2.62 (m, 2H), 2.11 (br, 1H), 1.88 (br, 1H), 1.72-1.67 (m, 3H), 1.61 (br, 1H), 1.43 (br, 1H), 1.32 (m, 3H), 1.00 (m, 6H)

### Example 314. (S)-quinuclidin-3-yl (5-(4-isopropylphenyl)-6-methoxy-2,2-dimethyl-2,3-dihydro-1H-inden-1-yl)carbamate

¹H-NMR (400 MHz, CDCl₃) δ 7.43 (d, 2H), 7.26 (d, 2H), 7.11 (s, 1H), 6.85 (d, 1H), 4.95 (m, 1H), 4.82 (m, 1H), 4.74 (br, 1H), 3.80 (d, 3H), 3.29 (m, 1H), 2.92 (m, 3H), 2.89-2.71 (m, 5H), 2.11 (br, 1H), 1.88 (br, 1H), 1.71 (br, 1H), 1.62 (br, 1H), 1.44 (br, 1H), 1.30 (m, 9H), 1.00 (m, 3H)

### Example 315. (S)-quinuclidin-3-yl (5-(4-isobutylphenyl)-6-methoxy-2,2-dimethyl-2,3-dihydro-1H-inden-1-yl)carbamate

¹H-NMR (400 MHz, CDCl₃) δ 7.41 (d, 2H), 7.17 (d, 2H), 7.11 (s, 1H), 6.85 (d, 1H), 4.95 (m, 1H), 4.82 (m, 1H), 4.74 (br, 1H), 3.80 (d, 3H), 3.29 (m, 1H), 2.90-2.71 (m, 7H), 2.50 (d, 2H), 2.11 (br, 1H), 1.92 (m, 2H), 1.71 (br, 1H), 1.62 (br, 1H), 1.44 (br, 1H), 1.30 (m, 3H), 1.00 (m, 3H), 0.99 (d, 6H)

### Example 316. (S)-quinuclidin-3-yl (5-(4-(tert-butyl)phenyl)-6-methoxy-2,2-dimethyl-2,3-dihydro-1H-inden-1-yl)carbamate

¹H-NMR (400 MHz, CDCl₃) δ 7.46 (m, 4H), 7.12 (s, 1H), 6.85 (d, 1H), 4.95 (m, 1H), 4.82 (m, 1H), 4.74 (br, 1H), 3.81 (d, 3H), 3.29 (m, 1H), 2.93-2.66 (m, 7H), 2.50 (d, 2H), 2.11 (br, 1H), 1.88 (br, 1H), 1.71 (br, 1H), 1.62 (br, 1H), 1.44 (br, 1H), 1.40 (s, 9H), 1.30 (m, 3H), 1.00 (m, 3H)

### Example 317. (S)-quinuclidin-3-yl (5-(4-cyclopropylphenyl)-6-methoxy-2,2-dimethyl-2,3-dihydro-1H-inden-1-yl)carbamate

¹H-NMR (400 MHz, CDCl₃) δ 7.39 (d, 2H), 7.10 (d, 3H), 6.85 (d, 1H), 4.95 (m, 1H), 4.82 (m, 1H), 4.74 (br, 1H), 3.81 (d, 3H), 3.29 (m, 1H), 2.93-2.66 (m, 7H), 2.50 (d, 2H), 2.11 (br, 1H), 1.90 (m, 1H), 1.88 (br, 1H), 1.71 (br, 1H), 1.62 (br, 1H), 1.44 (br, 1H), 1.30 (m, 3H), 1.00 (m, 3H), 0.98 (d, 2H), 0.75 (m, 2H)

### Example 318. (S)-quinuclidin-3-yl (5-(4-ethoxyphenyl)-6-methoxy-2,2-dimethyl-2,3-dihydro-1H-inden-1-yl)carbamate

¹H-NMR (400 MHz, CDCl₃) δ 7.42 (d, 2H), 7.09 (s, 1H), 6.93 (d, 2H), 6.81 (d, 1H), 4.94 (m, 2H), 4.81 (m, 1H), 4.09 (m, 2H), 3.80 (d, 3H), 3.28 (m, 1H), 2.90-2.671 (m, 7H), 2.11 (br, 1H), 1.90 (m, 1H), 1.88 (br, 1H), 1.71 (br, 1H), 1.62 (br, 1H), 1.43 (t, 4H), 1.26 (m, 3H), 0.97 (m, 3H)

### Example 319. (S)-quinuclidin-3-yl (6-methoxy-2,2-dimethyl-5-(4-propoxyphenyl)-2,3-dihydro-1H-inden-1-yl)carbamate

¹H-NMR (400 MHz, CDCl₃) δ 7.42 (d, 2H), 7.09 (s, 1H), 6.94 (d, 2H), 6.82 (d, 1H), 4.94 (m, 2H), 4.81 (m, 1H), 3.95 (m, 2H), 3.80 (d, 3H), 3.28 (m, 1H), 2.90-2.67 (m, 7H), 2.11 (br, 1H), 1.90 (m, 1H), 1.88 (br, 1H), 1.83 (m, 2H), 1.71 (br, 1H), 1.62 (br, 1H), 1.43 (br, 1H), 1.26 (m, 3H), 1.07 (t, 3H), 0.97 (m, 3H)

### Example 320. (S)-quinuclidin-3-yl (5-(4-isopropoxyphenyl)-6-methoxy-2,2-dimethyl-2,3-dihydro-1H-inden-1-yl)carbamate

¹H-NMR (400 MHz, CDCl₃) δ 7.42 (d, 2H), 7.09 (s, 1H), 6.90 (d, 2H), 6.85 (d, 1H), 4.93 (m, 2H), 4.81 (m, 1H), 4.59 (m, 1H), 3.80 (d, 3H), 3.28 (m, 1H), 2.90-2.72 (m, 7H), 2.10 (br, 1H), 1.86 (br, 1H), 1.71 (br, 1H), 1.60 (br, 1H), 1.43 (br, 1H), 1.37 (d, 6H), 1.28 (m, 3H), 0.97 (m, 3H)

### Example 321. (S)-quinuclidin-3-yl (5-(3,5-dimethyl-4-propoxyphenyl)-6-methoxy-2,2-dimethyl-2,3-dihydro-1H-inden-1-yl)carbamate

¹H-NMR (400 MHz, CDCl₃) δ 7.13 (s, 2H), 7.07 (s, 1H), 6.82 (d, 1H), 4.93 (m, 2H), 4.81 (m, 1H), 3.78 (m, 5H), 3.27 (m, 1H), 2.90-2.69 (m, 7H), 2.31 (s, 6H), 2.10 (br, 1H), 1.86 (br, 1H), 1.71 (br, 1H), 1.69 (m, 2H), 1.60 (br, 1H), 1.43 (br, 1H), 1.28 (m, 3H), 1.09 (t, 3H), 0.93 (m, 3H)

### Example 322. (S)-quinuclidin-3-yl (5-(3-chloro-4-isopropoxyphenyl)-6-methoxy-2,2-dimethyl-2,3-dihydro-1H-inden-1-yl)carbamate

¹H-NMR (400 MHz, CDCl₃) δ 7.52 (s, 1H), 7.33 (d, 1H), 7.07 (s, 1H), 6.96 (d, 1H), 6.83 (d, 1H), 4.93 (m, 2H), 4.81 (m, 1H), 4.58 (m, 1H), 3.78 (m, 3H), 3.27 (m, 1H), 2.90-2.70 (m, 7H), 2.10 (br, 1H), 1.86 (br, 1H), 1.71 (br, 1H), 1.60 (br, 1H), 1.43 (br, 1H), 1.40 (d, 6H), 1.28 (m, 3H), 0.96 (m, 3H)

### Example 323. (S)-quinuclidin-3-yl (5-(2-fluoro-4-methoxyphenyl)-6-methoxy-2,2-dimethyl-2,3-dihydro-1H-inden-1-yl)carbamate

¹H-NMR (400 MHz, CDCl₃) δ 7.24 (m, 1H), 7.03 (s, 1H), 6.87 (d, 1H), 6.75-6.67 (m, 2H), 4.96 (m, 2H), 4.81 (m, 1H), 3.80 (s, 3H), 3.78 (m, 3H), 3.27 (m, 1H), 2.90-2.70 (m, 7H), 2.10 (br, 1H), 1.85 (br, 1H), 1.70 (br, 1H), 1.58 (br, 1H), 1.41 (br, 1H), 1.28 (m, 3H), 0.97 (m, 3H)

### Example 324. (S)-quinuclidin-3-yl (5-(4-butoxyphenyl)-6-methoxy-2,2-dimethyl-2,3-dihydro-1H-inden-1-yl)carbamate

¹H-NMR (400 MHz, CDCl₃) δ 7.42 (d, 2H), 7.09 (s, 1H), 6.93 (d, 2H), 6.83 (d, 1H), 4.93 (m, 2H), 4.81 (m, 1H), 3.98 (m, 2H), 3.79 (m, 3H), 3.27 (m, 1H), 2.90-2.71 (m, 7H), 2.10 (br, 1H), 1.85 (br, 1H), 1.71 (m, 2H), 1.69 (br, 1H), 1.59 (br, 1H), 1.52 (m, 2H), 1.43 (br, 1H), 1.27 (m, 3H), 0.99 (m, 6H)

### Example 325. (S)-quinuclidin-3-yl (5-(4-isobutylphenyl)-6-methoxy-2,2-dimethyl-2,3-dihydro-1H-inden-1-yl)carbamate

¹H-NMR (400 MHz, CDCl₃) δ 7.42 (d, 2H), 7.09 (s, 1H), 6.93 (d, 2H), 6.83 (d, 1H), 4.93 (m, 2H), 4.81 (m, 1H), 3.79 (m, 5H), 3.27 (m, 1H), 2.90-2.71 (m, 7H), 2.10 (m, 2H), 1.85 (br, 1H), 1.69 (br, 1H), 1.59 (br, 1H), 1.43 (br, 1H), 1.27 (m, 3H), 0.99 (m, 6H)

### Example 326. (S)-quinuclidin-3-yl (5-(6-(cyclopropylmethoxy)pyridin-3-yl)-6-methoxy-2,2-dimethyl-2,3-dihydro-1H-inden-1-yl)carbamate

¹H-NMR (400 MHz, CDCl₃) δ 8.24 (s, 1H), 7.74 (d, 1H), 7.07 (s, 1H), 6.86 (d, 1H), 6.80 (d, 1H), 4.93 (m, 2H), 4.81 (m, 1H), 4.15 (m, 2H), 3.79 (m, 3H), 3.27 (m, 1H), 2.90-2.71 (m, 7H), 2.10 (m, 1H), 1.85 (br, 1H), 1.69 (br, 1H), 1.59 (br, 1H), 1.43 (br, 1H), 1.27 (m, 3H), 0.97 (m, 3H), 0.62 (m, 2H), 0.38 (m, 2H)

### Example 327. (S)-quinuclidin-3-yl (5-(5-chloro-2-methoxyphenyl)-6-methoxy-2,2-dimethyl-2,3-dihydro-1H-inden-1-yl)carbamate

¹H-NMR (400 MHz, CDCl₃) δ 7.25 (s, 1H) 7.19 (m, 1H), 7.00 (s, 1H), 6.89-6.82 (m, 2H), 4.93 (m, 2H), 4.81 (m, 1H), 3.79 (m, 6H), 3.29 (m, 1H), 2.92-2.65 (m, 7H), 2.10 (m, 1H), 1.85 (br, 1H), 1.69 (br, 1H), 1.59 (br, 1H), 1.43 (br, 1H), 1.29 (m, 3H), 0.98 (m, 3H)

### Example 328. (S)-quinuclidin-3-yl (5-(4-butylphenyl)-6-methoxy-2,2-dimethyl-2,3-dihydro-1H-inden-1-yl)carbamate

¹H-NMR (400 MHz, CDCl₃) δ 7.40 (d, 2H) 7.20 (d, 2H), 7.10 (s, 1H), 6.84 (d, 1H), 4.95 (s, 2H), 4.80 (br, 1H), 3.78 (d, 3H), 3.25 (m, 1H), 2.92-2.65 (m, 7H), 2.64 (m, 2H), 2.10 (m, 1H), 1.85 (br, 1H), 1.69 (br, 1H), 1.60 (m, 2H), 1.59 (br, 1H), 1.43 (m, 3H), 1.29 (m, 3H), 0.98 (m, 3H), 0.92 (t, 3H)

### Example 329. (S)-quinuclidin-3-yl (6-methoxy-5-(4-methoxy-3,5-dimethylphenyl)-2,2-dimethyl-2,3-dihydro-1H-inden-1-yl)carbamate

¹H-NMR (400 MHz, CDCl₃) δ 7.14 (s, 2H) 7.07 (s, 1H), 6.82 (d, 1H), 4.94 (s, 2H), 4.80 (br, 1H), 3.78 (d, 3H), 3.76 (s, 3H), 3.25 (m, 1H), 2.92-2.65 (m, 7H), 2.32 (s, 6H), 2.10 (m, 1H), 1.85 (br, 1H), 1.69 (br, 1H), 1.58 (br, 1H), 1.40 (br, 1H), 1.28 (m, 3H), 0.98 (m, 3H)

### Example 330. (S)-quinuclidin-3-yl (5-(4-ethoxy-3,5-dimethylphenyl)-6-methoxy-2,2-dimethyl-2,3-dihydro-1H-inden-1-yl)carbamate

¹H-NMR (400 MHz, CDCl₃) δ 7.14 (s, 2H) 7.07 (s, 1H), 6.83 (d, 1H), 4.94 (s, 2H), 4.80 (br, 1H), 3.89 (m, 2H), 3.78 (d, 3H), 3.25 (m, 1H), 2.92-2.65 (m, 7H), 2.31 (s, 6H), 2.10 (m, 1H), 1.85 (br, 1H), 1.69 (br, 1H), 1.59 (br, 1H), 1.43 (t, 4H), 1.28 (m, 3H), 0.98 (m, 3H)

### Example 331. (S)-quinuclidin-3-yl (5-(4-butoxy-3,5-dimethylphenyl)-6-methoxy-2,2-dimethyl-2,3-dihydro-1H-inden-1-yl)carbamate

¹H-NMR (400 MHz, CDCl₃) δ 7.13 (s, 2H), 7.07 (s, 1H), 6.83 (d, 1H), 4.93 (m, 2H), 4.80 (br, 1H), 3.80 (m, 3H), 3.25 (m, 1H), 2.92-2.71 (m, 7H), 2.69 (m, 2H), 2.31 (s, 6H), 2.10 (m, 1H), 1.85 (br, 1H), 1.77 (m, 2H), 1.69 (br, 1H), 1.60 (m, 2H), 1.59 (m, 3H), 1.43 (br, 1H), 1.29 (m, 3H), 0.98 (m, 6H)

### Example 332. (S)-quinuclidin-3-yl (6-methoxy-5-(2-methoxypyridin-4-yl)-2,2-dimethyl-2,3-dihydro-1H-inden-1-yl)carbamate

¹H-NMR (400 MHz, CDCl₃) δ 8.15 (d, 1H), 7.11 (s, 1H), 7.03 (d, 1H), 6.89 (s, 1H), 6.85 (d, 1H), 4.93 (m, 2H), 4.80 (br, 1H), 3.97 (s, 3H), 3.79 (d, 3H), 3.25 (m, 1H), 2.92-2.71 (m, 7H), 2.10 (br, 1H), 1.85 (br, 1H), 1.69 (br, 1H), 1.59 (br, 1H), 1.43 (br, 1H), 1.29 (m, 3H), 0.98 (m, 6H)

### Example 333. (S)-quinuclidin-3-yl (5-(3-fluorophenyl)-6-methoxy-2,2-dimethyl-2,3-dihydro-1H-inden-1-yl)carbamate

¹H-NMR (400 MHz, CDCl₃) δ 7.34 (m, 1H), 7.25 (m, 2H), 7.10 (s, 1H), 7.02 (m, 1H), 6.86 (m, 1H), 4.95 (s, 2H), 4.80 (br, 1H), 3.81 (m, 3H), 3.25 (m, 1H), 2.90-2.71 (m, 7H), 2.10 (br, 1H), 1.85 (br, 1H), 1.69 (br, 1H), 1.59 (br, 1H), 1.46 (br, 1H), 1.28 (m, 3H), 0.98 (m, 3H)

### Example 334. (S)-quinuclidin-3-yl (5-(3-ethylphenyl)-6-methoxy-2,2-dimethyl-2,3-dihydro-1H-inden-1-yl)carbamate

¹H-NMR (400 MHz, CDCl₃) δ 7.33 (s, 3H), 7.17 (m, 1H), 7.16 (s, 1H), 6.86 (m, 1H), 4.95 (m, 2H), 4.80 (br, 1H), 3.80 (m, 3H), 3.25 (m, 1H), 2.90-2.80 (m, 7H), 2.70 (m, 2H), 2.10 (br, 1H), 1.85 (br, 1H), 1.69 (br, 1H), 1.59 (br, 1H), 1.46 (br, 1H), 1.28 (m, 6H), 0.98 (m, 3H)

### Example 335. (S)-quinuclidin-3-yl (5-(3-isobutylphenyl)-6-methoxy-2,2-dimethyl-2,3-dihydro-1H-inden-1-yl)carbamate

¹H-NMR (400 MHz, CDCl₃) δ 7.32-7.27 (m, 3H), 7.11 (m, 2H), 6.85 (m, 1H), 4.95 (m, 2H), 4.80 (br, 1H), 3.79 (m, 3H), 3.25 (m, 1H), 2.90-2.72 (m, 7H), 2.53 (d, 2H), 2.10 (br, 1H), 1.90 (m, 1H), 1.85 (br, 1H), 1.70 (br, 1H), 1.59 (br, 1H), 1.46 (br, 1H), 1.28 (m, 3H), 0.98 (m, 3H), 0.90 (d, 6H)

### Example 336. (S)-quinuclidin-3-yl (5-(3-isopropylphenyl)-6-methoxy-2,2-dimethyl-2,3-dihydro-1H-inden-1-yl)carbamate

¹H-NMR (400 MHz, CDCl₃) δ 7.33 (m, 3H), 7.20 (m, 1H), 7.12 (s, 1H), 6.85 (m, 1H), 4.95 (m, 2H), 4.80 (br, 1H), 3.80 (m, 3H), 3.25 (m, 1H), 2.95 (m, 1H), 2.90-2.72 (m, 7H), 2.10 (br, 1H), 1.85 (br, 1H), 1.70 (br, 1H), 1.59 (br, 1H), 1.46 (br, 1H), 1.28 (m, 9H), 0.98 (m, 3H)

### Example 337. (S)-quinuclidin-3-yl (5-(3-(tert-butyl)phenyl)-6-methoxy-2,2-dimethyl-2,3-dihydro-1H-inden-1-yl)carbamate

¹H-NMR (400 MHz, CDCl₃) δ 7.52 (s, 1H), 7.33 (m, 3H), 7.12 (s, 1H), 6.86 (m, 1H), 4.95 (m, 2H), 4.80 (br, 1H), 3.80 (m, 3H), 3.25 (m, 1H), 2.90-2.73 (m, 7H), 2.10 (br, 1H), 1.85 (br, 1H), 1.70 (br, 1H), 1.59 (br, 1H), 1.46 (br, 1H), 1.37 (s, 9H), 1.28 (m, 3H), 0.98 (m, 3H)

### Example 338. (S)-quinuclidin-3-yl (6-methoxy-5-(3-methoxyphenyl)-2,2-dimethyl-2,3-dihydro-1H-inden-1-yl)carbamate

¹H-NMR (400 MHz, CDCl₃) δ 7.31 (m, 1H), 7.11-7.06 (m, 3H), 6.87 (m, 2H), 4.95 (m, 2H), 4.80 (br, 1H), 3.84 (s, 3H), 3.80 (m, 3H), 3.25 (m, 1H), 2.90-2.73 (m, 7H), 2.10 (br, 1H), 1.85 (br, 1H), 1.70 (br, 1H), 1.59 (br, 1H), 1.46 (br, 1H), 1.28 (m, 3H), 0.98 (m, 3H)

### Example 339. (S)-quinuclidin-3-yl (5-(3-ethoxyphenyl)-6-methoxy-2,2-dimethyl-2,3-dihydro-1H-inden-1-yl)carbamate

¹H-NMR (400 MHz, CDCl₃) δ 7.30 (m, 1H), 7.11 (s, 1H), 7.07 (m, 2H), 6.85 (m, 2H), 4.95 (m, 2H), 4.80 (br, 1H), 4.08 (m, 2H), 3.79 (m, 3H), 3.25 (m, 1H), 2.90-2.73 (m, 7H), 2.10 (br, 1H), 1.85 (br, 1H), 1.70 (br, 1H), 1.59 (br, 1H), 1.46 (br, 1H), 1.43 (t, 3H), 1.28 (m, 3H), 0.98 (m, 3H)

### Example 340. (S)-quinuclidin-3-yl (6-methoxy-2,2-dimethyl-5-(3-propoxyphenyl)-2,3-dihydro-1H-inden-1-yl)carbamate

¹H-NMR (400 MHz, CDCl₃) δ 7.28 (m, 1H), 7.11 (s, 1H), 7.06 (m, 2H), 6.86 (m, 2H), 4.95 (m, 2H), 4.80 (br, 1H), 3.96 (m, 2H), 3.79 (m, 3H), 3.25 (m, 1H), 2.90-2.73 (m, 7H), 2.10 (br, 1H), 1.85 (br, 1H), 1.80 (m, 2H), 1.70 (br, 1H), 1.59 (br, 1H), 1.46 (br, 1H), 1.28 (m, 3H), 0.98 (m, 6H)

### Example 341. (S)-quinuclidin-3-yl (5-(3-isopropoxyphenyl)-6-methoxy-2,2-dimethyl-2,3-dihydro-1H-inden-1-yl)carbamate

¹H-NMR (400 MHz, CDCl₃) δ 7.28 (m, 1H), 7.11 (s, 1H), 7.05 (m, 2H), 6.85 (d, 2H), 4.95 (m, 2H), 4.80 (br, 1H), 4.59 (m, 1H), 3.80 (m, 3H), 3.25 (m, 1H), 2.90-2.73 (m, 7H), 2.10 (br, 1H), 1.85 (br, 1H), 1.70 (br, 1H), 1.59 (br, 1H), 1.46 (br, 1H), 1.35 (d, 6H), 1.28 (m, 3H), 0.98 (m, 3H)

### Example 342. (S)-quinuclidin-3-yl (5-(3-butoxyphenyl)-6-methoxy-2,2-dimethyl-2,3-dihydro-1H-inden-1-yl)carbamate

¹H-NMR (400 MHz, CDCl₃) δ 7.28 (m, 1H), 7.10 (s, 1H), 7.05 (m, 2H), 6.86 (d, 2H), 4.95 (m, 2H), 4.80 (br, 1H), 3.98 (m, 2H), 3.80 (m, 3H), 3.25 (m, 1H), 2.90-2.73 (m, 7H), 2.10 (br, 1H), 1.85 (br, 1H), 1.78 (m, 2H), 1.70 (br, 1H), 1.59 (br, 1H), 1.55 (m, 2H), 1.46 (br, 1H), 1.28 (m, 3H), 0.98 (m, 6H)

### Example 343. (S)-quinuclidin-3-yl (5-(3-isobutoxyphenyl)-6-methoxy-2,2-dimethyl-2,3-dihydro-1H-inden-1-yl)carbamate

¹H-NMR (400 MHz, CDCl₃) δ 7.28 (m, 1H), 7.11 (s, 1H), 7.05 (m, 2H), 6.85 (d, 2H), 4.95 (m, 2H), 4.80 (br, 1H), 3.80 (m, 3H), 3.75 (d, 2H), 3.25 (m, 1H), 2.90-2.73 (m, 7H), 2.10 (m, 2H), 1.85 (br, 1H), 1.70 (br, 1H), 1.59 (br, 1H), 1.46 (br, 1H), 1.28 (m, 3H), 0.98 (m, 9H)

### Example 344. (S)-quinuclidin-3-yl (5-(2,5-dimethoxyphenyl)-6-methoxy-2,2-dimethyl-2,3-dihydro-1H-inden-1-yl)carbamate

¹H-NMR (400 MHz, CDCl₃) δ 7.03 (s, 1H), 7.03-6.80 (m, 4H), 4.95 (s, 2H), 4.80 (br, 1H), 3.78 (m, 6H), 3.73 (s, 3H), 3.25 (m, 1H), 2.90-2.73 (m, 7H), 2.10 (br, 1H), 1.85 (br, 1H), 1.70 (br, 1H), 1.59 (br, 1H), 1.46 (br, 1H), 1.28 (m, 3H), 0.98 (m, 3H)

### Example 345. (S)-quinuclidin-3-yl (5-(3,5-dimethoxyphenyl)-6-methoxy-2,2-dimethyl-2,3-dihydro-1H-inden-1-yl)carbamate

¹H-NMR (400 MHz, CDCl₃) δ 7.11 (s, 1H), 6.85 (m, 1H), 6.65 (s, 2H), 6.45 (s, 1H), 4.95 (s, 2H), 4.80 (br, 1H), 3.80 (m, 9H), 3.25 (m, 1H), 2.90-2.73 (m, 7H), 2.10 (br, 1H), 1.85 (br, 1H), 1.70 (br, 1H), 1.59 (br, 1H), 1.46 (br, 1H), 1.28 (m, 3H), 0.98 (m, 3H)

### Example 346. (S)-quinuclidin-3-yl (5-(2-chloro-4-methoxyphenyl)-6-methoxy-2,2-dimethyl-2,3-dihydro-1H-inden-1-yl)carbamate

¹H-NMR (400 MHz, CDCl₃) δ 7.17 (d, 1H), 7.00 (s, 1H), 6.96 (s, 1H), 6.84 (m, 2H), 4.95 (s, 2H), 4.80 (br, 1H), 3.82 (s, 3H), 3.75 (m, 3H), 3.25 (m, 1H), 2.90-2.73 (m, 7H), 2.10 (br, 1H), 1.85 (br, 1H), 1.70 (br, 1H), 1.59 (br, 1H), 1.46 (br, 1H), 1.28 (m, 3H), 0.98 (m, 3H)

### Example 347. (S)-quinuclidin-3-yl (5-(4-ethoxy-3-fluorophenyl)-6-methoxy-2,2-dimethyl-2,3-dihydro-1H-inden-1-yl)carbamate

¹H-NMR (400 MHz, CDCl₃) δ 7.30(m 1H), 7.18 (d, 1H), 7.08 (s, 1H), 6.98 (t, 1H), 6.84 (d, 1H), 4.95 (s, 2H), 4.80 (br, 1H), 4.14 (m, 2H), 3.79 (m, 3H), 3.25 (m, 1H), 2.90-2.73 (m, 7H), 2.10 (br, 1H), 1.85 (br, 1H), 1.70 (br, 1H), 1.59 (br, 1H), 1.47 (t, 3H), 1.45 (br, 1H), 1.28 (m, 3H), 0.98 (m, 3H)

### Example 348. (S)-quinuclidin-3-yl (5-(4-isopropoxy-3,5-dimethylphenyl)-6-methoxy-2,2-dimethyl-2,3-dihydro-1H-inden-1-yl)carbamate

¹H-NMR (400 MHz, CDCl₃) δ 7.13 (s, 2H), 7.08 (s, 1H), 6.82 (d, 1H), 4.95 (s, 2H), 4.80 (br, 1H), 4.22 (m, 1H), 3.78 (m, 3H), 3.25 (m, 1H), 2.90-2.73 (m, 7H), 2.30 (s, 6H), 2.10 (br, 1H), 1.85 (br, 1H), 1.70 (br, 1H), 1.59 (br, 1H), 1.45 (br, 1H), 1.30 (d, 6H), 1.28 (m, 3H), 0.98 (m, 3H)

### Examples 349 to 388

The titled compounds of Examples 349 to 388 were prepared in accordance with the same procedures as in Example 1, using (S)-quinuclidin-3-yl (5-bromo-6-fluoro-2,2-dimethyl-2,3-dihydro-1H-inden-1-yl)carbamate prepared in Preparation 7; and the corresponding substituted-boronic acids, respectively.

### Example 349. (S)-quinuclidin-3-yl (5-(4-ethylphenyl)-6-fluoro-2,2-dimethyl-2,3-dihydro-1H-inden-1-yl)carbamate

¹H-NMR (400 MHz, CDCl₃) δ 7.44 (d, 2H), 7.27 (d, 2H), 7.20 (d, 1H), 7.03-6.97 (m, 1H), 4.93 (m, 2H), 4.79 (br, 1H), 3.28 (m, 1H), 2.90-2.71 (m, 7H), 2.69 (m, 2H), 2.11 (br, 1H), 1.85 (br, 1H), 1.72 (br, 1H), 1.59 (br, 1H), 1.43 (br, 1H), 1.32 (m, 6H), 0.97 (m, 3H)

### Example 350. (S)-quinuclidin-3-yl (6-fluoro-2,2-dimethyl-5-(4-propylphenyl)-2,3-dihydro-1H-inden-1-yl)carbamate

¹H-NMR (400 MHz, CDCl₃) δ 7.44 (d, 2H), 7.27 (m, 2H), 7.00 (m, 1H), 7.03-6.97 (m, 1H), 4.92 (m, 2H), 4.79 (br, 1H), 3.28 (m, 1H), 2.90-2.71 (m, 7H), 2.63 (m, 2H), 2.11 (br, 1H), 1.85 (br, 1H), 1.72 (br, 1H), 1.68 (m, 2H), 1.60 (br, 1H), 1.43 (br, 1H), 1.32 (m, 6H), 0.97 (m, 3H)

### Example 351. (S)-quinuclidin-3-yl (6-fluoro-5-(4-isopropylphenyl)-2,2-dimethyl-2,3-dihydro-1H-inden-1-yl)carbamate

¹H-NMR (400 MHz, CDCl₃) δ 7.44 (d, 2H), 7.27 (m, 2H), 7.00 (m, 1H), 7.03-6.97 (m, 1H), 4.92 (m, 2H), 4.79 (br, 1H), 3.28 (m, 1H), 2.96 (m, 1H), 2.90-2.72 (m, 7H), 2.11 (br, 1H), 1.85 (br, 1H), 1.72 (br, 1H), 1.60 (br, 1H), 1.43 (br, 1H), 1.28 (m, 9H), 0.97 (m, 3H)

### Example 352. (S)-quinuclidin-3-yl (6-fluoro-5-(4-isobutylphenyl)-2,2-dimethyl-2,3-dihydro-1H-inden-1-yl)carbamate

¹H-NMR (400 MHz, CDCl₃) δ 7.44 (d, 2H), 7.22 (d, 3H), 7.03-6.97 (m, 1H), 4.92 (m, 2H), 4.79 (br, 1H), 3.28 (m, 1H), 2.90-2.72 (m, 7H), 2.52 (d, 2H), 2.11 (br, 1H), 1.94 (m, 1H), 1.85 (br, 1H), 1.72 (br, 1H), 1.60 (br, 1H), 1.43 (br, 1H), 1.30 (m, 3H), 0.98 (m, 3H), 0.94 (d, 6H)

### Example 353. (S)-quinuclidin-3-yl (5-(4-(tert-butyl)phenyl)-6-fluoro-2,2-dimethyl-2,3-dihydro-1H-inden-1-yl)carbamate

¹H-NMR (400 MHz, CDCl₃) δ 7.46 (s, 4H), 7.21 (d, 1H), 7.03-6.97 (m, 1H), 4.99-4.90 (m, 2H), 4.79 (br, 1H), 3.29 (m, 1H), 2.90-2.69 (m, 7H), 2.11 (br, 1H), 1.94 (m, 1H), 1.85 (br, 1H), 1.72 (br, 1H), 1.60 (br, 1H), 1.43 (br, 1H), 1.33 (s, 9H), 1.30 (m, 3H), 0.98 (m, 3H)

### Example 354. (S)-quinuclidin-3-yl (5-(4-cyclopropylphenyl)-6-fluoro-2,2-dimethyl-2,3-dihydro-1H-inden-1-yl)carbamate

¹H-NMR (400 MHz, CDCl₃) δ 7.42 (d, 2H), 7.20 (d, 1H), 7.13 (d, 2H), 7.02-6.99 (m, 1H), 4.98-4.88 (m, 2H), 4.79 (br, 1H), 3.28 (m, 1H), 2.88-2.68 (m, 7H), 2.11 (br, 1H), 1.95 (m, 1H), 1.85 (br, 1H), 1.72 (br, 1H), 1.60 (br, 1H), 1.43 (br, 1H), 1.29 (m, 3H), 1.00 (m, 2H), 0.98 (m, 3H), 0.75 (m, 2H)

### Example 355. (S)-quinuclidin-3-yl (6-fluoro-5-(4-methoxyphenyl)-2,2-dimethyl-2,3-dihydro-1H-inden-1-yl)carbamate

¹H-NMR (400 MHz, CDCl₃) δ 7.46 (d, 2H), 7.19 (d, 1H), 6.99 (m, 3H), 4.98 (m, 1H), 4.88 (m, 1H), 4.79 (br, 1H), 3.96 (s, 3H), 3.28 (m, 1H), 2.88-2.68 (m, 7H), 2.11 (br, 1H), 1.85 (br, 1H), 1.72 (br, 1H), 1.60 (br, 1H), 1.43 (br, 1H), 1.29 (m, 3H), 0.98 (m, 3H)

### Example 356. (S)-quinuclidin-3-yl (5-(4-ethoxyphenyl)-6-fluoro-2,2-dimethyl-2,3-dihydro-1H-inden-1-yl)carbamate

¹H-NMR (400 MHz, CDCl₃) δ 7.44 (d, 2H), 7.18 (d, 1H), 6.98 (m, 3H), 4.98 (m, 1H), 4.88 (m, 1H), 4.79 (br, 1H), 4.11 (m, 2H), 3.28 (m, 1H), 2.88-2.68 (m, 7H), 2.11 (br, 1H), 1.85 (br, 1H), 1.72 (br, 1H), 1.60 (br, 1H), 1.45 (t, 3H), 1.44 (br, 1H), 1.29 (m, 3H), 0.98 (m, 3H)

### Example 357. (S)-quinuclidin-3-yl (6-fluoro-2,2-dimethyl-5-(4-propoxyphenyl)-2,3-dihydro-1H-inden-1-yl)carbamate

¹H-NMR (400 MHz, CDCl₃) δ 7.44 (d, 2H), 7.18 (d, 1H), 6.98 (m, 3H), 4.98 (m, 1H), 4.88 (m, 1H), 4.79 (br, 1H), 3.97 (t, 2H), 3.28 (m, 1H), 2.88-2.68 (m, 7H), 2.11 (br, 1H), 1.85 (m, 3H), 1.72 (br, 1H), 1.60 (br, 1H), 1.45 (t, 3H), 1.44 (br, 1H), 1.29 (m, 3H), 1.05 (t, 3H), 0.98 (m, 3H)

### Example 358. (S)-quinuclidin-3-yl (6-fluoro-5-(4-isopropoxyphenyl)-2,2-dimethyl-2,3-dihydro-1H-inden-1-yl)carbamate

¹H-NMR (400 MHz, CDCl₃) δ 7.43 (d, 2H), 7.18 (d, 1H), 7.02 (m, 1H), 6.94 (d, 2H), 4.98 (m, 1H), 4.88 (m, 1H), 4.79 (br, 1H), 4.58 (m, 1H), 3.28 (m, 1H), 2.88-2.68 (m, 7H), 2.11 (br, 1H), 1.85 (br, 1H), 1.72 (br, 1H), 1.60 (br, 1H), 1.44 (br, 1H), 1.31 (d, 6H), 1.29 (m, 3H), 0.98 (m, 3H)

### Example 359. (S)-quinuclidin-3-yl (5-(3-chloro-4-isopropoxyphenyl)-6-fluoro-2,2-dimethyl-2,3-dihydro-1H-inden-1-yl)carbamate

¹H-NMR (400 MHz, CDCl₃) δ 7.54 (s, 1H), 7.37 (d, 1H), 7.18 (d, 1H), 7.02 (m, 2H), 4.98 (m, 2H), 4.79 (br, 1H), 4.57 (m, 1H), 3.28 (m, 1H), 2.88-2.68 (m, 7H), 2.11 (br, 1H), 1.85 (br, 1H), 1.72 (br, 1H), 1.60 (br, 1H), 1.44 (br, 1H), 1.42 (d, 6H), 1.28 (m, 3H), 0.98 (m, 3H)

### Example 360. (S)-quinuclidin-3-yl (6-fluoro-5-(2-fluoro-4-methoxyphenyl)-2,2-dimethyl-2,3-dihydro-1H-inden-1-yl)carbamate

¹H-NMR (400 MHz, CDCl₃) δ 7.28 (m, 1H), 7.14 (d, 1H), 7.00 (m, 1H), 6.75 (m, 2H), 4.98 (m, 2H), 4.79 (br, 1H), 3.84 (s, 3H), 3.28 (m, 1H), 2.88-2.68 (m, 7H), 2.11 (br, 1H), 1.85 (br, 1H), 1.72 (br, 1H), 1.60 (br, 1H), 1.43 (br, 1H), 1.29 (m, 3H), 0.98 (m, 3H)

### Example 361. (S)-quinuclidin-3-yl (5-(4-butoxyphenyl)-6-fluoro-2,2-dimethyl-2,3-dihydro-1H-inden-1-yl)carbamate

¹H-NMR (400 MHz, CDCl₃) δ 7.44(d, 12), 7.18 (d, 1H), 6.98 (m, 3H), 4.98 (m, 2H), 4.79 (br, 1H), 4.01 (t, 2H), 3.28 (m, 1H), 2.88-2.68 (m, 7H), 2.11 (br, 1H), 1.85 (br, 1H), 1.81 (m, 2H), 1.72 (br, 1H), 1.60 (br, 1H), 1.51 (m, 2H), 1.43 (br, 1H), 1.29 (m, 3H), 0.98 (m, 6H)

### Example 362. (S)-quinuclidin-3-yl (6-fluoro-5-(4-isobutoxyphenyl)-2,2-dimethyl-2,3-dihydro-1H-inden-1-yl)carbamate

¹H-NMR (400 MHz, CDCl₃) δ 7.44 (d, 2H), 7.18 (d, 1H), 6.97 (m, 3H), 4.98 (m, 2H), 4.79 (br, 1H), 3.77 (d, 2H), 3.28 (m, 1H), 2.88-2.68 (m, 7H), 2.11 (m, 3H), 1.85 (br, 1H), 1.81 (m, 2H), 1.72 (br, 1H), 1.60 (br, 1H), 1.51 (m, 2H), 1.43 (br, 1H), 1.29 (m, 3H), 1.05 (d, 6H), 0.98 (m, 6H)

### Example 363. (S)-quinuclidin-3-yl (5-(6-(cyclopropylmethoxy)pyridin-3-yl)-6-fluoro-2,2-dimethyl-2,3-dihydro-1H-inden-1-yl)carbamate

¹H-NMR (400 MHz, CDCl₃) δ 8.27 (s, 1H), 7.74 (d, 1H), 7.16 (d, 1H), 7.00 (m, 1H), 6.83 (d, 1H), 4.98 (m, 1H), 4.90 (m, 1H), 4.79 (br, 1H), 4.18 (d, 2H), 3.28 (m, 1H), 2.88-2.68 (m, 7H), 2.11 (br, 1H), 1.85 (br, 1H), 1.72 (br, 1H), 1.60 (br, 1H), 1.43 (br, 1H), 1.29 (m, 4H), 0.98 (m, 3H), 0.64 (m, 2H), 0.37 (m, 2H)

### Example 364. (S)-quinuclidin-3-yl (5-(5-chloro-2-methoxyphenyl)-6-fluoro-2,2-dimethyl-2,3-dihydro-1H-inden-1-yl)carbamate

¹H-NMR (400 MHz, CDCl₃) δ 7.31 (m, 2H), 7.09 (d, 1H), 6.97 (m, 1H), 6.89 (d, 1H), 4.98 (m, 1H), 4.90 (m, 1H), 4.79 (br, 1H), 3,78(s, 3H), 3.28 (m, 1H), 2.88-2.68 (m, 7H), 2.11 (br, 1H), 1.85 (br, 1H), 1.72 (br, 1H), 1.60 (br, 1H), 1.43 (br, 1H), 1.29 (m, 4H), 0.98 (m, 3H), 0.64 (m, 2H), 0.37 (m, 2H)

### Example 365. (S)-quinuclidin-3-yl (5-(4-butylphenyl)-6-fluoro-2,2-dimethyl-2,3-dihydro-1H-inden-1-yl)carbamate

¹H-NMR (400 MHz, CDCl₃) δ 7.42 (d, 2H) 7.25 (m, 3H), 6.97 (m, 1H), 4.95 (s, 1H), 4.85 (m, 1H), 4.80 (br, 1H), 3.25 (m, 1H), 2.92-2.65 (m, 7H), 2.64 (m, 2H), 2.10 (m, 1H), 1.85 (br, 1H), 1.69 (br, 1H), 1.60 (m, 2H), 1.59 (br, 1H), 1.43 (m, 3H), 1.29 (m, 3H), 0.98 (m, 6H)

### Example 366. (S)-quinuclidin-3-yl (6-fluoro-5-(4-methoxy-3,5-dimethylphenyl)-2,2-dimethyl-2,3-dihydro-1H-inden-1-yl)carbamate

¹H-NMR (400 MHz, CDCl₃) δ 7.17 (s, 4H), 7.98 (m, 1H), 4.95 (s, 1H), 4.85 (m, 1H), 4.80 (br, 1H), 3.77 (s, 3H), 3.25 (m, 1H), 2.92-2.65 (m, 7H), 2.34 (s, 6H), 2.10 (br, 1H), 1.85 (br, 1H), 1.69 (br, 1H), 1.58 (br, 1H), 1.40 (br, 1H), 1.28 (m, 3H), 0.98 (m, 3H)

### Example 367. (S)-quinuclidin-3-yl (5-(4-ethoxy-3,5-dimethylphenyl)-6-fluoro-2,2-dimethyl-2,3-dihydro-1H-inden-1-yl)carbamate

¹H-NMR (400 MHz, CDCl₃) δ 7.17 (s, 4H), 6.98 (m, 1H), 4.95 (s, 1H), 4.85 (m, 1H), 4.80 (br, 1H), 3.88 (d, 3H), 3.25 (m, 1H), 2.92-2.65 (m, 7H), 2.32 (s, 6H), 2.10 (br, 1H), 1.85 (br, 1H), 1.69 (br, 1H), 1.59 (br, 1H), 1.44 (t, 4H), 1.28 (m, 3H), 0.98 (m, 3H)

### Example 368. (S)-quinuclidin-3-yl (5-(4-butoxy-3,5-dimethylphenyl)-6-fluoro-2,2-dimethyl-2,3-dihydro-1H-inden-1-yl)carbamate

¹H-NMR (400 MHz, CDCl₃) δ 7.18 (s, 4H), 6.98 (m, 1H), 4.95 (s, 1H), 4.85 (m, 1H), 4.80 (br, 1H), 3.80 (m, 2H), 3.25 (m, 1H), 2.92-2.71 (m, 7H), 2.31 (s, 6H), 2.10 (m, 1H), 1.85 (br, 1H), 1.77 (m, 2H), 1.69 (br, 1H), 1.60 (m, 2H), 1.59 (m, 3H), 1.43 (br, 1H), 1.29 (m, 3H), 0.98 (m, 6H)

### Example 369. (S)-quinuclidin-3-yl (6-fluoro-5-(2-methoxypyridin-4-yl)-2,2-dimethyl-2,3-dihydro-1H-inden-1-yl)carbamate

¹H-NMR (400 MHz, CDCl₃) δ 8.19 (d, 1H), 7.23 (m, 1H), 7.03 (m, 1H), 6.89 (s, 1H), 4.95 (s, 1H), 4.85 (m, 1H), 4.80 (br, 1H), 3.97 (s, 3H), 3.25 (m, 1H), 2.92-2.71 (m, 7H), 2.10 (br, 1H), 1.85 (br, 1H), 1.69 (br, 1H), 1.59 (br, 1H), 1.43 (br, 1H), 1.29 (m, 3H), 0.98 (m, 3H)

### Example 370. (S)-quinuclidin-3-yl (6-fluoro-5-(3-fluorophenyl)-2,2-dimethyl-2,3-dihydro-1H-inden-1-yl)carbamate

¹H-NMR (400 MHz, CDCl₃) δ 7.38 (m, 1H), 7.36-7.19 (m, 3H), 7.05 (m, 2H), 4.95 (s, 1H), 4.85 (m, 1H), 4.80 (br, 1H), 3.25 (m, 1H), 2.90-2.71 (m, 7H), 2.10 (br, 1H), 1.85 (br, 1H), 1.69 (br, 1H), 1.59 (br, 1H), 1.46 (br, 1H), 1.28 (m, 3H), 0.98 (m, 3H)

### Example 371. (S)-quinuclidin-3-yl (5-(3-ethylphenyl)-6-fluoro-2,2-dimethyl-2,3-dihydro-1H-inden-1-yl)carbamate

¹H-NMR (400 MHz, CDCl₃) δ 7.34 (m, 3H), 7.20 (m, 2H), 7.00 (m, 1H), 4.95 (s, 1H), 4.85 (m, 1H), 4.80 (br, 1H), 3.25 (m, 1H), 2.90-2.70 (m, 7H), 2.68 (m, 2H), 2.10 (br, 1H), 1.85 (br, 1H), 1.69 (br, 1H), 1.59 (br, 1H), 1.46 (br, 1H), 1.28 (m, 6H), 0.98 (m, 3H)

### Example 372. (S)-quinuclidin-3-yl (6-fluoro-5-(3-isobutylphenyl)-2,2-dimethyl-2,3-dihydro-1H-inden-1-yl)carbamate

¹H-NMR (400 MHz, CDCl₃) δ 7.34 (m, 2H), 7.27 (m, 1H), 7.21 (m, 1H), 7.15 (m, 1H), 6.99 (m, 1H), 4.95 (s, 1H), 4.85 (m, 1H), 4.80 (br, 1H), 3.25 (m, 1H), 2.90-2.72 (m, 7H), 2.52 (d, 2H), 2.10 (br, 1H), 1.90 (m, 1H), 1.85 (br, 1H), 1.70 (br, 1H), 1.59 (br, 1H), 1.46 (br, 1H), 1.28 (m, 3H), 0.98 (m, 3H), 0.90 (d, 6H)

### Example 373. (S)-quinuclidin-3-yl (6-fluoro-5-(3-isopropylphenyl)-2,2-dimethyl-2,3-dihydro-1H-inden-1-yl)carbamate

¹H-NMR (400 MHz, CDCl₃) δ 7.37-7.34 (m, 3H), 7.22 (m, 2H), 6.99 (m, 1H), 4.95 (s, 1H), 4.85 (m, 1H), 4.80 (br, 1H), 3.25 (m, 1H), 2.95 (m, 1H), 2.90-2.72 (m, 7H), 2.10 (br, 1H), 1.85 (br, 1H), 1.70 (br, 1H), 1.59 (br, 1H), 1.46 (br, 1H), 1.28 (m, 9H), 0.98 (m, 3H)

### Example 374. (S)-quinuclidin-3-yl (5-(3-(tert-butyl)phenyl)-6-fluoro-2,2-dimethyl-2,3-dihydro-1H-inden-1-yl)carbamate

¹H-NMR (400 MHz, CDCl₃) δ 7.53 (s, 1H), 7.41 (m, 1H), 7.35 (m, 1H), 7.22 (d, 1H), 7.01 (m, 1H), 4.95 (s, 1H), 4.85 (m, 1H), 4.80 (br, 1H), 3.80 (m, 3H), 3.25 (m, 1H), 2.90-2.73 (m, 7H), 2.10 (br, 1H), 1.85 (br, 1H), 1.70 (br, 1H), 1.59 (br, 1H), 1.46 (br, 1H), 1.37 (s, 9H), 1.28 (m, 3H), 0.98 (m, 3H)

### Example 375. (S)-quinuclidin-3-yl (6-fluoro-5-(3-methoxyphenyl)-2,2-dimethyl-2,3-dihydro-1H-inden-1-yl)carbamate

¹H-NMR (400 MHz, CDCl₃) δ 7.35 (m, 1H), 7.22 (m, 1H), 7.11 (m, 1H), 7.09 (s, 1H), 7.01 (m, 1H), 6.91 (d, 1H), 4.95 (s, 1H), 4.85 (m, 1H), 4.80 (br, 1H), 3.85 (s, 3H), 3.25 (m, 1H), 2.90-2.73 (m, 7H), 2.10 (br, 1H), 1.85 (br, 1H), 1.70 (br, 1H), 1.59 (br, 1H), 1.46 (br, 1H), 1.28 (m, 3H), 0.98 (m, 3H)

### Example 376. (S)-quinuclidin-3-yl (5-(3-ethoxyphenyl)-6-fluoro-2,2-dimethyl-2,3-dihydro-1H-inden-1-yl)carbamate

¹H-NMR (400 MHz, CDCl₃) δ 7.35 (m, 1H), 7.22 (m, 1H), 7.11 (m, 1H), 7.09 (s, 1H), 7.01 (m, 1H), 6.89 (d, 1H), 4.95 (s, 1H), 4.85 (m, 1H), 4.80 (br, 1H), 4.09 (m, 2H), 3.25 (m, 1H), 2.90-2.73 (m, 7H), 2.10 (br, 1H), 1.85 (br, 1H), 1.70 (br, 1H), 1.59 (br, 1H), 1.46 (br, 1H), 1.45 (t, 3H), 1.28 (m, 3H), 0.98 (m, 3H)

### Example 377. (S)-quinuclidin-3-yl (6-fluoro-2,2-dimethyl-5-(3-propoxyphenyl)-2,3-dihydro-1H-inden-1-yl)carbamate

¹H-NMR (400 MHz, CDCl₃) δ 7.33 (m, 1H), 7.22 (d, 1H), 7.11 (m, 1H), 7.09 (s, 1H), 7.01 (m, 1H), 6.90 (d, 1H), 4.95 (s, 1H), 4.85 (m, 1H), 4.80 (br, 1H), 3.96 (m, 2H), 3.25 (m, 1H), 2.90-2.73 (m, 7H), 2.10 (br, 1H), 1.85 (br, 1H), 1.80 (m, 2H), 1.70 (br, 1H), 1.59 (br, 1H), 1.46 (br, 1H), 1.28 (m, 3H), 1.15 (t, 3H), 0.98 (m, 3H)

### Example 378. (S)-quinuclidin-3-yl (6-fluoro-5-(3-isopropoxyphenyl)-2,2-dimethyl-2,3-dihydro-1H-inden-1-yl)carbamate

¹H-NMR (400 MHz, CDCl₃) δ 7.32 (m, 1H), 7.22 (d, 1H), 7.11 (m, 1H), 7.09 (s, 1H), 7.01 (m, 1H), 6.87 (d, 1H), 4.95 (m, 2H), 4.80 (br, 1H), 4.57 (m, 1H), 3.25 (m, 1H), 2.90-2.73 (m, 7H), 2.10 (br, 1H), 1.85 (br, 1H), 1.70 (br, 1H), 1.59 (br, 1H), 1.46 (br, 1H), 1.35 (d, 6H), 1.28 (m, 3H), 0.98 (m, 3H)

### Example 379. (S)-quinuclidin-3-yl (5-(3-butoxyphenyl)-6-fluoro-2,2-dimethyl-2,3-dihydro-1H-inden-1-yl)carbamate

¹H-NMR (400 MHz, CDCl₃) δ 7.34 (m, 1H), 7.22 (d, 1H), 7.10-7.01 (m, 3H), 6.89 (d, 1H), 4.95 (s, 1H), 4.85 (m, 1H), 4.80 (br, 1H), 4.00 (m, 2H), 3.25 (m, 1H), 2.90-2.73 (m, 7H), 2.10 (br, 1H), 1.85 (br, 1H), 1.78 (m, 2H), 1.70 (br, 1H), 1.59 (br, 1H), 1.53 (m, 2H), 1.46 (br, 1H), 1.28 (m, 3H), 0.98 (m, 6H)

### Example 380. (S)-quinuclidin-3-yl (6-fluoro-5-(3-isobutoxyphenyl)-2,2-dimethyl-2,3-dihydro-1H-inden-1-yl)carbamate

¹H-NMR (400 MHz, CDCl₃) δ 7.33 (m, 1H), 7.22 (d, 1H), 7.11-6.91 (m, 3H), 6.89 (d, 1H), 4.95 (s, 1H), 4.85 (m, 1H), 4.80 (br, 1H), 3.76 (d, 2H), 3.25 (m, 1H), 2.90-2.73 (m, 7H), 2.10 (m, 2H), 1.85 (br, 1H), 1.70 (br, 1H), 1.59 (br, 1H), 1.46 (br, 1H), 1.28 (m, 3H), 1.05 (d, 6H), 0.97 (m, 3H)

### Example 381. (S)-quinuclidin-3-yl (5-(2,4-dimethoxyphenyl)-6-fluoro-2,2-dimethyl-2,3-dihydro-1H-inden-1-yl)carbamate

¹H-NMR (400 MHz, CDCl₃) δ 7.16(d 1H), 7.09 (d, 1H), 6.97 (m, 1H), 6.56 (m, 2H), 4.95 (s, 1H), 4.85 (m, 1H), 4.80 (br, 1H), 3.85 (s, 3H), 3.79 (s, 3H), 3.25 (m, 1H), 2.92-2.71 (m, 7H), 2.10 (br, 1H), 1.85 (br, 1H), 1.70 (br, 1H), 1.59 (br, 1H), 1.46 (br, 1H), 1.28 (m, 3H), 0.98 (m, 3H)

### Example 382. (S)-quinuclidin-3-yl (5-(2,5-dimethoxyphenyl)-6-fluoro-2,2-dimethyl-2,3-dihydro-1H-inden-1-yl)carbamate

¹H-NMR (400 MHz, CDCl₃) δ 7.12 (d, 1H), 7.01 (m, 1H), 6.96 (m, 2H), 6.87 (s, 1H), 4.95 (s, 1H), 4.85 (m, 1H), 4.80 (br, 1H), 3.79 (s, 3H), 3.76 (s, 3H), 3.25 (m, 1H), 2.93-2.72 (m, 7H), 2.10 (br, 1H), 1.85 (br, 1H), 1.70 (br, 1H), 1.59 (br, 1H), 1.46 (br, 1H), 1.28 (m, 3H), 0.98 (m, 3H)

### Example 383. (S)-quinuclidin-3-yl (5-(2,6-dimethoxyphenyl)-6-fluoro-2,2-dimethyl-2,3-dihydro-1H-inden-1-yl)carbamate

¹H-NMR (400 MHz, CDCl₃) δ 7.33 (m, 1H), 7.05 (d, 1H), 6.98 (m, 1H), 6.65 (d, 2H), 4.95 (s, 1H), 4.85 (m, 1H), 4.80 (br, 1H), 3.75 (s, 6H), 3.25 (m, 1H), 2.93-2.72 (m, 7H), 2.10 (br, 1H), 1.85 (br, 1H), 1.70 (br, 1H), 1.59 (br, 1H), 1.46 (br, 1H), 1.28 (m, 3H), 0.98 (m, 3H)

### Example 384. (S)-quinuclidin-3-yl (5-(3,5-dimethoxyphenyl)-6-fluoro-2,2-dimethyl-2,3-dihydro-1H-inden-1-yl)carbamate

¹H-NMR (400 MHz, CDCl₃) δ 7.20 (d, 1H), 7.02 (m, 1H), 6.65 (s, 2H), 6.48 (s, 1H), 4.95 (s, 1H), 4.85 (m, 1H), 4.80 (br, 1H), 3.83 (s, 6H), 3.25 (m, 1H), 2.89-2.72 (m, 7H), 2.10 (br, 1H), 1.85 (br, 1H), 1.70 (br, 1H), 1.59 (br, 1H), 1.46 (br, 1H), 1.28 (m, 3H), 0.98 (m, 3H)

### Example 385. (S)-quinuclidin-3-yl (5-(3,4-dimethoxyphenyl)-6-fluoro-2,2-dimethyl-2,3-dihydro-1H-inden-1-yl)carbamate

¹H-NMR (400 MHz, CDCl₃) δ 7.20 (d, 1H), 7.08-6.92 (m, 3H), 6.93 (d, 1H), 4.95 (s, 1H), 4.85 (m, 1H), 4.80 (br, 1H), 3.93 (s, 6H), 3.25 (m, 1H), 2.93-2.72 (m, 7H), 2.10 (br, 1H), 1.85 (br, 1H), 1.70 (br, 1H), 1.59 (br, 1H), 1.46 (br, 1H), 1.28 (m, 3H), 0.98 (m, 3H)

### Example 386. (S)-quinuclidin-3-yl (5-(2-chloro-4-methoxyphenyl)-6-fluoro-2,2-dimethyl-2,3-dihydro-1H-inden-1-yl)carbamate

¹H-NMR (400 MHz, CDCl₃) δ 7.21 (d, 1H), 7.06 (d, 1H), 7.03-7.00 (m, 2H), 6.86 (d, 1H), 4.95 (s, 1H), 4.85 (m, 1H), 4.80 (br, 1H), 3.84 (s, 3H), 3.25 (m, 1H), 2.90-2.72 (m, 7H), 2.10 (br, 1H), 1.85 (br, 1H), 1.70 (br, 1H), 1.59 (br, 1H), 1.46 (br, 1H), 1.28 (m, 3H), 0.98 (m, 3H)

### Example 387. (S)-quinuclidin-3-yl (5-(4-ethoxy-3-fluorophenyl)-6-fluoro-2,2-dimethyl-2,3-dihydro-1H-inden-1-yl)carbamate

¹H-NMR (400 MHz, CDCl₃) δ 7.29 (m, 1H), 7.23 (m, 1H), 7.17 (d, 1H), 7.01 (m, 2H), 4.95 (s, 1H), 4.85 (m, 1H), 4.80 (br, 1H), 4.15 (m, 2H), 3.25 (m, 1H), 2.90-2.73 (m, 7H), 2.10 (br, 1H), 1.85 (br, 1H), 1.70 (br, 1H), 1.59 (br, 1H), 1.47 (t, 3H), 1.45 (br, 1H), 1.28 (m, 3H), 0.98 (m, 3H)

### Example 388. (S)-quinuclidin-3-yl (6-fluoro-5-(4-isopropoxy-3,5-dimethylphenyl)-2,2-dimethyl-2,3-dihydro-1H-inden-1-yl)carbamate

¹H-NMR (400 MHz, CDCl₃) δ 7.19-7.00 (m, 3H), 6.97 (m, 1H), 4.95 (m, 1H), 4.85 (m, 1H), 4.80 (br, 1H), 4.21 (m, 1H), 3.25 (m, 1H), 2.90-2.73 (m, 7H), 2.31 (s, 6H), 2.10 (br, 1H), 1.85 (br, 1H), 1.70 (br, 1H), 1.59 (br, 1H), 1.45 (br, 1H), 1.30 (d, 6H), 1.28 (m, 3H), 0.98 (m, 3H)

### Examples 389 to 398

The titled compounds of Examples 389 to 398 were prepared in accordance with the same procedures as in Example 1, using (S)-quinuclidin-3-yl (5′-bromo-1′,3′-dihydrospiro[cyclopropane-1,2′-inden]-1′-yl)carbamate prepared in Preparation 8; and the corresponding substituted-phenylboronic acids, respectively.

### Example 389. (S)-quinuclidin-3-yl (5′-(4-fluorophenyl)-1′,3′-dihydrospiro[cyclopropane-1,2′-inden]-1′-yl)carbamate

¹H-NMR (400 MHz, CDCl₃) δ 7.54 (m, 2H), 7.42 (m, 2H), 7.38 (s, 1H), 7.13 (t, 2H), 5.02 (m, 1H), 4.93 (m, 1H), 4.74 (m, 1H), 3.25 (m, 1H), 3.10 (m, 1H), 2.91-2.72 (m, 6H), 2.09 (m, 1H), 1.82 (m, 1H), 1.70 (br, 1H), 1,60(m, 1H), 1.43 (br, 1H), 0.92 (m, 2H), 0.81 (m, 1H), 0.59 (m, 1H)

### Example 390. (S)-quinuclidin-3-yl (5′-(4-chlorophenyl)-1′,3′-dihydrospiro[cyclopropane-1,2′-inden]-1′-yl)carbamate

¹H-NMR (400 MHz, CDCl₃) δ 7.55 (d, 2H), 7.47 (m, 5H), 5.02 (m, 1H), 4.91 (m, 1H), 4.77 (m, 1H), 3.25 (m, 1H), 3.10 (m, 1H), 2.91-2.72 (m, 6H), 2.09 (m, 1H), 1.82 (m, 1H), 1.70 (br, 1H), 1,60(m, 1H), 1.43 (br, 1H), 0.92 (m, 2H), 0.81 (m, 1H), 0.59 (m, 1H)

### Example 391. (S)-quinuclidin-3-yl (5′-(4-(trifluoromethyl)phenyl)-1′,3′-dihydrospiro[cyclopropane-1,2′-inden]-1′-yl)carbamate

¹H-NMR (400 MHz, CDCl₃) δ 7.69 (s, 4H), 7.47 (m, 3H), 5.04 (m, 1H), 4.95 (m, 1H), 4.77 (m, 1H), 3.25 (m, 1H), 3.10 (m, 1H), 2.91-2.72 (m, 6H), 2.09 (m, 1H), 1.82 (m, 1H), 1.70 (br, 1H), 1,60(m, 1H), 1.43 (br, 1H), 0.91 (m, 2H), 0.81 (m, 1H), 0.59 (m, 1H)

### Example 392. (S)-quinuclidin-3-yl (5′-(4-(trifluoromethoxy)phenyl)-1′,3′-dihydrospiro[cyclopropane-1,2′-inden]-1′-yl)carbamate

¹H-NMR (400 MHz, CDCl₃) δ 7.58 (d, 2H), 7.46 (m, 2H), 7.40 (s, 1H), 7.29 (d, 2H), 5.03 (m, 1H), 4.94 (m, 1H), 4.76 (m, 1H), 3.25 (m, 1H), 3.09 (m, 1H), 2.95-2.76 (m, 6H), 2.09 (m, 1H), 1.82 (m, 1H), 1.72 (br, 1H), 1,62(m, 1H), 1.43 (br, 1H), 0.92 (m, 2H), 0.81 (m, 1H), 0.59 (m, 1H)

### Example 393. (S)-quinuclidin-3-yl (5′-(3-chlorophenyl)-1′,3′-dihydrospiro[cyclopropane-1,2′-inden]-1′-yl)carbamate

¹H-NMR (400 MHz, CDCl₃) δ 7.56 (s, 1H), 7.44 (m, 2H), 7.37 (m, 1H), 7.33 (m, 1H), 5.02 (m, 1H), 4.91 (m, 1H), 4.77 (m, 1H), 3.25 (m, 1H), 2.91-2.72 (m, 7H), 2.09 (m, 1H), 1.80 (m, 1H), 1.70 (br, 1H), 1,60(m, 1H), 1.43 (br, 1H), 0.91 (m, 2H), 0.80 (m, 1H), 0.59 (m, 1H)

### Example 394. (S)-quinuclidin-3-yl (5′-(3-(trifluoromethyl)phenyl)-1′,3′-dihydrospiro[cyclopropane-1,2′-inden]-1′-yl)carbamate

¹H-NMR (400 MHz, CDCl₃) δ 7.56 (s, 1H), 7.44 (m, 2H), 7.37 (m, 1H), 7.33 (m, 1H), 5.02 (m, 1H), 4.91 (m, 1H), 4.77 (m, 1H), 3.25 (m, 1H), 2.91-2.72 (m, 7H), 2.09 (m, 1H), 1.80 (m, 1H), 1.70 (br, 1H), 1,60(m, 1H), 1.43 (br, 1H), 0.91 (m, 2H), 0.80 (m, 1H), 0.59 (m, 1H)

### Example 395. (S)-quinuclidin-3-yl (5′-(4-(2-methoxyethoxy)phenyl)-1′,3′-dihydrospiro[cyclopropane-1,2′-inden]-1′-yl)carbamate

¹H-NMR (400 MHz, CDCl₃) δ 7.51 (d, 2H), 7.41 (m, 3H), 7.01 (d, 2H), 5.02 (m, 1H), 4.90 (m, 1H), 4.75 (m, 1H), 4.18 (m, 2H), 3.80 (m, 2H), 3.48 (s, 3H), 3.25 (m, 1H), 3.08 (m, 1H), 2.91-2.72 (m, 5H), 2.09 (m, 1H), 1.85 (br, 1H), 1.80 (m, 1H), 1.70 (br, 1H), 1,60(m, 1H), 1.43 (br, 1H), 0.91 (m, 2H), 0.81 (m, 1H), 0.59 (m, 1H)

### Example 396. (S)-quinuclidin-3-yl (5′-(3-(2-methoxyethoxy)phenyl)-1′,3′-dihydrospiro[cyclopropane-1,2′-inden]-1′-yl)carbamate

¹H-NMR (400 MHz, CDCl₃) δ 7.46 (m, 2H), 7.45 (s, 1H), 7.35 (t, 1H), 7.18 (m, 2H), 6.93 (d, 1H), 5.04 (m, 1H), 4.91 (m, 1H), 4.75 (m, 1H), 4.19 (m, 2H), 3.79 (m, 2H), 3.48 (s, 3H), 3.25 (m, 1H), 3.08 (m, 1H), 2.91-2.72 (m, 5H), 2.09 (m, 1H), 1.85 (br, 1H), 1.80 (m, 1H), 1.70 (br, 1H), 1,60(m, 1H), 1.43 (br, 1H), 0.91 (m, 2H), 0.81 (m, 1H), 0.59 (m, 1H)

### Example 397. (S)-quinuclidin-3-yl (5′-(4-(methoxymethoxy)phenyl)-1′,3′-dihydrospiro[cyclopropane-1,2′-inden]-1′-yl)carbamate

¹H-NMR (400 MHz, CDCl₃) δ 7.51 (d, 2H), 7.43 (d, 2H), 7.39 (s, 1H), 7.11 (d, 2H), 5.22 (s, 2H), 5.02 (m, 1H), 4.92 (m, 1H), 4.75 (m, 1H), 3.52 (s, 3H), 3.25 (m, 1H), 3.08 (m, 1H), 2.91-2.72 (m, 5H), 2.09 (m, 1H), 1.85 (br, 1H), 1.80 (m, 1H), 1.70 (br, 1H), 1,60(m, 1H), 1.43 (br, 1H), 0.91 (m, 2H), 0.79 (m, 1H), 0.59 (m, 1H)

### Example 398. (S)-quinuclidin-3-yl (5′-(3-(methoxymethoxy)phenyl)-1′,3′-dihydrospiro[cyclopropane-1,2′-inden]-1′-yl)carbamate

¹H-NMR (400 MHz, CDCl₃) δ 7.46 (m, 2H), 7.45 (s, 1H), 7.35 (t, 1H), 7.18 (m, 2H), 6.93 (d, 1H), 5.22 (s, 2H), 5.02 (m, 1H), 4.92 (m, 1H), 4.75 (m, 1H), 3.51 (s, 3H), 3.25 (m, 1H), 3.08 (m, 1H), 2.91-2.72 (m, 5H), 2.09 (m, 1H), 1.85 (br, 1H), 1.80 (m, 1H), 1.70 (br, 1H), 1,60(m, 1H), 1.43 (br, 1H), 0.91 (m, 2H), 0.80 (m, 1H), 0.59 (m, 1H)

### Examples 399 to 403

The titled compounds of Examples 399 to 403 were prepared in accordance with the same procedures as in Example 1, using (S)-quinuclidin-3-yl (5-bromo-3,3-dimethyl-2,3-dihydro-1H-inden-1-yl)carbamate prepared in Preparation 9; and the corresponding substituted-phenylboronic acids, respectively.

### Example 399. (S)-quinuclidin-3-yl (5-(3-chlorophenyl)-3,3-dimethyl-2,3-dihydro-1H-inden-1-yl)carbamate

¹H NMR (400 MHz, CD₃OD) δ 7.62-7.49 (m, 2H), 7.48-7.29 (m, 5H), 5.23 (d, 1H), 4.80 (brs, 1H), 4.14-4.08 (m, 1H), 3.29-3.24 (m, 1H), 2.92-2.72 (m, 5H), 2.43-2.37 (m, 1H), 2.12-2.05 (m, 1H), 2.00-1.93 (m, 1H), 1.88-1.75 (m, 2H), 1.70-1.63 (m, 1H), 1.54-1.43 (m, 4H), 1.26 (brs, 3H)

### Example 400. (S)-quinuclidin-3-yl (5-(2-chlorophenyl)-3,3-dimethyl-2,3-dihydro-1H-inden-1-yl)carbamate

¹H NMR (400 MHz, CD₃OD) δ 7.52-7.45 (m, 1H), 7.38-7.22 (m, 6H), 5.29-5.21 (m, 1H), 4.94-4.90 (m, 7H), 4.86-4.77 (m, 1H), 4.15-4.07 (m, 1H), 3.31-3.22 (m, 1H), 2.93-2.71 (m, 4H), 2.46-2.37 (m, 1H), 2.14-2.05 (m, 1H), 2.01-1.93 (m, 1H), 1.90-1.74 (m, 2H), 1.73-1.61 (m, 1H), 1.57-1.48 (m, 1H), 1.48-1.40 (m, 3H), 1.28-1.24 (m, 3H)

### Example 401. (S)-quinuclidin-3-yl (3,3-dimethyl-5-(3-(trifluoromethyl)phenyl)-2,3-dihydro-1H-inden-1-yl)carbamate

¹H NMR (400 MHz, CD₃OD) δ 7.89-7.84 (m, 2H), 7.66-7.63 (m, 2H), 7.53-7.46 (m, 2H), 7.35 (dd, 1H), 5.26 (t, 1H), 4.83-4.78 (m, 1H), 3.30-3.26 (m, 1H), 2.92-2.73 (m, 5H), 2.42 (ddd, 1H), 2.14-2.06 (m, 1H), 1.96 (dd, 1H), 1.89-1.76 (m, 2H), 1.71-1.65 (m, 1H), 1.55-1.46 (m, 4H), 1.30-1.27 (m, 3H)

### Example 402. (S)-quinuclidin-3-yl (3,3-dimethyl-5-(3-(trifluoromethoxy)phenyl)-2,3-dihydro-1H-inden-1-yl)carbamate

¹H NMR (400 MHz, CD₃OD) δ 7.62 (d, 1H), 7.56-7.43 (m, 4H), 7.36-7.24 (m, 2H), 5.24 (brs, 1H), 4.85-4.78 (m, 1H), 3.29-3.19 (m, 1H), 2.92-2.72 (m, 5H), 2.41 (dd, 1H), 2.10 (d, 1H), 2.03-1.94 (m, 1H), 1.88-1.76 (m, 2H), 1.71-1.64 (m, 1H), 1.54-1.44 (m, 4H), 1.28 (brs, 3H)

### Example 403. (S)-quinuclidin-3-yl (3,3-dimethyl-5-(2-(trifluoromethoxy)phenyl)-2,3-dihydro-1H-inden-1-yl)carbamate

¹H NMR (400 MHz, CD₃OD) δ 7.50-7.38 (m, 4H), 7.34-7.28 (m, 3H), 5.26 (t, 1H), 4.83-4.78 (m, 1H), 3.29-3.26 (m, 1H), 2.92-2.73 (m, 5H), 2.42(dt, 1H), 2.10 (dd, 1H), 2.00-1.96 (m, 1H), 1.88-1.77 (m, 2H), 1.70-1.66 (m, 1H), 1.52 (brs, 1H), 1.43 (s, 3H), 1.26 (s, 3H)

### Examples 404 and 405

The titled compounds of Examples 404 and 405 were prepared in accordance with the same procedures as in Example 1, using (S)-quinuclidin-3-yl (6-bromo-dihydrobenzofuran-3-yl)carbamate prepared in Preparation 10; and the corresponding substituted-phenylboronic acids, respectively.

### Example 404. (S)-quinuclidin-3-yl (6-(3-(trifluoromethyl)phenyl)-2,3-dihydrobenzofuran-3-yl)carbamate

¹H-NMR (400 MHz, CDCl₃) δ 7.78 (s, 1H), 7.30-7.11 (d, 1H), 7.62-7.60 (d, 1H), 7.56-7.52 (t, 1H), 7.45-7.41 (t, 1H), 7.17-7.14 (m, 1H), 7.06 (s, 1H), 5.42 (m, 2H), 4.76-4.72 (m, 2H), 4.44-4.41 (m, 1H), 3.25-3.19 (m, 1H), 2.82-2.69 (m, 5H), 2.03-2.00 (m, 1H), 1.77-1.65 (m, 2H), 1.59-1.57 (m, 1H), 1.45-1.30 (m, 1H)

### Example 405. (S)-quinuclidin-3-yl (6-(3-(trifluoromethoxy)phenyl)-2,3-dihydrobenzofuran-3-yl)carbamate

¹H-NMR (400 MHz, CDCl₃) δ 7.48-7.38 (m, 4H), 7.21-7.20 (d, 1H), 7.15-7.12 (m, 1H), 7.04 (s, 1H), 5.43-5.38 (m, 2H), 4.76-4.71 (m, 2H), 4.44-4.41 (m, 1H), 3.25-3.19 (m, 1H), 2.82-2.66 (m, 5H), 2.03-2.01 (m, 1H), 1.77-1.65 (m, 2H), 1.59-1.55 (m, 1H), 1.39 (m, 1H)

### Example 406. (S)-quinuclidin-3-yl (5-(3-fluorophenyl)-2,2-dimethyl-2,3-dihydrobenzofuran-3-yl)carbamate

The titled compound was prepared in accordance with the same procedures as in Example 1, using (S)-quinuclidin-3-yl (5-bromo-2,2-dimethyl-2,3-dihydrobenzofuran-3-yl)carbamate prepared in Preparation 11; and 3-fluorophenylboronic acid.

¹H-NMR (400 MHz, CDCl₃) δ 7.52-7.44 (m, 2H), 7.38-7.27 (m, 2H), 7.23-7.21 (d, 1H), 7.02-6.98 (t, 1H), 6.85-6.83 (d, 1H), 5.22-5.19 (m, 1H), 5.09-5.07 (m, 1H), 4.76-4.74 (m, 1H), 3.26-3.20 (m, 1H), 2.82-2.64 (m, 5H), 1.77-1.68 (m, 2H), 1.66-1.53 (m, 4H), 1.46-1.39 (m, 4H), 1.27 (m, 1H)

### Examples 407 to 411

The titled compounds of Examples 407 to 411 were prepared in accordance with the same procedures as in Example 1, using (S)-quinuclidin-3-yl (6-bromo-2,2-dimethyl-2,3-dihydrobenzofuran-3-yl)carbamate prepared in Preparation 12; and the corresponding substituted-phenylboronic acids, respectively.

### Example 407. (S)-quinuclidin-3-yl (6-(3-fluorophenyl)-2,2-dimethyl-2,3-dihydrobenzofuran-3-yl)carbamate

¹H-NMR (400 MHz, CDCl₃) δ 7.39-7.31 (m, 3H), 7.27-7.22 (t, 1H), 7.12-7.09 (t, 1H), 7.06-7.02 (t, 1H), 6.95 (s, 1H), 5.36-5.30 (m, 1H), 5.06-5.04 (d, 1H), 4.76-4.74 (m, 1H), 3.24-3.19 (m, 1H), 2.81-2.69 (m, 5H), 1.76-1.67 (m, 2H), 1.54-1.49 (m, 4H), 1.45-1.43 (m, 4H), 1.40-1.26 (m, 1H)

### Example 408. (S)-quinuclidin-3-yl (6-(3-chlorophenyl)-2,2-dimethyl-2,3-dihydrobenzofuran-3-yl)carbamate

¹H-NMR (400 MHz, CDCl₃) δ 7.53 (s, 1H), 7.43-7.31 (m, 4H), 7.1-7.09 (m, 1H), 6.95 (s, 1H), 5.18-5.14 (m, 1H), 5.07-5.05 (m, 1H), 4.77-4.75 (m, 1H), 3.26-3.21 (m, 1H), 2.89-2.65 (m, 5H), 1.77-1.73 (m, 2H), 1.69-1.68 (m, 4H), 1.56-1.44 (m, 4H), 1.40 (m, 1H)

### Example 409. (S)-quinuclidin-3-yl (2,2-dimethyl-6-(3-(trifluoromethyl)phenyl)-2,3-dihydrobenzofuran-3-yl)carbamate

¹H-NMR (400 MHz, CDCl₃) δ 7.79 (s, 1H), 7.73-7.71 (d, 1H), 7.62-7.60 (d, 1H), 7.57-7.53 (t, 1H), 7.41-7.37 (t, 1H), 7.15-7.12 (t, 1H), 6.98 (s, 1H), 5.23-5.19 (t, 1H), 5.08-5.06 (d, 1H), 4.77-4.75 (m, 1H), 3.26-3.21 (m, 1H), 2.89-2.65 (m, 5H), 1.78-1.69 (m, 2H), 1.57-1.53 (m, 3H), 1.46-1.40 (m, 4H), 1.40-1.27 (m, 1H)

### Example 410. (S)-quinuclidin-3-yl (2,2-dimethyl-6-(3-(trifluoromethoxy)phenyl)-2,3-dihydrobenzofuran-3-yl)carbamate

¹H-NMR (400 MHz, CDCl₃) δ 7.47-7.43 (m, 2H), 7.39-7.35 (m, 2H), 7.22-7.20 (d, 1H), 7.13-7.09 (t, 1H), 6.96 (s, 1H), 5.19-5.14 (t, 1H), 5.08-5.06 (d, 1H), 4.76-4.75 (m, 1H), 3.27-3.21 (m, 1H), 2.84-2.66 (m, 5H), 1.77-1.68 (m, 2H), 1.56-1.53 (m, 4H), 1.46-1.40 (m, 4H), 1.39-1.27 (m, 1H)

### Example 411. (S)-quinuclidin-3-yl (6-(3-(2-methoxyethoxy)phenyl)-2,2-dimethyl-2,3-dihydrobenzofuran-3-yl)carbamate

¹H-NMR (400 MHz, CDCl₃) δ 7.35-7.27 (m, 2H), 7.23-7.22 (m, 2H), 7.19-7.17 (m, 1H), 7.03 (m, 1H), 6.97 (s, 1H), 5.21-5.18 (m, 3H), 5.05-5.00 (m, 1H), 4.74-4.73 (m, 1H), 3.49 (s, 3H), 3.23-3.18 (m, 1H), 2.80-2.66 (m, 5H), 2.04 (m, 1H), 1.76-1.67 (m, 2H), 1.56-1.38 (m, 8H)

### Experimental Example 1: Evaluation of Inhibitory Activities Against GCS

The inhibitory activities of the compounds of the present invention against GCS were evaluated, as follows, according to the method described in the known literature (Hayashi Y et al., A sensitive and reproducible assay to measure the activity of glucosylceramide synthase and lactosylceramide synthase using HPLC and fluorescent substrates, Analytical Biochemistry 345 (2005) 181-186). Ibiglustat, known as a GCS inhibitor, was used as a control.

### (1) Materials

A549 cells (ATCC, CCL-185)
NBD C6-ceramide (Thermo Fisher, N1154)
UDP-glucose (Sigma, U4625)
Potassium chloride (Sigma, P9333)
UltraPure™ 0.5 M EDTA (Invitrogen, 15575-038)
BCA protein assay kit (Thermo Fisher, 23227)
Ibiglustat (Shanghai Systeam Biochem Co., ltd, Genz-682452)
HEPES (sigma, H3375)
Protease/phosphatase inhibitor cocktail (CST, 5872s)
DMEM (GIBCO, 11995-065)
FBS (GIBCO, 16000-044)
Antibiotic-Antimycotic (100X) (GIBCO, 15240-112)
200 mM L-glutamine (GIBCO, 25030081)
PBS (GIBCO, 10010-023)
0.25% Trypsin-EDTA (GIBCO, 25200-056)
Dimethyl sulfoxide (Sigma, 34869)
2-propanol, HPLC grade (Burdick & Jackson, AH323-4)
Hexane, HPLC grade (Burdick & Jackson, AH216-4)
Chloroform (Sigma, C₂₄₃₂)
Methanol (Merck, 1.06009.1011)

### (2) Protocol

### <1> Preparation of Cell Lysates

A549 cells (ATCC, CCL-185) were cultured in a DMEM medium supplemented with 10% fetal bovine serum (FBS), 1× antibiotic-antimycotic, and 1× L-glutamine, in an incubator at 37° C. and 5% CO₂. After the cells attached to the culture dish were washed with phosphate buffered saline (PBS), the cells were scraped off with a cell scraper and then centrifuged (4000 rpm, 3 min, 4° C.) to collect the cells in a 50 ml tube. The cell pellets were suspended in a lysis buffer (50 mM HEPES, pH 7.3, containing 1× the protease/phosphatase inhibitor cocktail), lysed by sonication, and then the lysate was centrifuged (13000 rpm, 10 min, 4° C.). The obtained supernatant was used for the quantitative analysis of proteins. The amount of proteins was measured using the BCA protein assay kit, using bovine serum albumin as a standard.

### <2> GCS Enzyme Reaction

Enzymatic reactions were initiated by sequentially adding the following reaction materials to a 96 deep-well plate. Thereafter, the enzymatic reactions were performed at 37° C. for 90 minutes.

Enzyme reaction mix (Total 50 ul)

| | Stock Con. | Final Con. | Volume (ul) |
|---|---|---|---|
| 1 | | | |
| Cpd. Mix | Cpd. 500-0.003 uM | 10000~0.056 nM | 1 |
| | (DMSO) | | |
| | Total | | 1 |
| 2 | | | |
| Sub Mix | 20 mM UDP-GIc | 500 uM | 1.25 |
| | 25 mM EDTA | 1 mM | 2 |
| | 500 uM NBD-Cer/BSA | 5 uM | 0.5 |
| | D.W | | 35.25 |
| | Total | | 39 |
| 3 | | | |
| Enzyme | 3.135 ug/ul A549 | 0.627 ug/ul | 10 |
| Mix | cell lysate | (31.35 ug) | |
| | Total | | 10 |

### <3> Lipid Extraction

The enzymatic reactions were stopped by adding 100 μl of chloroform/methanol (2:1, v/v). After vortexing for 15 seconds, each mixture was centrifuged (4000 rpm, 10 min, 18° C.). The lower layer (50 μl) was transferred to a new 96 deep-well plate and dried with a reduced concentrator.

### <4> HPLC Analysis

Lipids were dissolved in 100 μl of isopropyl alcohol/n-hexane/H₂O (55:44:1, v/v/v) and then transferred to a glass vial for HPLC (Agilent, 8004-HP-H/i3u). The sample (100 μl) was automatically loaded onto a normal-phase column (Intersil SIL 150A-5, 4.6×250 mm, GL Sciences, Japan) and then eluted at a flow rate of 2.0 ml/min with isopropyl alcohol/n-hexane/H₂O (55:44:1, v/v/v). The fluorescence thereof was measured with a fluorescence detector (Agilent, 1260 FLD Spectra), using 470 nm and 530 nm as excitation and emission wavelengths, respectively.

### <5> Data Analysis

Data analysis was performed by the following equations.

$$\% Area ( Sample ) = Area ( GlcCer ) / [ Area ( Cer ) + Area ( GlcCer ) ] \times 100$$

$$\% GCS activity = [ Area ( Sample ) - Area ( ibiglustat ) ] / [ Area ( DMSO ) - Area ( ibiglustat ) ] \times 100$$

The obtained % GCS activity data was analyzed with the software GraphPad Prism (Ver 5.01) to calculate the IC₅₀ values. The results are shown in Tables 1 to 4 below.

TABLE 1

| Example | IC₅₀ (nM) |
|---|---|
| 1 | 9.8 |
| 2 | 17.2 |
| 3 | 21.7 |
| 4 | 11.0 |
| 5 | 3.8 |
| 6 | 1.7 |
| 7 | 1.1 |
| 8 | 1.2 |
| 9 | 2.4 |
| 10 | 2.9 |
| 11 | 22.2 |
| 12 | 14.8 |
| 13 | 9.5 |
| 14 | 8.9 |
| 15 | 11.8 |
| 16 | 12.7 |
| 17 | 3.7 |
| 18 | 5.3 |
| 19 | 12.7 |
| 20 | 16.0 |
| 21 | 12.3 |
| 22 | 15.1 |
| 23 | 10.2 |
| 24 | 1.6 |
| 25 | 65.7 |
| 26 | 48.1 |
| 27 | 55.3 |
| 28 | 79.2 |
| 29 | 38.0 |
| 30 | 34.0 |
| 31 | 41.5 |
| 32 | 71.2 |
| 33 | 25.8 |
| 34 | 13.7 |
| 35 | 20.29 |
| 36 | 14.28 |
| 37 | 33.9 |
| 38 | 33.9 |
| 39 | 26.9 |
| 40 | 40.4 |
| 41 | 16.9 |
| 42 | 23.6 |
| 43 | 33.4 |
| 44 | 18.6 |
| 45 | 33.3 |
| 46 | 8.9 |
| 47 | 37.5 |
| 48 | 58.2 |
| 49 | 9.3 |
| 50 | 9.7 |
| 51 | 7.1 |
| 52 | 14.9 |
| 53 | 8.1 |
| 54 | 20.65 |
| 55 | 32.42 |
| 56 | 32.1 |
| 57 | 2.5 |
| 58 | 2.8 |
| 59 | 5.44 |
| 60 | 16.9 |
| 61 | 12.7 |
| 62 | 13.13 |
| 63 | 7.55 |
| 64 | 3.9 |
| 65 | 5.5 |
| 66 | 30.9 |
| 67 | 12.3 |
| 68 | 15.2 |
| 69 | 108.8 |
| 70 | 20.1 |
| 71 | 13.0 |
| 72 | 10.7 |
| 73 | 22.5 |
| 74 | 24.1 |
| 75 | 14.9 |
| 76 | 5.9 |
| 77 | 2.9 |
| 78 | 2.1 |
| 79 | 2.6 |
| 80 | 3.0 |
| 81 | 2.8 |
| 82 | 2.5 |
| 83 | 7.5 |
| 84 | 7.3 |
| 85 | 3.1 |
| 86 | 3.6 |
| 87 | 9.4 |
| 88 | 2.9 |
| 89 | 8.5 |
| 90 | 11.0 |
| 91 | 2.3 |
| 92 | 16.1 |
| 93 | 1.4 |
| 94 | 9.5 |
| 95 | 11.2 |
| 96 | 6.9 |
| 97 | 3.0 |
| 98 | 2.8 |
| 99 | 6.0 |
| 100 | 2.1 |
| 101 | 1.9 |
| 102 | 4.6 |
| 103 | 1.7 |
| 104 | 1.9 |
| 105 | 1.8 |
| 106 | 1.2 |
| 107 | 1.1 |
| 108 | 2.3 |
| 109 | 3.6 |
| 110 | 6.0 |
| 111 | 3.5 |
| 112 | 2.5 |
| 113 | 6.4 |
| 114 | 3.8 |
| 115 | 3.6 |
| 116 | 1.8 |
| 117 | 3.5 |
| 118 | 14.2 |
| 119 | 10.9 |
| 120 | 4.2 |

TABLE 2

| Example | IC₅₀ (nM) |
|---|---|
| 121 | 0.7 |
| 122 | 6.8 |
| 123 | 0.9 |
| 124 | 3.1 |
| 125 | 17.0 |
| 126 | 14.0 |
| 127 | 5.9 |
| 128 | 2.8 |
| 129 | 5.1 |
| 130 | 1.3 |
| 131 | 2.1 |
| 132 | 3.7 |
| 133 | 4.3 |
| 134 | 6.8 |
| 135 | 1.4 |
| 136 | 0.7 |
| 137 | 2.9 |
| 138 | 1.3 |
| 139 | 10.0 |
| 140 | 1.0 |
| 141 | 3.0 |
| 142 | 1.5 |
| 143 | 4.5 |
| 144 | 1.0 |
| 145 | 4.6 |
| 146 | 3.3 |
| 147 | 1.9 |
| 148 | 1.4 |
| 149 | 1.6 |
| 150 | 0.7 |
| 151 | 2.2 |
| 152 | 1.7 |
| 153 | 2.8 |
| 154 | 1.7 |
| 155 | 1.1 |
| 156 | 2.6 |
| 157 | 0.6 |
| 158 | 3.4 |
| 159 | 4.3 |
| 160 | 2.0 |
| 161 | 1.6 |
| 162 | 7.0 |
| 163 | 0.6 |
| 164 | 0.6 |
| 165 | 13.7 |
| 166 | 1.6 |
| 167 | 0.5 |
| 168 | 2.7 |
| 169 | 1.9 |
| 170 | 6.1 |
| 171 | 2.5 |
| 172 | 1.8 |
| 173 | 7.2 |
| 174 | 4.5 |
| 175 | 2.3 |
| 176 | 1.7 |
| 177 | 3.2 |
| 178 | 3.2 |
| 179 | 4.3 |
| 180 | 1.4 |
| 181 | 0.3 |
| 182 | 2.1 |
| 183 | 2.8 |
| 184 | 6.5 |
| 185 | 0.5 |
| 186 | 9.5 |
| 187 | 4.5 |
| 188 | 8.4 |
| 189 | 2.2 |
| 190 | 2.1 |
| 191 | 9.3 |
| 192 | 3.7 |
| 193 | 2.0 |
| 194 | 10.1 |
| 195 | 6.4 |
| 196 | 0.9 |
| 197 | 1.7 |
| 198 | 1.2 |
| 199 | 0.7 |
| 200 | 0.6 |
| 201 | 3.9 |
| 202 | 6.1 |
| 203 | 1.2 |
| 204 | 8.3 |
| 205 | 1.0 |
| 206 | 0.6 |
| 207 | 6.2 |
| 208 | 3.7 |
| 209 | 3.0 |
| 210 | 0.6 |
| 211 | 1.2 |
| 212 | 1.2 |
| 213 | 2.0 |
| 214 | 1.7 |
| 215 | 1.4 |
| 216 | 0.7 |
| 217 | 2.4 |
| 218 | 0.6 |
| 219 | 2.9 |
| 220 | 1.2 |
| 221 | 1.5 |
| 222 | 2.9 |
| 223 | 0.9 |
| 224 | 2.9 |
| 225 | 2.9 |
| 226 | 6.3 |
| 227 | 2.2 |
| 228 | 3.8 |
| 229 | 4.4 |
| 230 | 1.2 |
| 231 | 0.5 |
| 232 | 0.8 |
| 233 | 1.2 |
| 234 | 0.6 |
| 235 | 0.9 |
| 236 | 3.4 |
| 237 | 5.2 |
| 238 | 2.4 |
| 239 | 7.6 |
| 240 | 3.5 |

TABLE 3

| Example | IC₅₀ (nM) |
|---|---|
| 241 | 1.3 |
| 242 | 5.9 |
| 243 | 1.4 |
| 244 | 1.8 |
| 245 | 3.0 |
| 246 | 3.9 |
| 247 | 11.9 |
| 248 | 3.0 |
| 249 | 9.0 |
| 250 | 4.8 |
| 251 | 1.1 |
| 252 | 7.4 |
| 253 | 3.4 |
| 254 | 7.0 |
| 255 | 6.2 |
| 256 | 1.9 |
| 257 | 2.1 |
| 258 | 5.9 |
| 259 | 2.8 |
| 260 | 1.0 |
| 261 | 3.1 |
| 262 | 1.5 |
| 263 | 2.6 |
| 264 | 3.6 |
| 265 | 0.7 |
| 266 | 0.7 |
| 267 | 2.8 |
| 268 | 0.7 |
| 269 | 5.2 |
| 270 | 1.1 |
| 271 | 5.1 |
| 272 | 9.9 |
| 273 | 5.2 |
| 274 | 2.7 |
| 275 | 5.1 |
| 276 | 1.3 |
| 277 | 0.8 |
| 278 | 0.6 |
| 279 | 0.8 |
| 280 | 0.4 |
| 281 | 0.6 |
| 282 | 0.7 |
| 283 | 1.3 |
| 284 | 0.8 |
| 285 | 0.5 |
| 286 | 0.4 |
| 287 | 0.7 |
| 288 | 1.2 |
| 289 | 0.8 |
| 290 | 1.4 |
| 291 | 1.7 |
| 292 | 1.6 |
| 293 | 2.0 |
| 294 | 4.0 |
| 295 | 2.7 |
| 296 | 1.2 |
| 297 | 0.7 |
| 298 | 6.6 |
| 299 | 2.2 |
| 300 | 1.4 |
| 301 | 1.2 |
| 302 | 1.4 |
| 303 | 4.4 |
| 304 | 3.5 |
| 305 | 4.1 |
| 306 | 0.8 |
| 307 | 1.5 |
| 308 | 0.3 |
| 309 | 0.6 |
| 310 | 0.7 |
| 311 | 2.4 |
| 312 | 2.2 |
| 313 | 1.3 |
| 314 | 0.9 |
| 315 | 0.9 |
| 316 | 1.9 |
| 317 | 2.0 |
| 318 | 2.3 |
| 319 | 1.0 |
| 320 | 0.9 |
| 321 | 4.3 |
| 322 | 4.4 |
| 323 | 5.4 |
| 324 | 0.8 |
| 325 | 0.8 |
| 326 | 0.7 |
| 327 | 1.8 |
| 328 | 0.4 |
| 329 | 4.9 |
| 330 | 5.1 |
| 331 | 3.7 |
| 332 | 5.1 |
| 333 | 1.7 |
| 334 | 0.1 |
| 335 | 4.3 |
| 336 | 4.0 |
| 337 | 8.9 |
| 338 | 1.3 |
| 339 | 2.1 |
| 340 | 1.3 |
| 341 | 3.2 |
| 342 | 1.2 |
| 343 | 2.3 |
| 344 | 2.1 |
| 345 | 3.8 |
| 346 | 2.4 |
| 347 | 2.6 |
| 348 | 6.7 |
| 349 | 0.8 |
| 350 | 0.4 |
| 351 | 0.5 |
| 352 | 0.7 |
| 353 | 1.2 |
| 354 | 0.3 |
| 355 | 1.1 |
| 356 | 0.5 |
| 357 | 0.3 |
| 358 | 0.3 |
| 359 | 2.4 |
| 360 | 0.7 |

TABLE 4

| Example | IC₅₀ (nM) |
|---|---|
| 361 | 0.1 |
| 362 | 0.2 |
| 363 | 0.1 |
| 364 | 0.1 |
| 365 | 0.3 |
| 366 | 5.3 |
| 367 | 4.3 |
| 368 | 6.2 |
| 369 | 2.8 |
| 370 | 1.8 |
| 371 | 2.6 |
| 372 | 3.9 |
| 373 | 3.9 |
| 374 | 7.7 |
| 375 | 0.9 |
| 376 | 2.6 |
| 377 | 1.4 |
| 378 | 1.7 |
| 379 | 0.6 |
| 380 | 1.7 |
| 381 | 1.6 |
| 382 | 0.5 |
| 383 | 1.9 |
| 384 | 1.3 |
| 385 | 6.9 |
| 386 | 0.2 |
| 387 | 1.8 |
| 388 | 11.5 |
| 389 | 19.0 |
| 390 | 18.6 |
| 391 | 21.4 |
| 392 | 10.2 |
| 393 | 4.8 |
| 394 | 5.6 |
| 395 | 2.4 |
| 396 | 2.7 |
| 397 | 0.8 |
| 398 | 0.9 |
| 399 | 103.7 |
| 400 | 65.6 |
| 401 | 73 |
| 402 | 74.5 |
| 403 | 155.2 |
| 404 | 5.3 |
| 405 | 7.1 |
| 406 | 31.1 |
| 407 | 4.8 |
| 408 | 3.0 |
| 409 | 3.3 |
| 410 | 3.0 |
| 411 | 10.9 |

From the results of Tables 1 to 4, it can be seen that the compounds of the present invention exhibit excellent inhibitory activity against GCS.

### Experimental Example 2: Evaluation of Inhibitory Activities Against GM1 Production

GM1, which is the final product of sphingolipid metabolism, is expressed on the cell membrane and thus the detection thereof is easy. In addition, the amount of GM1 represents the conversion of ceramide to glucosylceramide. Therefore, the inhibitory activities of the compounds of the present invention against GM1 production were evaluated, as follows, according to the method described in the known literature (Dijkhuis et al., Gangliosides do not affect ABC transporter function in human neuroblastoma cells. The Journal of Lipid Research 47 (2006). 1187-1195). Ibiglustat, known as a GCS inhibitor, was used as a control.

### (1) Materials

Jurkat cells, Clone E6-1 (ATCC, TIB-152)
Cholera toxin subunit B (CTB), FITC (Sigma, C₁₆₅₅)
Ibiglustat (Shanghai Systeam Biochem Co., ltd, Genz-682452)
DMSO (Sigma, D2650)
Fixation buffer (BD, 554655)
RPMI 1640 (Gibco, A1049101)
FBS (Gibco, 16000-044)
Antibiotic-Antimycotic (100X) (Gibco, 15240-122)
FACS Sheath Fluid (BD, 342003)

Jurkat cells were cultured in RPMI 1640 medium supplemented with 10% FBS and 1X Antibiotic-Antimycotic. A washing solution was prepared by adding 10 ml of FBS to 490 ml of FACS Sheath Fluid. The CTB-FITC solution was prepared by diluting the CTB-FITC stock solution (10 mg/ml) with the washing solution to a final concentration of 2 g/ml.

### (2) Protocol

A cell suspension (1×10⁵ cells/ml) was prepared with the culture medium (RPMI 1640 medium supplemented with 10% FBS and 1X Antibiotic-Antimycotic). The cell suspension (200 μl) was added to each well of a 96-well plate (20,000 cells/well) and then the compounds were treated thereto in a final concentration of 0.05 to 3000 nM (3 fold, 11 points) per well. Each mixture was incubated in a CO₂ incubator at 37° C. for 72 hours. After centrifuging at 1500 rpm for 3 minutes to remove the medium, the cells were resuspended in 200 μl of the washing solution per well. After centrifuging at 1500 rpm for 3 minutes to remove the washing solution, the cells were resuspended in 200 μl of the 2 μg/ml CTB-FITC solution. The obtained suspension was incubated at 4° C. for 60 minutes, while not being exposed to light. After centrifuging at 1500 rpm for 3 minutes to remove the CTB-FITC solution, the cells were washed with 200 μl of the washing solution. The washing process was additionally repeated twice. The washed plate was centrifuged at 1500 rpm for 3 minutes to remove the washing solution and then the cells were completely resuspended in 200 μl of the fixation buffer. IC₅₀ was determined from the values obtained by quantifying FITC fluorescences with Guava™ easyCyte 5HT (Merck Milipore, 0500-4005).

Data analysis was performed by the following equations.

$$\% MFI ( median fluorescence intensity ) = ( fluorescence value of drug - treated group / fluorescence value of DMSO - treated group ) \times 100$$

$$\% Cells = ( cell concentration in the well / cell concentration in the DMSO - treated group ) \times 100$$

% MFI and % cell data were analyzed with the software GraphPad Prism (Ver 5.01) to calculate ICso values. The results are shown in Tables 5 to 8 below.

TABLE 5

| Example | IC₅₀ (nM) |
|---|---|
| 1 | 139.2 |
| 2 | 201.8 |
| 3 | 142.5 |
| 4 | 95.8 |
| 5 | 70.2 |
| 6 | 39.4 |
| 7 | 36.8 |
| 8 | 27.4 |
| 9 | 93.1 |
| 10 | 65.5 |
| 11 | 123.0 |
| 12 | 73.0 |
| 13 | 41.7 |
| 14 | 54.3 |
| 15 | 34.4 |
| 16 | 56.1 |
| 17 | 27.6 |
| 18 | 31.6 |
| 19 | 20.3 |
| 20 | 125.9 |
| 21 | 126.6 |
| 22 | 89.6 |
| 23 | 63.7 |
| 24 | 17.4 |
| 25 | 105.9 |
| 26 | 67.2 |
| 27 | 35.7 |
| 28 | 52.0 |
| 29 | 63.0 |
| 30 | 106.8 |
| 31 | 65.5 |
| 32 | 120.2 |
| 37 | 55.2 |
| 38 | 62.0 |
| 39 | 51.9 |
| 43 | 182.8 |
| 44 | 35.2 |
| 45 | 57.3 |
| 46 | 19.2 |
| 47 | 26.6 |
| 48 | 65.7 |
| 49 | 61.4 |
| 50 | 52.3 |
| 51 | 30.2 |
| 52 | 82.3 |
| 53 | 80.0 |
| 54 | 161 |
| 55 | 172.8 |
| 56 | 209.9 |
| 57 | 49.9 |
| 58 | 87.8 |
| 59 | 164.5 |
| 60 | 111.7 |
| 61 | 39.6 |
| 62 | 103 |
| 63 | 116.7 |
| 64 | 74.7 |
| 65 | 56.2 |
| 66 | 127.7 |
| 67 | 144.0 |
| 68 | 91.8 |
| 69 | 399.8 |
| 70 | 70.9 |
| 71 | 64.3 |
| 72 | 58.2 |
| 73 | 26.0 |
| 74 | 13.0 |
| 75 | 8.3 |
| 76 | 3.7 |
| 77 | 6.3 |
| 78 | 3.5 |
| 79 | 3.9 |
| 80 | 4.1 |
| 81 | 12.2 |
| 82 | 3.2 |
| 83 | 3.7 |
| 84 | 4.9 |
| 85 | 11.6 |
| 86 | 13.8 |
| 87 | 42.8 |
| 88 | 11.1 |
| 89 | 50.0 |
| 90 | 37.9 |
| 91 | 25.1 |
| 92 | 43.9 |
| 93 | 10.5 |
| 94 | 23.6 |
| 95 | 49.7 |
| 96 | 16.2 |
| 97 | 9.3 |
| 98 | 7.5 |
| 99 | 22.2 |
| 100 | 21.5 |
| 101 | 8.7 |
| 102 | 17.4 |
| 103 | 19.5 |
| 104 | 16.9 |
| 105 | 15.2 |
| 106 | 5.8 |
| 107 | 6.3 |
| 108 | 7.6 |
| 109 | 7.8 |
| 110 | 15.1 |
| 111 | 32.0 |
| 112 | 35.6 |
| 113 | 54.8 |
| 114 | 33.7 |
| 115 | 38.3 |
| 116 | 16.7 |
| 117 | 41.8 |
| 118 | 38.4 |
| 119 | 38.3 |
| 120 | 28.4 |
| 121 | 4.7 |
| 122 | 50.8 |
| 123 | 9.3 |
| 124 | 22.3 |
| 125 | 9.5 |
| 126 | 50.5 |
| 127 | 18.2 |

TABLE 6

| Example | IC₅₀ (nM) |
|---|---|
| 128 | 18.4 |
| 129 | 36.8 |
| 130 | 10.0 |
| 131 | 10.6 |
| 132 | 29.2 |
| 133 | 22.6 |
| 134 | 43.2 |
| 135 | 8.1 |
| 136 | 1.7 |
| 137 | 12.1 |
| 138 | 9.1 |
| 139 | 74.4 |
| 140 | 10.5 |
| 141 | 39.9 |
| 142 | 20.5 |
| 143 | 25.2 |
| 144 | 6.2 |
| 145 | 23.0 |
| 146 | 14.1 |
| 147 | 14.0 |
| 148 | 30.0 |
| 149 | 27.6 |
| 150 | 6.8 |
| 151 | 37.9 |
| 152 | 32.5 |
| 153 | 41.4 |
| 154 | 27.4 |
| 155 | 8.4 |
| 156 | 46.7 |
| 157 | 2.8 |
| 158 | 17.1 |
| 159 | 12.7 |
| 160 | 22.7 |
| 161 | 10.8 |
| 162 | 38.8 |
| 163 | 12.2 |
| 164 | 6.5 |
| 165 | 15.6 |
| 166 | 26.3 |
| 167 | 3.8 |
| 168 | 9.6 |
| 169 | 9.8 |
| 170 | 13.0 |
| 171 | 17.0 |
| 172 | 48.5 |
| 173 | 34.8 |
| 174 | 40.2 |
| 175 | 42.8 |
| 176 | 27.6 |
| 177 | 22.9 |
| 178 | 26.9 |
| 179 | 41.0 |
| 180 | 28.1 |
| 181 | 5.5 |
| 182 | 20.9 |
| 183 | 18.6 |
| 184 | 40.0 |
| 185 | 11.1 |
| 186 | 82.6 |
| 187 | 31.7 |
| 188 | 46.2 |
| 189 | 23.0 |
| 190 | 13.3 |
| 191 | 123.4 |
| 192 | 25.8 |
| 193 | 15.3 |
| 194 | 47.6 |
| 195 | 36.1 |
| 196 | 23.2 |
| 197 | 8.7 |
| 198 | 11.7 |
| 199 | 5.5 |
| 200 | 3.7 |
| 201 | 38.2 |
| 202 | 26.3 |
| 203 | 8.2 |
| 204 | 57.2 |
| 205 | 7.6 |
| 206 | 14.0 |
| 207 | 58.6 |
| 208 | 67.4 |
| 209 | 32.1 |
| 210 | 7.5 |
| 211 | 13.2 |
| 212 | 19.1 |
| 213 | 39.8 |
| 214 | 64.2 |
| 215 | 14.9 |
| 216 | 28.7 |
| 217 | 72.1 |
| 218 | 6.7 |
| 219 | 30.4 |
| 220 | 17.6 |
| 221 | 16.9 |
| 222 | 25.6 |
| 223 | 5.2 |
| 224 | 18.8 |
| 225 | 21.3 |
| 226 | 57.0 |
| 227 | 10.2 |
| 228 | 48.6 |
| 229 | 56.2 |
| 230 | 30.8 |
| 231 | 24.3 |
| 232 | 12.5 |
| 233 | 23.3 |
| 234 | 13.6 |
| 235 | 6.5 |
| 236 | 32.4 |
| 237 | 29.2 |
| 238 | 13.2 |
| 239 | 48.9 |
| 240 | 51.3 |
| 241 | 51.3 |
| 242 | 64.4 |
| 243 | 21.4 |
| 244 | 38.0 |
| 245 | 23.0 |
| 246 | 14.7 |
| 247 | 41.0 |

TABLE 7

| Example | IC₅₀ (nM) |
|---|---|
| 248 | 14.9 |
| 249 | 37.0 |
| 250 | 33.0 |
| 251 | 7.2 |
| 252 | 43.9 |
| 253 | 37.2 |
| 254 | 45.2 |
| 255 | 35.8 |
| 256 | 24.6 |
| 257 | 27.4 |
| 258 | 17.4 |
| 259 | 10.2 |
| 260 | 14.4 |
| 261 | 51.5 |
| 262 | 34.4 |
| 263 | 34.9 |
| 264 | 14.4 |
| 265 | 6.7 |
| 266 | 4.7 |
| 267 | 83.1 |
| 268 | 2.5 |
| 269 | 6.5 |
| 270 | 2.3 |
| 271 | 30.6 |
| 272 | 49.3 |
| 273 | 13.4 |
| 274 | 10.3 |
| 275 | 25.0 |
| 276 | 33.2 |
| 277 | 2.7 |
| 278 | 6.0 |
| 279 | 7.3 |
| 280 | 1.6 |
| 281 | 5.0 |
| 282 | 5.6 |
| 283 | 17.2 |
| 284 | 8.5 |
| 285 | 3.7 |
| 286 | 4.1 |
| 287 | 4.1 |
| 288 | 11.2 |
| 289 | 7.4 |
| 290 | 12.3 |
| 291 | 13.5 |
| 292 | 7.8 |
| 293 | 8.7 |
| 294 | 10.4 |
| 295 | 35.0 |
| 296 | 15.6 |
| 297 | 3.0 |
| 298 | 63.2 |
| 299 | 25.3 |
| 300 | 23.0 |
| 301 | 9.6 |
| 302 | 4.0 |
| 303 | 27.2 |
| 304 | 23.5 |
| 305 | 9.8 |
| 306 | 27.5 |
| 307 | 30.2 |
| 308 | 5.5 |
| 309 | 11.2 |
| 310 | 16.3 |
| 311 | 27.0 |
| 312 | 13.1 |
| 313 | 7.3 |
| 314 | 7.7 |
| 315 | 11.3 |
| 316 | 13.3 |
| 317 | 10.3 |
| 318 | 20.0 |
| 319 | 7.7 |
| 320 | 8.5 |
| 321 | 31.9 |
| 322 | 24.4 |
| 323 | 78.9 |
| 324 | 5.8 |
| 325 | 2.1 |
| 326 | 10.8 |
| 327 | 16.8 |
| 328 | 11.4 |
| 329 | 65.8 |
| 330 | 52.7 |
| 331 | 22.6 |
| 332 | 158.4 |
| 333 | 45 |
| 334 | 20.9 |
| 335 | 50.4 |
| 336 | 48 |
| 337 | 65 |
| 338 | 53.7 |
| 339 | 62.5 |
| 340 | 43.8 |
| 341 | 61.9 |
| 342 | 21.6 |
| 343 | 45.7 |
| 344 | 51 |
| 345 | 45.7 |
| 346 | 38.3 |
| 347 | 46.8 |
| 348 | 44.1 |
| 349 | 10.3 |
| 350 | 4.5 |
| 351 | 7.4 |
| 352 | 5.7 |
| 353 | 12.7 |
| 354 | 5.9 |
| 355 | 62.8 |
| 356 | 21.6 |
| 357 | 5.3 |
| 358 | 7.7 |
| 359 | 33.1 |
| 360 | 39.0 |
| 361 | 2.1 |
| 362 | 2.5 |
| 363 | 8.7 |
| 364 | 17.2 |
| 365 | 2.4 |
| 366 | 79.4 |
| 367 | 68.2 |

TABLE 8

| Example | IC₅₀ (nM) |
|---|---|
| 368 | 22.9 |
| 369 | 171.7 |
| 370 | 39.3 |
| 371 | 24.8 |
| 372 | 46.2 |
| 373 | 31.2 |
| 374 | 26.5 |
| 375 | 23 |
| 376 | 85.9 |
| 377 | 35.5 |
| 378 | 87.5 |
| 379 | 15.8 |
| 380 | 36.5 |
| 381 | 59.9 |
| 382 | 31.3 |
| 383 | 123.1 |
| 384 | 83.2 |
| 385 | 331.6 |
| 386 | 29.5 |
| 387 | 48.2 |
| 388 | 93.1 |
| 389 | 76.5 |
| 390 | 44.1 |
| 391 | 55.3 |
| 392 | 41.9 |
| 393 | 54.9 |
| 394 | 48.8 |
| 395 | 30.9 |
| 396 | 39.6 |
| 397 | 17.4 |
| 398 | 36.6 |
| 399 | 43.59 |
| 400 | 107.5 |
| 401 | 15.13 |
| 402 | 19.5 |
| 403 | 85.31 |
| 404 | 25.9 |
| 405 | 44.7 |
| 406 | 119.7 |
| 407 | 44.4 |
| 408 | 15.8 |
| 409 | 14.9 |
| 410 | 13.0 |
| 411 | 79.3 |

From the results of Tables 5 to 8, it can be seen that the compounds of the present invention exhibit excellent inhibitory activity against GM1 production.

1. A compound of Formula 5: 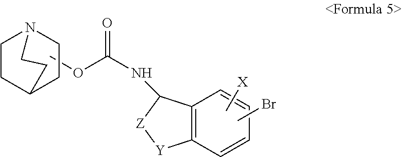
wherein,
Y and Z are, independently of each other, —CR₁R₂—; —NR₃—; —O—; or —S—,
R₁ and R₂ are, independently of each other, hydrogen; halogen; C₁-C₆ alkyl; C₁-C₆ alkyl having a nitrogen, oxygen, or sulfur atom; C₃-C₁₀ cycloalkyl; 3- to 12-membered heterocyclic; or C₁-C₆ alkoxy; or R₁ and R₂ form C₃-C₁₀ cycloalkyl together with the carbon atom to which they are attached,
R₃ is hydrogen; C₁-C₆ alkyl; C₁-C₆ alkyl having a nitrogen, oxygen, or sulfur atom; C₃-C₁₀ cycloalkyl; 3- to 12-membered heterocyclic; or C₁-C₆ alkoxy,
X is hydrogen; halogen; C₁-C₆ alkyl; C₁-C₆ alkyl substituted with 1 to 3 halogens; C₁-C₆ alkyl having a nitrogen, oxygen, or sulfur atom; C₁-C₆ alkoxy; or C₁-C₆ alkoxy substituted with 1 to 3 halogens.

2. The compound as claimed in claim 1, wherein
Y and Z are, independently of each other, —O— or —CR₁R₂—, and
R₁ and R₂ are, independently of each other, hydrogen or C₁-C₆ alkyl; or R₁ and R₂ form C₃-C₁₀ cycloalkyl together with the carbon atom to which they are attached.

3. The compound as claimed in claim 1, wherein Y is —O—; and Z is —CR₁R₂—.

4. The compound as claimed in claim 1, wherein X is hydrogen, halogen, or C₁-C₆ alkoxy.

5. The compound as claimed in claim 1, wherein the compound is (S)-quinuclidin-3-yl (5-bromo-2,2-dimethyl-2,3-dihydro-1H-inden-1-yl)carbamate.

6. A process for preparing a compound of Formula 1a or pharmaceutically acceptable salt thereof, the process of which comprises reacting a compound of Formula 5 with a A-W-boronic acid substituted with X₁, X₂, X₃, or X₄ to obtain a compound of Formula 1a; and optionally converting the compound of Formula 1a to a pharmaceutically acceptable salt thereof: 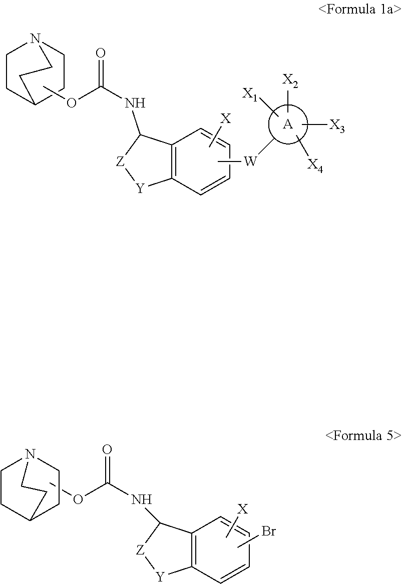
wherein,
Y and Z are, independently of each other, —CR₁R₂—; —NR₃—; —O—; or —S—,
R₁ and R₂ are, independently of each other, hydrogen; halogen; C₁-C₆ alkyl; C₁-C₆ alkyl having a nitrogen, oxygen, or sulfur atom; C₃-C₁₀ cycloalkyl; 3- to 12-membered heterocyclic; or C₁-C₆ alkoxy; or R₁ and R₂ form C₃-C₁₀ cycloalkyl together with the carbon atom to which they are attached,
R₃ is hydrogen; C₁-C₆ alkyl; C₁-C₆ alkyl having a nitrogen, oxygen, or sulfur atom; C₃-C₁₀ cycloalkyl; 3- to 12-membered heterocyclic; or C₁-C₆ alkoxy,
X is hydrogen; halogen; C₁-C₆ alkyl; C₁-C₆ alkyl substituted with 1 to 3 halogens; C₁-C₆ alkyl having a nitrogen, oxygen, or sulfur atom; C₁-C₆ alkoxy; or C₁-C₆ alkoxy substituted with 1 to 3 halogens,
W is a bond, —CH₂—, —O—, —NH—, —CH₂CH₂—, —CH═CH—, or —C≡C—,
A ring is 6- to 12-membered aryl; 5- to 12-membered heteroaryl; C₃-C₁₀ cycloalkyl; or 3- to 12-membered heterocyclic, and
X₁, X₂, X₃, and X₄ are, independently of each other, hydrogen; cyano; halogen; C₁-C₆ alkyl; C₁-C₆ alkoxy-C₁-C₆ alkyl; C₁-C₆ alkyl substituted with 1 to 3 halogens; C₃-C₁₀ cycloalkyl; 3- to 12-membered heterocyclic; C₁-C₆ alkoxy; C₁-C₆ alkoxy substituted with 1 to 3 halogens; C₁-C₆ alkoxy-C₁-C₆ alkoxy; morpholinyl-C₁-C₆ alkoxy; mono- or di-C₁-C₆ alkylamino-C₁-C₆ alkoxy; C₃-C₁₀ cycloalkyl-C₁-C₆ alkoxy; C₁-C₆ alkylthio; amino; mono- or di-C₁-C₆ alkylamino; C₁-C₆ alkylcarbonyl; hydroxy; or nitro.

7. The process as claimed in claim 6, wherein the reacting a compound of Formula 5 with a A-W-boronic acid substituted with X₁, X₂, X₃, or X₄ is carried out using a palladium catalyst.

8. The process as claimed in claim 6, wherein the reacting a compound of Formula 5 with a A-W-boronic acid substituted with X₁, X₂, X₃, or X₄ is carried out in the presence of an inorganic base.

9. The process as claimed in claim 6, wherein the compound of Formula 5 is obtained by coupling a compound of Formula 3 with quinuclidinol: 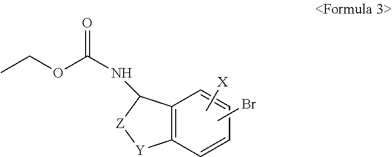
wherein, X, Y, and Z are the same as defined in the claim 6.

10. The process as claimed in claim 9, wherein the coupling is carried out through a condensation reaction including the removal of ethanol.

11. The process as claimed in claim 9, wherein the compound of Formula 3 is obtained by reacting a compound of Formula 2 or salt thereof with ethyl chloroformate: 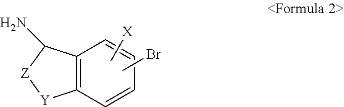
wherein, X, Y, and Z are the same as defined in the claim 6.

12. The process as claimed in claim 11, wherein the reacting a compound of Formula 2 or salt thereof with ethyl chloroformate is carried out in the presence of a base.

13. The process as claimed in claim 6, wherein the compound of Formula 5 is obtained by reacting a compound of Formula 7 with a compound of Formula 2: 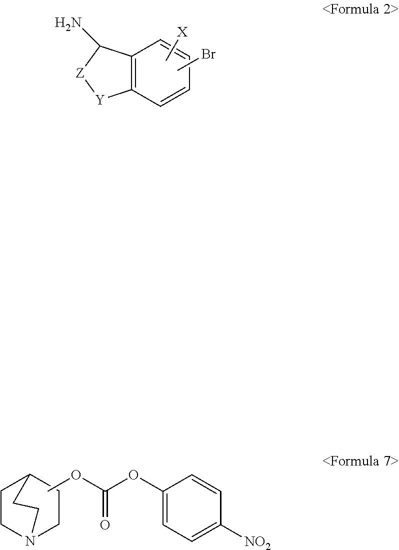
wherein, X, Y, and Z are the same as defined in the claim 6.

14. The process as claimed in claim 13, wherein the reacting a compound of Formula 7 with a compound of Formula 2 is carried out in the presence of a base.

15. The process as claimed in claim 13, wherein the compound of Formula 7 is obtained by reacting quinuclidinol with bis(4-nitrophenyl) carbonate.

16. The process as claimed in claim 13, wherein the compound of Formula 7 is obtained by reacting quinuclidinol with 4-nitrophenyl chloroformate.
